(19) United States
(12) Patent Application Publication
(10) Pub. No.: US 2022/0023281 A1
(43) Pub. Date: Jan. 27, 2022

(54) HETEROCYCLIC SPIRO-COMPOUNDS AS AM2 RECEPTOR INHIBITORS

(71) Applicant: THE UNIVERSITY OF SHEFFIELD, Sheffield, South Yorkshire (GB)

(72) Inventors: Gareth RICHARDS, Sheffield (GB); Timothy M. SKERRY, Sheffield (GB); Joseph P.A. HARRITY, Sheffield (GB); Jean-Olivier ZIRIMWABAGABO, Sheffield (GB); Matthew J. TOZER, Balsham (GB); Karl Richard GIBSON, Sandwich (GB); Roderick Alan PORTER, Ashwell (GB); Paul Alan GLOSSOP, Sandwich (GB)

(21) Appl. No.: 17/293,157

(22) PCT Filed: Nov. 15, 2019
(86) PCT No.: PCT/GB2019/053236

§ 371 Date: May 12, 2021

(30) Foreign Application Priority Data
Nov. 15, 2018 (GB) 1818651.0

(51) Int. Cl. A61K 31/4545; A61P 35/00; C07D 471/10; C07D 519/00; A61K 31/496; A61K 31/437; A61K 31/55; A61K 31/439; A61K 45/06; A61N 5/10

(52) U.S. Cl. CPC A61K 31/4545; A61P 35/00; C07D 471/10; C07D 519/00; A61K 31/496; A61N 2005/1098; A61K 31/55; A61K 31/439; A61K 45/06; A61N 5/10; A61K 31/437

(57) ABSTRACT

Disclosed are compounds of the formula (I) and pharmaceutically acceptable salts thereof: wherein HET, R¹, R², R³, R⁴, R⁵, L, L¹, X₁, X₂, X₃ and q are as defined herein. The compounds are inhibitors of adrenomedullin receptor subtype 2 (AM₂). Also disclosed are the compounds for use in the treatment of diseases modulated AM₂, including proliferative diseases such as cancer; pharmaceutical compositions comprising the compounds; methods for preparing the compounds; and intermediates useful in the preparation of the compounds.

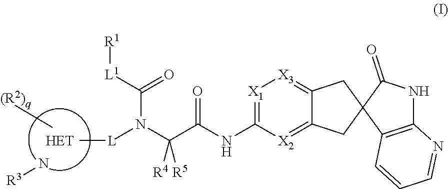

This invention relates to compounds which are AM₂ receptor inhibitors and to the use of the compounds as therapeutic agents in the treatment of conditions mediated by AM₂, for example in the treatment of proliferative disorders, including cancers such as pancreatic cancer. Also disclosed are pharmaceutical compositions comprising the compounds.

### BACKGROUND

Adrenomedullin (AM) is a hormone with important physiological functions, including the regulation of blood pressure. However, AM is dysregulated in a number of diseases and is implicated in the development and progression of a wide range of cancers, for example pancreatic cancer (Adrenomedullin is induced by hypoxia and enhances pancreatic cancer cell invasion. Keleg S, Kayed H, Jiang X, Penzel R, Giese T, Buchler M W, Friess H, Kleeff J. Int. J. Cancer. 2007 Jul. 1; 121(1):21-32; Adrenomedullin and cancer. Zudaire E, Martinez A, Cuttitta F. Regulatory Peptides. 2003 Apr. 15; 112(1-3):175-183; Adrenomedullin, a Multifunctional Regulatory Peptide. Hinson J P, Kapas S, Smith D M. Endocrine reviews. 2000; 21(2):138-167).

There are two cell surface receptor complexes for adrenomedullin, adrenomedullin receptor subtype 1 (AM₁) and adrenomedullin receptor subtype 2 (AM₂). These receptors are heteromeric structures comprising a G-protein-coupled receptor (GPCR) and an accessory protein known as a Receptor Activity Modifying Protein (RAMP). More specifically the AM₁ receptor is formed as a complex of the calcitonin like receptor (CLR) and RAMP2. The AM₂ receptor is formed by CLR and RAMP3. The AM₁ receptor has a high degree of selectivity for AM over the calcitonin gene related peptide (CGRP). By contrast, the AM₂ receptor shows less specificity for AM, having appreciable affinity for RCGRP (Hay et al. J. Mol. Neuroscience 2004; 22(1-2):105-113). The CLR/RAMP1 receptor CGRP, is a high-affinity receptor for calcitonin gene related peptide (CGRP), but it also binds AM with lower affinity (Hay et al. Pharmacological discrimination of calcitonin receptor: receptor activity-modifying protein complexes. Mol. Pharmacol. 2005; 67:1655-1665; Poyner et al. International Union of Pharmacology. XXXII. The mammalian calcitonin gene-related peptides, adrenomedullin, amylin, and calcitonin receptors. Pharmacol. Rev. 2002; 54:233-246).

Although AM₁ and AM₂ share the same GPCR, CLR, the effects of the two receptors are quite distinct. Adrenomedullin mediates important physiological functions through the AM₁ receptor, including regulation of blood pressure (Biological action of Adrenomedullin. Horio T & Yoshihara F. In: Nishikimi T. (eds); Adrenomedullin in Cardiovascular Disease. Springer, 2005, ISBN-10 0-387-25404-8: DOI.org/10.1007/0-387-25405-6-5).

In contrast, the AM₂ receptor is involved in numerous pro-tumourigenic actions through a number of different mechanisms including: stimulating cancer cell proliferation, protecting from stress induced apoptosis, promoting angiogenesis and increasing tumour invasiveness.

Adrenomedullin secreted by tumours leads to up-regulation of the AM₂ receptor in host tissues surrounding tumours. Host tissue expression of AM₂ is thought to be an important factor in the mechanism by which tumours promote angiogenesis and evade host defenses. This has been demonstrated in pancreatic tumours where AM₂ expression increases with tumour severity grade. Studies have shown that reduction in AM₂ expression either in tumours or in the host, or antagonism of the receptors with peptides or antibodies leads to reduction in cancer cell growth in-vitro and in-vivo (Ishikawa T et al. Adrenomedullin antagonist suppresses in-vivo growth of human pancreatic cancer cells in SCID mice by suppressing angiogenesis. Oncogene. 2003 Feb. 27; 22(8):1238-1242; Antolino et al. Pancreatic Cancer Can be Detected by Adrenomedullin in New Onset Diabetes Patients (PaCANOD). https://clinicaltrials.gov/ct2/show/NCT02456051; Antolino et al. Adrenomedullin in pancreatic carcinoma: A case-control study of 22 patients. Faculty of Medicine and Psychology, Sapienza University of Rome, Rome, Italy: DOI 10.15761/ICST.1000175).

Targeting of adrenomedullin and its receptors have been shown to be efficacious in animal xenograft experiments. Local injection of the AM peptide antagonist (AM22-52) directly into tumours in a pancreatic cancer model, reduced tumour size significantly compared to controls (Adrenomedullin antagonist suppresses in-vivo growth of human pancreatic cancer cells in SCID mice by suppressing angiogenesis. Ishikawa T et al. Oncogene. 2003; 22:1238-1242: DOI 10.1038/sj.onc.1206207).

Pancreatic cells overexpressing AM, implanted into mice produced significantly larger tumours, and cells whose native AM expression was knocked down, had smaller tumours. Furthermore, metastasis in animals with AM knockdown cells were almost absent (Ishikawa T et al. 2003).

In human cancers, AM₂ receptors are upregulated in host tissues surrounding tumours. WO2008/132453 discloses a mouse monoclonal antibody to hRAMP3 reduced tumour volume in a mouse model, suggesting interference with the known mechanisms of action of AM in tumours.

In clinical trials, elevated levels of serum AM have been observed in pancreatic carcinoma patients compared to controls regardless of tumour stage, differentiation, operability and presence of diabetes (A Star of Connection Between Pancreatic Cancer and Diabetes: Adrenomedullin. Görgülü K et al. Journal of the Pancreas. 2015; 16(5):408-412). High serum AM is therefore generally regarded to be an indicator of poor prognosis in pancreatic cancer.

Elevated serum AM levels accompanied by atypical development of type 2 diabetes has also been shown to be predictive of early pancreatic cancer (Kaafarani I et al. Targeting adrenomedullin receptors with systemic delivery of neutralizing antibodies inhibits tumour angiogenesis and suppresses growth of human tumour xenografts in mice. FASEB J. 2009 Jun. 22: DOI:10.1096/fj.08-127852).

Accordingly, inhibition of the AM₂ receptor is an attractive target for the treatment of proliferative conditions such as cancer, for example in the treatment of pancreatic cancer. The AM₂ receptor may play a role in regulating cell proliferation and/or apoptosis and/or in mediating interactions with host tissues including cell migration and metastasiz.

Pancreatic cancer is a devastating disease that kills most patients within 6 months of diagnosis. The one-year survival rate of less than 20% in pancreatic cancer is consistent with most patients being diagnosed at first presentation with advanced disease, at which point there is no effective life-extending therapy. Where diagnosis is early, surgical resection is the preferred treatment option and tumour resection is usually followed by chemotherapy (e.g. cytotoxic therapies, including gemcitabine or 5-fluorouracil and an EGF receptor tyrosine kinase inhibitor, erlotinib). However, due to difficulty in early diagnosis, the majority of the current therapies and management strategies focus on supportive chemotherapy with very limited expectation of life extension. Furthermore, pancreatic cancer is highly unusual from an immunological perspective meaning that current approaches to immuno-oncology therapies such as PDL-1 inhibitors are largely ineffective against pancreatic cancer (From bench to bedside a comprehensive review of pancreatic cancer immunotherapy. Kunk P R, Bauer T W, Slingluff C L, Rahma O E. Journal for ImmunoTherapy of Cancer. 2016; 4:14: DOI 10.1186/s40425-016-0119-z; Recent Advancements in Pancreatic Cancer Immunotherapy. Ma Y et al. Cancer Research Frontiers. 2016 May; 2(2):252-276: DOI 10.17980/2016.252). There is therefore a need for new treatments for pancreatic cancer.

WO 2008/127584 describes certain compounds that are stated to be CGRP (Calcitonin Gene-Related Peptide) antagonists useful in the treatment of migraines and headaches.

Certain peptide and antibody AM₂ receptor inhibitors are known such as AM₂₂₋₅₂ (Robinson et al. J. Pharmacology and Exp. Therapeutics. 2009; 331(2):513-521).

WO 2018/211275, published after the priority date of this application, describes compounds that are AM₂ receptor inhibitors.

However, there remains a need for new agents that are AM₂ receptor inhibitors. Suitably, an AM₂ inhibitor will be selective for the AM₂ receptor and in particular will exhibit little or no effects on the related AM₁ receptor. A selective AM₂ receptor is expected to provide a beneficial therapeutic effect, for example an anti-cancer effect, whilst having little or no effect on physiological effects mediated by the AM₁ receptor.

### BRIEF SUMMARY OF THE DISCLOSURE

In accordance with the present invention there is provided a compound of formula (I), or a pharmaceutically acceptable salt thereof:

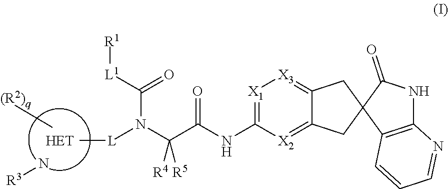

wherein
HET is a 4 to 9 membered saturated or partially saturated heterocyclyl containing 1 ring nitrogen heteroatom and optionally 1 additional ring heteroatom selected from O, S and N;
L is absent or is —C(R^{A})₂—;
each R^{A} is independently selected from: H and C₁₋₃ alkyl;
X₁ is N or CR^{B};
X₂ and X₃ are each independently N or CH, provided that no more than one of X₁, X₂ and X₃ is N;
L¹ is absent or is selected from: —O— and —N(R⁷)—
R¹ is selected from: H, C₁₋₆ alkyl, C₂₋₆ alkenyl, C₂₋₆ alkynyl, C₁₋₆ haloalkyl and Q¹-L²-, wherein said C₁₋₆ alkyl, C₂₋₆ alkenyl or C₂₋₆ alkynyl is optionally substituted by one or more R⁸;
Q¹ is selected from: C₃₋₁₂ cycloalkyl, C₃₋₁₂ cycloalkenyl, 4 to 12 membered heterocyclyl, C₆₋₁₀ aryl and 5 to 10 membered heteroaryl,

wherein said cycloalkyl, cycloalkenyl and heterocyclyl is optionally substituted by one or more R⁹, and

wherein said aryl and heteroaryl is optionally substituted by one or more R¹⁰;

L² is absent or is selected from: C₁₋₆ alkylene, C₂₋₆ alkenylene and C₂₋₆ alkynylene, wherein L² is optionally substituted by one or more R¹¹
R² is at each occurrence independently selected from: halo, ═O, C₁₋₄ alkyl, C₁₋₄ haloalkyl and —OR^{A12}, or
an R² group forms a C₁₋₆ alkylene bridge between the ring atom to which the R² group is attached and another available ring atom in HET;
   R³ is selected from: H and C₁₋₄ alkyl;
   R⁴ and R⁵ are independently selected from: H, C₁₋₄ alkyl and C₁₋₄ haloalkyl, or
R⁴ and R⁵ together with the carbon to which they are attached form a C₃₋₆ cycloalkyl;
   R⁶ is selected from: H, halo, C₁₋₆ alkyl and C₁₋₆ haloalkyl;
   R⁷ is selected from: H, C₁₋₆ alkyl, C₁₋₆ haloalkyl and —OR^{A1};
   R⁸, R⁹ and R¹¹ are at each occurrence independently selected from:
   halo, ═O, ═NR^{A2}, ═NOR^{A2}, —CN, —NO₂, C₁₋₆ alkyl, C₂₋₆ alkenyl, C₂₋₆ alkynyl, C₁₋₆ haloalkyl, -L³-Q², —OR¹⁶, —S(O)ₓR¹⁶ (wherein x is 0, 1, or 2), —NR¹⁶R^{B2}, —(O)R¹⁶, —C(O)R¹⁶, —(O)OR¹⁶, —NR^{B2}C(O)R¹⁶, —NR^{B2}C(O)OR¹⁶, —C(O)NR¹⁶R^{B2}, —OC(O)NR¹⁶R^{B2}, —NR^{B2}SO₂R¹⁶, —SO₂NR¹⁶R^{B2}, —NR^{A2}C(O)NR¹⁶R^{B2}, —NR^{A2}C(═NR^{A2})R^{B2}, —C(═NR^{A2})R^{B2}, —C(═NR^{A2})NR^{A2}R^{B2}, —NR^{A2}C(═NR^{A2})NR^{A2}R^{B2}, —NR^{A2}C(═NCN)NR^{A2}R^{B2}, —ONR^{A2}R^{B2} and —NR^{A2}OR^{B2},

wherein said C₁₋₆ alkyl, C₂₋₆ alkenyl and C₂₋₆ alkynyl is optionally substituted by 1 or more R¹², and

wherein R¹⁶ is selected from: H, C₁₋₆ alkyl and C₁₋₆ haloalkyl, wherein said C₁₋₆ alkyl is optionally substituted by one or more R¹³;

R¹⁰ is at each occurrence independently selected from:
halo, —CN, —NO₂, C₁₋₆ alkyl, C₂₋₆ alkenyl, C₂₋₆ alkynyl, C₁₋₆ haloalkyl, -L⁴-Q³, —OR¹⁷, —S(O)ₓR¹⁷ (wherein x is 0, 1, or 2), —NR¹⁷R^{B3}, —(O)R¹⁷, —O(O)R¹⁷, —(O)OR¹⁷, —NR^{B3}C(O)R¹⁷, NR^{B3}C(O)OR¹⁷, —C(O)NR¹⁷R^{B3}, —OC(O)NR¹⁷R^{B3}, —NR^{B3}SO₂R¹⁷, —SO₂NR¹⁷R^{B3}, —NR^{A3}C(O)NR¹⁷R^{B3}, —NR^{A3}C(═NR^{A3})R^{A3}, —C(═NR^{A3})R^{B3}, —C(═NR^{A3})NR^{A3}R^{B3}, —NR^{A3}C(═NR^{A3})NR^{A3}R^{B3}, —NR^{A3}C(═NCN)NR^{A3}R^{B3}, —ONR^{A3}R^{B3} and —NR^{A3}OR^{B3},

wherein said C₁₋₆ alkyl, C₂₋₆ alkenyl and C₂₋₆ alkynyl is optionally substituted by 1 or more R¹³, and

wherein R¹⁷ is selected from: H, C₁₋₆ alkyl and C₁₋₆ haloalkyl, wherein said C₁₋₆ alkyl is optionally substituted by one or more R¹⁹;

Q² and Q³ are at each occurrence independently selected from: C₃₋₁₂ cycloalkyl, C₃₋₁₂ cycloalkyl-C₁₋₃ alkyl, C₃₋₁₂ cycloalkenyl, C₃₋₁₂ cycloalkenyl-C₁₋₃ alkyl, 4 to 12 membered heterocyclyl, 4 to 12 membered heterocyclyl-C₁₋₃ alkyl, C₆₋₁₀ aryl, C₆₋₁₀ aryl-C₁₋₃ alkyl, 5 to 10 membered heteroaryl and 5 to 10 membered heteroaryl-C₁₋₃ alkyl,

wherein said C₃₋₁₂ cycloalkyl, C₃₋₁₂ cycloalkyl-C₁₋₃ alkyl, C₃₋₁₂ cycloalkenyl, C₃₋₁₂ cycloalkenyl-C₁₋₃ alkyl, 4 to 12 membered heterocyclyl and 4 to 12 membered heterocyclyl-C₁₋₃ alkyl is optionally substituted by one or more R¹⁴, and

wherein said C₆₋₁₀ aryl, C₆₋₁₀ aryl-C₁₋₃ alkyl, 5 to 10 membered heteroaryl and 5 to 10 membered heteroaryl-C₁₋₃ alkyl is optionally substituted by one or more R¹⁵;

L³ and L⁴ are independently absent or independently selected from: —O—, —CH₂O—, —NR^{A4}—, —CH₂NR^{A4}—, —S(O)ₓ—, —CH₂S(O), (wherein x is 0, 1 or 2), —C(═O)—, —CH₂C(═O)—, —NR^{A4}C(═O)—, —CH₂NR^{A4}C(═O)—, —C(═O)NR^{A4}—, —CH₂C(═O)NR^{A4}—, —S(O)₂NR^{A4}—, —CH₂S(O)₂NR^{A4}—, —NR^{A4}S(O)₂—, CH₂NR^{A4}S(O)₂—, —OC(═O)—, —CH₂OC(═O)—, —C(═O)O— and —CH₂—C(═O)O—;
R¹², R¹³, R¹⁴, R¹⁸ and R¹⁹ are at each occurrence independently selected from:
halo, ═O, —CN, —NO₂, C₁₋₄ alkyl, C₁₋₄ haloalkyl, -L⁵-Q⁴, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, —NR^{B5}C(O)OR^{A5}, —C(O)NR^{A5}R^{B5}, —NR^{B5}SO₂R^{A5} and —SO₂NR^{A5}R^{B5};

wherein said C₁₋₄alkyl is optionally substituted by 1 or 2 substituents selected from:

halo, ═O, —CN, —OR^{A6}, —NR^{A6}R^{B6} and —SO₂R^{A6};
R¹⁵ is at each occurrence independently selected from:
halo, —CN, —NO₂, C₁₋₄ alkyl, C₁₋₄ haloalkyl, -L⁶-Q⁵, —OR^{A7}, —S(O)₂R^{A7}, —NR^{A7}R^{B7}, —C(O)R^{A7}, —OC(O)R^{A7}, —C(O)OR^{A7}, —NR^{B7}C(O)R^{A7}, —NR^{B7}C(O)OR^{A7}, —C(O)NR^{A7}R^{B7}, —NR^{B7}SO₂R^{A7} and —SO₂NR^{A7}R^{B7}—;

wherein said C₁₋₄alkyl is optionally substituted by 1 or 2 substituents selected from:

halo, —CN, —OR^{A8}, —NR^{A8}R^{B8} and —SO₂R^{A8};
Q⁴ and Q⁵ are at each occurrence independently selected from: phenyl, phenyl-C₁₋₃ alkyl, 5- or 6-membered heteroaryl, 5- or 6-membered heteroaryl-C₁₋₃ alkyl-, C₃₋₆ cycloalkyl, C₃₋₆ cycloalkyl-C₁₋₃ alkyl-, 4 to 6-membered heterocyclyl and 4 to 6-membered heterocyclyl-C₁₋₃ alkyl,

wherein said C₃₋₆ cycloalkyl, C₃₋₆ cycloalkyl-C₁₋₃ alkyl-, 4 to 6-membered heterocyclyl and 4 to 6-membered heterocyclyl-C₁₋₃ alkyl of Q⁴ and Q⁵ are each independently optionally substituted by 1 or 2 substituents selected from: C₁₋₄ alkyl, C₁₋₄ haloalkyl, halo, ═O, —CN, —OR^{A9}, —NR^{A9}R^{B9}, —SO₂R^{A9} and C₁₋₄ alkyl substituted by 1 or 2 substituents selected from: halo, —CN, —OR^{A10}, —NR^{A10}R^{B10} and —SO₂R^{A10}, and

wherein said of phenyl, phenyl-C₁₋₃ alkyl, 5- or 6-membered heteroaryl and 5- or 6-membered heteroaryl-C₁₋₃ alkyl- of Q⁴ and Q⁵ are each independently optionally substituted by 1 or 2 substituents selected from: halo, C₁₋₄alkyl, C₁₋₄ haloalkyl, —CN, —OR^{A9}, —NR^{A9}R^{B9}, —SO₂R^{A9} and C₁₋₄ alkyl substituted by 1 or 2 substituents selected from: halo, —CN, —OR^{A10}, —NR^{A10}R^{B10} and —SO₂R^{A10};

L⁵ and L⁶ are independently absent or independently selected from: —O—, —NR^{A11}—, —S(O)₂—, —C(═O)—, —NR^{A11}C(═O)—, —C(═O)NR^{A11}—, —S(O)₂NR^{A11}—, —NR^{A11}S(O)₂—, —OC(═O)— and —C(═O)O—;
R^{A1}, R^{A2}, R^{B2}, R^{A3}, R^{B3}, R^{A4}, R^{A5}, R^{B5}, R^{A6}, R^{B6}, R^{A7}, R^{B7}, R^{A8}, R^{B8}, R^{A9}, R^{B9}, R^{A10}, R^{B10}, R^{A11} and R^{A12} are each independently selected from: H, C₁₋₄ alkyl and C₁₋₄ haloalkyl,

or any —NR^{A2}R^{B2}, —NR¹⁶R^{B2}, —NR^{A3}R^{B3}, —NR¹⁷R^{B3}, —NR^{A5}R^{B5}, —NR^{A6}R^{B6}, —NR^{A7}R^{B7}, —NR^{A8}R^{B8}, —NR^{A9}R^{B9} and —NR^{A10}R^{B10} within a substituent may form a 4 to 6 membered heterocyclyl, wherein said 4 to 6 membered heterocyclyl is optionally substituted by one or more substituents selected from: halo, ═O, C₁₋₄ alkyl and C₁₋₄ haloalkyl; and

q is an integer selected from 0, 1, 2, 3 and 4.

Also provided is a pharmaceutical composition comprising a compound of the invention, or a pharmaceutically acceptable salt thereof, and a pharmaceutically acceptable excipient.

Also provided is a compound of the invention, or a pharmaceutically acceptable salt thereof, for use as a medicament. In some embodiments the compound of the invention, or a pharmaceutically acceptable salt thereof, is for use in the treatment of a disease or medical condition mediated by adrenomedullin receptor subtype 2 receptors (AM₂).

Also provided is a method of treating a disease or medical condition mediated by AM₂ in a subject in need thereof, the method comprising administering to the subject an effective amount of a compound of the invention, or a pharmaceutically acceptable salt thereof.

In certain embodiments the compounds of the invention are for use in the treatment of proliferative diseases, for example cancer. In certain embodiments a compound of the invention is for use in the prevention or inhibition of cancer progression, for example by preventing or inhibiting cancer cell migration and/or preventing or inhibiting cancer metastasiz.

Also provided is a compound of the invention for use in the treatment of a cancer in which AM and or AM₂ is implicated in development or progression of the cancer. For example in some embodiments a compound of the invention may be for use in the treatment of a cancer selected from: pancreatic, colorectal, breast and lung cancer. In a particular embodiment a compound of the invention is for use in the treatment of pancreatic cancer. In certain embodiments a compound of the invention is for use in the treatment of a patient with a cancer, for example pancreatic cancer, wherein the expression of AM, AM₂, CLR and/or RAMP3 in the patient is elevated compared to controls. For example, the patient may have elevated serum levels of AM, AM₂, CLR and/or RAMP3.

The compounds of the invention may be used alone or in combination with one or more anticancer agents and/or radiotherapy as described herein.

### BRIEF DESCRIPTION OF THE DRAWINGS

FIG. 1 shows the effect a compound, SHF-1041, exemplified herein, in the xenograft mouse model described in the Examples. The mice were inoculated with CFPAC-1 cells (cells derived from a ductal adenocarcinoma (ex. ATCC)). The FIGURE shows the % tumour volume growth compared to control after 24 days of once-daily intraperitoneal (i.p.) dosing of SHF-1041 at doses of 5 mg/kg, 10 mg/kg and 20 mg/kg.

### DETAILED DESCRIPTION

### Definitions

Unless otherwise stated, the following terms used in the specification and claims have the following meanings set out below.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. For example, certain methods herein treat cancer by decreasing a symptom of cancer. Symptoms of cancer would be known or may be determined by a person of ordinary skill in the art. The term “treating” and conjugations thereof, include prevention of a pathology, condition, or disease (e.g. preventing the development of one or more symptoms of a cancer associated with AM₂.

The term “associated” or “associated with” in the context of a substance or substance activity or function associated with a disease (e.g. cancer) means that the disease (e.g. cancer) is caused by (in whole or in part), or a symptom of the disease is caused by (in whole or in part) the substance or substance activity or function. For example, a symptom of a disease or condition associated with AM₂ receptor pathway activity may be a symptom that results (entirely or partially) from an increase in the level of activity of AM₂ protein pathway. As used herein, what is described as being associated with a disease, if a causative agent, could be a target for treatment of the disease. For example, a disease associated with an increase in the level of activity of AM₂, may be treated with an agent (e.g. compound as described herein) effective for decreasing the level of activity of AM₂.

As defined herein, the term “inhibition”, “inhibit”, “inhibiting” and the like in reference to a protein-inhibitor (e.g. antagonist) interaction means negatively affecting (e.g. decreasing) the level of activity or function of the protein (e.g. a component of the AM₂) protein pathway relative to the level of activity or function of the protein pathway in the absence of the inhibitor). In some embodiments inhibition refers to reduction of a disease or symptoms of disease (e.g. cancer associated with an increased level of activity of AM₂. In some embodiments, inhibition refers to a reduction in the level of activity of a signal transduction pathway or signalling pathway associated with AM₂. Thus, inhibition may include, at least in part, partially or totally blocking stimulation, decreasing, preventing, or delaying activation, or inactivating, desensitizing, or down-regulating signal transduction or enzymatic activity or the amount of a protein (e.g. the AM₂ receptor). Inhibition may include, at least in part, partially or totally decreasing stimulation, decreasing activation, or deactivating, desensitizing, or down-regulating signal transduction or enzymatic activity or the amount of a protein (e.g. a component of an AM₂ protein pathway) that may modulate the level of another protein or modulate cell survival, cell proliferation or cell motility relative to a non-disease control.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The term “halo” or “halogen” refers to one of the halogens, group 17 of the periodic table. In particular the term refers to fluorine, chlorine, bromine and iodine. Preferably, the term refers to fluorine or chlorine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. For example, C₁₋₆ alkylene may be —CH₂—, —CH₂CH₂—, —CH₂CH(CH₃)—, —CH₂CH₂CH₂— or —CH₂CH(CH₃)CH₂—. The alkyl and alkylene groups may be unsubstituted or substituted by one or more substituents. Possible substituents are described herein. For example, substituents for an alkyl or alkylene group may be halogen, e.g. fluorine, chlorine, bromine and iodine, OH, C₁-C₄alkoxy, —NR′R″ amino, wherein R′ and R″ are independently H or alkyl. Other substituents for the alkyl group may alternatively be used.

The term “C₁₋₆ haloalkyl”, e.g. “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be, for example, —CX₃, —CHX₂, —CH₂CX₃, —CH₂CHX₂ or —CX(CH₃)CH₃ wherein X is a halo (e.g. F, Cl, Br or 1). A fluoroalkyl group, i.e. a hydrocarbon chain substituted with at least one fluorine atom (e.g. —CF₃, —CHF₂, —CH₂CF₃ or —CH₂CHF₂).

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂—, alkenyl” may be ethenyl, propenyl, butenyl, butadienyl, pentenyl, pentadienyl, hexenyl and hexadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example alkenylene may be —CH═CH—, —CH₂CH═CH—, —CH(CH₃)CH═CH— or —CH₂CH═CH—. Alkenyl and alkenylene groups may unsubstituted or substituted by one or more substituents. Possible substituents are described herein. For example, substituents may be those described above as substituents for alkyl groups.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example alkynylene may be —C≡C—, —CH₂C≡C—, —CH₂C≡CCH₂—, —CH(CH₃)CH≡C— or —CH₂C≡CCH₃. Alkynyl and alkynylene groups may unsubstituted or substituted by one or more substituents. Possible substituents are described herein. For example, substituents may be those described above as substituents for alkyl groups.

The term “C₃₋₁₂ cycloalkyl” includes a saturated hydrocarbon ring system containing 3 to 12 carbon atoms. The cycloalkyl group may be monocyclic or a fused, bridged or spiro saturated hydrocarbon ring system. The term “C₃₋₆ cycloalkyl” includes a saturated hydrocarbon ring system containing 3, 4, 5 or 6 carbon atoms. For example, the C₃-C₁₂ cycloalkyl may be cyclopropyl, cyclobutyl, cyclopentyl, cyclohexyl, bicyclo[1.1.1]pentane, bicyclo[2.1.1]hexane, bicyclo[2.2.1]heptane (norbornane), bicyclo[2.2.2]octane or tricyclo[3.3.1.1]decane (adamantyl). For example, the “C₃-C₆ cycloalkyl” may be cyclopropyl, cyclobutyl, cyclopentyl, cyclohexyl, bicyclo[2.1.1]hexane or bicyclo[1.1.1]pentane. Suitably the “C₃-C₆ cycloalkyl” may be cyclopropyl, cyclobutyl, cyclopentyl or cyclohexyl.

The term “C₃₋₁₂ cycloalkenyl” includes a hydrocarbon ring system containing 3 to 12 carbon atoms and at least one double bond (e.g. 1 or 2 double bonds). The cycloalkenyl group may be monocyclic or a fused, bridged or spiro hydrocarbon ring system. For example, C₃₋₁₂ cycloalkenyl may be cyclobutenyl, cyclopentenyl, cyclohexenyl,

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9- (e.g. a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen.

The term “bridged ring systems” includes ring systems in which two rings share more than two atoms, see for example Advanced Organic Chemistry, by Jerry March, 4th Edition, Wiley Interscience, pages 131-133, 1992. Suitably the bridge is formed between two non-adjacent carbon or nitrogen atoms in the ring system. The bridge connecting the bridgehead atoms may be a bond or comprise one or more atoms. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl”, “heteroaryl-Cₘ₋ₙ alkyl” and “cycloalkyl-Cₘ₋ₙ alkyl” are defined in the same way.

“—Cₘ₋ₙ alkyl substituted by —NRR” and “Cₘ₋ₙ alkyl substituted by —OR” similarly refer to an —NRR″ or —OR″ group covalently attached to a Cₘ₋ₙ alkylene group and wherein the group is linked to the remainder of the molecule via a carbon atom in the alkylene group.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five-membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six-membered heteroaryl groups include but are not limited to pyridyl, pyrazinyl, pyridazinyl, pyrimidinyl and triazinyl.

Particular examples of bicyclic heteroaryl groups containing a six-membered ring fused to a five-membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “oxo,” or “═O” as used herein, means an oxygen that is double bonded to a carbon atom.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

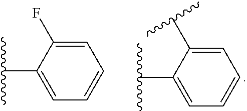

“Meta” substitution is a substitution pattern where two substituents are on carbons one carbon removed from each other, i.e. with a single carbon atom between the substituted carbons. In other words there is a substituent on the second atom away from the atom with another substituent. For example the groups below are meta substituted:

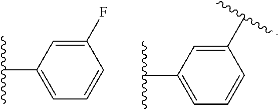

“Para” substitution is a substitution pattern where two substituents are on carbons two carbons removed from each other, i.e. with two carbon atoms between the substituted carbons. In other words there is a substituent on the third atom away from the atom with another substituent. For example the groups below are para substituted:

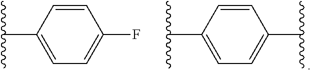

Reference to a —NRR′ group forming a 4 to 6 membered heterocyclyl refers to R and R′ together with the nitrogen atom to which they are attached forming a 4 to 6 membered heterocyclyl group. For example, an —NRR′ such as a —NR^{A2}R^{B2}, —NR¹⁶R^{B2}, —NR^{A3}R^{B3}, —NR¹⁷R^{B3}, —NR^{A5}R^{B5}, —NR^{A6}R^{B6}, —NR^{A7}R^{B7}, —NR^{A8}R^{B8}, —NR^{A9}R^{B9} and —NR^{A10}R^{B10} group may form:

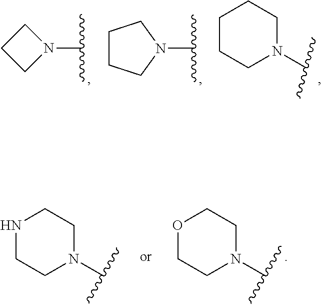

Similarly an —NRR′ group within a substituent may form a carbonyl-linked 4 to 6 membered heterocyclyl, for example a —C(O)NRR group may form:

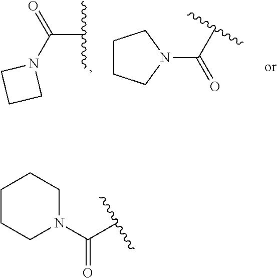

—NRR′ groups within substituents such as —OC(O)NRR′, —SO₂NRR′, —NRC(O)NRR′, —C(═NR^{A5})NRR′, —NRC(═NR)NRR′, and —NRC(═NCN)NRR′, may similarly form a 4 to 6 membered heterocyclyl within such substituents.

HET is a 4 to 9 membered saturated or partially saturated heterocyclyl containing 1 ring nitrogen heteroatom and optionally 1 additional ring heteroatom selected from O, S and N. The reference to the heterocyclyl “containing 1 ring nitrogen” refers to the N(R³) group in HET. Accordingly, HET optionally contains 1 additional ring heteroatom in addition to N(R³).

Reference to an R² group forming a C₁₋₆ alkylene bridge between the ring atom to which the R² group is attached and another available ring atom in HET include, for example:

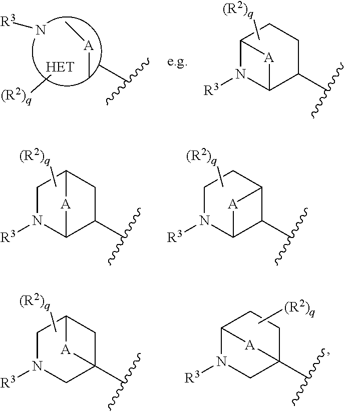

wherein A is C₁₋₆ alkylene, e.g. C₁₋₄ alkylene.

The alkylene bridge (e.g. -A- above) may be straight chained or branched, for example —CH₂—, —CH₂CH₂—, —CH(CH₃)— or —C(CH₃)₂—. Suitably A is methylene or ethylene. It may be that A is C₂₋₄ alkylene, particularly when HET is a 7, 8 or 9 membered ring. Where the alkylene bridge is shown as -A- herein as in, for example:

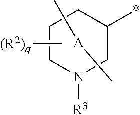

the terminal carbon atoms of the alkylene bridge are boned to 2 different available ring atoms in HET. Preferably the alkylene bridge is attached to non-adjacent ring atoms in HET. Unless stated otherwise, where an R² group forms an alkylene bridge in HET, q remains an integer selected from: 0, 1, 2, 3 and 4 (i.e. HET in optionally substituted by up to 4 R² groups in the bridged ring systems).

As will be recognised reference to HET “containing 1 ring nitrogen heteroatom” is referring to the NR³ group. In certain embodiments HET optionally 1 additional ring heteroatom selected from O, S and N in addition to the NR³ group.

The phrase “compound of the invention” means those compounds which are disclosed herein, both generically and specifically. Accordingly compounds of the invention include compounds of the formulae (I) (II), (III), (IV), (V), (VI), (VII) or (VIII) and the compounds in the Examples.

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 585 and, for example, is 575 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:

(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95% or at least 99%.

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess AM₂ inhibitory activity.

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ¹²⁵I, ³²P, ³⁵S and the like. The radionuclide that is used will depend on the specific application of that radio-labelled derivative. For example, for in-vitro competition assays, 3H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in-vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group.

Certain compounds of the invention may exist in solvated as well as unsolvated forms such as, for example, hydrated forms. It is to be understood that the invention encompasses all such solvated forms that possess AM₂ inhibitory activity.

It is also to be understood that certain compounds of the invention may exhibit polymorphism, and that the invention encompasses all such forms that possess AM₂ inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

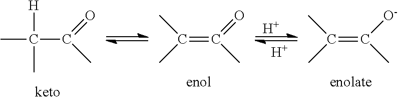

The in-vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug (i.e. a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in-vivo-cleavable ester derivatives that may be formed at a carboxy group or a hydroxy group in a compound of the invention and in-vivo-cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the formula I that possesses a carboxy group is, for example, an in-vivo-cleavable ester thereof. An in-vivo-cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆ alkyl esters such as methyl, ethyl and tert-butyl, C₁₋₆ alkoxymethyl esters such as methoxymethyl esters, C₁₋₆ alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically-acceptable pro-drug of a compound of the invention that possesses a hydroxy group is, for example, an in-vivo-cleavable ester or ether thereof. An in-vivo-cleavable ester or ether of a compound of the invention containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀, alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the invention that possesses a carboxy group is, for example, an in-vivo-cleavable amide thereof, for example an amide formed with an amine such as ammonia, a C₁₋₄ alkylamine such as methylamine, a (C₁₋₄ alkyl)₂amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically-acceptable pro-drug of a compound of the invention that possesses an amino group is, for example, an in-vivo-cleavable amide or carbamate derivative thereof. Suitable pharmaceutically-acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically-acceptable carbamates from an amino group include, for example acyloxyalkoxycarbonyl and benzyloxycarbonyl groups. COMPOUNDS

The following paragraphs are applicable to the compounds of the invention.

In certain embodiments the compound of formula (I) HET is bonded to the -L-N(C(═O)L¹R¹)— group in formula (I) via a ring carbon atom in HET.

In certain embodiments the compound of formula (I) is a compound according to formula (II), or a pharmaceutically acceptable salt thereof:

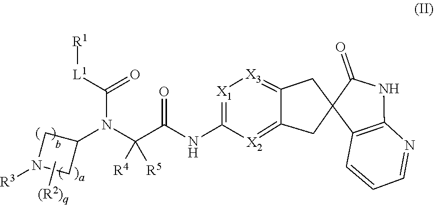

wherein
a is an integer selected from 0, 1 and 2;
b is an integer selected from 1, 2, 3 and 4.

In certain embodiments the compound of formula (I) is a compound according to formula (III), or a pharmaceutically acceptable salt thereof:

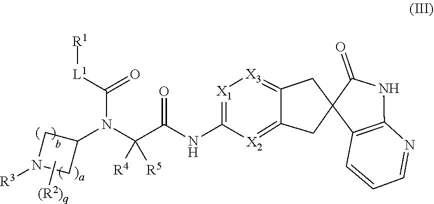

wherein b is an integer selected from 1, 2 and 3.

In certain embodiments the compound of formula (I) is a compound according to formula (IV), or a pharmaceutically acceptable salt thereof:

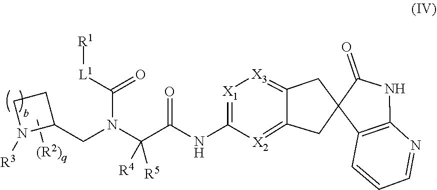

wherein b is an integer selected from 1, 2 and 3.

In certain embodiments the compound of formula (I) is a compound according to formula (V), or a pharmaceutically acceptable salt thereof:

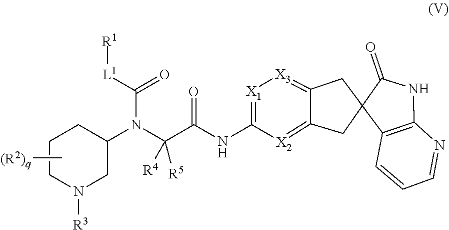

In certain embodiments the compound of formula (I) is a compound according to formula (VI), or a pharmaceutically acceptable salt thereof:

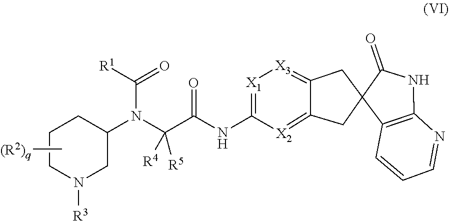

In certain embodiments the compound of formula (I) is a compound according to formula (VII), or a pharmaceutically acceptable salt thereof:

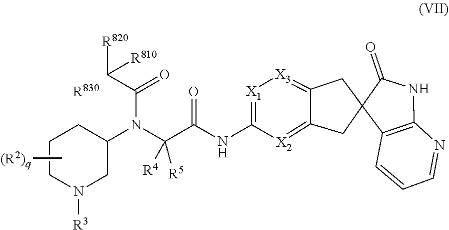

wherein
R⁸¹⁰ is selected from: C₁₋₆ alkyl, C₁₋₆ haloalkyl and C₃₋₆ cycloalkyl-C₁₋₃ alkyl,
R⁸²⁰ and R⁸³⁰ are each independently selected from: halo, C₁₋₆ alkyl and C₁₋₆ haloalkyl,

or R⁸²⁰ and R⁸³⁰ together with the carbon atom to which they are attached form a C₃₋₆ cycloalkyl or 4 to 7 membered heterocyclyl containing 1 or 2 heteroatoms selected from O, S and N,

wherein said C₃₋₆ cycloalkyl or 4 to 7 membered heterocyclyl is optionally substituted by one or more R⁹.

In certain embodiments the compound of formula (I) is a compound according to formula (VIII), or a pharmaceutically acceptable salt thereof:

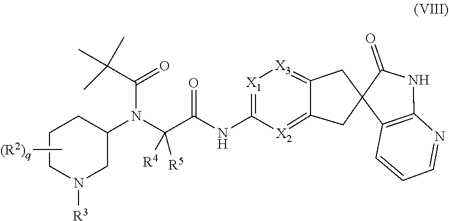

In certain embodiments in any of the compounds of formula (I), (II), (III), (IV), (V), (VI), (VII) or (VIII) R⁴ is H or C₁₋₃ alkyl and R⁵ is H.

In certain embodiments in any of the compounds of formula (I), (II), (III), (IV), (V), (VI), (VII) or (VIII) R⁴ is H or methyl and R⁵ is H.

In certain embodiments in any of the compounds of formula (I), (II), (III), (IV), (V), (VI), (VII) or (VIII) R⁴ is C₁₋₃ alkyl (e.g. methyl) and R⁵ is H.

In certain embodiments in any of the compounds of formula (I), (II), (III), (IV), (V), (VI), (VII) or (VIII) R⁴ and R⁵ are H.

In certain embodiments in any of the compounds of formula (I), (II), (III), (IV), (V), (VI), (VII) or (VIII) R³ is H.

In certain embodiments in any of the compounds of formula (I), (II), (III), (IV), (V), (VI), (VII) or (VIII) q is 0, 1 or 2 and R² is C₁₋₃ alkyl (e.g. methyl).

In certain embodiments in any of the compounds of formula (I), (II), (III), (IV), (V), (VI), (VII) or (VIII) q is 0.

In certain embodiments in any of the compounds of formula (I), (II), (III), (IV), (V), (VI), (VII) or (VIII) X₂ and X₃ are CH and X, is CR⁶ or N.

In certain embodiments in any of the compounds of formula (I), (II), (III), (IV), (V), (VI), (VII) or (VIII) X₁, X₂ and X₃ are CH.

In certain embodiments compounds of the invention include, for example, compounds of formulae (I), (II), (III), (IV), (V), (VI), (VII) or (VIII), or a pharmaceutically acceptable salt thereof, wherein, unless otherwise stated, each of R¹, R², R³, R⁴, R⁵, X₁, X₂, X₃, L, L¹, HET and q has any of the meanings defined hereinbefore or in any of paragraphs (1) to (117) hereinafter:—
1. HET is a 4 to 7 membered saturated or partially saturated heterocyclyl ring containing 1 ring nitrogen heteroatom and optionally 1 additional ring heteroatom selected from O and N, wherein HET is bonded to the remainder of the formula (I) via a ring carbon atom in HET.
2. HET is a 4 to 7 membered saturated heterocyclyl ring containing 1 ring nitrogen heteroatom and optionally 1 additional ring heteroatom selected from O and N, wherein HET is bonded to the remainder of the formula (I) via a ring carbon atom in HET.
3. HET is a 4 to 7 membered partially saturated heterocyclyl ring containing 1 ring nitrogen heteroatom and optionally 1 additional ring heteroatom selected from O and N, wherein HET is bonded to the remainder of the formula (I) via a ring carbon atom in HET.
4. HET is a 4 to 7 membered saturated heterocyclyl ring containing 1 ring nitrogen heteroatom (i.e. NR³), wherein HET is bonded to the remainder of the formula (I) via a ring carbon atom in HET.
5. HET is a 4 to 7 membered partially saturated heterocyclyl ring containing 1 ring nitrogen heteroatom (i.e. NR³), wherein HET is bonded to the remainder of the formula (I) via a ring carbon atom in HET.
6. HET is selected from: azetidinyl, pyrrolidinyl, piperidinyl, piperazinyl, morpholinyl, homopiperidinyl, homopiperazinyl and homomorpholinyl,
   wherein:
   (i) HET is bonded to the remainder of the formula (I) via a ring carbon atom in HET;
   (ii) the ring nitrogen atom(s) in HET is substituted by R³; and
   (iii) HET is optionally substituted by 1, 2, 3 or 4 R², or an R² group forms a C₁₋₄ alkylene bridge between the ring atom to which the R² group is attached and another available ring atom in HET.
7. HET is selected from: azetidinyl, pyrrolidinyl, piperidinyl and homopiperidinyl,
   wherein:
   (i) HET is bonded to the remainder of the formula (I) via a ring carbon atom in HET;
   (ii) the ring nitrogen atom in HET is substituted by R³; and
   (iii) HET is optionally substituted by 1, 2, 3 or 4 R², or an R² group forms a C₁₋₄ alkylene bridge between the ring atom to which the R² group is attached and another available ring atom in HET.
8. HET is homopiperidinyl,
   wherein:
   (i) HET is bonded to the remainder of the formula (I) via a ring carbon atom in HET;
   (ii) the ring nitrogen atom in HET is substituted by R³; and
   (iii) HET is optionally substituted by 1, 2, 3 or 4 R², or an R² group forms a C₁₋₄ alkylene bridge between the ring atom to which the R² group is attached and another available ring atom in HET.
9. HET is:

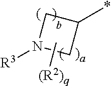

wherein a is an integer selected from 0, 1 and 2 (suitably a is 1 or 2), b is an integer selected from 1, 2, 3 and 4, and * shows the point of attachment to the remainder of the compound.
10. HET is:

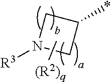

wherein a is an integer selected from 0, 1 and 2, b is an integer selected from 1, 2, 3 and 4, the sum a+b is from 2 to 7, and * shows the point of attachment to the remainder of the compound.
11. HET is:

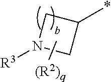

wherein b is an integer selected from 1, 2, 3 and 4, and * shows the point of attachment to the remainder of the compound.
12. HET is:

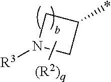

wherein b is an integer selected from 2, 3 and 4, and * shows the point of attachment to the remainder of the compound.
13. HET is selected from

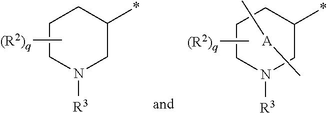

wherein A is C₁₋₄ alkylene and * shows the point of attachment to the remainder of the compound.
14. HET is

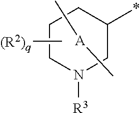

wherein A is C₁₋₄ alkylene and * shows the point of attachment to the remainder of the compound.
15. HET is selected from:

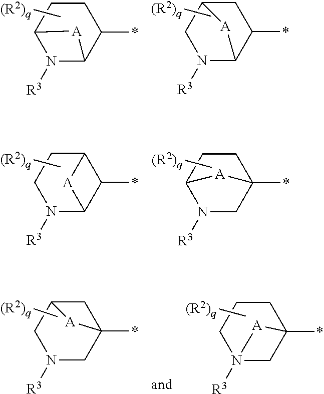

wherein A is C₁₋₄ alkylene, and * shows the point of attachment to the remainder of the compound.
16. HET is selected from:

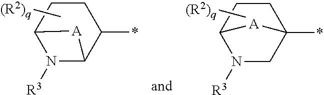

wherein A is C₁₋₄ alkylene, and * shows the point of attachment to the remainder of the compound.
17. HET is selected from:

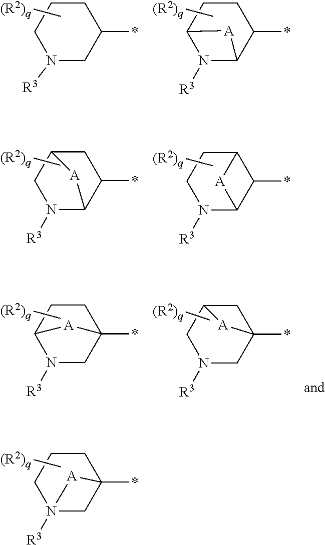

wherein A is C₁₋₄ alkylene, and * shows the point of attachment to the remainder of the compound.
18. HET is

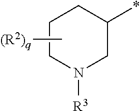

wherein * shows the point of attachment to the remainder of the compound.
19. HET is

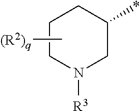

wherein * shows the point of attachment to the remainder of the compound.
20. HET is selected from

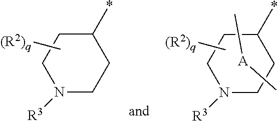

wherein A is C₁₋₄ alkylene, and * shows the point of attachment to the remainder of the compound.
21. HET is selected from:

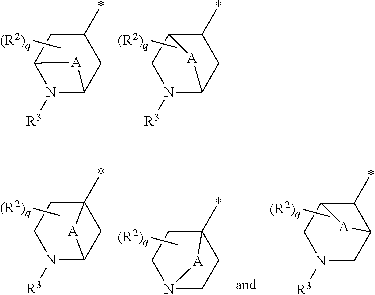

wherein A is C₁₋₄ alkylene, and * shows the point of attachment to the remainder of the compound.
22. HET is selected from:

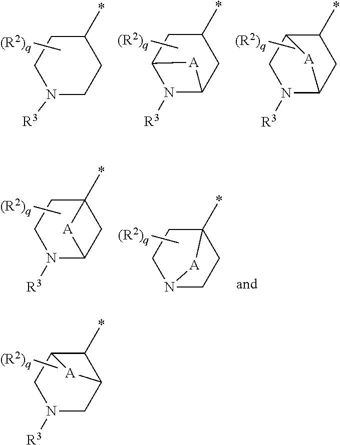

wherein A is C₁₋₄ alkylene, and * shows the point of attachment to the remainder of the compound.
23. HET is

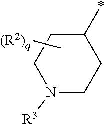

24. HET is selected from:

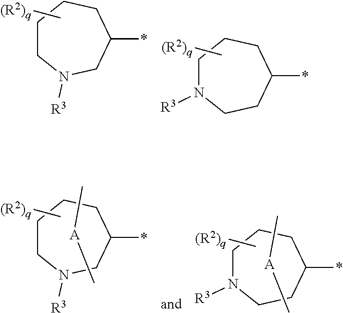

wherein A is C₁₋₄ alkylene (preferably C₂₋₄ alkylene), and * shows the point of attachment to the remainder of the compound.
25. HET is selected from:

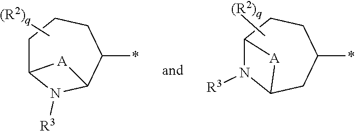

wherein A is C₁₋₄ alkylene (preferably C₂₋₄ alkylene), and * shows the point of attachment to the remainder of the compound.
26. HET is

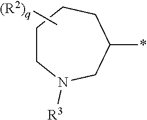

wherein * shows the point of attachment to the remainder of the compound.
27. HET is

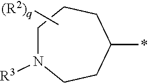

wherein * shows the point of attachment to the remainder of the compound.
28. HET is:

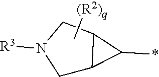

wherein * shows the point of attachment to the remainder of the compound.
29. HET is selected from:

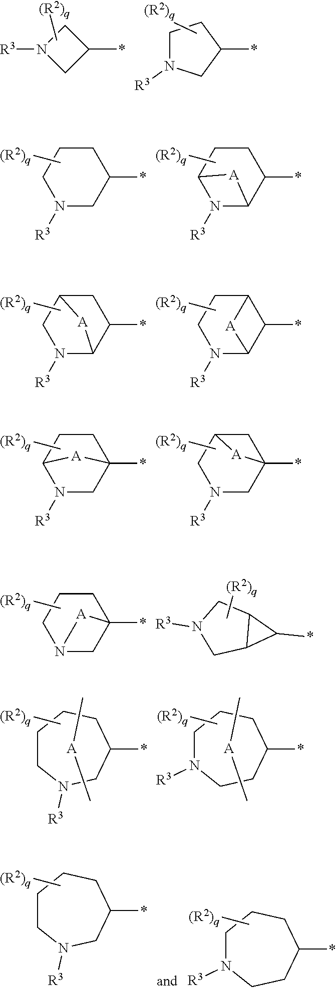

wherein A is C₁₋₄ alkylene, and * shows the point of attachment to the remainder of the compound.
30. HET is selected from:

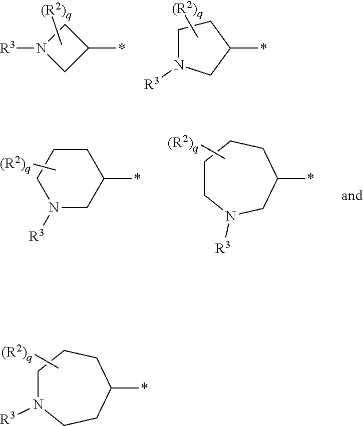

wherein A is C₁₋₄ alkylene, and * shows the point of attachment to the remainder of the compound.
31. HET is selected from:

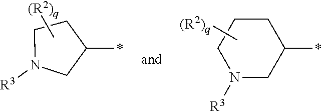

wherein * shows the point of attachment to the remainder of the compound.
32. HET is selected from:

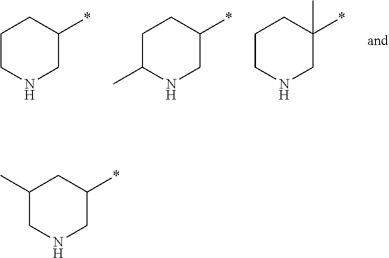

wherein * shows the point of attachment to the remainder of the compound.
33. HET is selected from:

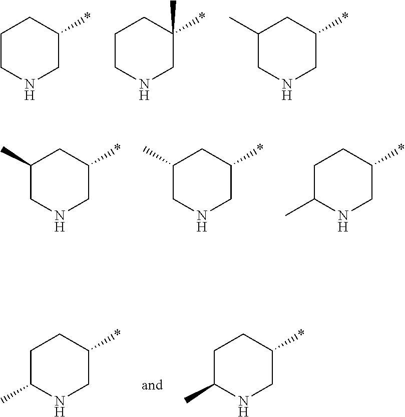

wherein * shows the point of attachment to the remainder of the compound.
34. Het is:

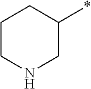 [SMILES: *C1CCCNC1]

wherein * shows the point of attachment to the remainder of the compound.
35. HET is:

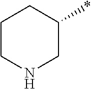 [SMILES: *[C@H]1CCCNC1]

wherein * shows the point of attachment to the remainder of the compound.
36. HET is as defined in any one of (1) to (31) above; R³ is H or methyl; q is an integer selected from 0, 1 and 2; and R² is at each occurrence independently selected from: C₁₋₃ alkyl (e.g. methyl).
37. HET is as defined in any one of (1) to (31) above; R³ is H; q is an integer selected from 0, 1 and 2; and R² is at each occurrence independently selected from: C₁₋₃ alkyl (e.g. methyl).
38. HET is as defined in any one of (1) to (31) above; R³ is H; and q is 0
39. R² is at each occurrence independently selected from: ═O and C₁₋₄ alkyl.
40. R² is at each occurrence independently selected from: C₁₋₃ alkyl.
41. q is an integer selected from 0, 1 and 2.
42. q is 0.
43. R³ is selected from: H and C₁₋₃ alkyl.
44. R³ is selected from: H and methyl.
45. R³ is H.
46. L is absent or —CH₂—.
47. L is absent.
48. L is —CH₂—.
49. L is absent and HET is as defined in any one of (1) to (38) above.
50. L is —CH₂— and HET is as defined in any one of (1) to (38) above.
51. L is —C(R^{A})₂— (e.g. —CH₂—) and HET is selected from:

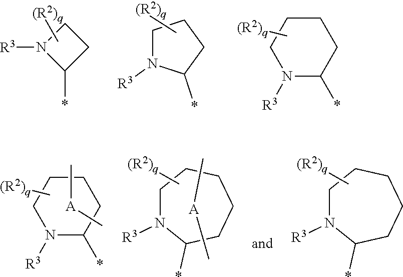

wherein A is C₁₋₄ alkylene, and * shows the point of attachment to L.
52. R¹ is selected from: C₁₋₆ alkyl, C₁₋₆ haloalkyl and Q¹-L²-, wherein said C₁₋₆ alkyl is optionally substituted by one or more R⁸;
   Q¹ is selected from: C₃₋₁₂ cycloalkyl, 4 to 12 membered saturated or partially saturated heterocyclyl containing 1 or 2 ring heteroatoms selected from O, S and N,
      wherein said cycloalkyl and heterocyclyl is optionally substituted by one or more R⁹,
   L² is absent or is selected from C₁₋₄ alkylene;
   R⁸ and R⁹ are at each occurrence independently selected from:
   halo, ═O, —CN, C₁₋₆ alkyl, C₂₋₆ alkenyl, C₁₋₆ haloalkyl, -L³-Q², —OR¹⁶, —SO₂R¹⁶, —NR¹⁶R^{B2}, —C(O)R¹⁶, —OC(O)R¹⁶, —C(O)OR¹⁶, —NR^{B2}C(O)R¹⁶, —NR^{B2}C(O)OR¹⁶, —C(O)NR¹⁶R^{B2}, —NR^{B2}SO₂R¹⁶, —SO₂NR¹⁶R^{B2} and —NR^{A2}C(O)NR¹⁶R^{B2},
      wherein said C₁₋₆ alkyl and C₂₋₆ alkenyl is optionally substituted by 1 or more R¹², and
      wherein R¹⁶ is selected from: H, C₁₋₆ alkyl and C₁₋₆ haloalkyl, wherein said C₁₋₆ alkyl is optionally substituted by one or more R¹⁸;
   Q² is at each occurrence independently selected from: C₃₋₆ cycloalkyl, C₃₋₆ cycloalkyl-C₁₋₃ alkyl, 4 to 7 membered heterocyclyl, 4 to 7 membered heterocyclyl-C₁₋₃ alkyl, phenyl, phenyl-C₁₋₃ alkyl, 5 or 6 membered heteroaryl and 5 or 6 membered heteroaryl-C₁₋₃ alkyl,
      wherein said C₃₋₆ cycloalkyl, C₃₋₆ cycloalkyl-C₁₋₃ alkyl, 4 to 7 membered heterocyclyl and 4 to 7 membered heterocyclyl-C₁₋₃ alkyl is optionally substituted by one or more R¹⁴, and
      wherein said phenyl, phenyl-C₁₋₃ alkyl, 5 or 6 membered heteroaryl and 5 or 6 membered heteroaryl-C₁₋₃ alkyl is optionally substituted by one or more R¹⁵;
   L³ is absent or selected from: —O—, —CH₂O—*, —NR^{A4}—, —CH₂NR^{A4}—*, —SO₂—, —CH₂SO₂—*, —C(═O)—, —CH₂C(═O)—*, —NR^{A4}C(═O)—, —CH₂NR^{A4}C(═O)—*, —C(═O)NR^{A4}—, —CH₂C(═O)NR^{A4}—*, —S(O)₂NR^{A4}—, —CH₂S(O)₂NR^{A4}—*, —NR^{A4}S(O)₂—, —CH₂NR^{A4}S(O)₂—*, —OC(═O)—, —CH₂OC(═O)—*, —C(═O)O— and —CH₂—C(═O)O—*, wherein * shows the point of attachment to -Q²;
   R¹², R¹⁴ and R¹⁸ are at each occurrence independently selected from:
   halo, ═O, —CN, C₁₋₄ alkyl, C₁₋₄ haloalkyl, -L⁵-Q⁴, —OR^{A5}, —S(O)₂R^{A5}, —NR^{A5}R^{B5}, C(O)R^{A5}, —OC(O)R^{A5}, —C(O)OR^{A5}, —NR^{B5}C(O)R^{A5}, —C(O)NR^{A5}R^{B5}, —NR^{B5}SO₂R^{A5} and —SO₂NR^{A5}R^{B5};
   R¹⁵ is at each occurrence independently selected from:
   halo, —CN, C₁₋₄ alkyl, C₁₋₄ haloalkyl, -L⁶-Q⁵, —OR^{A7}, —S(O)₂R^{A7}, —NR^{A7}R^{B7}, —C(O)R^{A7}, —OC(O)R^{A7}, —C(O)OR^{A7}, —NR^{B7}C(O)R^{A7}, —C(O)NR^{A7}R^{B7}, —NR^{B7}SO₂R^{A} and —SO₂NR^{A7}R^{B7},
      wherein said C₁₋₄ alkyl is optionally substituted by 1 or 2 substituents selected from: halo, —CN, —OR^{A8}, —NR^{A8}R^{B8} and —SO₂R^{A8};
   Q⁴ and Q⁵ are at each occurrence independently selected from: phenyl, phenyl-C₁₋₃ alkyl, 5- or 6-membered heteroaryl, 5- or 6-membered heteroaryl-C₁₋₃ alkyl-, C₃₋₆ cycloalkyl, C₃₋₆ cycloalkyl-C₁₋₃ alkyl-, 4 to 6-membered heterocyclyl and 4 to 6-membered heterocyclyl-C₁₋₃ alkyl,
      wherein said C₃₋₆ cycloalkyl, C₃₋₆ cycloalkyl-C₁₋₃ alkyl-, 4 to 6-membered heterocyclyl and 4 to 6-membered heterocyclyl-C₁₋₃ alkyl of Q⁴ and Q⁵ are each independently optionally substituted by 1 or 2 substituents selected from: C₁₋₄ alkyl, C₁₋₄ haloalkyl, halo and ═O, and
      wherein said of phenyl, phenyl-C₁₋₃ alkyl, 5- or 6-membered heteroaryl and 5- or 6-membered heteroaryl-C₁₋₃ alkyl- of Q⁴ and Q⁵ are each independently optionally substituted by 1 or 2 substituents selected from: halo, C₁₋₄ alkyl, C₁₋₄ haloalkyl, —CN, —OR^{A9}, —NR^{A9}R^{B9} and —SO₂R^{A9}
   L⁵ and L⁶ are independently absent or independently selected from: —O—, —NR^{A11}—, —S(O)₂—, —C(═O)—, —NR^{A11}C(═O)—, —C(═O)NR^{A11}—, —S(O)₂NR^{A11}—, —NR^{A11}S(O)₂—, —OC(═O)— and —C(═O)O—.
53. R¹ is selected from: C₁₋₆ alkyl, C₁₋₆ haloalkyl and Q¹-L²-, wherein said C₁₋₆ alkyl is optionally substituted by one or more R⁸;
   Q¹ is selected from: C₃₋₁₂ cycloalkyl, 4 to 12 membered saturated or partially saturated heterocyclyl containing 1 or 2 ring heteroatoms selected from 0, S and N,
      wherein said cycloalkyl and heterocyclyl is optionally substituted by one or more R⁹,
   L² is absent or is selected from C₁₋₄ alkylene;
   R⁸ and R⁹ are at each occurrence independently selected from:
   halo, ═O, —CN, C₁₋₆ alkyl, C₁₋₆ haloalkyl, -L³-Q², —OR^{16A}, —SO₂R¹⁶, —NR^{16A}R^{B2}, —C(O)R¹⁶, —C(O)NR^{16A}R^{B2}, —SO₂NR^{16A}R^{B2} and —C(O)OR^{16A},
      wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},
      wherein R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, —C(O)NR^{A5}R^{B5}, —NR^{B5}SO₂R^{A5} and —SO₂NR^{A5}R^{B5},
      R^{16A} is selected from: H, C₁₋₆ alkyl, C₁₋₆ haloalkyl,
         C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
         C₂₋₆ alkyl substituted by 1 substituent selected from: —OR^{A5} and —NR^{A5}R^{B5}.
   Q² is at each occurrence independently selected from: C₃₋₆ cycloalkyl, C₃₋₆ cycloalkyl-C₁₋₃ alkyl, 4 to 7 membered heterocyclyl, 4 to 7 membered heterocyclyl-C₁₋₃ alkyl, phenyl, phenyl-C₁₋₃ alkyl, 5 or 6 membered heteroaryl and 5 or 6 membered heteroaryl-C₁₋₃ alkyl,
      wherein said 4 to 7 membered heterocyclyl has 1 or 2 heteroatoms selected from O, S and N,
      wherein said C₃₋₆ cycloalkyl, C₃₋₆ cycloalkyl-C₁₋₃ alkyl, 4 to 7 membered heterocyclyl and 4 to 7 membered heterocyclyl-C₁₋₃ alkyl is optionally substituted by one or more R¹⁴, and
      wherein said phenyl, phenyl-C₁₋₃ alkyl, 5 or 6 membered heteroaryl and 5 or 6 membered heteroaryl-C₁₋₃ alkyl is optionally substituted by one or more R¹⁵;
   L³ is absent or selected from: —O—, —NR^{A4}—, —SO₂—, —C(═O)—, —NR^{A4}C(═O)—, —C(═O)NR^{A4}—, —S(O)₂NR^{A4}— and —NR^{A4}S(O)₂—;
   R¹⁴ and are at each occurrence independently selected from:
      halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5} and
   R¹⁵ is at each occurrence independently selected from:
      halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7}.
54. R¹ is selected from: C₁₋₆ alkyl, C₁₋₆ haloalkyl and Q¹-L²-, wherein said C₁₋₆ alkyl is optionally substituted by one or more R⁸;
   Q¹ is selected from: C₃₋₁₂ cycloalkyl, 4 to 7 membered saturated or partially saturated heterocyclyl containing 1 or 2 ring heteroatoms selected from 0, S and N,
      wherein said cycloalkyl and heterocyclyl is optionally substituted by one or more R⁹,
   L² is absent or is selected from C₁₋₄ alkylene;
   R⁸ and R⁹ are at each occurrence independently selected from:
   halo, ═O, —CN, C₁₋₆ alkyl, C₁₋₆ haloalkyl, -L³-Q², —OR^{16A}, —SO₂R¹⁶, —NR^{16A}R^{B2}, —C(O)R¹⁶, —C(O)NR^{16A}R^{B2}, —SO₂NR^{16A}R^{B2} and —C(O)OR^{16A},
      wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},
      wherein R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, C(O)OR A⁵, —NR^{B5}C(O)R A⁵, —C(O)NR^{A5}R^{B5}, —NR^{B5}SO₂R^{A5} and —SO₂NR^{A5}R^{B5},
      R^{16A} is selected from: H, C₁₋₆ alkyl, C₁₋₆ haloalkyl,
         C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
         C₂₋₆ alkyl substituted by 1 substituent selected from: —OR^{A5} and —NR^{A5}R^{B5};
   Q² is at each occurrence independently selected from: C₃₋₆ cycloalkyl, C₃₋₆ cycloalkyl-C₁₋₃ alkyl, Q⁷, Q⁷-C₁₋₃ alkyl, phenyl, phenyl-C₁₋₃ alkyl, 5 or 6 membered heteroaryl and 5 or 6 membered heteroaryl-C₁₋₃ alkyl,
      wherein Q⁷ is selected from: azetidinyl, oxetanyl, pyrrolidinyl, tetrahydrofuranyl, piperidinyl, piperazinyl, tetrahydropyranyl and morpholinyl,
      wherein said C₃₋₆ cycloalkyl, C₃₋₆ cycloalkyl-C₁₋₃ alkyl, Q⁷ and Q⁷-C₁₋₃ alkyl, is optionally substituted by one or more R¹⁴, and
      wherein said phenyl, phenyl-C₁₋₃ alkyl, 5 or 6 membered heteroaryl and 5 or 6 membered heteroaryl-C₁₋₃ alkyl is optionally substituted by one or more R¹⁵;
   L³ is absent or is selected from: —O—, —NR^{A4}—, —SO₂—, —C(═O)—, —NR^{A4}C(═O)—, —C(═O)NR^{A4}—, —S(O)₂NR^{A4}—, —NR^{A4}S(O)₂— and —C(O)O—;
   R¹⁴ and are at each occurrence independently selected from:
   halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and
   R¹⁵ is at each occurrence independently selected from:
   halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7}.
55. R¹ is selected from: C₁₋₆ alkyl, C₁₋₆ haloalkyl, C₃₋₆ cycloalkyl and 4 to 7 membered saturated heterocyclyl containing 1 or 2 heteroatoms selected from O, S and N;
   wherein said C₁₋₆ alkyl is optionally substituted by 1 or 2 substituents independently selected from: halo, and —OR^{A5},
   wherein said C₃₋₆ cycloalkyl and 4 to 7 membered saturated heterocyclyl is optionally substituted by 1 or more (e.g. 1 or 2) substituents selected from: halo, ═O, C₁₋₄ alkyl, C₁₋₄ haloalkyl, -L³-Q², —C(O)R^{A2} and —C(O)NR^{A2}R^{B2},
   L³ is absent or is, —C(═O)—,
   Q² is at each occurrence independently selected from: C₃₋₆ cycloalkyl, C₃₋₆ cycloalkyl-C₁₋₃ alkyl, Q⁷, Q⁷-C₁₋₃ alkyl, phenyl, phenyl-C₁₋₃ alkyl, 5 or 6 membered heteroaryl and 5 or 6 membered heteroaryl-C₁₋₃ alkyl,
   wherein Q⁷ is selected from: azetidinyl, oxetanyl pyrrolidinyl, tetrahydrofuranyl, piperidinyl, piperazinyl, tetrahydropyranyl and morpholinyl,
   wherein said C₃₋₆ cycloalkyl, C₃₋₆ cycloalkyl-C₁₋₃ alkyl, 4 to 6 membered heterocyclyl and 4 to 6 membered heterocyclyl-C₁₋₃ alkyl is optionally substituted by one or more (e.g. 1 or 2) substituents selected from: halo, ═O, C₁₋₄ alkyl, C₁₋₄ haloalkyl, —C(O)R^{A5} and —C(O)NR^{A5}R^{B5},
   wherein said phenyl, phenyl-C₁₋₃ alkyl, 5 or 6 membered heteroaryl and 5 or 6 membered heteroaryl-C₁₋₃ alkyl is optionally substituted by one or more (e.g. 1 or 2) substituents selected from: halo, C₁₋₄ alkyl, C₁₋₄ haloalkyl, —OR^{A7} and —NR^{A7}R^{B7}.
56. R¹ is selected from: C₁₋₆ alkyl, —C₁₋₄ alkyl-CN, —C₁₋₄ alkyl-OR^{A2}, C₁₋₆ haloalkyl, C₃₋₆ cycloalkyl, C₃₋₆ cycloalkyl-C₁₋₃ alkyl, 4 to 6 membered heterocyclyl, 4 to 6 membered heterocyclyl-C₁₋₃ alkyl, phenyl, phenyl-C₁₋₃ alkyl, 5 or 6 membered heteroaryl and 5 or 6 membered heteroaryl-C₁₋₃ alkyl;
   wherein said 4 to 6 membered heterocyclyl is selected from: azetidinyl, pyrrolidinyl, piperidinyl, piperazinyl, oxetanyl, tetrahydrofuranyl, tetrahydropyranyl and morpholinyl,
   wherein said 5 or 6 membered heteroaryl is selected from: furyl, pyrrolyl, thienyl, oxazolyl, isoxazolyl, imidazolyl, pyrazolyl, thiazolyl, isothiazolyl, pyridyl, pyridazinyl, pyrimidinyl and pyrazinyl,
   wherein said C₃₋₆ cycloalkyl, is optionally substituted by one or more (e.g. 1 or 2) substituents selected from: ═O, halo, C₁₋₄ alkyl and C₁₋₄ haloalkyl,
   wherein said heterocyclyl is optionally substituted by one or more (e.g. 1 or 2) substituents selected from: ═O, halo, C₁₋₄ alkyl, C₁₋₄ haloalkyl, —C(O)R^{A5} and —C(O)NR^{A5}R^{B5}.
   wherein said phenyl or heteroaryl is optionally substituted by one or more (e.g. 1 or 2) substituents selected from: halo, C₁₋₄ alkyl, C₁₋₄ haloalkyl, —OR^{A5} and —NR^{A5}R^{B5}.
57. R¹ is selected from: C₁₋₄ alkyl and C₁₋₄ haloalkyl, wherein said C₁₋₄ alkyl is optionally substituted by one or more substituents (for example 1 or 2) selected from: halo, —CN, —OR^{A2} and —NR^{A2}R^{B2}.
58. R¹ is C₃₋₆ cycloalkyl optionally substituted by one or more (for example 1 or 2) substituents selected from: halo, C₁₋₄ alkyl, C₁₋₄ haloalkyl, and ═O.
59. R¹ is selected from: azetidinyl, pyrrolidinyl, piperidinyl and piperazinyl, wherein said azetidinyl, pyrrolidinyl, piperidinyl or piperazinyl is bonded to the group -L¹-C(O)— by a ring carbon atom and wherein the ring nitrogen atom in the azetidinyl, pyrrolidinyl, piperidinyl or piperazinyl is optionally substituted by a group selected from:
   C₁₋₄ alkyl, C₁₋₄ haloalkyl, —C₂₋₄ alkyl-OR^{A5}, —C₂₋₄ alkyl-NR^{A5}R^{B5}, —C₁₋₄ alkyl-C(O)NR^{A5}R^{B5}, —C₁₋₄ alkyl-C(O)OR^{A5}, —S(O)₂R^{B5}, —C(O)R^{16A}, —C(O)NR^{16A}R^{B2}, and R¹⁹¹;
   and said azetidinyl, pyrrolidinyl, piperidinyl or piperazinyl is optionally substituted on a ring carbon by one or more (e.g. 1 or 2) substituents selected from: halo, ═O, C₁₋₄ alkyl and C₁₋₄ haloalkyl;
   R¹⁹¹ is selected from: C₃₋₆ cycloalkyl, C₃₋₆ cycloalkyl-C₁₋₃ alkyl-, azetidinyl-C₁₋₃ alkyl-, pyrrolidinyl-C₁₋₃ alkyl-, piperidinyl-C₁₋₃ alkyl-, piperazinyl-C₁₋₃ alkyl-, morpholinyl-C₁₋₃ alkyl-, phenyl, phenyl-C₁₋₃ alkyl-, pyrazolyl, pyridyl, pyrimidyl, pyrazinyl, pyridazinyl, pyrazolyl-C₁₋₃ alkyl-, pyridyl-C₁₋₃ alkyl-, pyrimidyl-C₁₋₃ alkyl-, pyrazinyl-C₁₋₃ alkyl- and pyridazinyl-C₁₋₃ alkyl-;
   R^{16A} is selected from: H, C₁₋₄ alkyl, C₁₋₄ haloalkyl, C₃₋₆ cycloalkyl, C₃₋₆ cycloalkyl-C₁₋₃ alkyl, azetidinyl, pyrrolidinyl, piperidinyl, piperazinyl, morpholinyl, azetidinyl-C₁₋₃ alkyl-, pyrrolidinyl-C₁₋₃ alkyl-, piperidinyl-C₁₋₃ alkyl-, piperazinyl-C₁₋₃ alkyl-, phenyl, phenyl-C₁₋₃ alkyl-, pyrazolyl, pyridyl, pyrimidyl, pyrazinyl, pyridazinyl, pyrazolyl-C₁₋₃ alkyl-, pyridyl-C₁₋₃ alkyl-, pyrimidyl-C₁₋₃ alkyl-, pyrazinyl-C₁₋₃ alkyl- and pyridazinyl-C₁₋₃ alkyl- (for example, it may be that R^{16A} is not H);
wherein R¹⁹¹ and R^{16A} are each independently optionally substituted one or more (e.g. 1 or 2) substituents independently selected from: halo, C₁₋₄ alkyl, C₁₋₄ haloalkyl, —OR^{A5}, —NR^{A5}R^{B5}, —C(O)R^{A5}, —C(O)OR^{A5} and —C(O)NR^{A5}R^{B5}.
60. L¹ is absent and R¹ is selected from: azetidinyl, pyrrolidinyl, piperidinyl, piperazinyl, and morpholinyl, wherein said azetidinyl, pyrrolidinyl, piperidinyl, piperazinyl or morpholinyl is bonded to the —C(O)— group by a ring nitrogen atom; and wherein said azetidinyl, pyrrolidinyl, piperidinyl, piperazinyl or morpholinyl is optionally substituted on a ring carbon by one or more (e.g. 1 or 2) substituents selected from: halo, ═O, C₁₋₄ alkyl and C₁₋₄ haloalkyl.
61. R¹ is a 4 to 7 membered heterocyclyl, for example a saturated 4 to 7 membered heterocyclyl selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl, each of which is optionally substituted by one or more substituents (for example 1 or 2) selected from: halo, C₁₋₄ alkyl, C₁₋₄ haloalkyl, ═O, —C(O)R^{16A}, —C(O)OR^{16A}, —C(O)NR^{A2}R^{B2}, —SO₂R^{16A}, —SO₂Q²², —SO₂CH₂Q²², —C(O)Q²², —C(O)CH₂Q²², —C(O)NR^{A4}Q²², —C(O)NR^{A4}CH₂Q²², —SO₂NR^{A2}R^{B2}, —SO₂NR^{A4}Q²² and —SO₂NR^{A4}CH₂Q²²;
   R^{16A} is selected from: H, C₁₋₄ alkyl and C₁₋₄ alkyl substituted by —OR^{A5}, —S(O)₂R^{A5}, —NR^{A5}R^{B5}, —C(O)R^{A5}, —C(O)OR^{A5}, —C(O)NR^{A5}R^{B5} (for example it may be that R^{16A} is not H),
   Q²² is selected from: C₃₋₆ cycloalkyl, azetidinyl, pyrrolidinyl, piperidinyl, piperazinyl, morpholinyl, phenyl and 5 or 6-membered heteroaryl,
      wherein Q²² is optionally substituted by one or more (e.g. 1 or 2) substituents selected from: halo, C₁₋₄ alkyl, C₁₋₄ haloalkyl, —OR^{A5}, —NR^{A5}R^{B5}, —C(O)R^{A5}, —C(O)NR^{A5}R^{B5} and —C(O)OR^{A5}.
62. R¹ is selected from: phenyl or a 5 or 6-membered heteroaryl containing ring nitrogen and optionally 1, or 2 heteroatoms independently selected from: O, S and N, and wherein R¹ is optionally substituted by one or more substituents (for example 1, 2 or 3) selected from: halo, —CN, C₁₋₄ alkyl, C₁₋₄ haloalkyl, —OR^{A3}, —NR^{A3}R^{B3} and —SO₂R^{A3}.
63. R¹ is selected from: phenyl, thienyl, furanyl, pyrrolyl, imidazolyl, pyrazolyl, oxazolyl, isoxazole, thiazolyl, isothiazolyl, pyridyl, pyrimidyl and pyrazinyl, each of which is optionally substituted by one or more substituents (for example 1, 2 or 3) selected from: halo, —CN, C₁₋₄ alkyl, C₁₋₄ haloalkyl, —OR^{A3}, —NR^{A3}R^{B3} and —SO₂R^{A3}.
64. R¹ is a group of the formula:

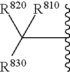

wherein
R⁸¹⁰ is selected from: halo, —OR^{A2}, —NR^{A2}R^{B2}, —CN, C₁₋₆ alkyl, C₁₋₆ haloalkyl and C₃₋₆ cycloalkyl-C₁₋₃ alkyl;
R⁸²⁰ is selected from: halo, C₁₋₆ alkyl, C₁₋₆ haloalkyl, Q¹⁰, Q¹¹ and Q¹²;
R⁸³⁰ is selected from: halo, C₁₋₆ alkyl and C₁₋₆ haloalkyl;
   or R⁸²⁰ and R⁸³⁰ together with the carbon atom to which they are attached form a C₃₋₆ cycloalkyl or 4 to 12 membered heterocyclyl, wherein said C₃₋₆ cycloalkyl or 4 to 12 membered heterocyclyl are each independently optionally substituted by one or more R⁹;
Q¹⁰ is selected from: C₃₋₆ cycloalkyl and C₃₋₆ cycloalkyl-C₁₋₃ alkyl,
Q¹¹ is selected from: 4 to 12 membered heterocyclyl and 4 to 12 membered heterocyclyl-C₁₋₃ alkyl,
Q¹² is selected from: phenyl, phenyl-C₁₋₃ alkyl, 5 or 6 membered heteroaryl, and 5 or 6 membered heteroaryl-C₁₋₃ alkyl;
   wherein Q¹⁰ and Q¹¹ are each independently optionally substituted by one or more R⁹, and
wherein Q¹² is optionally substituted by one or more R¹⁰.
65. R¹ is a group of the formula:

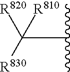

wherein
R⁸¹⁰ is selected from: halo, —OR^{A2}, —NR^{A2}R^{B2}, —CN, C₁₋₆ alkyl, C₁₋₆ haloalkyl and C₃₋₆ cycloalkyl-C₁₋₃ alkyl;
R⁸²⁰ and R⁸³⁰ are independently selected from: halo, C₁₋₆ alkyl and C₁₋₆ haloalkyl;
   or R⁸²⁰ and R⁸³⁰ together with the carbon atom to which they are attached form cyclopropyl, cyclobutyl, cyclopentyl, cyclohexyl, oxetanyl, tetrahydrofuranyl, tetrahydropyranyl, azetidinyl, pyrrolidinyl or piperidinyl, each or which is optionally substituted by one or more (for example 1 or 2) substituents selected from: ═O, halo, C₁₋₄ alkyl, C₁₋₄ haloalkyl, —OR^{A2}, —NR^{A2}R^{B2}, —SO₂R^{A2}, —C(O)R^{A2}, —C(O)OR^{A2}, —C(O)NR^{A2}R^{B2}, and —SO₂NR^{A2}R^{B2}.
66. R¹ is a group of the formula:

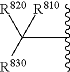

wherein
R⁸¹⁰ is selected from: halo, —OR^{A2}, —NR^{A2}R^{B2}, —CN, C₁₋₆ alkyl, C₁₋₆ haloalkyl and C₃₋₆ cycloalkyl-C₁₋₃ alkyl; and
R⁸²⁰ and R⁸³⁰ are independently selected from: halo, C₁₋₆ alkyl and C₁₋₆ haloalkyl.
67. R¹ is selected from:

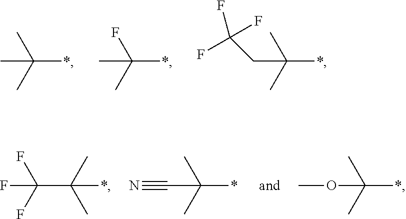

wherein * shown the point of attachment to the -L¹-C(O)— group.
68. R¹ is selected from:

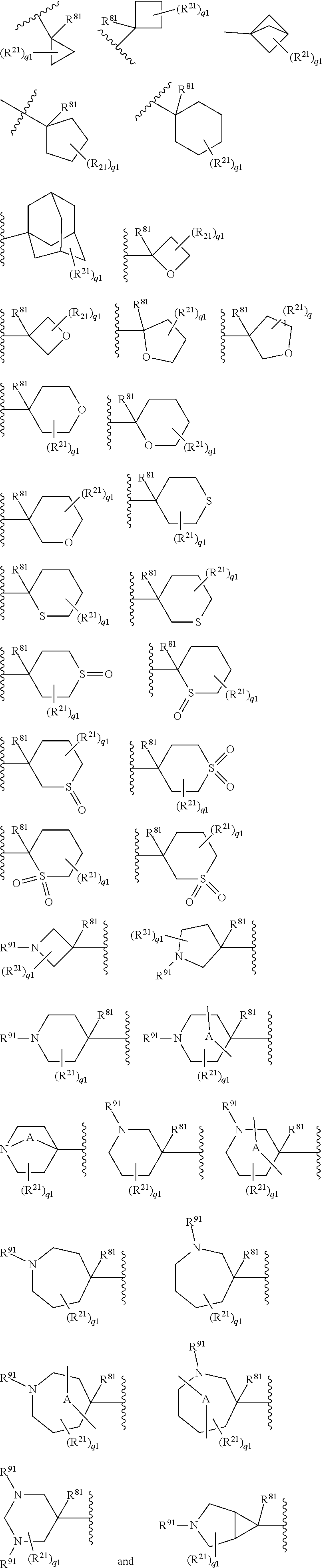

wherein
A is C₁₋₆ alkylene;
R²¹ at each occurrence is independently selected from: halo, ═O, —OH, —OC₁₋₄ alkyl, C₁₋₄ alkyl and C₁₋₄ haloalkyl;
R⁸¹ is selected from: H, C₁₋₄ alkyl, C₂₋₄ alkenyl, C₁₋₄ haloalkyl, and C₃₋₆ cycloalkyl-C₁₋₃ alkyl,
   wherein said C₁₋₄ alkyl is optionally substituted by one or more (e.g. 1 or 2) substituents independently selected from: halo, —CN, —OR^{A5}, —NR^{A5}R^{B5}, —SO₂R^{A5} and C₃₋₆ cycloalkyl;
R⁹¹ is selected from: H, C₁₋₆ alkyl, C₁₋₆ haloalkyl, -L³-Q², —SO₂R¹⁶, —C(O)R¹⁶, —C(O)NR^{16A}R^{B2}, —SO₂NR^{16A}R^{B2} and —C(O)OR^{16A},
   wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5}.
R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, C(O)NR^{A5}R^{B5}, —NR^{B5}SO₂R^{A5} and —SO₂NR^{A5}R^{B5},
R^{16A} is selected from:
   H, C₁₋₆ alkyl, C₁₋₆ haloalkyl,
   C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
   C₂₋₆ alkyl substituted by 1 substituent selected from: —OR^{A5} and —NR^{A5}R^{B5};
Q² is at each occurrence independently selected from: C₃₋₆ cycloalkyl, C₃₋₆ cycloalkyl-C₁₋₃ alkyl, 4 to 6 membered heterocyclyl, 4 to 6 membered heterocyclyl-C₁₋₃ alkyl, phenyl, phenyl-C₁₋₃ alkyl, 5 or 6 membered heteroaryl and 5 or 6 membered heteroaryl-C₁₋₃ alkyl,
   wherein said 4 to 6 membered heterocyclyl contains 1 or 2 heteroatoms selected from: O, S and N,
   wherein said C₃₋₆ cycloalkyl, C₃₋₆ cycloalkyl-C₁₋₃ alkyl, 4 to 6 membered heterocyclyl and 4 to 6 membered heterocyclyl-C₁₋₃ alkyl is optionally substituted by one or more R¹⁴, and
   wherein said phenyl, phenyl-C₁₋₃ alkyl, 5 or 6 membered heteroaryl and 5 or 6 membered heteroaryl-C₁₋₃ alkyl is optionally substituted by one or more R¹⁵;
L³ is absent or is selected from: —SO₂—, —C(═O)—, *—C(═O)NR^{A4}—, *—S(O)₂NR^{A4}—, —NR^{A4}S(O)₂— and *—C(O)O—, wherein * indicates the point of attachment to the ring nitrogen in R¹;
R¹⁴ at each occurrence is independently selected from: halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and
R¹⁵ at each occurrence is independently selected from: halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7}; and
q1 is an integer selected from: 0, 1, 2, 3 and 4;
provided that when L³ is absent Q² is bonded to the ring nitrogen atom in R¹ via a ring carbon atom in Q².
69. R¹ is selected from:

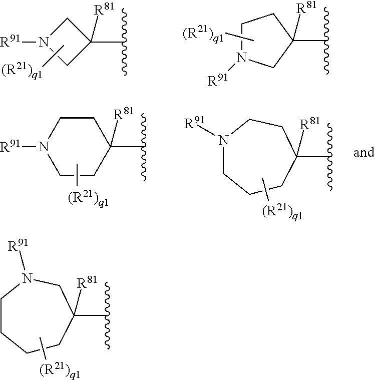

wherein R²¹, R⁸¹, R⁹¹ and q1 are as defined in (68) above.
70. R¹ is:

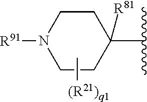

wherein R²¹, R⁸¹, R⁹¹ and q1 are as defined in (68) above.
71. R¹ is as defined is any of (68) to (70), wherein:
   R⁹¹ is selected from: H, C₁₋₆ alkyl, C₁₋₆ haloalkyl, -L³-Q², —SO₂R¹⁶, —C(O)R¹⁶, —C(O)NR^{16A}R^{B2}, —SO₂NR^{16A}R^{B2} and —C(O)OR^{16A}.
      wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},
   R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R A⁵, C(O)NR^{A5}R^{B5}, —NR^{B5}SO₂R^{A5} and —SO₂NR^{A5}R^{B5},
   R^{16A} is selected from:
      H, C₁₋₆ alkyl, C₁₋₆ haloalkyl,
      C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
      C₂₋₆ alkyl substituted by 1 substituent selected from: —OR^{A5} and —NR^{A5}R^{B5}
   Q² is selected from:
   Q⁶, Q⁶-C₁₋₃ alkylene-, Q⁷, Q⁷-C₁₋₃ alkylene-, Q⁸ and Q⁸-C₁₋₃ alkylene-,
   wherein
   Q⁶ is C₃₋₆ cycloalkyl;
   Q⁷ is selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl;
   Q³ is selected from: phenyl, pyrrolyl, furanyl, thienyl, imidazolyl, oxazolyl, oxadiazolyl, isoxazolyl, thiazolyl, isothiazolyl, pyrazolyl, pyridyl, pyrazinyl, pyridazinyl and pyrimidinyl;
      wherein said Q⁶, Q⁶-C₁₋₃ alkylene-, Q⁷ and Q⁷-C₁₋₃ alkylene- are each optionally substituted by 1 to 4 R¹⁴, and Q³ and Q⁸-C₁₋₃ alkylene- are each optionally substituted by 1 to 4 R¹⁵;
   L³ is absent or is selected from: —SO₂—, —C(═O)—, *—C(═O)NR^{A4}—, *—S(O)₂NR^{A4} and *—C(O)O—, wherein * indicates the point of attachment to the ring nitrogen in R¹;
   R¹⁴ at each occurrence is independently selected from: halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and
   R¹⁵ at each occurrence is independently selected from: halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7};
   provided that when L³ is absent Q² is bonded to the ring nitrogen atom in R¹ via a ring carbon atom in Q².
72. R¹ is as defined is any of (68) to (70), wherein:
   R⁹¹ is selected from: H, C₁₋₆ alkyl, C₁₋₆ haloalkyl, -L³-Q², —SO₂R¹⁶, —C(O)R¹⁶, —C(O)NR^{16A}R^{B2}, —SO₂NR^{16A}R^{B2} and —C(O)OR^{16A},
      wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5}.
   R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5} and —NR^{A5}R^{B5},
   R^{16A} is selected from:
      H, C₁₋₆ alkyl, C₁₋₆ haloalkyl,
      C₁₋₆ alkyl substituted by 1 substituent selected from: —CN, and —S(O)₂R^{A5}, and
      C₂₋₆ alkyl substituted by 1 substituent selected from: —OR^{A5} and —NR^{A5}R^{B5}
   Q² is selected from:
   Q⁶, Q⁶-C₁₋₃ alkylene-, Q⁷, Q⁷-C₁₋₃ alkylene-, Q⁸ and Q⁸-C₁₋₃ alkylene-,
   wherein
   Q⁶ is C₃₋₆ cycloalkyl;
   Q⁷ is selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl;
   Q³ is selected from: phenyl, pyrrolyl, furanyl, thienyl, imidazolyl, oxazolyl, oxadiazolyl, isoxazolyl, thiazolyl, isothiazolyl, pyrazolyl, pyridyl, pyrazinyl, pyridazinyl and pyrimidinyl;
      wherein said Q⁶, Q⁶-C₁₋₃ alkylene- are each optionally substituted by 1 to 4 R¹⁴¹, said Q⁷ and Q⁷-C₁₋₃ alkylene- are each optionally substituted by 1 to 4 R¹⁴², and said Q³ and Q⁸-C₁₋₃ alkylene- are each optionally substituted by 1 to 4 R¹⁵;
   L³ is absent or is selected from: —SO₂—, —C(═O)—, *—C(═O)NR^{A4}— and *—S(O)₂NR^{A4}—, wherein * indicates the point of attachment to the ring nitrogen in R¹;
   R¹⁴¹ at each occurrence is independently selected from: halo, ═O, and C₁₋₄ alkyl;
   R¹⁴² at each occurrence is independently selected from: halo, ═O, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}.
   and
   R¹⁵ at each occurrence is independently selected from: halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7};
      provided that when L³ is absent Q² is bonded to the ring nitrogen atom in R¹ via a ring carbon atom in Q².
73. R¹ is as defined is any of (68) to (72), wherein L³ is absent or is —C(O)—.
74. R¹ is as defined is any of (68) to (73), wherein R²¹ at each occurrence is independently selected from: halo, ═O and C₁₋₄ alkyl.
75. R¹ is as defined is any of (68) to (74), wherein q1 is 0.
76. R¹ is as defined is any of (68) to (75), wherein R⁸¹ is selected from: H, C₁₋₄ alkyl, C₁₋₄ haloalkyl and C₃₋₆ cycloalkyl-C₁₋₃alkyl.
77. R¹ is as defined is any of (68) to (75), wherein R⁸¹ is selected from: C₁₋₄ alkyl, C₁₋₄ haloalkyl and C₃₋₆ cycloalkyl-C₁₋₃alkyl.
78. R¹ is as defined is any of (68) to (75), wherein R⁸¹ is C₁₋₄ alkyl (e.g. methyl or ethyl).
79. R¹ is as defined is any of (68) to (75), wherein R⁸¹ is H.
80. R¹ is as defined is any of (68) to (79), wherein R⁹¹ is H or is selected from:

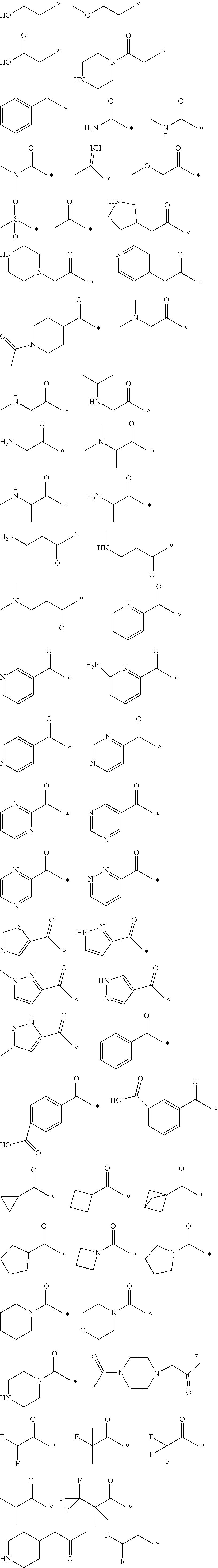

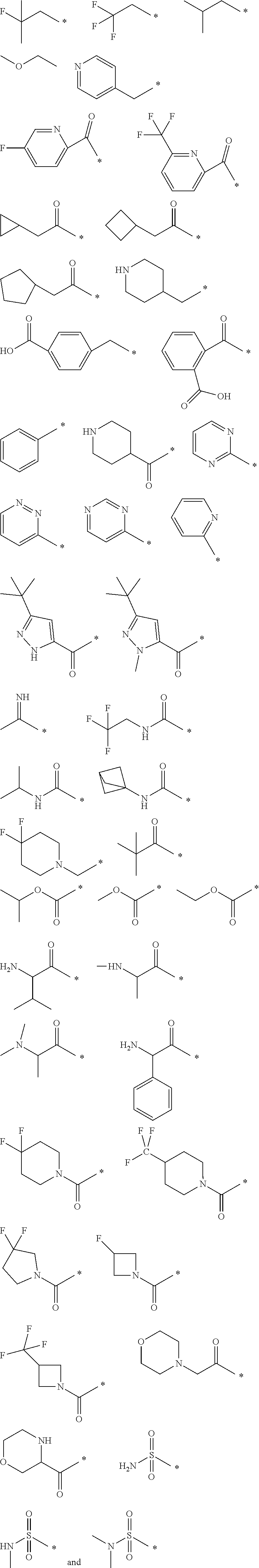

wherein * indicates the point of attachment of the R⁹¹ group to the ring nitrogen in R¹.
81. R¹ is as defined is any of (68) to (80), wherein R⁹¹ is not H.
82. R¹ is as defined is any of (68) to (80), wherein R⁹¹ is H.
83. L¹ is absent and R¹ is selected from:

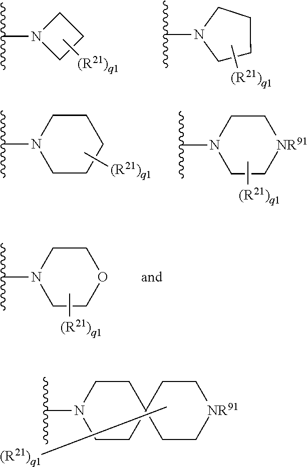

wherein R²¹ at each occurrence is independently selected from: halo, ═O and C₁₋₄ alkyl;
R⁹¹ is as defined in (71), (72), (80), (81) or (82); and
q1 is an integer selected from 0, 1, 2, 3 and 4.
84. R¹ is selected from:

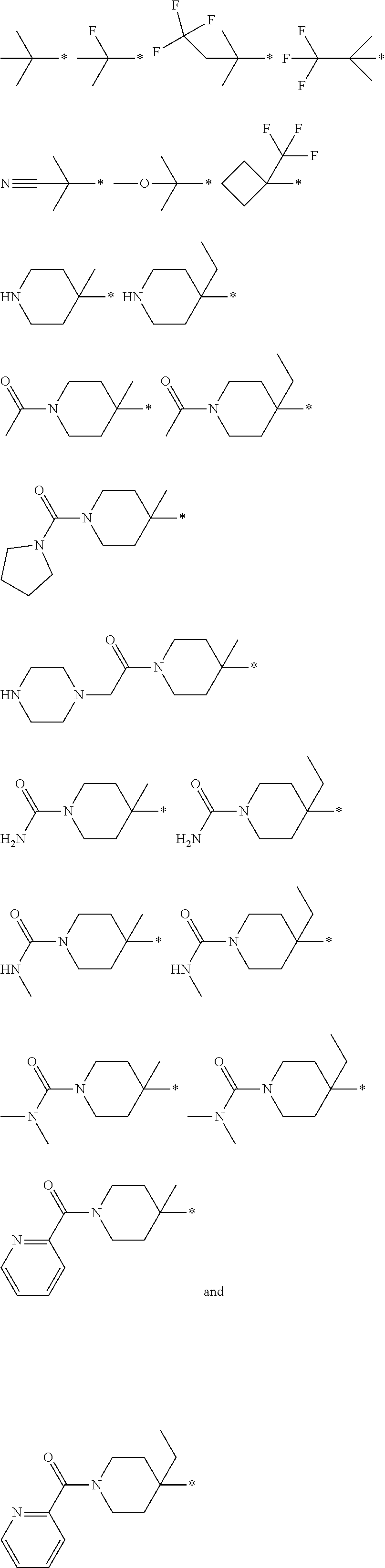

wherein * shows the point of attachment to -L¹-C(O)—.
85. R¹ is selected from:

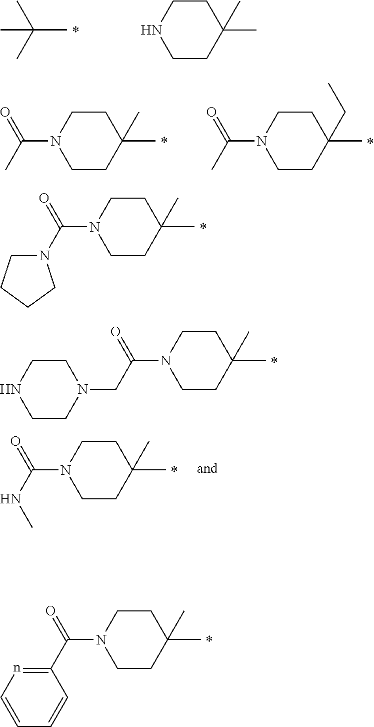

wherein * shows the point of attachment to -L¹-C(O)—
86. R¹ is

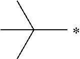

87. L¹ is absent.
88. L¹ is —O—.
89. L¹ is —N(R⁷)—.
90. L¹ is absent and R¹ is as defined in any of (52) to (86).
91. L¹ is —O— and R¹ is as defined in any one of (52) to (59), (61) to (82) or (84) to (86) above.
92. L¹ is —N(R⁷)— and HET is as defined in any one of (52) to (58), (61) to (82) or (84) to (86) above.
93. R⁴ is selected from: H and C₁₋₃ alkyl and R⁵ is H.
94. R⁴ is C₁₋₃ alkyl (e.g. R⁴ is example methyl) and R⁵ is H.
95. R⁴ and R⁵ together with the carbon to which they are attached form a C₃₋₆ cycloalkyl, for example cyclopropyl or cyclobutyl.
96. R⁴ and R⁵ are both C₁₋₄ alkyl.
97. R⁴ and R⁵ are both methyl.
98. R⁴ and R⁵ are both H.
99. X, is N.
100. X, is CR⁶
101. X, is CR⁶ and R⁶ is selected from: H, halo, C₁₋₄ alkyl and C₁₋₄ haloalkyl.
102. X, is CR⁶ and R⁶ is selected from: halo, C₁₋₄ alkyl and C₁₋₄ haloalkyl.
103. X, is CR⁶ and R⁶ is selected from: H, fluoro, methyl, ethyl and CF₃.
104. X, is CR⁶ and R⁶ is selected from: F, methyl, ethyl or CF₃.
105. X, is CR⁶ and R⁶ is selected from: halo and C₁₋₄ alkyl.
106. X, is CR⁶ and R⁶ is selected from: H and C₁₋₄ alkyl.
107. X, is CR⁶ and R⁶ is H.
108. X, is CR⁶ and R⁶ is C₁₋₄ alkyl.
109. X, is CR⁶ and R⁶ is methyl.
110. X, is CR⁶ and R⁶ is halo.
111. X, is CR⁶ and R⁶ is fluoro.

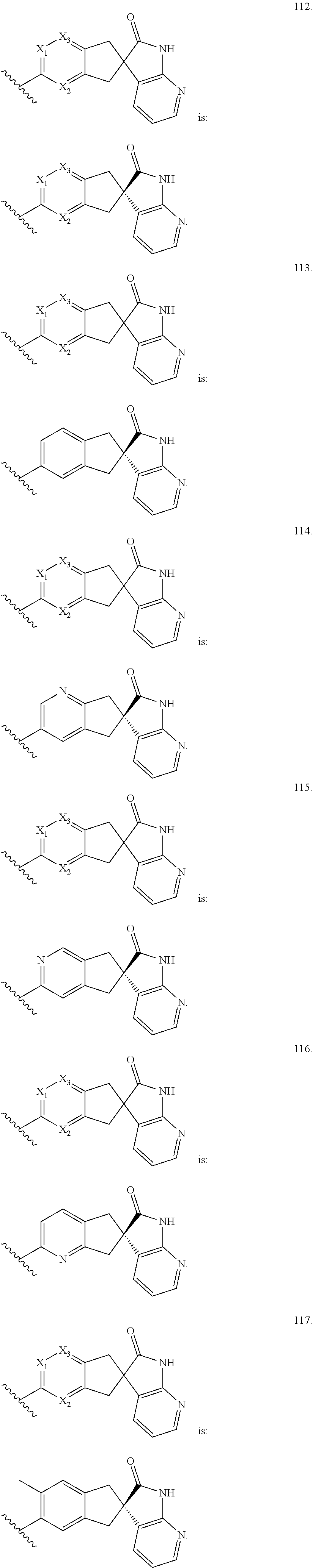

In certain embodiments there is provided a compound of the formula (I), wherein: HET is selected from:

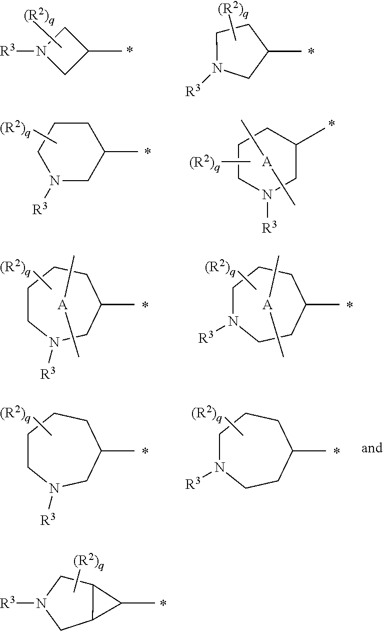

wherein A is C₁₋₄ alkylene and * shows the point of attachment to the remainder of the compound;
R³ is H or C₁₋₃ alkyl (preferably R³ is H);
R² is C₁₋₃ alkyl; q is 0, 1 or 2 (preferably q is 0);
L and L¹ are absent;
R¹ is as defined in any one of (52) to (86) above; and
R⁴, R⁵, X₁, X₂ and X₃ are as defined for formula (I).

In certain embodiments there is provided a compound of the formula (I), wherein: HET is selected from:

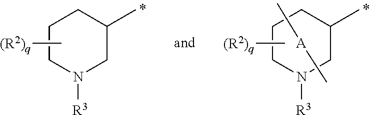

wherein A is C₁₋₄ alkylene and * shows the point of attachment to the remainder of the compound;
R³ is H or C₁₋₃ alkyl (preferably R³ is H);
R² is C₁₋₃ alkyl;
q is 0, 1 or 2 (preferably q is 0);
L and L¹ are absent;
R¹ is as defined in any one of (52) to (86) above; and
R⁴, R⁵, X₁, X₂ and X₃ are as defined for formula (I).

In certain embodiments there is provided a compound of the formula (I), wherein:
R¹ is as defined in (54) above;
L and L¹ are absent;
HET is as defined in any one of (1) to (38) above; and
R⁴, R⁵, X₁, X₂ and X₃ are as defined for formula (I).

In certain embodiments there is provided a compound of the formula (I), wherein:
R¹ is as defined in (61) above;
L and L¹ are absent;
HET is as defined in any one of (1) to (38) above; and
R⁴, R⁵, X₁, X₂ and X₃ are as defined for formula (I).

In certain embodiments there is provided a compound of the formula (I), wherein:
R¹ is as defined in (68) above;
L and L¹ are absent;
HET is as defined in any one of (1) to (38) above; and
R⁴, R⁵, X₁, X₂ and X₃ are as defined for formula (I).

In certain embodiments there is provided a compound of the formula (I), wherein:
R¹ is as defined in (69) above;
L and L¹ are absent;
HET is as defined in any one of (1) to (38) above; and
R⁴, R⁵, X₁, X₂ and X₃ are as defined for formula (I).

In certain embodiments there is provided a compound of the formula (I), wherein:
R¹ is as defined in (84) above;
L and L¹ are absent;
HET is as defined in any one of (1) to (38) above; and
R⁴, R⁵, X₁, X₂ and X₃ are as defined for formula (I).

In certain embodiments there is provided a compound of the formula (I), wherein:
R¹ is selected from:

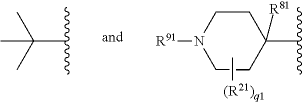

R⁹¹ is selected from: H, C₁₋₆ alkyl, C₁₋₆ haloalkyl, -L³-Q², —SO₂R¹⁶, —C(O)R¹⁶, —C(O)NR^{16A}R^{B2}, —SO₂NR^{16A}R^{B2} and —C(O)OR^{16A},
   wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},
R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, —C(O)NR^{A5}R^{B5}, —NR^{B5}SO₂R^{A5} and —SO₂NR^{A5}R^{B5},
R^{16A} is selected from:
   H, C₁₋₆ alkyl, C₁₋₆ haloalkyl,
   C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
   C₂₋₆ alkyl substituted by 1 substituent selected from: —OR^{A5} and —NR^{A5}R^{B5};
Q² is selected from:
Q⁶, Q⁶-C₁₋₃ alkylene-, Q⁷, Q⁷-C₁₋₃ alkylene-, Q³ and Q⁸-C₁₋₃ alkylene-,
wherein
Q⁶ is C₃₋₆ cycloalkyl;
Q⁷ is selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl;
Q³ is selected from: phenyl, pyrrolyl, furanyl, thienyl, imidazolyl, oxazolyl, oxadiazolyl, isoxazolyl, thiazolyl, isothiazolyl, pyrazolyl, pyridyl, pyrazinyl, pyridazinyl and pyrimidinyl;
   wherein said Q⁶, Q⁶-C₁₋₃ alkylene-, Q⁷ and Q⁷-C₁₋₃ alkylene- are each optionally substituted by 1 to 4 R¹⁴, and Q⁸ and Q⁸-C₁₋₃ alkylene- are each optionally substituted by 1 to 4 R¹⁵;
L³ is absent or is selected from: —SO₂—, —C(═O)—, *—C(═O)NR^{A4}—, *—S(O)₂NR^{A4} and *—C(O)O—, wherein * indicates the point of attachment to the ring nitrogen in R¹;
R¹⁴ at each occurrence is independently selected from: halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and
R¹⁵ at each occurrence is independently selected from: halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7};
provided that when L³ is absent Q² is bonded to the ring nitrogen atom in R¹ via a ring carbon atom in Q²;
R²¹ at each occurrence is independently selected from: halo, ═O and C₁₋₄ alkyl;
R⁸¹ is selected from: H, C₁₋₄ alkyl, C₁₋₄ haloalkyl and C₃₋₆ cycloalkyl-C₁₋₃alkyl;
L and L¹ are absent;
HET is as defined in any one of (1) to (38) above;
q1 is an integer selected from 0, 1 and 2;
and
R⁴, R⁵, X₁, X₂ and X₃ are as defined for formula (I).

Suitably in this embodiment L³ is absent or is —C(═O)—.

Suitably in this embodiment R⁸¹ is selected from: C₁₋₄ alkyl, C₁₋₄ haloalkyl and C₃₋₆ cycloalkyl-C₁₋₃alkyl.

Suitably in this embodiment R⁸¹ is selected from: C₁₋₄ alkyl, C₁₋₄ haloalkyl and C₃₋₆ cycloalkyl-C₁₋₃alkyl; and L³ is absent or is —C(═O)—.

It may be that in this embodiment R⁸¹ is not H.

It may be that in this embodiment R⁸¹ is H.

It may be that in this embodiment R⁹¹ is not H.

It may be that in this embodiment R⁹¹ is H.

Suitably in this embodiment R⁹¹ is selected from: —C(O)R¹⁶, —C(O)NR^{16A}R^{B2}; R¹⁶ is C₁₋₄ alkyl; R^{16A} is selected from: H and C₁₋₄alkyl; and R^{B2} is selected from: H and C₁₋₄alkyl.

In certain embodiments there is provided a compound of the formula (I), wherein:
R¹ is tert-butyl;
L and L¹ are absent;
HET is as defined in any one of (1) to (38) above; and
R⁴, R⁵, X₁, X₂ and X₃ are as defined for formula (I).

In certain embodiments there is provided a compound of the formula (I), (II), (III), (IV), (V), (VI), (VII) or (VIII) wherein the group:

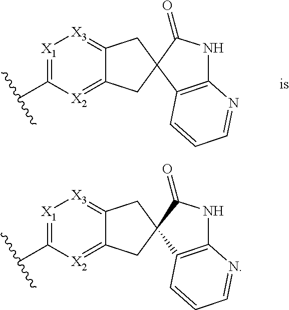

In certain embodiments there is provided a compound of the formula (I), (II), (III), (IV), (V), (VI), (VII) or (VIII) wherein the group:

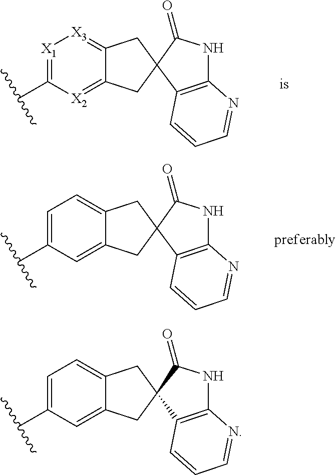

In certain embodiments the compound of formula (I) is a compound according to formula (IIa), or a pharmaceutically acceptable salt thereof:

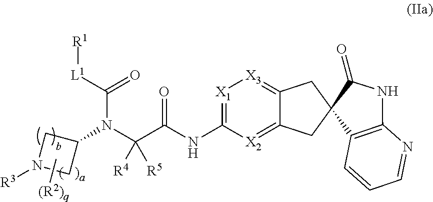

wherein
a is an integer selected from 0, 1 and 2;
b is an integer selected from 1, 2, 3 and 4; and
R¹, R², R³, R⁴, R⁵, L¹, X1, X₂, X₃ and q are as defined for formula (I).

In certain embodiments the compound of formula (I) is a compound according to formula (IIIa), or a pharmaceutically acceptable salt thereof:

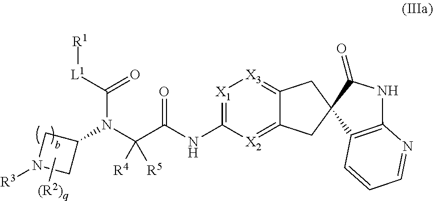

wherein b is an integer selected from 1, 2, 3 and 4; and
R¹, R², R³, R⁴, R⁵, L¹, X₁, X₂, X₃ and q are as defined for formula (I) (including any of the values in (1) to (117) above).

In certain embodiments the compound of formula (I) is a compound according to formula (Va), or a pharmaceutically acceptable salt thereof:

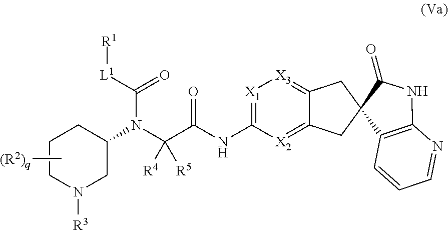

wherein
R¹, R², R³, R⁴, R⁵, L¹, X₁, X₂, X₃ and q are as defined for formula (I) (including any of the values in (1) to (117) above).

In certain embodiments the compound of formula (I) is a compound according to formula (VIa), or a pharmaceutically acceptable salt thereof:

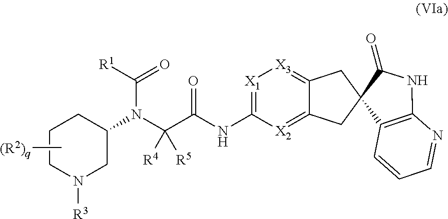

wherein
R¹, R², R³, R⁴, R⁵, X₁, X₂, X₃ and q are as defined for formula (I) (including any of the values in (1) to (117) above)

In certain embodiments the compound of formula (I) is a compound according to formula (VIIa), or a pharmaceutically acceptable salt thereof:

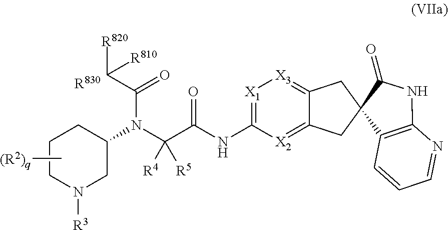

wherein
R¹, R², R³, R⁴, R⁵, X₁, X₂, X₃ and q are as defined for formula (I) and R⁸¹⁰, R⁸²⁰ and R⁸³⁰ are as defined for formula (VII) (including any of the values in (1) to (117) above).

In certain embodiments the compound of formula (I) is a compound according to formula (VIIIa), or a pharmaceutically acceptable salt thereof:

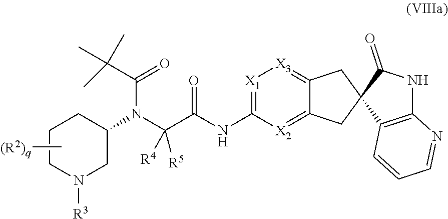

wherein R², R³, R⁴, R⁵, X₁, X₂, X₃ and q are as defined for formula (I) (including any of the values in (1) to (117) above)

In certain embodiments in a compound of the formula (II), (IIa), (III), (IIIa), (IV), (V), (Va), (VI) or (VIa), or a pharmaceutically acceptable salt thereof, R¹ is selected from:

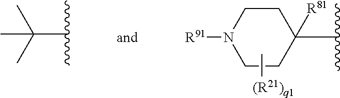

R⁹¹ is selected from: H, C₁₋₆ alkyl, C₁₋₆ haloalkyl, -L³-Q², —SO₂R¹⁶, —C(O)R¹⁶, —C(O)NR^{16A}R^{B2}, —SO₂NR^{16A}R^{B2} and —C(O)OR^{16A},

wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},

R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, —C(O)NR^{A5}R^{B5}, —NR^{B5}SO₂R^{A5} and —SO₂NR^{A5}R^{B5},
R^{16A} is selected from:
H, C₁₋₆ alkyl, C₁₋₆ haloalkyl,
C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
C₂₋₆ alkyl substituted by 1 substituent selected from: —OR^{A5} and —NR^{A5}R^{B5}
   Q² is selected from:
   Q⁶, Q⁶-C₁₋₃ alkylene-, Q⁷, Q⁷-C₁₋₃ alkylene-, Q⁸ and Q⁸-C₁₋₃ alkylene-,
   wherein
   Q⁶ is C₃₋₆ cycloalkyl;
   Q⁷ is selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl;
   Q⁸ is selected from: phenyl, pyrrolyl, furanyl, thienyl, imidazolyl, oxazolyl, oxadiazolyl, isoxazolyl, thiazolyl, isothiazolyl, pyrazolyl, pyridyl, pyrazinyl, pyridazinyl and pyrimidinyl;

wherein said Q⁶, Q⁶-C₁₋₃ alkylene-, Q⁷ and Q⁷-C₁₋₃ alkylene- are each optionally substituted by 1 to 4 R¹⁴, and Q⁸ and Q⁸-C₁₋₃ alkylene- are each optionally substituted by 1 to 4 R¹⁵;

L³ is absent or is selected from: —SO₂—, —C(═O)—, *—C(═O)NR^{A4}—, *—S(O)₂NR^{A4} and *—C(O)O—, wherein * indicates the point of attachment to the ring nitrogen in R¹;

R¹⁴ at each occurrence is independently selected from: halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and

R¹⁵ at each occurrence is independently selected from: halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7};

provided that when L³ is absent Q² is bonded to the ring nitrogen atom in R¹ via a ring carbon atom in Q²;

R²¹ at each occurrence is independently selected from: halo, ═O and C₁₋₄ alkyl;
R⁸¹ is selected from: H, C₁₋₄ alkyl, C₁₋₄ haloalkyl and C₃₋₆ cycloalkyl-C₁₋₃alkyl; and
q1 is an integer selected from 0, 1 and 2.

Suitably in these embodiments of compounds of the formula (II), (IIa), (III), (IIIa), (IV), (V), (Va), (VI) and (VIa) L³ is absent or is —C(═O)—.

Suitably in these embodiments of compounds of the formula (II), (IIa), (III), (IIIa), (IV), (V), (Va), (VI) and (VIa) R⁸¹ is selected from: C₁₋₄ alkyl, C₁₋₄ haloalkyl and C₃₋₆ cycloalkyl-C₁₋₃alkyl (e.g R⁸¹ is methyl or ethyl).

Suitably in these embodiments of compounds of the formula (II), (IIa), (III), (IIIa), (IV), (V), (Va), (VI) and (VIa) R⁸¹ is selected from: C₁₋₄ alkyl, C₁₋₄ haloalkyl and C₃₋₆ cycloalkyl-C₁₋₃alkyl; and L³ is absent or is —C(═O)—.

It may be that in these embodiments of compounds of the formula (II), (IIa), (III), (IIIa), (IV), (V), (Va), (VI) and (VIa) R⁹¹ is not H.

It may be that in these embodiments of compounds of the formula (II), (IIa), (III), (IIIa), (IV), (V), (Va), (VI) and (VIa) R⁹¹ is H.

It may be that in these embodiments of compounds of the formula (II), (IIa), (Iii), (IIIa), (IV), (V), (Va), (VI) and (VIa) R⁹¹ is selected from: —C(O)R¹⁶, —C(O)NR^{16A}R^{B2}; R¹⁶ is C₁₋₄ alkyl; R^{16A} is selected from: H and C₁₋₄ alkyl; and R^{B2} is selected from: H and C₁₋₄ alkyl

In certain embodiments in a compound of the formula (II), (IIa), (III), (IIIa), (IV), (V), (Va), (VI) or (VIa), or a pharmaceutically acceptable salt thereof, R¹ is selected from: C₁₋₆ alkyl, C₁₋₆ haloalkyl, C₃₋₆ cycloalkyl and 4 to 7 membered heterocyclyl containing 1 or 2 ring heteroatoms selected from O, S and N;

wherein said C₁₋₆ alkyl, C₃₋₆ cycloalkyl and 4 to 7 membered heterocyclyl is optionally substituted by one or more (e.g. 1 or 2) substituents selected from: halo, ═O, —CN, C₁₋₄ alkyl, C₁₋₄ haloalkyl —OH, —O(C₁₋₄ alkyl), —C(═O)(C₁₋₄ alkyl), —C(═O)NH(C₁₋₄ alkyl), —C(═O)N(C₁₋₄ alkyl)₂, Q²⁰, Q²⁰-C(═O)—, Q²⁰NHC(═O)—, Q²⁰N(C₁₋₄ alkyl)C(═O)—,

Q²⁰ is selected from, C₃₋₆ cycloalkyl, C₃₋₆ cycloalkyl-CH₂—, 4-7 membered heterocyclyl, 4-7 membered heterocyclyl-CH₂—, 5 or 6 membered heteroaryl, 5 or 6 membered heteroaryl-CH₂—, phenyl and benzyl, wherein said C₃₋₆ cycloalkyl, C₃₋₆ cycloalkyl-CH₂—, 4-7 membered heterocyclyl, 4-7 membered heterocyclyl-CH₂— in Q²⁰ is optionally substituted by one or more substituents selected from: halo, ═O, C₁₋₄ alkyl and C₁₋₄ haloalkyl; and wherein said 5 or 6 membered heteroaryl, 5 or 6 membered heteroaryl-CH₂—, phenyl and benzyl is optionally substituted by one or more substituents selected from: halo, C₁₋₄ alkyl and C₁₋₄ haloalkyl.

Suitably in this embodiment L¹ is absent. Suitably in this embodiment q is 0.

In certain embodiments in a compound of the formula (II), (IIa), (III), (IIIa), (IV), (V), (Va), (VI) or (VIa), or a pharmaceutically acceptable salt thereof, R¹ is selected from: C₁₋₆ alkyl, C₁₋₆ haloalkyl, C₃₋₆ cycloalkyl and heterocyclyl, wherein said heterocyclyl is selected from pyrrolidinyl, piperidinyl, piperazinyl and tetrahydropyranyl;

wherein said C₁₋₆ alkyl, C₃₋₆ cycloalkyl and heterocyclyl is optionally substituted by one or more (e.g. 1 or 2) substituents selected from: halo, —CN, C₁₋₄ alkyl, C₁₋₄ haloalkyl, —OH, —O(C₁₋₄ alkyl), —C(═O)(C₁₋₄ alkyl), —C(═O)NH(C₁₋₄ alkyl), —C(═O)N(C₁₋₄ alkyl)₂, Q²¹, Q²¹-C(═O)—, Q²¹NHC(═O)—, Q²¹N(C₁₋₄ alkyl)C(═O)—,

Q²¹ is selected from: heterocyclyl, heterocyclyl-CH₂—, 5 or 6 membered heteroaryl, 5 or 6 membered heteroaryl-CH₂—, phenyl, benzyl, wherein the heterocyclyl represented by Q²¹ is selected from: pyrrolidinyl, piperidinyl, piperazinyl and tetrahydropyranyl; and wherein Q²¹ is optionally substituted by one or more substituents selected from: halo, C₁₋₄ alkyl and C₁₋₄ haloalkyl.

Suitably in this embodiment L¹ is absent. Suitably in this embodiment q is 0.

In certain embodiments in a compound of the formula (II), (IIa), (III), (IIIa), (IV), (V), (Va), (VI) and (VIa), or a pharmaceutically acceptable salt thereof, wherein:

R¹ is as defined in any one of (52) to (86) above;
R² is C₁₋₃ alkyl or ═O;

### R³ is H;

X₁, X₂ and X₃ are CH;
R₄ is H or C₁₋₃ alkyl,

### R⁵ is H; and

q is an integer selected from: 0, 1 and 2.

Suitably in these embodiments q is 0.

In certain embodiments in a compound of the formula (VII) and (VIIa), or a pharmaceutically acceptable salt thereof:

R⁸¹⁰ is selected from C₁₋₆ alkyl, C₁₋₆ haloalkyl and C₃₋₆ cycloalkyl-C₁₋₃ alkyl,
R⁸²⁰ and R⁸³⁰ are each independently selected from: halo, C₁₋₆ alkyl and C₁₋₆ haloalkyl,

or R⁸²⁰ and R⁸³⁰ together with the carbon atom to which they are attached form a C₃₋₆ cycloalkyl or 4 to 7 membered heterocyclyl selected from:
azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl, each of which is optionally substituted by one or more substituents (for example 1 or 2) selected from: halo, C₁₋₄ alkyl, C₁₋₄ haloalkyl, ═O, —C(O)R^{16A}, —C(O)OR^{16A}, —C(O)NR^{A2}R^{B2}, —SO₂R^{16A}, —SO₂Q²², —SO₂CH₂Q²², —C(O)Q²², —C(O)CH₂Q²², —C(O)NR^{B2}Q²², —C(O)NR^{B2}CH₂Q²², —SO₂NR^{A2}R^{B2}, —SO₂NR^{B2}Q²² and SO₂NR^{B2}CH₂Q²².
   R^{16A} is selected from: C₁₋₄ alkyl and C₁₋₄ alkyl substituted by —OR^{A5}, —S(O)₂R^{A5}, —NR^{A5}R^{B5}, —C(O)R^{A5}, —C(O)OR^{A5}, —C(O)NR^{A5}R^{B5},
   Q²² is selected from: C₃₋₆ cycloalkyl, azetidinyl, pyrrolidinyl, piperidinyl, piperazinyl, morpholinyl, phenyl and 5 or 6-membered heteroaryl,

wherein Q²² is optionally substituted by one or more (e.g. 1 or 2) substituents selected from: halo, C₁₋₄ alkyl, C₁₋₄ haloalkyl, —OR^{A5}, —NR^{A5}R^{B5}, —C(O)R^{A5}, —C(O)NR^{A5}R^{B5} and —C(O)OR^{A5}.

In certain embodiments in a compound of the formula (VII) and (VIIa), or a pharmaceutically acceptable salt thereof:

R⁸¹ is selected from: C₁₋₆ alkyl and C₁₋₆ haloalkyl; and
R⁸² and R⁸³ are each independently selected from: halo, C₁₋₆ alkyl and C₁₋₆ haloalkyl.

In certain embodiments in a compound of the formula (VII) and (VIIa), or a pharmaceutically acceptable salt thereof, R⁸¹, R⁸² and R⁸³ are each independently selected from: halo, C₁₋₆ alkyl and C₁₋₆ haloalkyl.

In certain embodiments in a compound of the formula (VII) and (VIIa), or a pharmaceutically acceptable salt thereof, the group —C(R⁸¹)(R⁸²)(R⁸³) is tert-butyl.

In certain embodiments in a compound of the formula (VII), (VIIa), (VIII) or (VIIIa), or a pharmaceutically acceptable salt thereof described herein, it may be that X₁, X₂ and X₃ are CH.

In certain embodiments in a compound of the formula (VII), (VIIa), (VIII) or (VIIIa), or a pharmaceutically acceptable salt thereof described herein, it may be that:

R² is C₁₋₃ alkyl or ═O;

### R³ is H;

X₁, X₂ and X₃ are CH;
R₄ is H or C₁₋₃ alkyl;

### R⁵ is H; and

q is an integer selected from: 0, 1 and 2 (e.g. q is 0).

In another embodiment there is provided a compound selected from List 1, or a pharmaceutically acceptable salt, or prodrug thereof:

### List 1

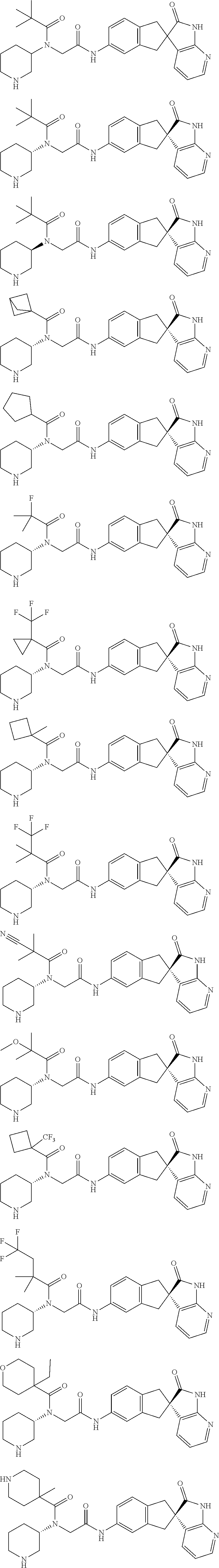

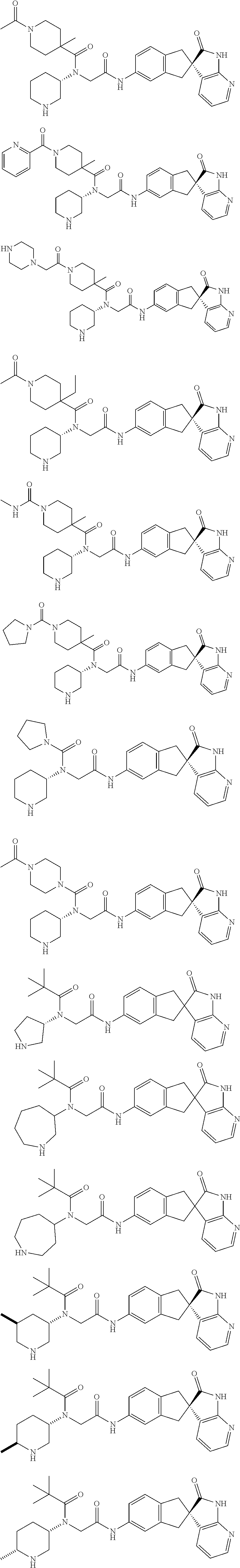

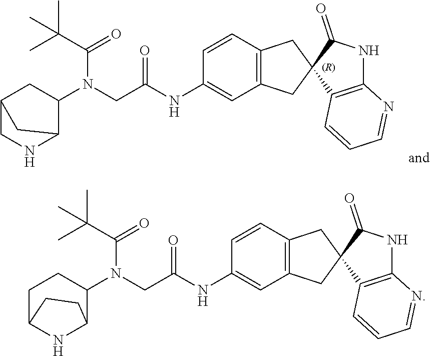

In another embodiment there is provided a compound selected from any one of the Examples herein, or a pharmaceutically acceptable salt, or prodrug thereof.

Particular compounds of the invention are those which have an pIC₅₀ of greater than or equal to 7 when tested in the AM₂ receptor cAMP/Agonist-Antagonist competition assay described in the Examples.

### Pharmaceutical Compositions

In accordance with another aspect, the present invention provides a pharmaceutical composition comprising a compound of the invention, or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 750 mg/kg, 1 mg/kg to 600 mg/kg, 1 mg/kg to 550 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous, subcutaneous, intramuscular or intraperitoneal administration, a dose in the range, for example, 0.1 mg/kg to 30 mg/kg body weight will generally be used. In certain embodiments the compound of the invention is administered intravenously, for example in a daily dose of from 1 mg/kg to 750 mg/kg, 1 mg/kg to 600 mg/kg, 1 mg/kg to 550 mg/kg, or 5 mg/kg to 550 mg/kg, for example at about 10, 20, 30, 40, 50, 60, 70, 80, 90, 100, 125, 150, 175, 180, 200, 225, 250, 275, 300, 350, 400, 450, 500, 540, 550 or 575 mg/kg. Similarly, for administration by inhalation, a dose in the range, for example, 0.05 mg/kg to 25 mg/kg body weight will be used. Suitably the compound of the invention is administered orally, for example in the form of a tablet, or capsule dosage form. The daily dose administered orally may be, for example a total daily dose selected from 1 mg to 1000 mg, 5 mg to 1000 mg, 10 mg to 750 mg or 25 mg to 500 mg. Typically, unit dosage forms will contain about 0.5 mg to 0.5 g of a compound of this invention. In a particular embodiment the compound of the invention is administered parenterally, for example by intravenous administration. In another particular embodiment the compound of the invention is administered orally.

### Therapeutic Uses and Applications

In accordance with another aspect, the present invention provides a compound of the invention, or a pharmaceutically acceptable salt thereof, for use as a medicament.

A further aspect of the invention provides a compound of the invention, or a pharmaceutically acceptable salt thereof, for use in the treatment of a disease or medical condition mediated by adrenomedullin receptor subtype 2 receptors (AM₂).

Also provided is the use of a compound of the invention, or a pharmaceutically acceptable salt therefor in the manufacture of a medicament for the treatment of a disease or medical condition mediated by AM₂.

Also provided is a method of treating a disease or medical condition mediated by AM₂ in a subject in need thereof, the method comprising administering to the subject an effective amount of a compound of the invention, or a pharmaceutically acceptable salt thereof.

In the following sections of the application reference is made to a compound of the invention, or a pharmaceutically acceptable salt thereof for use in the treatment of certain diseases or conditions. It is to be understood that any reference herein to a compound for a particular use is also intended to be a reference to (i) the use of the compound of the invention, or pharmaceutically acceptable salt thereof, in the manufacture of a medicament for the treatment of that disease or condition; and (ii) a method of treating the disease or condition in a subject, the method comprising administering to the subject a therapeutically effective amount of the compound of the invention, or pharmaceutically acceptable salt thereof.

The disease of medical condition mediated by AM₂ may be any of the diseases or medical conditions listed in this application, for example a proliferative disease, particularly cancer.

The subject to which the compound of the invention is administered may be a warm-blooded mammal, for example human or animal. In particular embodiments the subject or patient is a human. In other embodiments the subject is an animal, for example a rat, mouse, dog, cat, a primate or a horse.

The association of AM and the AM₂ receptor with diseases in humans and animals is set out in the Background of the Invention. This disclosure and the associated references provide further support for the therapeutic uses of the compounds of the invention. As such the supporting references linking AM, the AM₂ receptor and its inhibition also form part of the disclosure of the utility of the compounds of the invention in the treatment and prevention of the medical conditions described herein.

The role of AM₂ is has distinct roles in diseases such as cancer. Accordingly the inhibition of AM₂ may be advantageous. The AM₂ receptor is a complex formed by the GPCR, calcitonin-like receptor (CLR) and RAMP3. The related AM₁ receptor is formed by CLR and RAMP2 and mediates a number of important physiological functions including blood pressure. Accordingly it is preferred that a compound of the invention selectively inhibits AM₂ and has little or no effect on the function of AM₁.

RAMP1 and RAMP3 also interact with the calcitonin receptor (CTR) to form two functional amylin receptors (AMY receptors). CTR and RAMP1 form the AMY, receptor, whilst CTR and RAMP3 form the AMY₃ receptor. Amylin has important roles in glycaemic control, by virtue of its co-secretion with insulin in response to changes in blood glucose, and its specific functions to slow rises in serum glucose by slowing gastric emptying, slowing of release of digestive enzymes and bile, and increasing feelings of satiety to reduce or inhibit further food intake. It also reduces secretion of glucagon, thereby reducing the production of new glucose and its release into the bloodstream. Amylin is also known to stimulate bone formation by direct anabolic effects on osteoblasts. These functions are achieved by Amylin's actions on the amylin receptors. Of these, it is believed that the AMY₁R and AMY₃R are responsible for these homeostatic functions. The AMY₂ receptor (formed by CTR and RAMP2) is not known to have physiological functions of significance. Blockade of blood glucose control is not a desirable function, and in cancer patients, reductions in appetite and failure to maintain normal levels of blood glucose would be seen as undesirable effects in a drug. Accordingly, preferred compounds of the invention selectively inhibit AM₂ over AMY, and/or AMY₃. Particular compounds of the invention are expected to provide potent AM₂ antagonists suitable for therapeutic use, whilst having little or no antagonistic effects on the AM₁ receptor because of its important role in blood pressure regulation. Suitably compounds of the invention have little or no effect on the CTR/RAMP3 AMY₃ receptor that is involved in physiological regulation of energy metabolism.

In embodiments a compound of the invention is 10-fold, 50-fold or-100 fold more active against AM₂ compared to one or more of AM₁, AMY₁ and/or AMY₃. In certain embodiments the compound of the invention selectively inhibits AM₂ compared to AM₁ and/or AMY₃. For example, the IC₅₀ of a compound of the invention in the AM₂ cell-based assay described in the Examples is 10-fold, 50-fold or 100-fold lower than the IC₅₀ in one or more corresponding assay using cell lines which express AM₁, AMY, or AMY₃ receptors.

Suitably the compounds of the invention selectively inhibit the AM₂ receptor over other receptors to which AM binds, for example by exhibiting 5-fold, 10-fold, 50-fold or 100-fold selectivity for the AM₂ receptor over other receptors to which AM binds.

### Proliferative Diseases

A further aspect of the invention provides a compound of the invention, or a pharmaceutically acceptable salt thereof, for use in the treatment of a proliferative disease. The proliferative disease may be malignant or non-malignant.

AM₂ is upregulated and plays a critical role in primary cancer and metastasiz. Accordingly in an embodiment there is provided a compound of the invention for use in the treatment of cancer, which may be non-metastatic or metastatic. The cancer is suitably a solid tumour, however, a compound of the invention may also be useful in the treatment of a haematological (“liquid”) cancers and effects associated with such cancers. There is evidence that haematological cancers express AM, and that its role in stimulating angiogenesis is important in disease progression (Kocemba K et al. The hypoxia target adrenomedullin is aberrantly expressed in multiple myeloma and promotes angiogenesis, Leukemia. 2013; 27:1729-1737: DOI 10.1038/leu.2013.76). Inhibiting AM₂ in the microenvironment of a tumour may be beneficial in preventing or inhibiting angiogenesis and disease progression associated with a cancer such as multiple myeloma.

Compounds of the invention may useful in the treatment and/or prevention of, for example:

Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumour), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma and non-small cell carcinoma of the lung, lung adenocarcinoma, squamous cell carcinoma, large cell carcinoma, bronchioloalveolar carcinoma, and mesothelioma), neuroendocrine tumours (including but not limited to carcinoids of the gastrointestinal tract, breast, and other organs), adrenocortical carcinoma, thyroid carcinoma, pancreatic carcinoma, breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumour including serous tumour, endometrioid tumour and mucinous cystadenocarcinoma, sex-cord-stromal tumour), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumours, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumour), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumours), testicular carcinoma, osteogenic carcinoma, epithelial carcinoma, sarcomatoid carcinoma, nasopharyngeal carcinoma, laryngeal carcinoma; oral and oropharyngeal squamous carcinoma;
Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumour (mixed connective tissue types) and other soft tissue sarcomas;
Solid tumours of the nervous system including medulloblastoma, craniopharyngioma, ependymoma, pinealoma, hemangioblastoma, acoustic neuroma, oligodendroglioma, meningioma, neuroblastoma and schwannoma;
Melanoma, uveal melanoma and retinoblastoma;
Myeloma and multiple myeloma;
Hematopoietic tumours, including: myelogenous and granulocytic leukaemia (malignancy of the myeloid and granulocytic white blood cell series); lymphatic, lymphocytic, and lymphoblastic leukaemia (malignancy of the lymphoid and lymphocytic blood cell series); polycythemia vera and erythremia (malignancy of various blood cell products, but with red cells predominating); myelofibrosis; and
Lymphomas, including: Hodgkin and Non-Hodgkin lymphomas.

In an embodiment a compound of the invention, or a pharmaceutically acceptable salt thereof is for use in the treatment of a solid tumour, for example any of the solid tumours listed above. In a particular embodiment a compound of the invention, or a pharmaceutically acceptable salt thereof is for use in the treatment of a cancer selected from: pancreatic, colorectal, breast, lung and bone cancer.

In another embodiment the compound of the invention, or a pharmaceutically acceptable salt thereof, is for use in the treatment of hormone dependent prostate cancer.

In another embodiment the compound of the invention, or a pharmaceutically acceptable salt thereof, is for use in the treatment of a breast cancer selected from Luminal A breast cancer (hormone-receptor positive (estrogen-receptor and/or progesterone-receptor positive), HER2 negative and low levels of the protein Ki-67); Luminal B breast cancer (hormone-receptor positive (estrogen-receptor and/or progesterone-receptor positive), and either HER2 positive or HER2 negative with high levels of Ki-67); triple negative breast cancer (i.e. the tumour is estrogen receptor-negative, progesterone receptor-negative and HER2-negative); HER2 positive breast cancer or normal-like breast cancer (classifications as defined in Table 1 of Dai et al. Am. J. Cancer Research. 2015; 5(10):2929-2943).

In an embodiment a compound of the invention, or a pharmaceutically acceptable salt thereof is for use in the treatment of a cancer selected from: pancreatic cancer, triple negative breast cancer (i.e. the tumour is estrogen receptor-negative, progesterone receptor-negative and HER2-negative), hormone refractory prostate cancer and non-small cell lung cancer.

In embodiments the compounds of the invention provide an anti-cancer effect on a cancer (for example any of the cancers disclosed herein) selected from one or more of an anti-proliferative effect, a pro-apoptotic effect, an anti-mitotic effect an anti-angiogenic effect, inhibition of cell migration, inhibition or prevention of tumour invasion and/or preventing or inhibiting metastasiz.

Compounds of the invention may be used to prevent or inhibit the progression of a cancer. A compound of the invention may be for use in slowing, delaying or stopping cancer progression. The progress of a cancer is typically determined by assigning a stage to the cancer. Staging is typically carried out by assigning a number from I to IV to the cancer, with I being an isolated cancer and IV being an advanced stage of the disease where the cancer that has spread to other organs. The stage generally takes into account the size of a tumour, whether it has invaded adjacent organs, the number of lymph nodes it has spread to, and whether the cancer has metastasized. Preventing or inhibiting progression of the cancer is particularly important for preventing the spread of the cancer, for example the progression from Stage I to Stage II where the cancer spreads locally, or the progression from Stage III to Stage IV where the cancer metastasizes to other organs.

It may be that a compound of the invention is for use in the treatment of a cancer wherein the cancer is a primary cancer, which may be a second primary cancer.

It may be that a compound of the invention is for use in the prevention or inhibition of occurrence of a second primary cancer.

It may be that a compound of the invention is for use in the treatment of a cancer wherein the cancer is refractory (resistant) to chemotherapy and/or radio therapy. The cancer may be resistant at the beginning of treatment or it may become resistant during treatment.

It may be that a compound of the invention is for use in the treatment of a cancer wherein the cancer is a recurrent cancer, which may be local, regional or distant. A recurrent cancer is a cancer which returns after initial treatment and after a period of time during which the cancer cannot be detected. The same cancer may return in the same tissue or in a different part of the body.

It may be that a compound of the invention is for use in the prevention or inhibition of recurrence of a cancer.

It may be that a compound of the invention is for use in the treatment of a cancer wherein the cancer is a metastatic or secondary cancer.

It may be that a compound of the invention is for use in the prevention or inhibition of cancer metastasis. The treatment of a metastatic cancer may be the same or different to the therapy previously used to treat the primary tumour. For example, in certain embodiments, a primary tumour may be surgically resected and a compound of the invention is for use in preventing the spread of cancer cells that may remain following surgery, or which may have already escaped the primary tumour. In other embodiments, the primary tumour may be treated using radiotherapy. In yet other embodiments, the primary tumour may be treated by chemotherapy. Combination therapies are commonly used to treat cancer to improve the treatment and, typically, maximise the length and depth of the remission. Any of the combination therapies disclosed herein may be used with a compound of the invention.

When the primary tumour has already metastasized and a secondary tumour has established, a compound of the invention may be used to treat the secondary tumour. This may involve both treatment of the secondary tumour and prevention of that secondary tumour metastasizing. Reference to metastasis herein is intended to encompass metastasis of any of the tumours disclosed herein. Generally, the secondary tumour will be in a different tissue to that of the primary tumour. For example the secondary tumour may be a secondary tumour in bone. In a particular embodiment a compound of the invention is for use in the treatment of a secondary tumour in bone, for example for use in the treatment of a secondary bone tumour, wherein the primary tumour is a breast or prostate tumour.

### Pancreatic Tumours

In an embodiment a compound of the invention, or a pharmaceutically acceptable salt thereof is for use in the treatment of a pancreatic tumour, especially a malignant pancreatic tumour. The term “pancreatic tumour” encompasses exocrine and endocrine tumours which may be benign or malignant. Exocrine tumours are the most prevalent forms of pancreatic cancer and account for about 95% of cases. Exocrine cancers include, for example ductal adenocarcinomas (PDAC), acinar cell carcinoma, papillary tumours (for example intraductal papillary-mucinous neoplasm (IPMN)), mucinous tumours (for example Mucinous cystadenocarcinoma), solid tumours and serous tumours. Pancreatic endocrine tumours are rare and develop as a result of abnormalities in islet cells within the pancreas. Examples of pancreatic endocrine tumours include gastrinoma (Zollinger-Ellison Syndrome), glucagonoma, insulinoma, somatostatinoma, VIPoma (Verner-Morrison Syndrome), nonfunctional islet cell tumour and multiple endocrine neoplasia type-1 (MEN1 also known as Wermer Syndrome). In a particular embodiment the compound is for use in the treatment of pancreatic cancer, particularly a pancreatic cancer selected from: pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumours. In another particular embodiment the pancreatic cancer is pancreatic adenocarcinoma.

It may be that the compound of the invention is for use in the treatment of pancreatic cancer in a patient wherein the tumour is resectable. In this embodiment a compound of the invention is administered to the patient as an adjunctive therapy following surgical resection of the tumour.

In some embodiments, the compounds of the invention are for use in the treatment of early stage pancreatic cancer. In some embodiments, the pancreatic cancer is late stage pancreatic cancer. In some embodiments, the pancreatic cancer is advanced pancreatic cancer. In some embodiments, the pancreatic cancer is locally advanced pancreatic cancer. In some embodiments, the pancreatic cancer is recurrent pancreatic cancer. In some embodiments, the pancreatic cancer is non-metastatic pancreatic cancer. In some embodiments, the pancreatic cancer is metastatic pancreatic cancer. In some embodiments, the pancreatic cancer is a primary pancreatic cancer. In some embodiments, the primary pancreatic tumour has metastasized. In some embodiments, the pancreatic cancer has reoccurred after remission. In some embodiments, the pancreatic cancer is progressive pancreatic cancer. In some embodiments, the pancreatic cancer is pancreatic cancer in remission.

In some embodiments the treatment of pancreatic cancer is an adjuvant treatment. An adjuvant treatment may be one in which the patient has had a history of pancreatic cancer, and generally (but not necessarily) been responsive to a therapy, which includes, but is not limited to, surgical resection, radiotherapy and/or chemotherapy; however, because of their history of cancer, the patient is considered to be at risk of development of the disease. Treatment or administration in the adjuvant setting refers to a subsequent mode of treatment.

In some embodiments, the treatment of pancreatic cancer may be a neoadjuvant treatment. By “neo-adjuvant” is meant that a compound of the invention is for use in the treatment of the patient before a primary/definitive therapy for the pancreatic cancer. In some embodiments the compounds of the invention are for use in the treatment of pancreatic cancer in a patient, wherein the patient has not previously been treated for pancreatic cancer.

In some embodiments the compounds of the invention are for use in the treatment of pancreatic cancer in a patient who has previously been treated, or is being concurrently treated, for the pancreatic cancer. The prior or concurrent treatment may include a chemotherapy agent for example a treatment selected from: gemcitabine, gemcitabine with Nab-paclitaxel (Abraxane™); 5-fluorouracil (5-FU), capecitabine, the combination treatment FOLFIRINOX (leucovorin, 5-FU, irinotecan and oxaliplatin), a combination of oxaliplatin and 5-FU (also known as FOLFOX) and a combination of gemcitabine and capecitabine. In some embodiments, the prior treatment comprises gemcitabine and/or erlotinib. In some embodiments, the prior treatment comprises 5-FU.

In some embodiments a compound of the invention is for use in the second or third-line treatment of a patient with pancreatic cancer. For example, wherein the patient has been prior treated with a first and/or second therapy that has failed or substantially failed.

It may be that the compound of the invention is for use in the treatment of pancreatic cancer which is refractory to conventional chemotherapy, for example in the treatment of pancreatic cancer refractory to gemcitabine and/or 5FU.

In some embodiments a compound of the invention is used in combination with another anti-cancer agent in the treatment of pancreatic cancer. Any of the combination treatment disclosed herein may be used.

In embodiments the compounds of the invention are for use in the treatment of pancreatic cancer in a patient, wherein the patient has developed atypical type 2 diabetes.

### Sézary Syndrome

Sézary syndrome is a rare cutaneous T-cell lymphoma. It is an aggressive cancer characterized by skin lesions, including widespread pruritic erythroderma and the presence of cancerous T cells (Sézary cells) in the blood, skin and/or lymph nodes. Subjects with Sézary syndrome also have enlarged lymph nodes (lymphadenopathy). The prognosis for patients diagnosed with Sézary syndrome is poor, with a 5-year survival rate of 30 to 40% (Agar et al. J. Clin. Oncol., 2010; 28:4730e9).

Current treatments for Sézary syndrome are limited and include conventional chemotherapy agents (e.g. anti-metabolites such as gemcitabine, methotrexate or pentostatin; topoisomerase inhibitors such as doxorubicin and liposomal forms thereof such as doxil; angiogenesis inhibitors such as lenalidomide; and alkylating agents such as cyclophosphamide); retinoids (e.g. bexarotene); HDAC inhibitors (e.g. romidepsin or vorinostat); immunotherapies, including anti-CD52 antibodies (e.g. alemtuzumab); antibody-drug conjugates (e.g. brentuximab vedotin); interferon-α or interlukin-2 therapy (e.g. denileukin difitox); phototherapy or radio therapy. There remains a need for new treatments for Sézary syndrome.

Prasad et. al. (Journal of Investigative Dermatology, 2016, 136, 1490-1499) identified certain somatic point mutations and somatic copy number variations, including duplication of RAMP3. As discussed herein, RAMP3 is also a component of the AM2 receptor. As illustrated in the Examples, herein, the inventors have identified that AM2 receptor inhibitor compounds are effective in reducing the viability of Sézary cells. The compounds of the invention may therefore be effective as a treatment for Sézary syndrome.

Accordingly also provided is a compound of the invention or a pharmaceutically acceptable salt thereof, for use in the treatment or prevention of Sézary syndrome. Also provided is a method of treating or preventing Sézary syndrome in a subject, the method comprising administering to the subject a therapeutically effective amount of a compound of the invention or a pharmaceutically acceptable salt thereof. In certain embodiments the compound of the invention is used as a monotherapy to treat Sézary syndrome. In certain other embodiments the compound of the invention is used in combination with another therapeutic agent, for example one or more of the anti-cancer agents and/or radiotherapies described herein. In particular embodiments the compound of the invention is used in combination with one or more of the existing treatments for Sézary syndrome, including one or more of the Sézary syndrome treatments described above.

### Benign Proliferative Disease

A compound of the invention, or a pharmaceutically acceptable salt thereof the invention may be for use in the treatment of a benign proliferative disease. The benign disease may be a benign tumour, for example hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, moles, uterine fibroids, thyroid adenomas, adrenocortical adenomas or pituitary adenomas

### Patient Selection and Biomarkers

Serum AM is up-regulated in a number of cancers, for example human pancreatic cancer. AM is also upregulated in tissue sections from pancreatic cancer patients, compared with normal tissue and pancreatitis. Additionally, the AM₂ receptor, or components thereof (i.e. CLR and/or RAMP3) are expressed in the majority of pancreatic tumours (Keleg et al. 2007). Pancreatic cancer patients have increased numbers of secreted exosomes containing AM. Evidence suggests these AM containing exosomes cause the paraneoplastic β-cell dysfunction that is frequently associated with the development of pancreatic cancer (Javeed et al 2015). Accordingly, a compound of the invention is expected to be beneficial in the treatment of a cancer, for example pancreatic cancer, wherein AM is upregulated in a biological sample compared to a reference sample. The biological sample may be, for example, a serum sample or a tissue sample, for example a tumour biopsy.

A compound of the invention is expected to be beneficial in the treatment of a cancer, for example pancreatic cancer, wherein AM₂ is upregulated in a biological sample compared to a reference sample. A compound of the invention is expected to be beneficial in the treatment of a cancer, for example pancreatic cancer, wherein components of AM₂; namely CLR and/or RAMP3 are upregulated in a biological sample compared to a reference sample, whether independently or in concert. The biological sample may be, for example, a serum sample or a tissue sample, for example a tumour biopsy. Additionally, in the case of RAMP3, expression of which is elevated in the healthy tissue surrounding tumours (Brekhman, V et al., The FASEB Journal. 2011; 25(1): 55-65), the tissue sample may be from healthy tissue immediately surrounding tumour tissue. This tissue may display no other signs of cancerous or pre-cancerous condition, other than elevation of RAMP3 expression relative to a reference sample.

Since elevated expression of AM, AM₂, CLR, and/or RAMP3 when compared with controls may be indicative of a cancer, particularly early-stage pancreatic cancer, patients can be subdivided into distinct, clinically useful groups based on their gene expression profiles. In particular, elevated expression of one or more of these biomarkers is predictive of therapeutic responsiveness to compounds of the invention. An ability to determine the patients which will respond well to treatment with compounds of the invention enables the appropriate treatment to be administered to each patient in an efficient manner, without the necessity for lengthy trial and error and the associated side effects of unnecessary, inappropriate or untimely treatment.

Accordingly, the invention provides a method of predicting or determining therapeutic responsiveness to treatment with compounds of the invention, comprising the steps of:

(a) analysing a biological sample obtained from a subject to determine the expression levels of one or more biomarkers, wherein the biomarkers are selected from AM and/or AM₂ and/or CLR and/or RAMP3; and

(b) comparing the expression levels of the biomarkers determined in (a) with one or more reference values, wherein an increase in the expression levels of the one or more biomarkers in the sample(s) from the subject compared to the one or more reference values is indicative of therapeutic responsiveness to treatment with compounds of the invention and/or is indicative of the presence of a cancer, for example early-stage pancreatic cancer.

It will be appreciated that any of the biomarkers indicative of a cancer, for example early stage pancreatic cancer, that is AM and/or AM₂ and/or CLR and/or RAMP3 may be selected for analysis, whether independently or in combination, to determine therapeutic responsiveness to compounds of the invention.

Normally, the expression level of AM in a sample (for example a serum sample or a tumour sample) will be analysed and compared with one or more reference values. Preferably, the expression level of AM and/or AM₂ in a sample (for example a serum sample or a tumour sample) will be analysed and compared with one or more reference values. Preferably, the expression level of AM in a serum sample will be analysed and compared with one or more reference values.

Equally, the expression level of AM₂ receptor components, CLR or RAMP3 in a sample, (for example a tumour sample or circulating tumour cells) will be analysed and compared with one or more reference values. Additionally, circulating tumour cell free tumour DNA may be analysed in order to determine the presence of circulating tumour cell free tumour DNA coding for AM, AM₂, CLR or RAMP3, which may reveal or provide advance indication of potential expression of the one or more biomarkers.

An increase in the expression levels of the one or more biomarkers in the sample(s) from the subject compared to the one or more reference values is predictive of sensitivity to and/or therapeutic responsiveness to compounds of the invention. Preferably, an increase in the expression levels of AM in a serum sample from a subject compared to one or more reference values is predictive of sensitivity to and/or therapeutic responsiveness to compounds of the invention. Preferably, an increase in the expression levels of AM₂ in a serum sample from a subject compared to one or more reference values is predictive of sensitivity to and/or therapeutic responsiveness to compounds of the invention. More preferably, an increase in the expression levels of AM and AM₂ in a serum sample or a tumour sample from a subject compared to one or more reference values is predictive of sensitivity to and/or therapeutic responsiveness to compounds of the invention.

### Biomarkers

Throughout, biomarkers in the biological sample(s) from the subject are said to be differentially expressed and indicative of for example, early stage pancreatic cancer, where their expression levels are significantly up-regulated compared with one or more reference values. Depending on the individual biomarker, early stage pancreatic cancer may be diagnosed in a biological sample by an increase in expression level, scaled in relation to sample mean and sample variance, relative to those of one or more control samples or one or more reference values. Clearly, variation in the sensitivity of individual biomarkers, subject and samples means that different levels of confidence are attached to each biomarker. Biomarkers of the invention may be said to be significantly up-regulated (or elevated) when after scaling of biomarker expression levels in relation to sample mean and sample variance, they exhibit a 2-fold change compared with one or more control samples or one or more reference values. Preferably, said biomarkers will exhibit a 3-fold change or more compared with one or more control samples or one or more reference values. More preferably biomarkers of the invention will exhibit a 4-fold change or more compared with one or more control samples or one or more reference values. That is to say, in the case of increased expression level (up-regulation relative to reference values), the biomarker level will be more than double that of the reference value or that observed in the one or more control samples. Preferably, the biomarker level will be more than 3 times the level of the one or more reference values or that in the one or more control samples. More preferably, the biomarker level will be more than 4 times the level of the one or more reference values or that in the one or more control samples.

### Biomarker Reference Sequences

### AM

As used herein “AM” designates “adrenomedullin”. A reference sequence of full-length human AM mRNA transcript is available from the GenBank database under accession number NM_001124, version NM_001124.2.

### AM₂

As used herein “AM₂” designates the “adrenomedullin receptor subtype 2”. A reference sequence of full-length human AM₂ mRNA transcript is available from the GenBank database under accession number NM_001253845, version NM_001253845.1.

### CLR

As used herein “CLR” designates the “calcitonin-like receptor”. A reference sequence of full-length human CLR mRNA transcript variant 1 is available from the NCBI-GenBank database under accession number NM_005795, version NM_005795.5. A reference sequence of full-length human CLR mRNA transcript variant 2 is available from the GenBank database under accession number NM_214095, version NM_214095.1.

### RAMP3

As used herein “RAMP3” designates the “receptor activity modifying protein 3”. A reference sequence of full-length human RAMP3 mRNA transcript is available from the NCBI-GenBank database under accession number NM_005856, version NM_005856.2.

All accession and version numbers of the reference sequences of biomarkers disclosed herein were obtained from the NCBI-GenBank database (Flat File Release 218.0) available at https://www.ncbi.nlm.nih.gov/genbank/.

### Reference Values

Throughout, the term “reference value” may refer to a pre-determined reference value, for instance specifying a confidence interval or threshold value for the diagnosis or prediction of the susceptibility of a subject to early stage pancreatic cancer. Preferably, “reference value” may refer to a pre-determined reference value, specifying a confidence interval or threshold value for the prediction of sensitivity to and/or therapeutic responsiveness to a compound of the invention. Alternatively, the reference value may be derived from the expression level of a corresponding biomarker or biomarkers in a ‘control’ biological sample, for example a positive (e.g. cancerous or known pre-cancerous) or negative (e.g. healthy) control. Furthermore, the reference value may be an ‘internal’ standard or range of internal standards, for example a known concentration of a protein, transcript, label or compound. Alternatively, the reference value may be an internal technical control for the calibration of expression values or to validate the quality of the sample or measurement techniques. This may involve a measurement of one or several transcripts within the sample which are known to be constitutively expressed or expressed at a known level. Accordingly, it would be routine for the skilled person to apply these known techniques alone or in combination in order to quantify the level of biomarker in a sample relative to standards or other transcripts or proteins or in order to validate the quality of the biological sample, the assay or statistical analysis.

### Biological Samples

Typically, the biological sample of the invention will be selected from a serum sample, a tissue sample or a tumour tissue sample. Normally, the biological sample of the invention will be a serum sample. Elevated levels of AM and/or AM₂ expression may be detectable in the serum of a subject with early-stage pancreatic cancer. Elevated expression levels of AM and/or AM₂ and/or CLR and/or RAMP3 expression may be detectable in the cells of a tumour sample of a subject with a cancer, for example early-stage pancreatic cancer. These cells may be, for example derived from a biopsy of a tumour or may be circulating tumour cells. Similarly, circulating tumour cell free tumour DNA may usefully be analysed for the presence of DNA encoding any of the one or more biomarkers, in particular that of the AM₂ receptor components, CLR and/or RAMP3, which may indicate or foreshadow the potential expression of the one or more biomarkers. In the case of RAMP3 expression, elevated levels of RAMP3, indicative of a cancer, for example early-stage pancreatic cancer, may be detectable in a sample of tissue taken from the area surrounding tumour tissue of a subject with early-stage pancreatic cancer. Such tissue may be otherwise asymptomatic.

Suitably, methods of the invention may make use of a range of biological samples taken from a subject to determine the expression level of a biomarker selected from AM and/or AM₂ and/or CLR and/or RAMP3.

Elevated levels of AM and/or AM₂ expression in serum and/or tissue and/or tumour tissue samples when compared with one or more reference values or reference serum and/or tissue and/or tumour tissue samples is indicative of early-stage pancreatic cancer. Elevated levels of CLR and/or RAMP3 expression in tumour tissue samples when compared with one or more reference values or reference tumour tissue samples is indicative of early-stage pancreatic cancer. Elevated levels of AM and/or AM₂ and/or CLR and/or RAMP3 expression in a biological sample when compared with one or more reference values or reference biological samples may suitably be discerned at the transcript (mRNA) and/or protein level. Most conveniently, elevated levels of AM and/or AM₂ and/or CLR and/or RAMP3 expression in biological samples when compared with one or more reference values or control biological samples are detectable at the transcript (mRNA) level.

Suitably, the biomarkers are selected from the group consisting of: biomarker protein; and nucleic acid molecule encoding the biomarker protein. It is preferred that the biomarker is a nucleic acid molecule, and particularly preferred that it is an mRNA molecule.

It is preferred that the levels of the biomarkers in the biological sample are investigated using specific binding partners. Suitably the binding partners may be selected from the group consisting of: complementary nucleic acids; aptamers; antibodies or antibody fragments. Suitable classes of binding partners for any given biomarker will be apparent to the skilled person.

Suitably, the levels of the biomarkers in the biological sample may be detected by direct assessment of binding between the target molecules and binding partners.

Conveniently, the levels of the biomarkers in the biological sample are detected using a reporter moiety attached to a binding partner. Preferably, the reporter moiety is selected from the group consisting of: fluorophores; chromogenic substrates; and chromogenic enzymes.

### Binding Partners

Expression levels of the biomarkers in a biological sample may be investigated using binding partners which bind or hybridize specifically to the biomarkers or a fragment thereof. In relation to the present invention the term ‘binding partners’ may include any ligands, which are capable of binding specifically to the relevant biomarker and/or nucleotide or peptide variants thereof with high affinity. Said ligands include, but are not limited to, nucleic acids (DNA or RNA), proteins, peptides, antibodies, antibody-conjugates, synthetic affinity probes, carbohydrates, lipids, artificial molecules or small organic molecules such as drugs. In certain embodiments the binding partners may be selected from the group comprising: complementary nucleic acids; aptamers; antibodies or antibody fragments. In the case of detecting mRNAs, nucleic acids represent highly suitable binding partners.

In the context of the invention, a binding partner which binds specifically to a biomarker should be taken as requiring that the binding partner should be capable of binding to at least one such biomarker in a manner that can be distinguished from non-specific binding to molecules that are not biomarkers. A suitable distinction may, for example, be based on distinguishable differences in the magnitude of such binding.

In preferred embodiments of the methods of the invention, the biomarker is a nucleic acid, preferably an mRNA molecule, and the binding partner is selected from the group comprising; complementary nucleic acids or aptamers.

Suitably, the binding partner may be a nucleic acid molecule (typically DNA, but it can be RNA) having a sequence which is complementary to the sequence the relevant mRNA or cDNA against which it is targeted. Such a nucleic acid is often referred to as a ‘probe’ (or a reporter or an oligo) and the complementary sequence to which it binds is often referred to as the ‘target’. Probe-target hybridization is usually detected and quantified by detection of fluorophore-, silver-, or chemiluminescence-labelled targets to determine relative abundance of nucleic acid sequences in the target.

Probes can be from 25 to 1000 nucleotides in length. However, lengths of 30 to 100 nucleotides are preferred, and probes of around 50 nucleotides in length are commonly used with success in complete transcriptome analysis.

Whilst the determination of suitable probes can be difficult, e.g. in very complex arrays, there are many commercial sources of complete transcriptome arrays available, and it is routine to develop bespoke arrays to detect any given set of specific mRNAs using publicly available sequence information. Commercial sources of microarrays for transcriptome analysis include Illumina and Affymetrix.

It will be appreciated that effective nucleotide probe sequences may be routinely designed to any sequence region of the biomarker transcripts of AM (NM_001124.2), AM₂ (NM_001253845.1), CLR (CLR variant 1: NM_005795.5, CLR variant 2: NM_214095.1) or RAMP3 (NM_005856.2) or a variant thereof in order to specifically detect, and measure expression thereof. The person skilled in the art will appreciate that the effectiveness of the particular probes chosen will vary, amongst other things, according to the platform used to measure transcript abundance, the sequence region that the probe binds to and the hybridization conditions employed.

Alternatively, the biomarker may be a protein, and the binding partner may suitably be selected from the group comprising; antibodies, antibody-conjugates, antibody fragments or aptamers. Such a binding partner will be capable of specifically binding to an AM, AM₂, CLR or RAMP3 protein in order to detect and measure the expression thereof.

Polynucleotides encoding any of the specific binding partners of biomarkers of the invention recited above may be isolated and/or purified nucleic acid molecules and may be RNA or DNA molecules.

Throughout, the term “polynucleotide” as used herein refers to a deoxyribonucleotide or ribonucleotide polymer in single- or double-stranded form, or sense or anti-sense, and encompasses analogues of naturally occurring nucleotides that hybridize to nucleic acids in a manner similar to naturally occurring nucleotides. Such polynucleotides may be derived from Homo sapiens, or may be synthetic or may be derived from any other organism.

Commonly, polypeptide sequences and polynucleotides used as binding partners in the present invention may be isolated or purified. By “purified” is meant that they are substantially free from other cellular components or material, or culture medium. “Isolated” means that they may also be free of naturally occurring sequences which flank the native sequence, for example in the case of nucleic acid molecule, isolated may mean that it is free of 5′ and 3′ regulatory sequences.

In preferred embodiments of methods of the invention, the nucleic acid is mRNA. There are numerous suitable techniques known in the art for the quantitative measurement of mRNA transcript levels in a given biological sample. These techniques include but are not limited to; “Northern” RNA blotting, Real Time Polymerase Chain Reaction (RTPCR), Quantitative Polymerase Chain Reaction (qPCR), digital PCR (dPCR), multiplex PCR, Reverse Transcription Quantitative Polymerase Chain Reaction (RT-qPCR) or by high-throughput analysis such as hybridization microarray, Next Generation Sequencing (NGS) or by direct mRNA quantification, for example by “Nanopore” sequencing. Alternatively, “tag based” technologies may be used, which include but are not limited to Serial Analysis of Gene Expression (SAGE). Commonly, the levels of biomarker mRNA transcript in a given biological sample may be determined by hybridization to specific complementary nucleotide probes on a hybridization microarray or “chip”, by Bead Array Microarray technology or by RNA-Seq where sequence data is matched to a reference genome or reference sequences.

In a preferred embodiment, where the nucleic acid is mRNA, the present invention provides a method of predicting or determining therapeutic responsiveness to treatment with compounds of the invention, wherein the levels of biomarker transcript(s) are determined by PCR. A variety of suitable PCR amplification-based technologies are well known in the art. PCR applications are routine in the art and the skilled person will be able to select appropriate polymerases, buffers, reporter moieties and reaction conditions. Preferably mRNA transcript abundance will be determined by qPCR, dPCR or multiplex PCR. Nucleotide primer sequences may routinely be designed to any sequence region of the biomarker transcripts of AM (NM_001124.2), AM₂ (NM_001253845.1), CLR (CLR variant 1: NM_005795.5, CLR variant 2: NM_214095.1) or RAMP3 (NM_005856.2) or a variant thereof, by methods which are well-known in the art. Consequently, the person skilled in the art will appreciate that effective primers can be designed to different regions of the transcript or cDNA of biomarkers selected from AM, AM₂, CLR or RAMP3, and that the effectiveness of the particular primers chosen will vary, amongst other things, according to the region selected, the platform used to measure transcript abundance, the biological sample and the hybridization conditions employed. It will therefore be appreciated that providing they allow specific amplification of the relevant cDNA, in principle primers targeting any region of the transcript may be used in accordance with the present invention. However, the person skilled in the art will recognise that in designing appropriate primer sequences to detect biomarker expression, it is required that the primer sequences be capable of binding selectively and specifically to the cDNA sequences of biomarkers corresponding to AM (NM_001124.2), AM₂ (NM_001253845.1), CLR (CLR variant 1: NM_005795.5, CLR variant 2: NM_214095.1) or RAMP3 (NM_005856.2) or fragments or variants thereof. Suitable binding partners are preferably nucleic acid primers adapted to bind specifically to the cDNA transcripts of biomarkers, as discussed above. Depending on the sample involved, preferably primers will be provided that specifically target either AM, AM₂, CLR or RAMP3.

Many different techniques known in the art are suitable for detecting binding of the target sequence and for high-throughput screening and analysis of protein interactions. According to the present invention, appropriate techniques include (either independently or in combination), but are not limited to; co-immunoprecipitation, bimolecular fluorescence complementation (BiFC), dual expression recombinase based (DERB) single vector system, affinity electrophoresis, pull-down assays, label transfer, yeast two-hybrid screens, phage display, in-vivo crosslinking, tandem affinity purification (TAP), ChIP assays, chemical crosslinking followed by high mass MALDI mass spectrometry, strep-protein interaction experiment (SPINE), quantitative immunoprecipitation combined with knock-down (QUICK), proximity ligation assay (PLA), bio-layer interferometry, dual polarisation interferometry (DPI), static light scattering (SLS), dynamic light scattering (DLS), surface plasmon resonance (SPR), fluorescence correlation spectroscopy, fluorescence resonance energy transfer (FRET), isothermal titration calorimetry (ITC), microscale thermophoresis (MST), chromatin immunoprecipitation assay, electrophoretic mobility shift assay, pull-down assay, microplate capture and detection assay, reporter assay, RNase protection assay, FISH/ISH co-localization, microarrays, microsphere arrays or silicon nanowire (SiNW)-based detection. Where biomarker protein levels are to be quantified, preferably the interactions between the binding partner and biomarker protein will be analysed using antibodies with a fluorescent reporter attached.

In certain embodiments of the invention, the expression level of a particular biomarker may be detected by direct assessment of binding of the biomarker to its binding partner. Suitable examples of such methods in accordance with this embodiment of the invention may utilise techniques such as electro-impedance spectroscopy (EIS) to directly assess binding of binding partners (e.g. antibodies) to target biomarkers (e.g. biomarker proteins).

In certain embodiments of the invention, the binding partner may be an antibody, antibody-conjugate or antibody fragment, and the detection of the target molecules utilises an immunological method. In certain embodiments of the methods or devices, the immunological method may be an enzyme-linked immunosorbent assay (ELISA) or utilise a lateral flow device.

A method of the invention may further comprise quantification of the amount of the target molecule indicative of expression of the biomarkers present in the biological sample from a subject. Suitable methods of the invention, in which the amount of the target molecule present has been quantified, and the volume of the patient sample is known, may further comprise determination of the concentration of the target molecules present in the patient sample which may be used as the basis of a qualitative assessment of the subject's condition, which may, in turn, be used to suggest a suitable course of treatment for the subject, for example, treatment with one or more of the compounds of the invention.

### Reporter Moieties

In certain embodiments of the present invention the expression levels of the protein in a biological sample may be determined. In some instances, it may be possible to directly determine expression, e.g. as with GFP or by enzymatic action of the protein of interest (POI) to generate a detectable optical signal. However, in some instances it may be chosen to determine physical expression, e.g. by antibody probing, and rely on separate test to verify that physical expression is accompanied by the required function.

In certain embodiments of the invention, the expression levels of a particular biomarker will be detectable in a biological sample by a high-throughput screening method, for example, relying on detection of an optical signal, for instance using reporter moieties. For this purpose, it may be necessary for the specific binding partner to incorporate a tag, or be labelled with a removable tag, which permits detection of expression. Such a tag may be, for example, a fluorescence reporter molecule. Such a tag may provide a suitable marker for visualisation of biomarker expression since its expression can be simply and directly assayed by fluorescence measurement in-vitro or on an array. Alternatively, it may be an enzyme which can be used to generate an optical signal. Tags used for detection of expression may also be antigen peptide tags. Similarly, reporter moieties may be selected from the group consisting of fluorophores; chromogenic substrates; and chromogenic enzymes. Other kinds of label may be used to mark a nucleic acid binding partner including organic dye molecules, radiolabels and spin labels which may be small molecules.

Conveniently, the levels of a biomarker or several biomarkers may be quantified by measuring the specific hybridization of a complementary nucleotide probe to the biomarker of interest under high-stringency or very high-stringency conditions.

Conveniently, probe-biomarker hybridization may be detected and quantified by detection of fluorophore-, silver-, or chemiluminescence-labelled probes to determine relative abundance of biomarker nucleic acid sequences in the sample. Alternatively, levels of biomarker mRNA transcript abundance can be determined directly by RNA sequencing or nanopore sequencing technologies.

The methods of the invention may make use of molecules selected from the group consisting of: the biomarker protein; and nucleic acid encoding the biomarker protein.

### Nucleotides and Hybridization Conditions

Throughout, the term “polynucleotide” as used herein refers to a deoxyribonucleotide or ribonucleotide polymer in single- or double-stranded form, or sense or anti-sense, and encompasses analogues of naturally occurring nucleotides that hybridize to nucleic acids in a manner similar to naturally occurring nucleotides.

The person skilled in the art would regard it as routine to design nucleotide probe sequences to any sequence region of the biomarker transcripts or cDNA sequences corresponding to AM (NM_001124.2), AM₂ (NM_001253845.1), CLR (CLR variant 1: NM_005795.5, CLR variant 2: NM_214095.1) or RAMP3 (NM_005856.2) or a fragment or variant thereof. This is also the case with nucleotide primers used where detection of expression levels is determined by PCR-based technology.

Of course the person skilled in the art will recognise that in designing appropriate probe sequences to detect biomarker expression, it is required that the probe sequences be capable of binding selectively and specifically to the transcripts or cDNA sequences of biomarkers corresponding to AM (NM_001124.2), AM₂ (NM_001253845.1), CLR (CLR variant 1: NM_005795.5, CLR variant 2: NM_214095.1) or RAMP3 (NM_005856.2) or fragments or variants thereof. The probe sequence will therefore be hybridizable to that nucleotide sequence, preferably under stringent conditions, more preferably very high stringency conditions. The term “stringent conditions” may be understood to describe a set of conditions for hybridization and washing and a variety of stringent hybridization conditions will be familiar to the skilled reader. Hybridization of a nucleic acid molecule occurs when two complementary nucleic acid molecules undergo an amount of hydrogen bonding to each other known as Watson-Crick base pairing. The stringency of hybridization can vary according to the environmental (i.e. chemical/physical/biological) conditions surrounding the nucleic acids, temperature, the nature of the hybridization method, and the composition and length of the nucleic acid molecules used. Calculations regarding hybridization conditions required for attaining particular degrees of stringency are discussed in Sambrook et al. (2001, Molecular Cloning: A Laboratory Manual, Cold Spring Harbor Laboratory Press, Cold Spring Harbor, N.Y.); and Tijssen (1993, Laboratory Techniques in Biochemistry and Molecular Biology—Hybridization with Nucleic Acid Probes Part I, Chapter 2, Elsevier, N.Y.). The Tm is the temperature at which 50% of a given strand of a nucleic acid molecule is hybridized to its complementary strand.

In any of the references herein to hybridization conditions, the following are exemplary and not limiting:

Very High Stringency (allows sequences that share at least 90% identity to hybridize)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (allows sequences that share at least 80% identity to hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (allows sequences that share at least 50% identity to hybridize)
Hybridization: 6×SSC at RT to 55° C. for 16-20 hours
Wash at least twice: 2×-3×SSC at RT to 55° C. for 20-30 minutes each.

In a further aspect, the present invention relates to a method of treating or preventing cancer in a subject, said method comprising administering a therapeutically effective amount of an AM₂ inhibitor, for example a compound of the invention, to said subject, wherein said subject has a cancer associated with expression of AM and/or CLR and/or RAMP3. Without wishing to be bound by theory it is possible that expression of AM by a tumour may interact with AM₂ receptors in healthy tissue resulting in, for example metastasis and/or angiogenesis and progression of the cancer. Accordingly the expression of AM and/or CLR and/or RAMP3 may be in the tumour or in healthy tissues, for example in healthy tissues surrounding a tumour.

Optionally, the method may comprise determining the levels of AM and/or CLR and/or RAMP3 in a biological sample of said subject, and administering a compound of the invention to said subject when the level AM and/or CLR and/or RAMP3 is determined to be expressed or expressed at increased levels in the biological sample relative to one or more reference values.

In a further aspect, the present invention relates to a method of identifying a subject having increased likelihood of responsiveness or sensitivity to an AM₂ inhibitor, for example a compound of the invention, comprising determining the level of one or more of AM, CLR and RAMP3 in a biological sample of the subject;

wherein increased levels of AM, CLR and/or RAMP3 compared to one or more reference values indicates an increased likelihood of responsiveness or sensitivity to an AM₂ inhibitor in the subject.

### Combination Therapies

The compounds of the invention may be used alone to provide a therapeutic effect. The compounds of the invention may also be used in combination with one or more additional anti-cancer agent and/or radiotherapy.

Such chemotherapy may include one or more of the following categories of anti-cancer agents:

(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nab paclitaxel (albumin-bound paclitaxel), docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine:
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), afatinib, vandetanib, osimertinib and rociletinib) erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors, for example dalotuzumab; aurora kinase inhibitors and cyclin dependent kinase inhibitors such as CDK2 and/or CDK4 inhibitors; CCR2, CCR4 or CCR6 antagonists; RAF kinase inhibitors such as those described in WO2006043090, WO2009077766, WO2011092469 or WO2015075483; and Hedgehog inhibitors, for example vismodegib.
(v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a VEGF receptor tyrosine kinase inhibitor such as vandetanib, vatalanib, sunitinib, axitinib, pazopanib and cabozantinib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); toll-like receptor modulators for example TLR-7 or TLR-9 agonists; PD-1, PD-L1, PD-L2 and CTL4-A modulators (for example Nivolumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab), CAR-T cell therapies; and
(viii) cytotoxic agents for example fludaribine (fludara), cladribine, pentostatin (Nipent™);
(ix) targeted therapies, for example PI3K inhibitors, for example idelalisib and perifosine; SMAC (second mitochondriaderived activator of caspases) mimetics, also known as Inhibitor of Apoptosis Proteins (IAP) antagonists (IAP antagonists). These agents act to supress IAPs, for example XIAP, cIAP1 and cIAP2, and thereby re-establish cellular apoptotic pathways. Particular SMAC mimetics include Birinapant (TL32711, TetraLogic Pharmaceuticals), LCL161 (Novartis), AEG40730 (Aegera Therapeutics), SM-164 (University of Michigan), LBW242 (Novartis), ML101 (Sanford-Burnham Medical Research Institute), AT-406 (Ascenta Therapeutics/University of Michigan), GDC-0917 (Genentech), AEG35156 (Aegera Therapeutic), and HGS1029 (Human Genome Sciences); and agents which target ubiquitin proteasome system (UPS), for example, bortezomib, carfilzomib, marizomib (NPI-0052) and MLN9708; a CXCR4 antagonist, for example plerixafor or BL-8040;
(x) PARP inhibitors, for example niraparib (MK-4827), talazoparib (BMN-673), veliparib (ABT-888); olaparib, CEP 9722, and BGB-290
(xi) chimeric antigen receptors, anticancer vaccines and arginase inhibitors;
(xii) agents which degrade hyaluronan, for example the hyaluronidase enzyme PEGPH20

The additional anti-cancer agent may be a single agent or one or more of the additional agents listed herein.

Particular anti-cancer agents which may be used together with a compound of the invention include for example erlotinib, cabozantinib, bevacizumab, dalotuzumab, olaparib, PEGPH20, vismodegib, paclitaxel (including nab paclitaxel), gemcitabine, oxaliplatin, irinotecan, leucovorin and 5-fluorouracil. In some embodiments the additional anti-cancer agent selected from capecitabine, gemcitabine and 5-fluorouracil (5FU).

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention there is provided a compound of the invention as defined hereinbefore and an additional anti-cancer agent as defined hereinbefore, for use in the conjoint treatment of cancer.

According to a further aspect of the invention there is provided a pharmaceutical product comprising a compound of the invention as defined hereinbefore and an additional anti-cancer agent as defined hereinbefore for the conjoint treatment of cancer.

According to a further aspect of the invention there is provided a method of treatment of a human or animal subject suffering from a cancer comprising administering to the subject a therapeutically effective amount of a compound of the invention, or a pharmaceutically acceptable salt thereof simultaneously, sequentially or separately with an additional anti-cancer agent as defined hereinbefore.

According to a further aspect of the invention there is provided a compound of the invention, or a pharmaceutically acceptable salt thereof for use simultaneously, sequentially or separately with an additional anti-cancer agent as defined hereinbefore, in the treatment of a cancer.

The compound of the invention may also be used be used in combination with radiotherapy. Suitable radiotherapy treatments include, for example X-ray therapy, proton beam therapy or electron beam therapies. Radiotherapy may also encompass the use of radionuclide agents, for example ¹³¹I, ³²P, ⁹⁰Y, ⁸⁹Sr, ¹⁵³Sm or ²²³Ra. Such radionuclide therapies are well known and commercially available.

According to a further aspect of the invention there is provided a compound of the invention, or a pharmaceutically acceptable salt thereof as defined hereinbefore for use in the treatment of cancer conjointly with radiotherapy.

According to a further aspect of the invention there is provided a method of treatment of a human or animal subject suffering from a cancer comprising administering to the subject a therapeutically effective amount of a compound of the invention, or a pharmaceutically acceptable salt thereof simultaneously, sequentially or separately with radiotherapy.

### Biological Assays

The biological effects of the compounds may be assessed using one of more of the assays described herein in the Examples.

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃.OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

Also provided is a process for the preparation of a compound of formula (I), or a pharmaceutically acceptable salt thereof, the process comprising coupling a compound of the formula (IX), or a salt thereof:

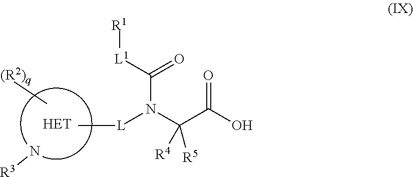

wherein HET, R¹, R², R³, R⁴, R⁵, L, L¹ and q have any of the meanings defined herein, except that any functional group is protected if necessary, with a compound of the formula (X), or a salt thereof:

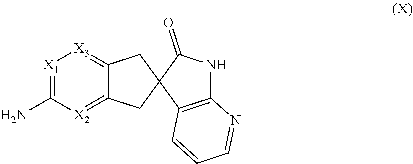

wherein X₁, X₂ and X₃ have any of the meanings defined herein, except that any functional group is protected if necessary;
and optionally thereafter carrying out one or more of the following procedures:
converting a compound of formula (I) into another compound of formula (I)
removing any protecting groups
forming a pharmaceutically acceptable salt.

In one embodiment in the compound of formula (X), X₂ and X₃ are CH; and X, is CR⁶, wherein R⁶ has any of the meanings defined herein (e.g. R⁶ is H), except that any functional group is protected if necessary.

The coupling reaction may be performed using well-known methods, for example by reacting the acid of formula (IX), or an activated derivative thereof, with the amine of formula (X) in the presence of a suitable coupling agent, for example: a carbodiimide (e.g. dicyclohexylcarbodiimide (DCC), or N-ethyl-N′-(3-dimethylaminopropyl)carbodiimide hydrochloride (EDCI)) optionally in combination with an additive such as hydroxybenzotriazole (HOBt) or 1-hydroxy7-azabenzotriazole (HOAt); a uronium or aminium salt e.g. 1-[bis(dimethylamino)methylene]-1H-1,2,3-triazolo[4,5-b]pyridinium 3-oxid hexafluorophosphate, (HATU), 2-(1H-benzotriazol-1-yl)-1,1,3,3-tetramethyluronium hexafluorophosphate (HBTU) or 2-(1H-benzotriazole-1-yl)-1,1,3,3-tetramethylaminium tetrafluoroborate (TBTU).

The acid of the formula (IX) may be activated by, for example, forming an acid halide. When the compound of formula (IX) is in the form of an acid halide it may be possible to react the compound directly with amine of formula (X) without the need for a coupling agent.

The reaction is suitably performed in a suitable solvent (e.g. DMF) and in the presence of a base, preferably a tertiary amine such as N,N-diisopropylethylamine.

Compounds of the formula (IX) and (X) may be prepared using analogous methods to those described in the Examples.

Also provided is a process for the preparation of a compound of formula (I), or a pharmaceutically acceptable salt thereof, the process comprising coupling a compound of the formula (XI), or a salt thereof:

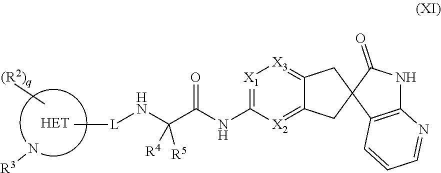

wherein HET, R², R³, R⁴, R⁵, L, X₁, X₂, X₃ and q have any of the meanings defined herein, except that any functional group is protected if necessary, with a compound of the formula with an acid of the formula: R¹L¹C(O)OH or an activated derivative thereof (e.g. an acid halide), wherein R¹ and L¹ have any of the meanings defined herein, except that any functional group is protected if necessary;
and thereafter carrying out one or more of the following procedures:
converting a compound of formula (I) into another compound of formula (I)
removing any protecting groups
forming a pharmaceutically acceptable salt.

Suitably L is absent.

The coupling may be carried out using analogous methods to those described above for the coupling of the compounds of formulae (IX) and (X).

The reaction is suitably performed in the presence of a solvent, for example a polar protic solvent such as N,N-dimethylformamide. The reaction is suitably performed in the presence of a tertiary organic amine base such as N,N-diisopropylethylamine. Compounds of the formula (XI) may be prepared using analogous conditions to those described in the Examples. Compounds of the formula R¹L¹C(O)R″ are commercially available or can be prepared using well-known methods.

Compounds of the formula (I) wherein R³ is H may be prepared by deprotecting a compound of the formula (XII), or a salt thereof:

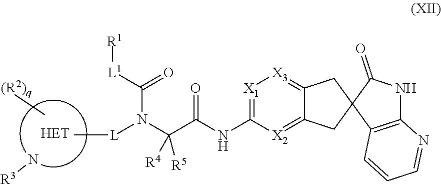

wherein HET, R¹, R², R³, R⁴, R⁵, L, L¹, X₁, X₂, X₃ and q have any of the meanings defined herein; and Pg is an amino protecting group.

Suitable amino protecting groups include, for example, those disclosed herein such as tert-butoxycarbonyl (BOC), benzyloxycarbonyl (CBz), and 9-fluorenylmethoxycarbonyl (Fmoc). Preferably Pg is BOC. The amino protecting group can be removed by conventional methods, for example treatment with a suitable acid or base.

Certain intermediates described herein are novel and form a further aspect of the invention. Accordingly, also provided is a compound of the formula (IX), (XI) or (XII).

In some embodiments the compound of the formula (IX) is a compound of the formula (IXa):

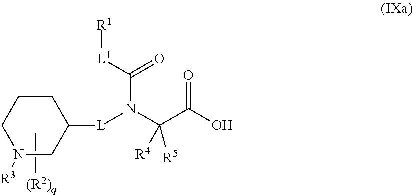

wherein R¹, R², R³, R⁴, R⁵, L, L¹ and q have any of the meanings defined herein, except that any functional group is protected if necessary.

In some embodiments the compound of the formula (IX) is a compound of the formula (IXb):

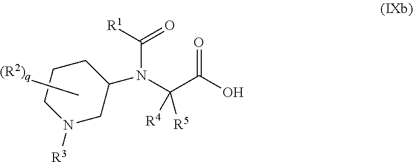

wherein R¹, R², R³, R⁴, R⁵ and q have any of the meanings defined herein, except that any functional group is protected if necessary.

In some embodiments the compound of the formula (IX) is a compound of the formula (IXc):

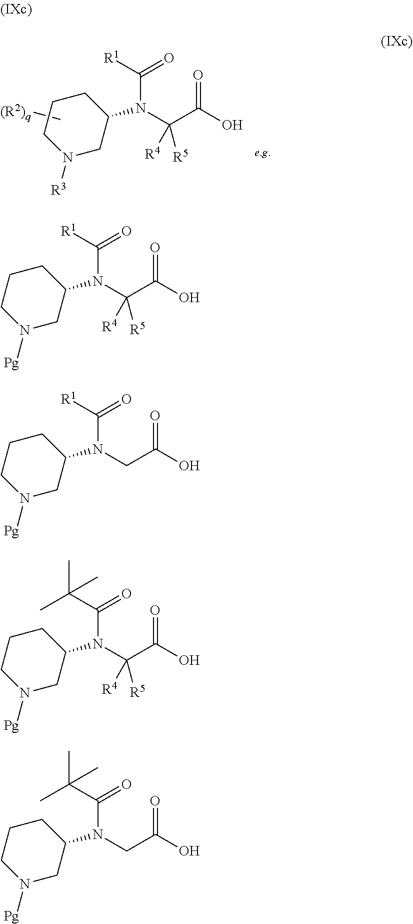

wherein R¹, R², R³, R⁴, R⁵ and q have any of the meanings defined herein, except that any functional group is protected if necessary; and Pg is an amino protecting group as defined herein (e.g. BOC).

In some embodiments the compound of the formula (XI) is a compound of the formula (XIa):

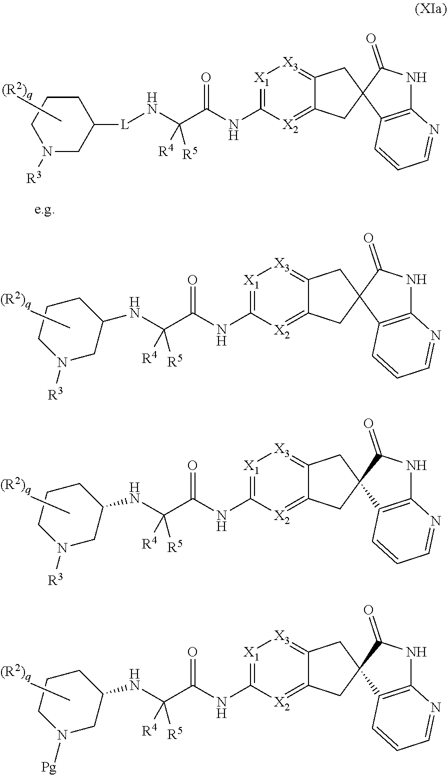

wherein R², R³, R⁴, R⁵, L and q have any of the meanings defined herein, except that any functional group is protected if necessary; and Pg is an amino protecting group as defined herein (e.g. BOC).

In some embodiments the compound of the formula (XII) is a compound of the formula (XIIa):

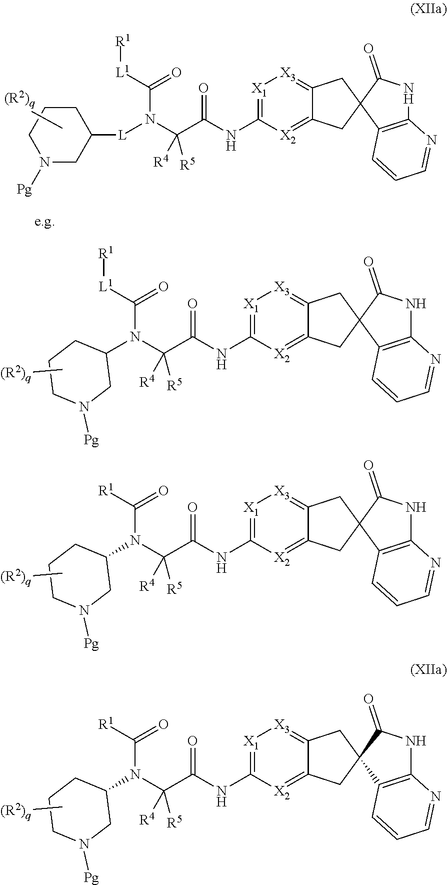

wherein R¹, R², R³, R⁴, R⁵, L, L¹, X₁, X₂, X₃ and q have any of the meanings defined herein and Pg is an amino protecting group as defined herein (e.g. BOC).

In certain embodiments in the compounds of the formula (IX), (IXa), (IXb), (IXc) (XI), (XIa), (XII) and (XIIa) L and L¹ are absent.

In certain embodiments in the compounds of the formula (IX), (IXa), (XII) and (XIIa) R¹ are has any of the values defined in (52) to (86) above.

In certain embodiments in the compounds of the formula (XI) and (XII) HET has any of the values defined in (1) to (38) above.

In certain embodiments in the compounds of the formula (XI), (XIa), (XII) and (XIIa)

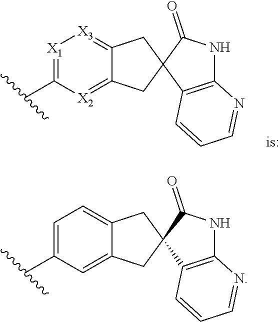

### EXAMPLES

### Abbreviations

BINAP—2,2′-bis(diphenylphosphino)-1,1′-binaphthyl
Bn—benzyl
Boc—tert-butoxycarbonyl
CBz—benzyloxycarbonyl
CPME—cyclopentyl methyl ether
DCM—dichloromethane
DEA—diethylamine
DIEA—N,N-diisopropylethylamine
DIPA—diisopropylamine
DMAc—dimethylacetamide
DMF—N,N-dimethylformamide
DMP—Dess-Martin periodinane
DMSO—dimethylsulfoxide
EDCI—1-ethyl-3-(3-dimethylaminopropyl)carbodiimide hydrochloride salt
ee—enantiomeric excess
eq.—equivalent(s)
Ghosez's Reagent—1-chloro-N,N,2-trimethyl-1-propenylamine
HATU—1-[bis(dimethylamino)methylene]-1H-1,2,3-triazolo[4,5-b]pyridinium 3-oxide hexafluorophosphate
HOAt—1-hydroxy-7-azabenotriazole
HPLC—high performance liquid chromatography
IPA—isopropanol
LC-MS—liquid chromatograph-mass spectrometer
LDA—lithium diisopropylamide
MeCN—acetonitrile
MS—mass spectrometry
NBS—N-bromosuccinimide
NMM—N-methylmorpholine
NMR—nuclear magnetic resonance
o/n—overnight
Pd/C—palladium-on-carbon
Piv—pivaloyl
Prep—preparative
pTSA—p-toluene sulfonic acid
Py—pyridine
rt—retention time
RT—room temperature
SEM—trimethylsilylethoxymethyl
SPE—solid phase extraction
Su—succinimide
TBAB—tetrabutylammonium bromide
TEA—triethylamine
TFA—trifluoroacetic acid
TFAA—trifluoroacetic anhydride
THF—tetrahydrofuran
TLC—Thin-layer Chromatograph

Reagents and Conditions

Unless syntheses are given, reagents and starting materials were obtained from commercial sources. All reactions, unless otherwise stated, were carried out under an inert atmosphere of either nitrogen or argon.

Compound Names

The exemplified compounds were named using ChemDraw Ultra 12.0 from CambridgeSoft. Other compounds, particularly commercial reagents, either use names generated by ChemDraw Ultra 12.0 or names commonly found in online databases and catalogues.

Analytical Methods

Method 1: (5-95AB_R_220&254): Instrument: SHIMADZU LC-MS-2020; Column: Kinetex® 30×2.1 mm, 5 μm S/N: H17-247175; Run Time: 1.55 min; Solvents A) 0.0375% TFA in water (v/v) B) 0.01875% TFA in acetonitrile (v/v). The gradient runs with 5% B. Gradient: 5-95% B with A 0.8 min, hold at 95% B to 1.21 min; 5% B at 1.21 min and hold at 5% B to 1.55 min at 1.5 mL/min, 50° C.

Method 2: (5-95AB_R_220&254.M): Instrument: Agilent 1200\G61 10A; Column: Chromolith® Flash RP-18e 25×2.0 mm; Run Time: 1.50 min; Solvents A) 0.0375% TFA in water (v/v) B) 0.01875% TFA in acetonitrile (v/v). The gradient runs with 5% B. Gradient: 5-95% B with A 0.8 min, hold at 95% B to 1.20 min; 5% B at 1.21 min and hold at 5% B to 1.50 min at 1.5 mL/min, 50° C.

Method 3: (WUXIAB00.M): Instrument: Agilent 1200 LC & Agilent 6110 MSD; Column: Agilent ZORBAX 5 μm SB-Aq, 2.1×50 mm; Run Time: 4.50 min; Solvents A) 0.0375% TFA in water (v/v) B) 0.01875% TFA in acetonitrile (v/v). The gradient runs with 0% B to 0.4 min. Gradient: 0-80% B with A 3.4 min. Gradient: 80-100% B with A 3.9 min; 0% B at 3.91 min and hold at 0% B to 4.50 min at 0-3.91 min, flow rate: 1.5 mL/min; 3.91-4.5 min, flow rate: 0.6 mL/min; 50° C.

Method 4: (0-60AB_4MIN_220&254.lcm): Instrument: SHIMADZU LC-MS-2020; Column: Kinetex® 30×2.1 mm, 5 μm S/N: H17-247175; Run Time: 1.55 min; Solvents A) 0.0375% TFA in water (v/v) B) 0.01875% TFA in acetonitrile (v/v). The gradient runs with 0% B. Gradient: 0-60% B with A 3 min, hold at 60% B to 3.5 min; 0% B at 3.51 min and hold at 0% B to 4.00 min at 0.8 mL/min, 50° C.

Method 5: (0-60AB_0-R_220&254.lcm): Instrument: SHIMADZU LC-MS-2020; Column: Kinetex® 30×2.1 mm, 5 μm S/N: H17-247175; Run Time: 1.55 min; Solvents A) 0.0375% TFA in water (v/v) B) 0.01875% TFA in acetonitrile (v/v). The gradient runs with 0% B. Gradient: 0-60% B with A 0.6 min, hold at 60% B to 1.21 min; 0% B at 1.21 min and hold at 0% B to 1.55 min at 1.5 mL/min, 50° C.

Method 6: (5-95AB_4 min_220&254): Instrument: SHIMADZU LC-MS-2020; Column: Kinetex® 30×2.1 mm, 5 μm S/N: H17-247175; Run Time: 1.55 min; Solvents A) 0.0375% TFA in water (v/v) B) 0.01875% TFA in acetonitrile (v/v). The gradient runs with 5% B. Gradient: 5-95% B with A 3.0 min, hold at 95% B to 3.5 min; 5% B at 3.51 min and hold at 5% B to 4.00 min at 0.8 mL/min, 50° C.

Method 7: (5-95AB_R_220&254_50): Instrument: SHIMADZU LC-MS-2020; Column: Chromolith® Flash RP-18E 25-2 MM; Run Time: 1.55 min; Solvents A) 0.0375% TFA in water (v/v) B) 0.01875% TFA in acetonitrile (v/v). The gradient runs with 5% B. Gradient: 5-95% B with A 0.8 min, hold at 95% B to 1.21 min; 5% B at 1.21 min and hold at 5% B to 1.55 min at 1.5 mL/min, 50° C.

Method 8: (WUXIAB10.M): Instrument: Agilent 1200 LC & Agilent 6110 MSD; Column: Agilent ZORBAX 5 μm SB-Aq, 2.1×50 mm; Run Time: 4.50 min; Solvents A) 0.0375% TFA in water (v/v) B) 0.01875% TFA in acetonitrile (v/v). The gradient runs with 10% B to 0.4 min. Gradient: 10-100% B with A 3.4 min, hold at 100% B to 3.9 min; 10% B at 3.91 min and hold at 10% B to 4.50 min at 0-3.91 min, flow rate: 0.8 mL/min; 3.91-4.5 min, flow rate: 1.0 mL/min; 50° C.

Method 9: (WUXIAB01.M): Instrument: Agilent 1200 LC & Agilent 6110 MSD; Column: Agilent ZORBAX 5 μm SB-Aq, 2.1×50 mm; Run Time: 4.50 min; Solvents A) 0.0375% TFA in water (v/v) B) 0.01875% TFA in acetonitrile (v/v). The gradient runs with 1% B to 0.4 min. Gradient: 1-90% B with A 3.4 min. Gradient: 90-100% B with A 3.9 min; 1% B at 3.91 min and hold at 1% B to 4.50 min at 0-3.91 min, flow rate: 0.8 mL/min; 3.91-4.5 min, flow rate: 1.0 mL/min; 50° C.

Method 10: (5-95CD_R_220&254_POS): Instrument: SHIMADZU LC-MS-2020; Column: Xbridge C18 30×3.0 mm, 5 μm; Run Time: 1.50 min; Solvents A) 0.025% ammonium hydroxide in water (v/v) B) acetonitrile. The gradient runs with 5% B. Gradient: 5-95% B with A 1.2 min, hold at 95% B to 1.60 min; 5% B at 1.61 min and hold at 5% B to 2.0 min at 2.0 mL/min, 40° C.

Method 11: (5-95AB_R_220&254_50): Instrument: Agilent 1200\G6110A; Column: Kinetexat 5 μm EVO C18 30×2.1 mm; Run Time: 1.50 min; Solvents A) 0.0375% TFA in water (v/v) B) 0.01875% TFA in acetonitrile (v/v). The gradient runs with 5% B. Gradient: 5-95% B with A 0.8 min, hold at 95% B to 1.20 min; 5% B at 1.21 min and hold at 5% B to 1.50 min at 1.5 mL/min, 50° C.

Method 12: (0-60AB_R_220&254): Instrument: SHIMADZU LC-MS-2020; Column: Chromolith® Flash RP-18E 25-2 MM; Run Time: 1.5 min; Solvents A) 0.0375% TFA in water (v/v) B) 0.01875% TFA in acetonitrile (v/v). The gradient runs with 0% B. Gradient: 0-60% B with A 0.8 min, hold at 60% B to 1.21 min; 5% B at 1.21 min and hold at 5% B to 1.55 min at 1.5 mL/min, 50° C.

Method 13: (0-60AB_0-R_220&254): Instrument: Agilent 1100\G1956A; Column: Kinetex® 5 μm EVO C18 30×2.1 mm; Run Time: 1.5 min; Solvents A) 0.0375% TFA in water (v/v) B) 0.01875% TFA in acetonitrile (v/v). The gradient runs with 0% B. Gradient: 0-60% B with A 0.8 min, hold at 60% B to 1.21 min; 5% B at 1.21 min and hold at 5% B to 1.5 min at 1.5 mL/min, 50° C.

Method 14: (5-95AB_4MIN_220&254): Instrument: Agilent 1200\G6110A; Column: Kinetex@ 5 μm EVO C18 30×2.1 mm; Run Time: 4.0 min; Solvents A) 0.0375% TFA in water (v/v) B) 0.01875% TFA in acetonitrile (v/v). The gradient runs with 5% B. Gradient: 5-95% B with A 3.0 min, hold at 95% B to 3.5 min; 5% B at 3.51 min and hold at 5% B to 4.00 min at 0.8 mL/min, 50° C.

Method 15: (0-60AB_4MIN_220&254): Instrument: Agilent 1200\G6410B; Column: Zorbax Extend C-18, 2.1×50 mm, 5 μm; Run Time: 4.0 min; Solvents A) 0.0375% TFA in water (v/v) B) 0.0188% TFA in acetonitrile (v/v). The gradient runs with 10% B. Gradient: 10-80% B with A 4.2 min. Gradient: 80-90% B with A 5.3 min; 10% B at 5.31 min and hold at 10% B to 7 min at 1 mL/min, 40° C.

Method 16: (5-95CD_4MIN_220&254_POS): Instrument: SHIMADZU LC-MS-2020; Column: Kinetex® EVO C18 2.1×30 mm, 5 μm; Run Time: 4.0 min; Solvents A) 0.025% ammonium hydroxide in water (v/v) B) acetonitrile. The gradient runs with 5% B. Gradient: 5-95% B with A 3.0 min, hold at 95% B to 3.5 min; 5% B at 3.51 min and hold at 5% B to 4.0 min at 0.8 mL/min, 40° C.

Method 17: (10-80CD_2MIN_220&254): Instrument: Agilent 1200\G6110A; Column: XBridge C18 2.1×50 mm, 5 μm; Run Time: 2.0 min; Solvents A) 0.025% ammonium hydroxide in water (v/v) B) acetonitrile. The gradient runs with 10% B. Gradient: 10-80% B with A 1.2 min, hold at 95% B to 1.6 min; 10% B at 1.61 min and hold at 10% B to 2.0 min at 1.2 mL/min, 40° C.

Supercritical fluid chromatography (SFC) analysis was performed on a Shimadzu LC-30AD instrument. Column: kromasil 3-Cellucoat 50×4.6 mm, particle size 3 μm. Method: mobile phase solvent A: carbon dioxide, phase solvent B: methanol (0.05% DEA), B in A from 0% to 95%, flow rate: 3.0 mL/min; wavelength: 220 nm

NMR

All NMR spectra were obtained using Bruker Avance 400 MHz spectrometers running ACD/Spectrus Processor.

Synthesis of Intermediate A

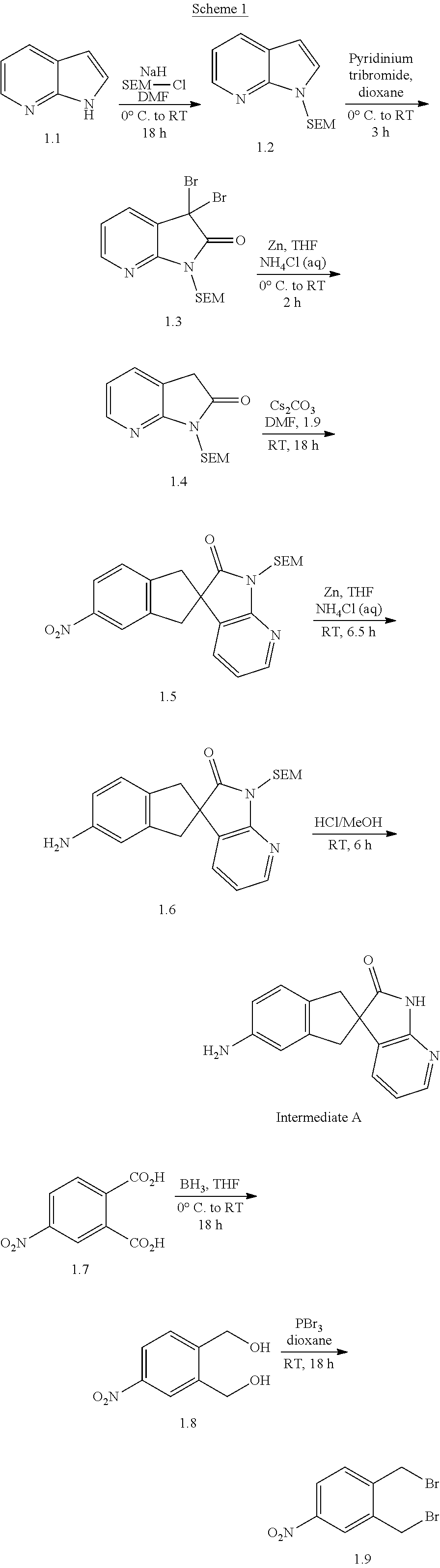

### 1-((2-(Trimethylsilyl)ethoxy)methyl)-1H-pyrrolo[2,3-b]pyridine 1.2

A solution of 7-azaindole 1.1 (95 g, 804 mmol) in dimethylformamide (500 mL) was cooled to 0° C., and then sodium hydride (38.6 g, 965 mmol) was added in several small portions, maintaining internal temperature below 10° C. The suspension was stirred at 0-5° C. for 1 h. 2-(Trimethylsilyl)ethoxymethyl chloride (171 mL, 965 mmol) was then added dropwise at 5-10° C. The yellow suspension was stirred at room temperature for 18 h. The mixture was quenched by slow addition of water until effervescence ceased, then diluted up to a total of 1.5 L with further water. This mixture was extracted with ethyl acetate (2×1.5 L). The combined organic extracts were washed with water (2×1 L) and brine (2×1 L), then dried over magnesium sulfate and evaporated to provide compound 1.2 as an amber-coloured oil (199 g, 99% yield, 96% purity). ¹H NMR (CDCl₃, 300 MHz): δ −0.08 (s, 1H), 0.89 (m, 2H), 3.52 (m, 2H), 5.68 (s, 2H), 6.50 (dd, 1H), 7.08 (dd, 1H), 7.34 (d, 1H), 7.90 (dd, 1H), 8.33 (dd, 1H). LC-MS (249 [M+H]⁺).

### 3,3-Dibromo-1-((2-(trimethylsilyl)ethoxy)methyl)-1H-pyrrolo[2,3-b]pyridin-2(3H)-one 1.3

A mechanically-stirred suspension of pyridinium tribromide (646 g, 2.02 mol) in 1,4-dioxane (900 mL) was cooled to 10-15° C. using an ice/water bath, and a solution of 1.2 (100 g, 403.2 mmol) in 1,4-dioxane (500 mL) was added dropwise (NOTE: no significant exotherm is observed, but the reaction is kept cool to minimise formation of polymeric by-products). After stirring for 2 h at 10-15° C., the mixture was partitioned between water (1.5 L) and ethyl acetate (1.5 L). The ethyl acetate layer was collected and washed with water (2×1 L), saturated aqueous sodium bicarbonate solution (1 L), sodium thiosulfate solution (1M solution, 1 L), and brine (2×1 L). The ethyl acetate layer was dried over magnesium sulfate and evaporated to provide compound 1.3 (144 g, 85% yield, 89% purity). ¹H NMR (CDCl₃, 300 MHz): δ −0.03 (s, 9H), 0.97 (dd, 2H), 3.70 (dd, 2H), 5.32 (s, 2H), 7.15 (dd, 1H), 7.87 (dd, 1H), 8.30 (dd, 1H). LC-MS (421, 423, 425 [M+H]⁺).

### 1-((2-(Trimethylsilyl)ethoxy)methyl)-1H-pyrrolo[2,3-b]pyridin-2(3H)-one 1.4

To a mechanically-stirred solution of 1.3 (144 g, 341 mmol) in tetrahydrofuran (2 L) was added saturated aqueous ammonium chloride solution (0.5 L). The suspension was cooled in an ice/salt/water bath to 5-10° C., and zinc powder (223 g, 3.41 mol) was then added in portions. After half of the zinc had been added, the internal temperature peaked at 24° C., and no further significant exotherm was noted upon addition of the remaining zinc. After stirring for 2 h at room temperature, the mixture was filtered through a pad of Celite® to remove excess zinc, washing with ethyl acetate (1 L). The filtrate was diluted with water (1.2 L), effecting precipitation of zinc bromide salts. This suspension was filtered through a further pad of Celite® The organic layer was separated from the filtrate and washed with water (0.8 L) and brine (2×0.8 L), dried over magnesium sulfate, and evaporated to give a dark red oil. The crude material was purified by dry-flash chromatography (0-30% ethyl acetate in heptane) to provide compound 1.4 (53.7 g, 55% yield, 88% purity). ¹H NMR (CDCl₃, 300 MHz): δ −0.03 (s, 9H), 0.98 (dd, 2H), 3.59 (s, 2H), 3.69 (dd, 2H), 5.25 (s, 2H), 6.97 (dd, 1H), 7.50 (dd, 1H), 8.22 (d, 1H). LC-MS (265 [M+H]⁺).

### (4-Nitro-1,2-phenylene)dimethanol 1.8

A mechanically-stirred solution of borane-tetrahydrofuran complex (1M in THF, 1.23 L, 1.23 mol) was cooled to 0° C. A solution of 4-nitrophthalic acid (100 g, 472 mmol) in tetrahydrofuran (1 L) was added dropwise over a period of ca. 45 min, maintaining the internal temperature below 10° C. The cooling bath was then removed, and the mixture stirred overnight at room temperature. The stirred mixture was then once again cooled to 0° C., and methanol added slowly to destroy excess borane (until effervescence was no longer observed). The mixture was concentrated to 25-30% volume, and then diluted to 1 L by addition of water. The mixture was adjusted to pH 10 by addition of 2M aqueous sodium hydroxide, and then extracted with ethyl acetate (5×1 L). The combined organic extracts were dried over magnesium sulfate, and evaporated to provide compound 1.8 (85.5 g, 98% yield, 98% purity). ¹H NMR (CDCl₃, 300 MHz): δ 4.60 (m, 4H), 5.44 (q, 2H), 7.67 (d, 1H), 8.09 (dd, 1H), 8.23 (dd, 1H). LC-MS (182 [M−H]⁻).

### 1,2-Bis(bromomethyl)-4-nitrobenzene 1.9

A suspension of the diol 1.8 (95.5 g, 522 mmol) in dioxane (2 L) was cooled to 0° C., and phosphorous tribromide (54 mL, 574 mmol) added dropwise. Cooling was then removed, and the mixture allowed to stir overnight at room temperature. The mixture was then poured carefully into a stirred solution of saturated sodium bicarbonate (1.5 L) and extracted with ethyl acetate (3×1 L). The organic extracts were dried over magnesium sulfate, and evaporated to provide compound 1.9 (154 g, 96% yield, 98% purity). ¹H NMR (CDCl₃, 300 MHz): δ 4.66 (s, 2H), 4.67 (s, 2H), 7.56 (d, 1H), 8.16 (dd, 1H), 8.25 (d, 1H).

### 5-Nitro-1′-((2-(trimethylsilyl)ethoxy)methyl)-1,3-dihydrospiro[indene-2,3′-pyrrolo[2,3-b]pyridin]-2′(1′H)-one 1.5

To a mechanically-stirred solution of compound 1.4 (55 g, 208 mmol) in dimethylformamide (1.65 L) was added 1.9 (70.8 g, 229 mmol). Caesium carbonate (238 g, 729 mmol) was then added in one portion. This suspension was stirred for 16 h at room temperature, then filtered through a Celite® pad, washing the filter cake with ethyl acetate (2 L). The filtrate was washed with water (3×1 L) and brine (1 L), then dried over magnesium sulfate and evaporated to a deep red oil (96 g). This was purified by dry flash chromatography (eluting with 9:1 heptane/ethyl acetate, followed by 17:3 heptane/ethyl acetate, 8:2 heptane/ethyl acetate, 3:1 heptane/ethyl acetate, 7:3 heptane/ethyl acetate, and 13:7 heptane/ethyl acetate) to give a yellow/orange powder (60.1 g), which was triturated with diethyl ether to afford compound 1.5 (45 g, 53% yield, 97% purity). ¹H NMR (CDCl₃, 300 MHz): δ −0.01 (s, 9H), 0.99 (dd, 2H), 3.18 (dd, 2H), 3.71 (m, 4H), 5.30 (s, 2H), 6.88 (dd, 2H), 7.08 (dd, 1H), 7.43 (d, 1H), 8.09 (m, 2H), 8.23 (dd, 1H). LC-MS (411 [M+H]⁺).

### 5-Amino-1′-((2-(trimethylsilyl)ethoxy)methyl)-1,3-dihydrospiro[indene-2,3′-pyrrolo[2,3-b]pyridin]-2′(1′H)-one 1.6

To a mechanically-stirred solution of 1.5 (70 g, 170.3 mmol) in tetrahydrofuran (1.1 L) was added saturated ammonium chloride solution (300 mL), followed by zinc powder (111 g, 1.70 mol), added in three portions. Internal temperature rose initially from 22° C. to 33° C., then cooling slowly over 1 h to ambient temperature. LC-MS analysis after 2.5 h indicated a mixture of product and hydroxylamine/nitroso intermediates. An additional 35 g zinc powder and 100 mL saturated ammonium chloride solution were added. After an additional 3.5 h, reduction was complete. The mixture was filtered through a pad of Celite®, washing the filter cake with ethyl acetate (1 L). The filtrate was washed with water (3×1 L), dried over magnesium sulfate, and evaporated to give an orange solid, which was triturated with diethyl ether to provide compound 1.6 as a pale yellow powder (48.8 g). Repurification of the trituration liquors by flash chromatography (eluting 1:1 heptane/ethyl acetate), and a further trituration with diethyl ether gave an additional 3 g of 1.6, giving a total of 51.8 g of compound 1.6 (80% yield, 95% purity). ¹H NMR (CDCl₃, 300 MHz): δ −0.02 (s, 9H), 0.98 (m, 2H), 2.91 (d, 2H), 3.56 (dd, 2H), 3.69 (m, 2H), 5.29 (s, 2H), 6.59 (m, 2H), 6.82 (dd, 1H), 7.02 (d, 1H), 7.09 (dd, 1H), 8.18 (dd, 1H). LC-MS (382 [M+H]⁺).

Intermediate A

### 5-Amino-1,3-dihydrospiro[indene-2,3′-pyrrolo[2,3-b]pyridin]-2′ (1′H)-one

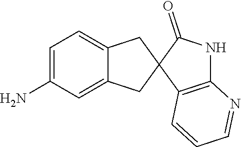 [SMILES: Nc1ccc2c(c1)CC1(C2)C(=O)Nc2ncccc21]

A solution of 1.6 (51.8 g, 136 mmol) in freshly-prepared hydrogen chloride in methanol [prepared to approximately 15% concentration (w/v)] was heated to reflux for 6 h. Once reaction was complete, heating was stopped, and the solution allowed to cool to room temperature overnight. The mixture was concentrated in vacuo to a thick orange liquid, then diluted with 300 mL water, and the pH adjusted to 9 with saturated sodium carbonate solution. The aqueous mixture was extracted with dichloromethane (3×500 mL), and 9:1 dichloromethane/methanol (3×500 mL). The combined organics were dried over magnesium sulfate and evaporated to an orange solid, which was triturated with 2:1 dichloromethane/ethyl acetate (ca. 60 mL) to provide Intermediate A as a pale orange powder (21.5 g, 63% yield, 97% purity.). 1H NMR (DMSO-d₆, 300 MHz): δ 2.84 (dd, 2H), 3.18 (dd, 2H), 4.94 (s, NH₂), 6.41 (m, 2H), 6.81 (dd, 1H), 6.86 (d, 1H), 7.08 (dd, 1H), 8.01 (dd, 1H), 11.03 (s, NH). LC-MS Method 10: rt 0.751 (252 [M+H]⁺).

Synthesis of Intermediate B

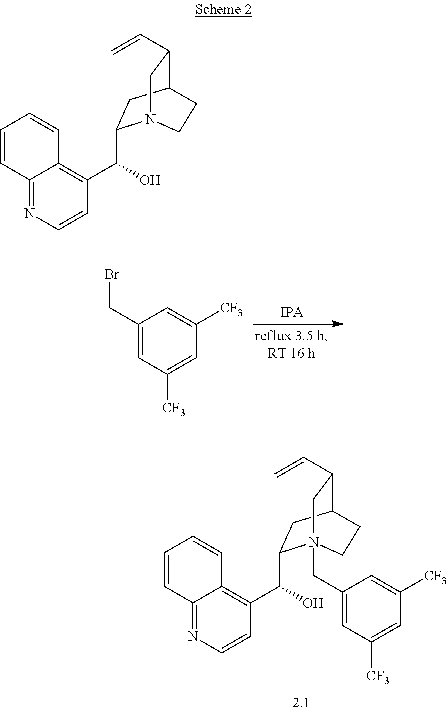 [SMILES: C=CC1CN2CCC1CC2[C@H](O)c1ccnc2ccccc12 ; FC(F)(F)c1cc(CBr)cc(C(F)(F)F)c1 ; C=CC1C[N+]2(Cc3cc(C(F)(F)F)cc(C(F)(F)F)c3)CCC1CC2[C@H](O)c1ccnc2ccccc12]

A solution of 3,5-bis(trifluoromethyl)benzyl bromide (5.00 g, 17.0 mmol) and cinchonidine (5.50 g, 17.8 mmol) in isopropanol was heated at reflux for 3.5 h. After cooling to room temperature, the reaction mixture was slowly poured into diethyl ether (250 mL) with stirring. The precipitated solids were filtered and washed with diethyl ether (150 mL) and pentane (100 mL) to afford compound 2.1 (8.60 g, 84%). ¹H NMR (CD₃OD, 400 MHz) δ 1.48 (m, 1H), 1.91 (m, 1H), 2.12 (m, 1H), 2.31 (m, 2H), 2.76 (s, br, 1H), 3.41 (t, 1H), 3.50 (dd, 1H), 3.71 (m, 1H), 4.02 (t, 1H), 4.58 (m, 1H), 5.03 (d, 1H), 5.19 (m, 2H), 5.37 (d, 1H), 5.71 (ddd, 1H), 6.67 (s, 1H), 7.98 (dddd, 2H), 8.15 (dd, 1H), 8.27 (s, 1H), 8.34 (d, 1H), 8.98 (d, 1H); [α]_{D}²³=−139.5° (c 8.9, MeOH).

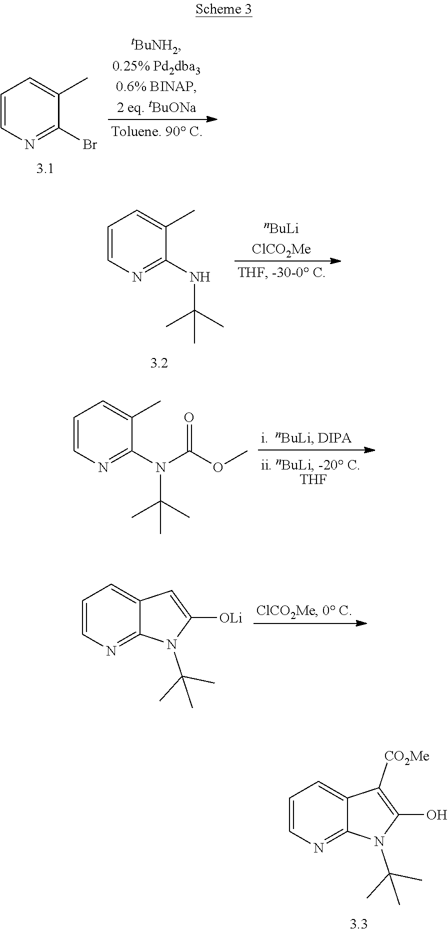

### N-tert-Butyl-3-methyl-pyridin-2-amine 3.2

A mixture of compound 3.1 (20.00 g, 116 mmol) and sodium tert-butoxide (22.35 g, 232 mmol) in toluene (200 mL) was degassed under vacuum and purged with nitrogen for three times. 2-Methylpropan-2-amine (12.75 g, 174 mmol), Pd₂(dba)₃ (266 mg, 0.29 mmol) and BINAP (434 mg, 0.70 mmol) were added at 25° C., and the mixture was degassed under vacuum and purged with nitrogen three times. The mixture was stirred at 25° C. for 10 min and then heated to 100° C. with stirring for 16 h under nitrogen. The mixture was poured into water (400 mL) and extracted with ethyl acetate (3×400 mL). The organic phases were combined, washed with brine (2×400 mL) and dried over anhydrous sodium sulfate. After filtration and concentration, the residue was dissolved with ethyl acetate (200 mL) and poured into water (200 mL). The mixture was adjusted to pH3 by adding 1M hydrochloric acid and extracted with ethyl acetate (2×200 mL). The organic phases were discarded, and the aqueous phase adjusted to pH9 with saturated aqueous sodium bicarbonate. The aqueous phase was extracted with ethyl acetate (3×200 mL). The organic phases were combined were washed with brine (200 mL) and dried over anhydrous sodium sulfate. After filtration and concentration, the crude product was purified by silica gel column chromatography, diluted with petroleum ether:ethyl acetate=1:0 to 50:1 to provide compound 3.2 (28.30 g, 73% yield, 98.9% purity) as a yellow oil. ¹H NMR (CDCl₃, 400 MHz) δ 1.50 (s, 9H), 2.04 (s, 3H), 4.00 (br. s, 1H), 6.44-6.48 (m, 1H), 7.17 (dd, 1H), 8.00 (d, 1H). LC-MS Method 1: rt 0.214 min (165.2 [M+H]⁺).

### Methyl 1-tert-butyl-2-hydroxy-pyrrolo[2,3-b]pyridine-3-carboxylate 3.3

To a solution of compound 3.2 (27.5 g, 167 mmol) in tetrahydrofuran (150 mL) was added 2.5M n-BuLi (73.67 mL, 184 mmol) dropwise under nitrogen at −40° C. The mixture was stirred at −10° C. for 0.5 h. Then methyl chloroformate (17.40 g, 184 mmol) was added slowly to the mixture at −40° C. The mixture was stirred at 10° C. for 1.5 h. The reaction temperature of the reaction was kept at −40° C. and 2.5M n-BuLi (46.88 mL, 117 mmol) was added dropwise. The mixture was stirred at −40° C. for 0.5 h. Diisopropylamine (23.72 g, 234 mmol) was added to the mixture under nitrogen at −40° C. and followed by 2.5M n-BuLi (107.16 mL, 267 mmol). The mixture was stirred at −40° C. for 0.5 h, and then at 20° C. for another 10 h. After the reaction completed, the mixture was cooled to 0° C. and methyl chloroformate (20.57 g, 218 mmol) added. The mixture was stirred at 0° C. for 1 h. The mixture was adjusted to until pH 3˜4 by adding 1M hydrochloric acid. The mixture was extracted with ethyl acetate (2×200 mL). The extracts were combined, washed with brine (100 mL) and dried over anhydrous sodium sulfate. After filtration and concentration, the residue was purified by silica gel column chromatography, diluted with petroleum ether:ethyl acetate=100:1 to 50:1 to afford compound 3.3 as a red solid (36 g, 78% yield, 97% purity). ¹H NMR (CDCl₃, 400 MHz) δ 1.92 (s, 9H), 3.96 (s, 3H), 7.08 (dd, 1H), 7.89 (d, 1H), 8.14 (dd, 1H), 11.80 (br. s, 1H). LC-MS Method 1: rt 0.887 min, (249.1 [M+H]⁺).

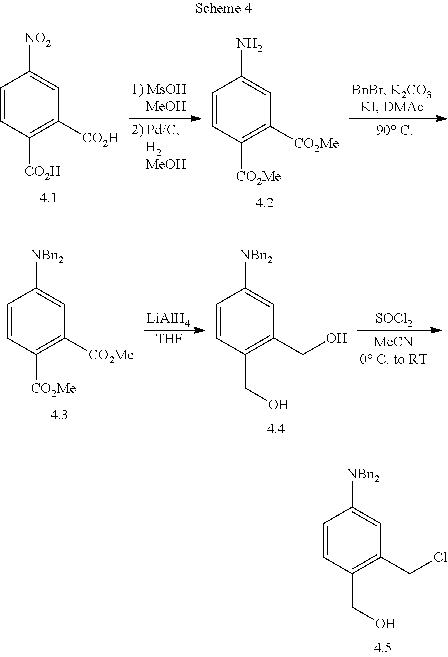

### Dimethyl 4-nitrobenzene-1,2-dicarboxylate 4.1

To a solution of 4-nitrophthalic acid (50.0 g, 237 mmol) in methanol (500 mL) was added methanesulfonic acid (34.14 g, 355 mmol). The mixture was stirred at 80° C. for 16 h. The mixture was concentrated under vacuum and the residue was dissolved in ethyl acetate (500 mL). The solution was washed with saturated aqueous solution of sodium bicarbonate (2×500 mL), brine (500 mL) and dried over sodium sulfate. After filtration and concentration, compound 4.1 (102.0 g, crude) was obtained as a yellow solid. ¹H NMR (CDCl₃, 400 MHz) δ 3.97 (d, 6H), 7.86 (d, 2H), 8.41 (dd, 1H), 6.64 (d, 1H).

### Dimethyl 4-aminobenzene-1,2-dicarboxylate 4.2

To a solution of compound 4.1 (37 g, 155 mmol) in methanol (500 mL) was added 10% Pd/C (2 g) under nitrogen. Then the mixture was degassed under vacuum and purged three times with hydrogen. The resulting mixture was stirred at 20° C. for 10 h. The reaction mixture was filtered, and the filter liquid was concentrated in vacuum to provide compound 4.2 (30 g, crude) as a yellow solid. ¹H NMR (CD₃OD, 400 MHz) δ 3.78 (s, 3H), 3.84 (s, 3H), 6.66-6.70 (m, 2H), 7.62 (d, 1H). LC-MS Method 1: rt 0.723 min, (178.1, [M-OMe+H]⁺; 232.1 (M+Na)⁺).

### Dimethyl 4-(dibenzylamino)benzene-1,2-dicarboxylate 4.3

To a solution of compound 4.2 (90.0 g, 430 mmol) in dimethylacetamide (500 mL) was added sodium iodide (12.9 g, 86.0 mmol), potassium carbonate (208.10 g, 1.51 mol) and benzyl chloride (163.4 g, 1.29 mol). The mixture was stirred at 90° C. for 15 h. The reaction mixture was filtered, and the filtrate poured into water (1 L). The mixture extracted with ethyl acetate (3×1 L). The organic phases were combined were washed with brine (3×1 L) and dried over anhydrous sodium sulfate. After filtration and concentration, the residue was purified by silica gel column chromatography, triturated with petroleum ether:ethyl acetate=50:1˜25:1 to provide compound 4.3 (180.0 g, 97% yield) as yellow oil. ¹H NMR (CDCl₃, 400 MHz) δ 3.95 (s, 3H), 3.79 (s, 3H), 4.62 (s, 4H), 6.74 (dd, 1H), 6.83 (d, 1H), 7.20 (d, 4H), 7.28-7.35 (m, 4H), 7.36-7.38 (m, 2H), 7.74 (d, 1H). LC-MS Method 1: rt 1.038 min, (390.3 [M+H]⁺).

### [4-(Dibenzylamino)-2-(hydroxymethyl)phenyl]methanol 4.4

To a solution of compound 4.3 (44.0 g, 113 mmol) in tetrahydrofuran (500 mL) was added lithium aluminium hydride (7.74 g, 204 mmol) in portions over 1 h at −20° C. and the mixture stirred at 10° C. for 16 h. The reaction was quenched by cooling the mixture to 0° C. and adding water (10 mL), 10% aqueous sodium hydroxide (10 mL), water (10 mL) and sodium sulfate (50 g). The mixture was filtered, and the filtrate collected. The filter cake was washed with tetrahydrofuran (5×100 mL). The organic phases were combined and concentrated under reduced pressure to provide compound 4.4 (35.2 g, 93% yield) as a light-yellow solid. ¹H NMR (CDCl₃, 400 MHz) δ 2.97 (br. s, 2H), 4.57 (s, 2H), 4.59 (s, 2H), 4.69 (s, 4H), 6.65 (dd, 1H), 6.77 (d, 1H), 7.12 (d, 1H), 7.24-7.27 (d, 2H), 7.28-7.29 (m, 2H), 7.33-7.39 (m, 6H). LC-MS Method 1: rt 0.855 min, (334.1 [M+H]⁺).

### [4-(Dibenzylamino)-2-(hydroxymethyl)phenyl]methanol 4.5

A solution of thionyl chloride (83.1 g, 698 mmol) in acetonitrile (228 mL) was cooled to 0° C., and compound 4.4 (76.0 g, 228 mmol) was added in portions while keeping the internal temperature below 18° C. The reaction mixture was stirred at 25° C. for 10 min. The mixture was diluted with MTBE (1 L), and standing for 2 h at 0° C. The crystals were collected by filtration and dried under vacuum to give compound 4.5 (68.0 g, 74% yield, HCl salt) as yellow solid. 1H NMR (DMSO-d₆, 400 MHz) δ 4.43-4.77 (m, 8H), 6.62-6.63 (m, 1H), 6.87 (s, 1H), 7.22-7.32 (m, 11H). LC-MS Method 1: rt 1.012 min, (352.2 [M+H]⁺).

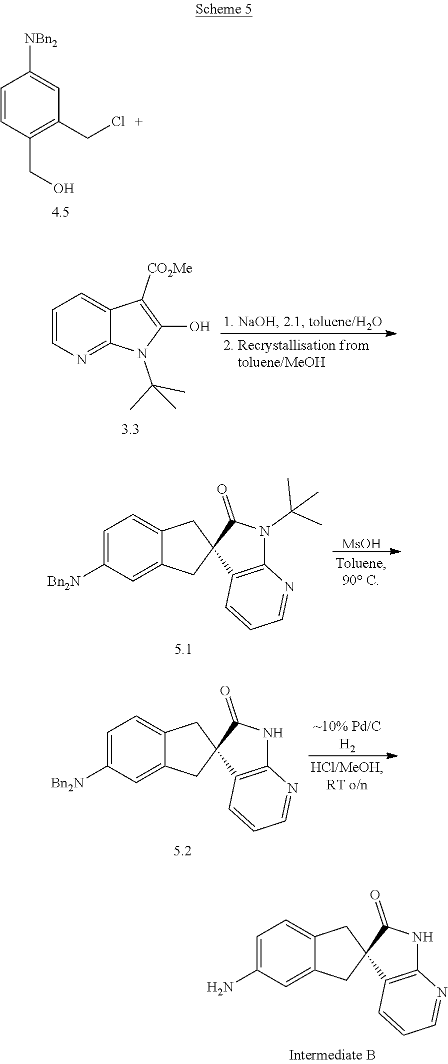

### (R)-1′-(tert-Butyl)-5-(dibenzylamino)-1,3-dihydrospiro[indene-2,3′-pyrrolo[2,3-b]pyridin]-2′(1′H)-one 5.1

To a solution of NaOH (72 g, 1.80 mol) in water (60 mL) at room temperature was added toluene (130 mL) and compound 4.5 (4.7 g, 12.08 mmol). The reaction mixture was stirred at room temperature while bubbling argon through the solution for 5 min. Compound 3.3 (3.00 g, 12.1 mmol) was added in three portions over 10 min. Argon continued to be bubbled through the stirring solution for 15 min and compound 2.1 (700 mg, 1.2 mmol) was added in one portion at room temperature. This mixture was stirred at room temperature for 3 h under bubbling argon. Water (˜300 mL) was added [note: exothermic reaction] and the mixture stirred for ˜15 min while warming to room temperature. The two layers were separated, and the aqueous layer extracted with ethyl acetate. The combined extracts were washed with water, dried over MgSO₄, filtered and evaporated to give the crude product of ˜90% purity, 83% ee. This product was dissolved in toluene (60 mL) at 60° C. Once totally dissolved, the mixture was warmed to room temperature and MeOH (180 mL) was added. The mixture was stirred at room temperature for 16 h, and the resulting crystals were collected by filtration and washed with MeOH to give the product (61%, 96% ee). The product was recrystallised using toluene (50 mL) and MeOH (120 mL) to give compound 5.1 (3.1 g, 52% yield, >99% ee). ¹H NMR (CDCl₃, 400 MHz) δ 8.14 (m, 1H), 7.30 (m, 10H), 7.05 (m, 2H), 6.78 (m, 1H), 6.67 (s, br, 2H), 4.67 (s, br, 4H), 3.48 (d, 2H), 2.87 (dd, 2H), 1.82 (s, 9H); LC-MS Method 1: rt 1.215 min, (488.27 [M+H]⁺); Chiral HPLC: Phenomenex® Lux 3 μm Cellulose-1 column; ⁿhexane:isopropanol, 95:5; flow rate=1.0 mL/min; detection at 254 nm.

### (3R)-5′-(Dibenzylamino)spiro[1H-pyrrolo[2,3-b]pyridine-3,2′-indane]-2-one 5.2

Compound 5.1 (26.8 g, 55.0 mmol) was dissolved with methanesulfonic acid (67.00 mL) at 20° C. and toluene (10 mL) added. The resulting mixture was stirred at 90° C. for 3 h. LC-MS showed the starting material was consumed completely and desired MS was detected. The mixture was poured into water (100 mL) and adjusted to pH10 with sodium carbonate. The mixture was extracted with ethyl acetate (3×100 mL). The organic phases were combined, washed with brine (100 mL) and dried over sodium sulfate. After filtration and concentration, the residue was purified by silica gel column chromatography, triturated with petroleum ether:ethyl acetate=5:1 to 0:1 to provide compound 5.2 as a yellow solid (20 g, 83% yield). ¹H NMR (DMSO-d₆, 400 MHz) δ 2.96 (d, 2H), 3.22 (d, 2H), 4.67 (s, 4H), 6.54 (dd, 1H), 6.63 (s, 1H), 6.68 (dd, 1H), 6.98 (d, 1H), 7.19-7.35 (m, 11H), 8.09 (d, 1H), 11.03 (s, 1H). LC-MS Method 2: rt 0.884 min, (432.2 [M+H]⁺).

Intermediate B

### (R)-5-Amino-1,3-dihydrospiro[indene-2,3′-pyrrolo[2,3-b]pyridin]-2′(1′H)-one

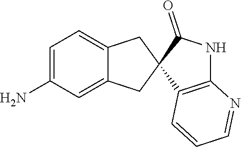 [SMILES: Nc1ccc2c(c1)C[C@@]1(C2)C(=O)Nc2ncccc21]

To a solution of compound 5.2 (20 g, 46.35 mmol) in methanol (200 mL) was added 10% Pd/C (1.5 g) and methanesulfonic acid (7.15 g, 74.4 mmol). The mixture was degassed under vacuum and purged three times with hydrogen. The mixture was stirred at 20° C. for 16 h under hydrogen balloon, filtered and the filtrate concentrated under reduced pressure to give a residue. The residue was dissolved with tetrahydrofuran (100 mL), saturated aqueous sodium carbonate added until pH=8 and the mixture filtered to give pink solid. The solid was dissolved was tetrahydrofuran (100 mL), dried over sodium sulfate. After filtration and concentration, Intermediate B (10.78 g, 83% yield, 90.2% purity) was obtained as a light-yellow solid. ¹H NMR (DMSO-d₆, 400 MHz) δ 2.92 (dd, 2H), 3.33 (dd, 2H), 4.95 (s, 2H), 6.44-6.48 (m, 2H), 6.84-6.92 (m, 2H), 7.13 (d, 1H), 8.05 (d, 1H), 11.04 (s, 1H). LC-MS Method 10: rt 0.751 min, (252.11 [M+H]⁺); chiral HPLC: Phenomenex® Lux 3 μm Cellulose-1 column; ⁿhexane:isopropanol, 40:60; flow rate=0.5 mL/min; detection at 220 nm.

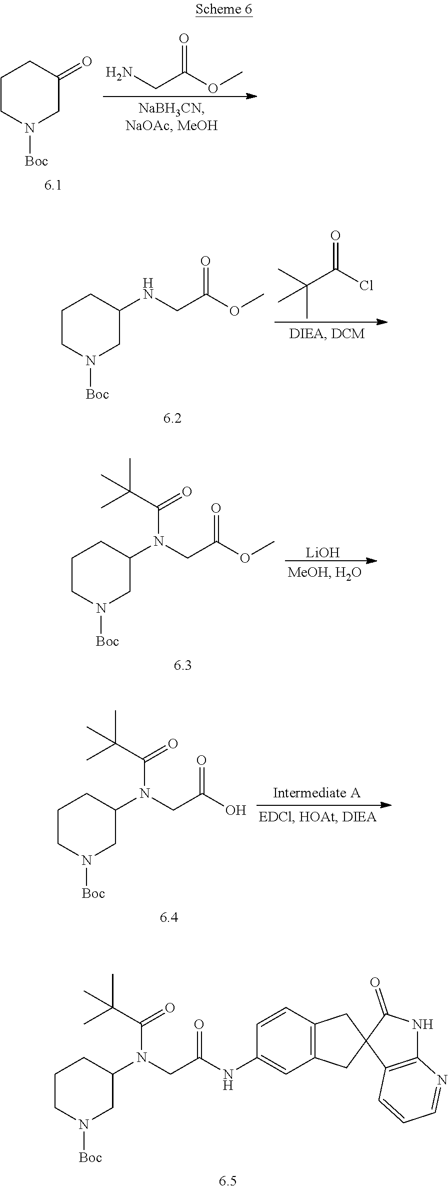

### tert-Butyl 3-[(2-methoxy-2-oxo-ethyl)amino]piperidine-1-carboxylate 6.2

A mixture of compound 6.1 (1.00 g, 5.02 mmol), methyl 2-aminoacetate (756 mg, 6.02 mmol, HCl salt) and sodium acetate (617 mg, 7.53 mmol) in methanol (10 mL) was stirred at 20° C. for 2 h, sodium cyanoborohydride (946 mg, 15.06 mmol) was added and stirring continued for 12 h. The reaction mixture was poured into water (50 mL), extracted with ethyl acetate (3×50 mL). The organic phases were combined, washed with brine (2×40 mL) and dried over anhydrous sodium sulfate. After filtration and concentration, the residue was purified by silica gel column chromatography, eluting with petroleum ether:ethyl acetate=10:1˜1:1 to provide compound 6.2 (580 mg, 42% yield) as yellow oil. ¹H NMR (CDCl₃, 400 MHz) δ 1.46 (s, 9H), 1.63-1.80 (m, 3H), 1.87-1.96 (m, 1H), 2.43-3.04 (m, 3H), 3.41-3.53 (m, 2H), 3.74 (s, 3H), 3.75-3.82 (m, 1H), 3.84-4.11 (m, 1H).

### tert-Butyl 3-(N-(2-methoxy-2-oxoethyl)pivalamido)piperidine-1-carboxylate 6.3

To a solution of compound 6.2 (580 mg, 2.13 mmol) and DIEA (1.48 mL, 8.52 mmol) in dichloromethane (6 mL) was added 2,2-dimethylpropanoyl chloride (308 mg, 2.56 mmol) dropwise at 0° C. The mixture was stirred at 20° C. for 30 min under nitrogen, poured into water (40 mL) and extracted with ethyl acetate (3×40 mL). The organic phases were combined, washed with brine (3×40 mL) and dried over anhydrous sodium sulfate. After filtration and concentration, the residue was purified by silica gel column, eluting with petroleum ether:ethyl acetate=20:1˜5:1 to provide compound 6.3 (580 mg, 76% yield) as a white solid. ¹H NMR (CDCl₃, 400 MHz) δ 1.32 (s, 9H), 1.45 (s, 9H), 1.50-1.62 (m, 2H), 1.75-1.84 (m, 1H), 1.97-2.05 (m, 1H), 2.44-2.75 (m, 2H), 3.72 (s, 3H), 3.75-3.95 (m, 2H), 3.97-4.09 (m, 1H), 4.12-4.35 (m, 2H).

### 2-(N-(1-(tert-Butoxycarbonyl)piperidin-3-yl)pivalamido)acetic acid 6.4

To a solution of compound 6.3 (300 mg, 0.84 mmol) in tetrahydrofuran (3 mL) and methanol (2 mL) was added a solution of lithium hydroxide monohydrate (141 mg, 3.37 mmol) in water (1 mL) at 20° C. Then the resulting mixture was stirred at 20° C. for 12 h, poured into water (20 mL) and washed with ethyl acetate (20 mL). The aqueous phase was adjusted to pH4 with 1M hydrochloric acid. The resulting mixture was extracted with ethyl acetate (3×20 mL). The organic phases were combined, washed with brine (2×20 mL) and dried over anhydrous sodium sulfate. After filtration and concentration, compound 6.4 (280 mg, 97% yield) was obtained as a colourless gum. ¹H NMR (CDCl₃, 400 MHz) δ 1.33 (s, 9H), 1.47 (s, 9H), 1.51-1.67 (m, 2H), 1.74-1.84 (m, 1H), 1.96-2.04 (m, 1H), 2.44-2.80 (m, 2H), 3.90 (q, 2H), 3.99-4.09 (m, 1H), 4.13-4.34 (m, 2H).

### tert-Butyl 3-(N-(2-oxo-2-((2′-oxo-1,1′,2′,3-tetrahydrospiro[indene-2,3′-pyrrolo[2,3-b]pyridine]-5-yl)amino)ethyl)pivalamido)piperidine-1-carboxylate 6.5

To a solution of compound 6.4 (68 mg, 0.20 mmol), EDCI (50 mg, 0.26 mmol) and HOAt (35 mg, 0.26 mmol) in DMF (1.5 mL) was added DIEA (90 mg, 0.70 mmol) at 20° C. Intermediate A (50 mg, 0.20 mmol) was added and the mixture was stirred at 20° C. for 12 h. The reaction mixture was added to water (15 mL) and filtered. The precipitate was washed with water (10 mL), dissolved with MeCN (20 mL) and concentrated in vacuo to give compound 6.5 (84 mg, 66% yield, 90.1% purity) as a yellow solid. ¹H NMR (DMSO-d₆, 400 MHz) δ 1.22 (s, 9H), 1.39 (s, 9H), 1.62-1.91 (m, 4H), 2.51-2.54 (m, 4H), 3.05 (t, 2H), 3.76-4.00 (m, 4H), 4.01-4.17 (m, 1H), 6.85 (dd, 1H), 7.10-7.24 (m, 2H), 7.31 (t, 1H), 7.66 (d, 1H), 8.06 (dd, 1H), 9.92 (s, 1H), 11.08 (s, 1H). LC-MS Method 6: rt 2.302 min, [M+Na]⁺=598.4.

### Example 1

### N-(2-Oxo-2-((2′-oxo-1,1′,2′,3-tetrahydrospiro[indene-2,3′-pyrrolo[2,3-b] pyridin]-5-yl)amino)ethyl)-N-(piperidin-3-yl)pivalamide

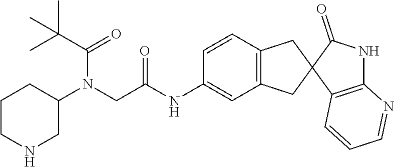 [SMILES: CC(C)(C)C(=O)N(CC(=O)Nc1ccc2c(c1)CC1(C2)C(=O)Nc2ncccc21)C1CCCNC1]

To a solution of compound 6.5 (70 mg, 0.12 mmol) in dichloromethane (2 mL) was added zinc bromide (274 mg, 1.22 mmol) at 20° C. The mixture was stirred at 20° C. for 16 h. The reaction mixture was dissolved in methanol (20 mL). Saturated aqueous sodium carbonate was added to the mixture until pH=8. Then the resulting mixture was extracted with ethyl acetate (4×40 mL). The organic phases were combined, washed with brine (3×30 mL) and dried over anhydrous sodium sulfate. After filtration and concentration, the residue was purified by prep-HPLC (column: Gemini 150×25 mm, 5 μm; mobile phase: [solvent A: water (0.05% ammonia hydroxide v/v), solvent B: MeCN]; B %: 29-59%, 12 min). After lyophilisation, Example 1 was obtained as a white solid (14 mg, 24% yield, 100% purity). ¹H NMR (CD₃OD, 400 MHz) δ 1.34 (s, 9H), 1.56-1.76 (m, 2H), 1.78-1.86 (m, 1H), 1.94-2.02 (m, 1H), 2.46 (td, 1H), 2.69 (t, 1H), 2.88-2.97 (m, 1H), 3.06 (dd, 2H), 3.11-3.18 (m, 1H), 3.51 (dd, 2H), 3.92-4.09 (m, 2H), 4.16-4.27 (m, 1H), 6.88 (dd, 1H), 7.12 (dd, 1H), 7.22 (d, 1H), 7.34-7.40 (m, 1H), 7.55 (s, 1H), 8.05 (dd, 1H). LC-MS Method 16: rt 1.891 min, [M+H]⁺ 476.3.

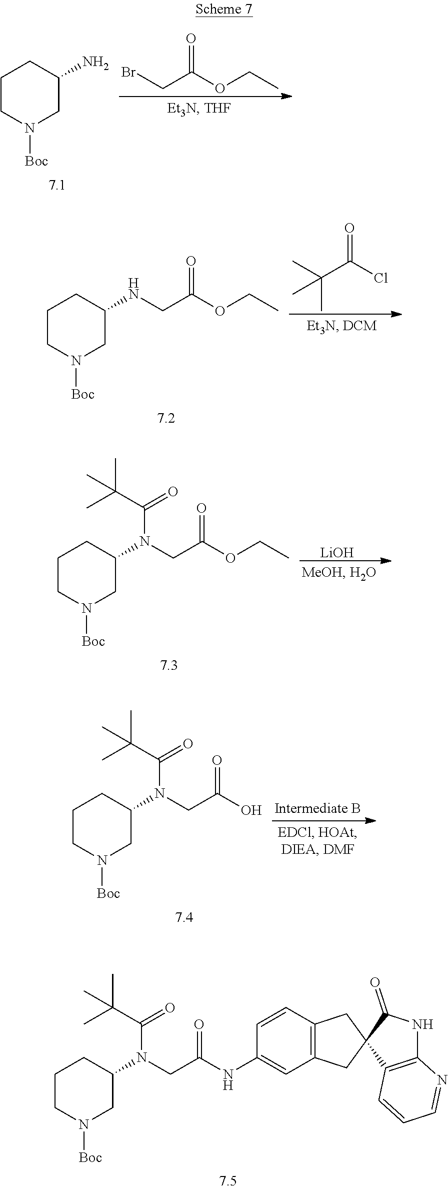

### (S)-tert-Butyl 3-((2-ethoxy-2-oxoethyl)amino)piperidine-1-carboxylate 7.2

To a solution of (S)-3-amino-1-boc-piperidine (7.1) (800 mg, 3.99 mmol) and triethylamine (0.7 mL) in tetrahydrofuran (6 mL) was added ethyl 2-bromoacetate (700 mg, 4.19 mmol). The mixture was stirred at 15° C. for 16 h, poured into water (50 mL) and extracted with ethyl acetate (3×50 mL). The organic phases were combined, washed with brine (100 mL) and dried over sodium sulfate. After filtration and concentration, the residue was purified by silica gel column (petroleum ether:ethyl acetate=5:1 to 2:1) to afford compound 7.2 (1.00 g, 87% yield) as colourless oil. ¹H NMR (CDCl₃, 400 MHz) δ 1.19-1.30 (m, 4H), 1.38 (s, 9H), 1.54-1.65 (m, 2H), 1.83-1.86 (m, 1H), 2.47-2.50 (m, 3H), 3.33-3.43 (m, 2H), 3.70 (dt, 1H), 3.78-4.02 (m, 1H), 4.12 (q, 2H).

### (S)-tert-Butyl 3-(N-(2-ethoxy-2-oxoethyl)pivalamido)piperidine-1-carboxylate 7.3

To a solution of compound 7.2 (280 mg, 0.98 mmol) in dichloromethane (5 mL) was added diisopropylethylamine (253 mg, 1.96 mmol) and pivalolyl chloride (141 mg, 1.17 mmol). The mixture was stirred at 20° C. for 12 h, diluted with dichloromethane (20 mL) and washed with water (20 mL). The organic phase was dried over sodium sulfate. After filtration and concentration, compound 7.3 (360 mg, 99% yield) was obtained as a white solid. ¹H NMR (CDCl₃, 400 MHz) δ 1.25 (t, 3H), 1.33 (s, 9H), 1.45 (s, 9H), 1.53-1.56 (m, 2H), 1.78-1.80 (m, 1H), 2.01-2.03 (m, 1H), 2.46-2.52 (m, 1H), 2.58-2.68 (m, 1H), 3.84 (d, 2H), 4.05-4.14 (m, 2H), 4.17 (q, 2H), 4.24-4.27 (m, 1H).

### (S)-2-(N-(1-(tert-Butoxycarbonyl)piperidin-3-yl)pivalamido)acetic acid 7.4

To a solution of compound 7.3 (360 mg, 0.97 mmol) in methanol (10 mL) and water (2 mL) was added hydrated lithium hydroxide (122 mg, 2.92 mmol). The mixture was stirred at 20° C. for 1 h, poured into water (20 mL) and extracted with ethyl acetate (2×20 mL). The aqueous phases were adjusted to pH=3-4 with 1M hydrochloric acid and extracted with ethyl acetate (2×50 mL). The organic phases were combined and dried over sodium sulfate. After filtration and concentration, compound 7.4 (300 mg, 90% yield) was obtained as yellow oil. ¹H NMR (CDCl₃, 400 MHz) δ 1.33 (s, 9H), 1.45 (s, 9H), 1.59-1.68 (m, 2H), 1.79-1.83 (m, 1H), 1.99-2.05 (m, 1H), 2.56-2.74 (m, 2H), 3.85-3.98 (m, 2H), 4.02-4.22 (m, 3H).

### (S)-tert-Butyl 3-(N-(2-oxo-2-(((R)-2′-oxo-1,1′,2′,3-tetrahydrospiro[indene-2,3′-pyrrolo [2,3-b]pyridin]-5-yl)amino)ethyl)pivalamido)piperidine-1-carboxylate 7.5

To a solution of compound 7.4 (80 mg, 0.23 mmol) and Intermediate B (59 mg, 0.23 mmol) in DMF (2 mL) was added EDCI (90 mg, 0.47 mmol), HOAt (64 mg, 0.47 mmol) and diisopropylethylamine (60 mg, 0.47 mmol). The mixture was stirred at 20° C. for 2 h, diluted with ethyl acetate (25 mL) and washed with water (20 mL). The organic phase was dried over sodium sulfate. After filtration and concentration, the residue was purified by prep-HPLC (column: Gemini 150×25 mm. 5 μm; mobile phase: [solvent A: water (0.05% ammonia hydroxide v/v), solvent B: MeCN]; B %: 42-72%, 12 min). After extraction with ethyl acetate, compound 7.5 (50 mg, 37% yield, 100% purity) was obtained as a white solid. ¹H NMR (CD₃OD, 400 MHz) δ 1.34 (s, 9H), 1.46 (s, 9H), 1.54-1.57 (m, 1H), 1.79-1.82 (m, 2H), 2.01-2.04 (m, 1H), 2.61-2.72 (m, 1H), 2.83-2.93 (m, 1H), 3.07 (dd, 2H), 3.52 (dd, 2H), 4.03-4.25 (m, 4H), 4.28-4.31 (m, 1H), 6.88 (dd, 1H), 7.13 (dd, 1H), 7.23 (d, 1H), 7.38 (d, 1H), 7.56 (s, 1H), 8.05 (dd, 1H).

### Example 2

### N-(2-Oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)pivalamide

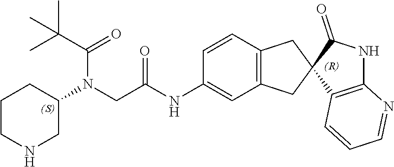

To a solution of compound 7.5 (50 mg, 0.087 mmol) in dichloromethane (5 mL) was added zinc bromide (293 mg, 1.30 mmol). The mixture was stirred at 20° C. for 12 h, diluted with methanol (5 mL), adjusted to pH8-9 with saturated aqueous sodium bicarbonate and extracted with ethyl acetate (3×25 mL). The organic phases were combined and dried over sodium sulfate. After filtration and concentration, the residue was purified by prep-HPLC (column: Phenomenex Synergi C18 150×25 mm, 10 μm; mobile phase: [solvent A: water (0.1% TFA), solvent B: MeCN]; B %: 10-40%, 9 min). After lyophilisation, Example 2 (5.6 mg, TFA salt, 98.6% purity) was obtained as a yellow solid. ¹H NMR (CD₃OD, 400 MHz) δ 1.33 (s, 9H), 1.84-2.10 (m, 4H), 2.92 (t, 1H), 3.08 (d, 2H), 3.36 (m, 1H), 3.49-3.53 (m, 2H), 3.58-3.59 (m, 1H), 4.10-4.20 (m, 2H), 4.65-4.71 (m, 2H), 6.89 (dd, 1H), 7.14 (dd, 1H), 7.24 (d, 1H), 7.40 (d, 1H), 7.54 (s, 1H), 8.06 (dd, 1H). LC-MS Method 8: rt 1.941 min, (476 [M+H]⁺), 98.6% purity.

### Example 3

### N-(2-Oxo-2-(((R)-2′-oxo-1,1′,2′,3-tetrahydrospiro[indene-2,3′-pyrrolo[2,3-b]pyridin]-5-yl)amino)ethyl)-N—((R)-piperidin-3-yl)pivalamide

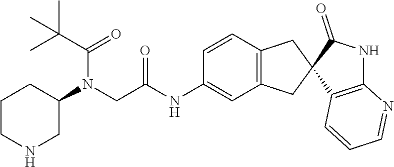 [SMILES: CC(C)(C)C(=O)N(CC(=O)Nc1ccc2c(c1)C[C@@]1(C2)C(=O)Nc2ncccc21)[C@@H]1CCCNC1]

Example 3 was prepared from (R)-3-amino-1-boc-piperidine in analogous fashion to Example 2. The final compound was purified by prep-HPLC (column: Boston pH-lex 150×25 mm, 10 μm; mobile phase: [solvent A: water (0.1% TFA), solvent B: MeCN]; B %: 18-38%, 8 min). After lyophilisation, Example 3 was obtained as a white solid (58 mg, TFA salt, 64% yield, 96.5% purity, 94.31% ee). ¹H NMR (CD₃OD, 400 MHz) δ 1.33 (s, 9H), 1.77-2.15 (m, 4H), 2.85-2.95 (m, 1H), 3.06-3.10 (dd, 2H), 3.15-3.26 (m, 1H), 3.33-3.40 (m, 1H), 3.48-3.58 (m, 3H), 3.98-4.42 (m, 3H), 6.91 (dd, 1H), 7.16-7.17 (m, 1H), 7.23 (d, 1H), 7.34-7.36 (m, 1H), 7.59 (s, 1H), 8.06 (dd, 1H). LC-MS Method 6: rt 1.355 min, [M+H]⁺=476.3.

Synthesis of Intermediate C

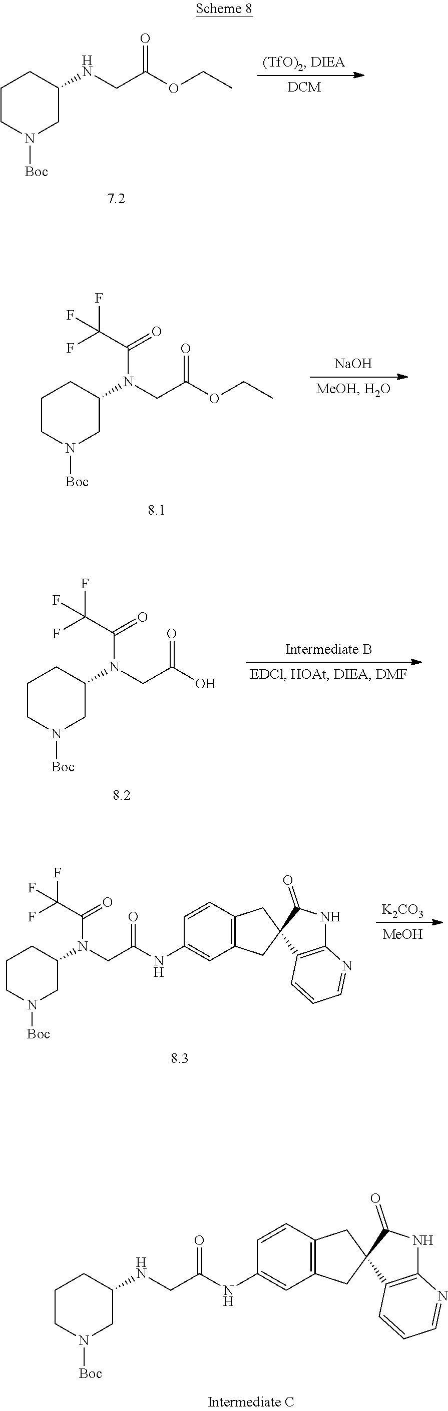

### tert-Butyl (3S)-3-[(2-ethoxy-2-oxo-ethyl)-(2,2,2-trifluoroacetyl)amino]piperidine-1-carboxylate 8.1

To a solution of compound 7.2 (3.50 g, 12.22 mmol) and DIEA (3.95 g, 30.56 mmol) in dichloromethane (40 mL) was added trifluoroacetic anhydride (3.08 g, 14.67 mmol) at 0° C. The mixture was stirred at 20° C. for 0.5 h, poured into water (60 mL) and extracted with ethyl acetate (3×40 mL). The organic phases were combined, washed with 0.1M hydrochloric acid (40 mL) and brine (2×40 mL), and dried over anhydrous sodium sulfate. After filtration and concentration, the residue was purified with silica gel column, eluting with petroleum ether:ethyl acetate=20:1 to 5:1 to give compound 8.1 as yellow oil (3.60 g, 77% yield). ¹H NMR (CDCl₃, 400 MHz) δ 1.29 (t, 3H), 1.46 (s, 9H), 1.52-1.59 (m, 2H), 1.77-1.82 (m, 1H), 2.01-2.09 (m, 1H), 2.53 (t, 1H), 2.68 (t, 1H), 3.83-3.93 (m, 1H), 3.96-4.14 (m, 4H), 4.23-4.29 (m, 2H).

### 2-[[(3S)-1-tert-Butoxycarbonyl-3-piperidyl]-(2,2,2-trifluoroacetyl)amino]acetic acid 8.2

To a solution of compound 8.1 (3.60 g, 9.41 mmol) in methanol (30 mL) was added a solution of sodium hydroxide (377 mg, 9.41 mmol) in water (10 mL) at 20° C. The mixture was stirred at 20° C. for 12 h, poured into water (60 mL) and washed with ethyl acetate (80 mL). The aqueous phase was adjusted to pH4 with 1M hydrochloric acid and extracted with ethyl acetate (3×80 mL). The organic phases were combined, washed with brine (60 mL) and dried over anhydrous sodium sulfate. After filtration and concentration, compound 8.2 was obtained as a yellow gum (1.70 g, 51% yield). ¹H NMR (CDCl₃, 400 MHz) δ 1.46 (s, 9H), 1.54-1.63 (m, 2H), 1.76-1.84 (m, 1H), 1.93-2.04 (m, 1H), 2.49-2.80 (m, 2H), 3.85-3.93 (m, 1H), 3.96-4.12 (m, 3H), 4.17-4.25 (m, 1H).

### (S)-tert-Butyl 3-(2,2,2-trifluoro-N-(2-oxo-2-(((R)-2′-oxo-1,1′,2′,3-tetrahydrospiro[indene-2,3′-pyrrolo[2,3-b]pyridin]-5-yl)amino)ethyl)acetamido)piperidine-1-carboxylate 8.3

To a solution of compound 8.2 (627 mg, 1.77 mmol), EDCI (441 mg, 2.30 mmol) and HOAt (313 mg, 2.30 mmol) in DMF (10 mL) was added DIEA (687 mg, 5.31 mmol) and Intermediate B (445 mg, 1.77 mmol) at 20° C. The mixture was stirred at 20° C. for 12 h, poured into water (40 mL) and extracted with ethyl acetate (3×40 mL). The organic phases were combined, washed with brine (3×40 mL) and dried over anhydrous sodium sulfate. After filtration and concentration, the residue was purified with silica gel column, eluting with petroleum ether:ethyl acetate=10:1 to 1:3 to give compound 8.3 (910 mg, 86% yield, 98% purity) as a yellow solid. ¹H NMR (CD₃OD, 400 MHz) δ 1.43-1.49 (m, 9H), 1.51-1.58 (m, 1H), 1.74-1.91 (m, 2H), 2.01-2.09 (m, 1H), 2.61-2.76 (m, 1H), 2.87-3.00 (m, 1H), 3.02-3.14 (m, 2H), 3.52 (dd, 2H), 3.82-3.93 (m, 1H), 3.96-4.06 (m, 1H), 4.20-4.39 (m, 3H), 6.88 (dd, 1H), 7.10-7.18 (m, 1H), 0.7.20-7.28 (m, 1H), 7.38 (d, 1H), 7.57 (d, 1H), 8.05 (d, 1H). LC-MS Method 1: rt 0.916 min, (588.3 [M+H]⁺).

Intermediate C

### tert-Butyl (3S)-3-[[2-oxo-2-[[(3R)-2-oxospiro[1H-pyrrolo[2,3-b]pyridine-3,2′-indane]-5′-yl]amino]ethyl]amino]piperidine-1-carboxylate

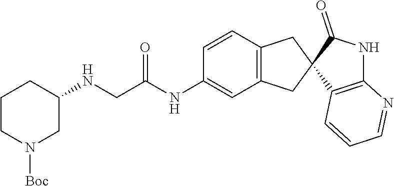 [SMILES: CC(C)(C)OC(=O)N1CCC[C@H](NCC(=O)Nc2ccc3c(c2)C[C@@]2(C3)C(=O)Nc3ncccc32)C1]

To a mixture of compound 8.3 (1.30 g, 1.52 mmol) in methanol (20 mL) and water (6 mL) was added potassium carbonate (632 mg, 4.57 mmol) at 20° C. The mixture was stirred at 20° C. for 12 h, diluted with ethyl acetate (30 mL) and poured into water (30 mL). 1M Hydrochloric acid was added until the pH=3. The organic phase was removed. The aqueous phase was basified with sodium bicarbonate until pH=8 and extracted with ethyl acetate (3×30 mL). The organic phases were combined, washed with brine (2×30 mL) and dried over anhydrous sodium sulfate. After filtration and concentration, the residue was purified with silica gel column, eluting with petroleum ether:ethyl acetate=5:1 to 0:1 to give Intermediate C as a white solid (6 50 mg, 86% yield, 99.5% purity). ¹H NMR (CD₃OD, 400 MHz) δ 1.39-1.53 (m, 11H), 1.69-1.80 (m, 1H), 1.96-2.01 (m, 1H), 2.53-2.62 (m, 1H), 2.70-2.93 (m, 1H), 2.94-3.03 (m, 1H), 3.08 (dd, 2H), 3.45 (s, 2H), 3.52 (dd, 2H), 3.66-3.82 (m, 1H), 3.86-4.02 (m, 1H), 6.88 (dd, 1H), 7.15 (dd, 1H), 7.25 (d, 1H), 7.42 (d, 1H), 7.60 (s, 1H), 8.05 (dd, 1H). LC-MS Method 1: rt 0.699 min, (492.2 [M+H]⁺).

General Route A

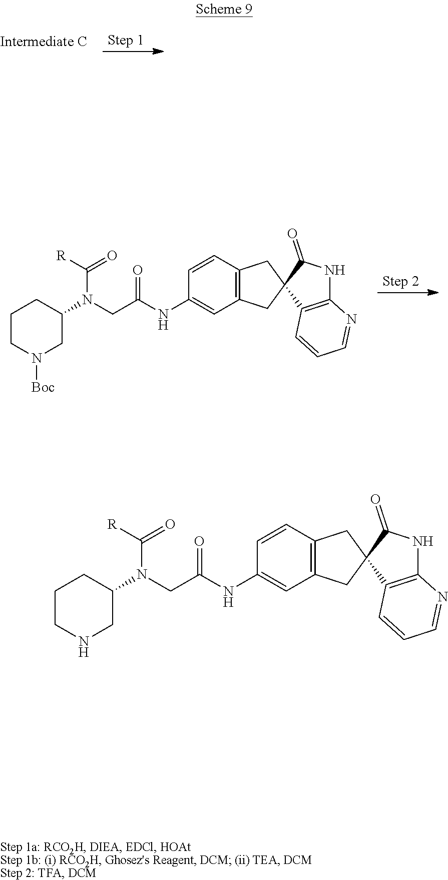

Step 1a: To a solution of the carboxylic acid (1.5˜2.0 eq.) in DMF (1-5 mL) was added EDCI (1.5˜2.0 eq.), HOAt (1.5˜2.0 eq.) and DIEA (1.5˜2.0 eq.) at room temperature. Then Intermediate C (25-70 mg, 0.075-0.105 mmol, 1 eq.) was added. The resulting mixture was stirred at room temperature for 2˜16 h. TLC or LC-MS detected the reaction. When the reaction was finished, the mixture was poured into water (10 mL) and extracted with ethyl acetate (20 mL). The combined organic phases were washed with 1M hydrochloric acid (10 mL), brine (10 mL) and dried over sodium sulfate. After filtration and concentration, the crude product was used directly for the next step or purified by silica gel column chromatography.

Step 1b: To a solution of the carboxylic acid (2.0-4.0 eq.) in dichloromethane (1˜5 mL) was added Ghosez's Reagent (1-chloro-N,N,2-trimethyl-1-propenylamine) at room temperature. The mixture was stirred for 4 h and added to a solution of Intermediate C (25-70 mg, 0.075-0.105 mmol) and TEA (4.0˜8.0 eq.) at 0° C. The resulting mixture stirred at room temperature for 16 h. TLC or LC-MS detected the reaction. When the reaction was finished, the mixture was poured into water (10 mL) and extracted with ethyl acetate (20 mL). The organic phases were combined, washed with 1M hydrochloric acid (10 mL), brine (10 mL) and dried over sodium sulfate. After filtration and concentration, the crude product was used directly for the next step or purified by silica gel column chromatography.

Step 2: The product from Step 1 in a solution of TFA/DCM (1/5, 1˜5 mL) was stirred for 0.5˜2 h. The reaction was monitored by TLC or LC-MS. When the reaction was finished, the mixture was concentrated under vacuum. The residue was purified with prep-HPLC and lyophilisation to provide the final product.

### Example 4

### N-(2-Oxo-2-(((R)-2′-oxo-1,1′,2′,3-tetrahydrospiro[indene-2,3′-pyrrolo [2,3-b]pyridin]-5-yl)amino)ethyl)-N—((S)-piperidin-3-yl)bicyclo[1.1.1]pentane-1-carboxamide

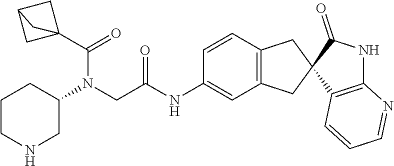 [SMILES: O=C(CN(C(=O)C12CC(C1)C2)[C@H]1CCCNC1)Nc1ccc2c(c1)C[C@@]1(C2)C(=O)Nc2ncccc21]

General Route A, using Intermediate C (40 mg) and Step 1a, after which the product was used without purification. Purification after Step 2 by prep-HPLC (column: Phenomenex Synergi C18 150×25 mm, 10 μm; mobile phase: [solvent A: water (0.1% TFA), solvent B: MeCN]; B %: 12-42%, 10 min). After lyophilisation, Example 4 was obtained as a white solid (27 mg, 56% yield, TFA salt, 99.7% purity). ¹H NMR (CD₃OD, 400 MHz) δ 1.84-1.93 (m, 4H), 2.15-2.31 (m, 6H), 2.41-2.52 (m, 1H), 2.88-2.92 (m, 1H), 3.06-3.18 (m, 3H), 3.35-3.56 (m, 4H), 3.99-4.18 (m, 1H), 4.39-4.57 (m, 2H), 6.91-6.93 (m, 1H), 7.15-7.27 (m, 2H), 7.40-7.42 (m, 1H), 7.52-7.58 (m, 1H), 8.05-8.07 (m, 1H). LC-MS Method 4: rt 1.884 min, (486.1[M+H]⁺).

### Example 5

### N-(2-Oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)cyclopentanecarboxamide

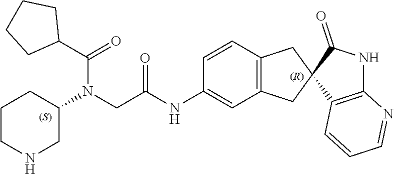

General Route A, using Intermediate C (40 mg) and Step 1a, after which the product was used without purification. Purification after Step 2 by prep-HPLC (column: Luna C18 150×25 mm, 5 μm; mobile phase: [solvent A: water (0.1% TFA), solvent B: MeCN]; B %: 12-42%, 9 min). After lyophilisation, Example 5 was obtained as a white solid (27 mg, 42% yield, TFA salt, 95.6% purity.). ¹H NMR (CD₃OD, 400 MHz) δ 1.59-2.02 (m, 12H), 2.85-2.93 (m, 1.5H), 3.05-3.17 (m, 3.5H), 3.33-3.55 (m, 4H), 4.03-4.19 (m, 1H), 4.34-4.51 (m, 2H), 6.89-6.93 (m, 1H), 7.15-7.26 (m, 2H), 7.40 (t, 1H), 7.55 (d, 1H), 8.06 (d, 1H). LC-MS Method 4: rt1.973 min, (488.2 [M+H]⁺).

### Example 6

### 2-Fluoro-2-methyl-N-[2-oxo-2-[[(3R)-2-oxospiro[1H-pyrrolo[2,3-b]pyridine-3,2′-indane]-5′-yl]amino]ethyl]-N-[(3S)-3-piperidyl]propanamide

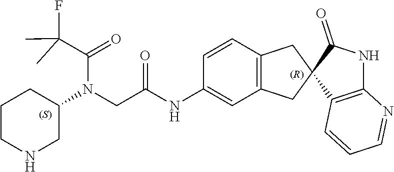

General Route A, using Intermediate C (40 mg) and Step 1b, after which the product was used without purification. Purification after Step 2 by prep-HPLC (column: Phenomenex Synergi C18 150×25 mm, 10 μm; mobile phase: [solvent A: water (0.1% TFA), solvent B: MeCN]; B %: 5-35%, 10 min). After lyophilisation, Example 6 was obtained as a white solid (20 mg, 40% yield, TFA salt, 99.3% purity). ¹H NMR (CD₃OD, 400 MHz) δ 1.55-1.73 (m, 6H), 1.77-2.13 (m, 4H), 2.85-2.96 (m, 1H), 3.04-3.13 (m, 2H), 3.14-3.28 (m, 1H), 3.32-3.37 (m, 1H), 3.46-3.60 (m, 3H), 4.04-4.21 (m, 1H), 4.35-4.48 (m, 1.5H), 4.68-4.79 (m, 0.5H), 6.87-6.94 (m, 1H), 7.13-7.21 (m, 1H), 7.21-7.27 (m, 1H), 7.34-7.44 (m, 1H), 7.51-7.59 (m, 1H), 8.05 (dd, 1H). LC-MS Method 6: rt 1.519 min, (480.3 [M+H]⁺).

### Example 7

### N-(2-Oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)-1-(trifluoromethyl)cyclopropanecarboxamide

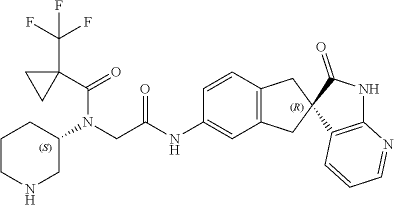

General Route A, using Intermediate C (40 mg) and Step 1b, after which the product was used without purification. Purification after Step 2 by prep-HPLC (column: Boston Prime C18 150×30 mm, 5 μm; mobile phase: [solvent A: water (0.1% TFA), solvent B: MeCN]; B %: 12-42%, 9 min). After lyophilisation, Example 7 was obtained as a white solid (23 mg, 44% yield, TFA salt, 99.2% purity). ¹H NMR (CD₃OD, 400 MHz) δ 1.33-1.46 (m, 4H), 1.88-2.01 (m, 4H), 2.91-2.99 (m, 1H), 3.03-3.25 (m, 3H), 3.36-3.57 (m, 4H), 4.05-4.25 (m, 1.5H), 4.62-4.78 (m, 1.5H), 6.92 (dd, 1H), 7.18 (d, 1H), 7.23-7.31 (m, 1H), 7.41 (d, 1H), 7.52-7.71 (m, 1H), 8.08 (dd, 1H). LC-MS Method 8: rt 1.944 min, (528.2 [M+H]⁺).

### Example 8

### 1-Methyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)cyclobutanecarboxamide

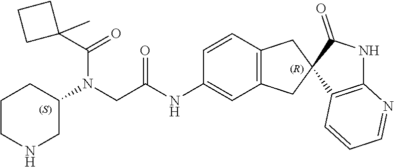

General Route A, using Intermediate C (40 mg) and Step 1b, after which the product was used without purification. Purification after Step 2 by prep-HPLC (column: Boston Prime C18 150×30 mm, 5 μm; mobile phase: [solvent A: water (0.1% TFA), solvent B: MeCN]; B %: 12-42%, 9 min). After lyophilisation, Example 8 was obtained as a white solid (12 mg, 22% yield, TFA salt, 99.1% purity). ¹H NMR (CD₃OD, 400 MHz) δ 1.46-1.52 (d, 3H), 1.68-2.09 (m, 8H), 2.46-2.60 (m, 2H), 2.90 (t, 1H), 3.05-3.19 (m, 3H), 3.32-3.58 (m, 4H), 3.89-4.15 (m, 3H), 6.89 (dd, 1H), 7.22 (t, 1H), 7.26 (t, 1H), 7.45 (t, 1H), 7.59 (d, 1H), 8.06 (d, 1H). LC-MS Method 6: rt 1.570 min, (488.3 [M+H]⁺).

### Example 9

### 3,3,3-Trifluoro-2,2-dimethyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)propanamide

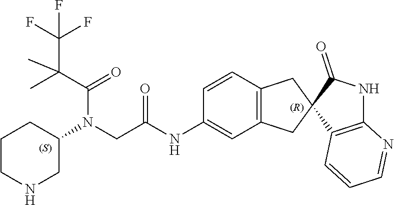

General Route A, using Intermediate C (40 mg) and Step 1b, after which the product was used without purification. Purification after Step 2 by prep-HPLC (column: Luna C18 150×25 mm, 5 μm; mobile phase: [solvent A: water (0.1% TFA), solvent B: MeCN]; B %: 12-42%, 9 min). After lyophilisation, Example 9 was obtained as a white solid (30 mg, 38% yield, TFA salt, 100% purity). ¹H NMR (CD₃OD, 400 MHz) δ 1.59 (s, 6H), 1.73-1.89 (m, 1H), 1.91-2.13 (m, 3H), 2.91 (td, 1H), 3.09 (dd, 2H), 3.21-3.29 (m, 1H), 3.33-3.38 (m, 1H), 3.46-3.61 (m, 3H), 4.00-4.48 (m, 3H), 6.90 (dd, 1H), 7.15 (dd, 1H), 7.24 (d, 1H), 7.40 (dd, 1H), 7.55 (s, 1H), 8.06 (dd, 1H). LC-MS Method 4: rt 1.988 min, (430.1 [M+H]⁺).

### Example 10

### 2-Cyano-2-methyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)propenamide

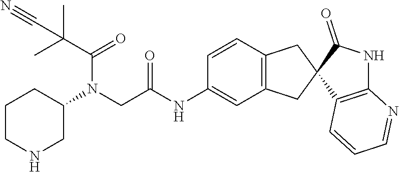 [SMILES: CC(C)(C#N)C(=O)N(CC(=O)Nc1ccc2c(c1)C[C@@]1(C2)C(=O)Nc2ncccc21)[C@H]1CCCNC1]

General Route A, using Intermediate C (60 mg) and Step 1b, after which the product was used without purification. Purification after Step 2 by prep-HPLC (column: Luna C18 150×25 mm, 5 μm; mobile phase: [solvent A: water (0.075% TFA), solvent B: MeCN]; B %: 5-35%, 9 min). After lyophilisation, Example 10 was obtained as a white solid (14 mg, 38% yield, TFA salt, 97.2% purity). ¹H NMR (CD₃OD, 400 MHz) δ 1.58-1.73 (m, 6H), 1.82-2.29 (m, 4H), 2.85-3.00 (m, 1H), 3.08 (d, 2H), 3.18-3.29 (m, 1H), 3.34-3.44 (m, 1H), 3.52 (dd, 2H), 3.57-3.74 (m, 1H), 4.00-4.29 (m, 2H), 4.50-4.72 (m, 1H), 6.90 (dd, 1H), 7.15 (d, 1H), 7.24 (d, 1H), 7.36-7.44 (m, 1H), 7.49-7.61 (m, 1H), 8.06 (dd, 1H). LC-MS Method 4: rt 1.764 min, [M+H]⁺ 487.1.

### Example 11

### 2-Methoxy-2-methyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)propenamide

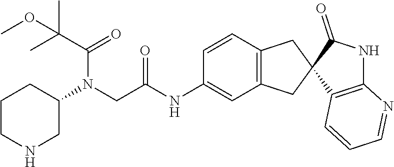 [SMILES: COC(C)(C)C(=O)N(CC(=O)Nc1ccc2c(c1)C[C@@]1(C2)C(=O)Nc2ncccc21)[C@H]1CCCNC1]

General Route A, using Intermediate C (60 mg) and Step 1b, after which the product was used without purification. Purification after Step 2 by prep-HPLC (column: Luna C18 150×25 mm, 5 μm; mobile phase: [solvent A: water (0.075% TFA), solvent B: MeCN]; B %: 5-35%, 9 min). After lyophilisation, Example 11 was obtained as a white solid (31 mg, 53% yield, TFA salt, 98.3% purity). ¹H NMR (CD₃OD, 400 MHz) δ 1.35-1.52 (m, 6H), 1.75-2.18 (m, 4H), 2.91 (t, 1H), 3.01-3.24 (m, 4H), 3.34-3.42 (m, 3H), 3.47-3.66 (m, 3H), 3.90-4.28 (m, 2H), 4.65-4.77 (m, 0.5H), 5.24-5.33 (m, 0.5H), 6.90 (t, 1H), 7.11-7.29 (m, 2H), 7.39 (d, 1H), 7.56 (d, 1H), 8.06 (d, 1H). LC-MS Method 6: rt 1.499 min, [M+H]⁺.

General Route B

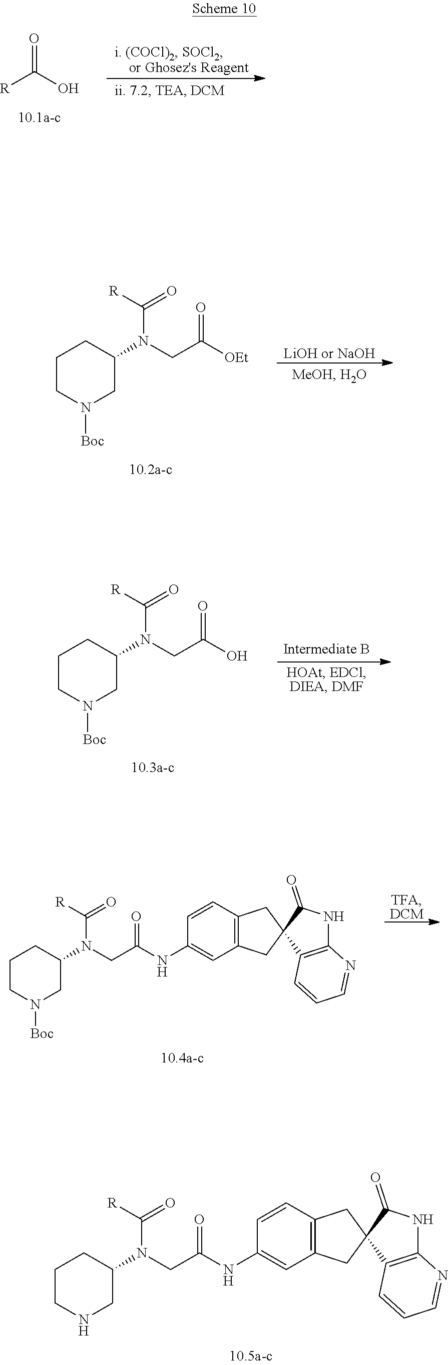

### (S)-tert-Butyl 3-(N-(2-ethoxy-2-oxoethyl)-1-(trifluoromethyl)cyclobutanecarboxamido) piperidine-1-carboxylate 10.2a

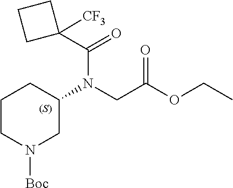

To a solution of 1-(trifluoromethyl)cyclobutanecarboxylic acid (10.1a) (150 mg, 0.89 mmol) in dichloromethane (4 mL) was added oxalyl chloride (0.2 mL) and DMF (6.39 mg, 0.087 mmol). The mixture was stirred at 15° C. for 1 h and concentrated at 15° C. The residue was dissolved with dichloromethane (2 mL) and added to a solution of compound 7.2 (150 mg, 0.52 mmol) and triethylamine (0.3 mL) in dichloromethane (4 mL) at 0° C. The mixture was stirred at 15° C. for 2 h, poured into water (40 mL) and extracted with ethyl acetate (3×40 mL). The organic phases were combined, washed with brine (100 mL) and dried over sodium sulfate. After filtration and concentration, the residue was purified by silica gel column chromatography (petroleum ether:ethyl acetate=10:1˜3:1) to afford compound 10.2a as a white solid (130 mg, 57% yield). ¹H NMR (CDCl₃, 400 MHz) δ 1.29 (t, 3H), 1.47 (s, 9H), 1.50-1.60 (m, 2H), 1.74-1.95 (m, 2H), 1.99-2.18 (m, 2H), 2.43-2.67 (m, 4H), 2.72-2.86 (m, 2H), 3.44-3.50 (m, 1H), 3.90-3.99 (m, 2H), 4.06-4.35 (m, 4H).

### (S)-2-(N-(1-(tert-butoxycarbonyl)piperidin-3-yl)-1-(trifluoromethyl)cyclobutanecarboxamido)acetic acid 10.3a

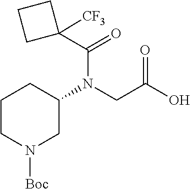 [SMILES: CC(C)(C)OC(=O)N1CCC[C@H](N(CC(=O)O)C(=O)C2(C(F)(F)F)CCC2)C1]

To a solution of compound 10.2a (130 mg, 0.31 mmol) in methanol (3 mL) and water (1 mL) was added sodium hydroxide (62 mg, 1.54 mmol). The mixture was stirred at 15° C. for 6 h, adjusted to pH4 with 1M hydrochloric acid and extracted with ethyl acetate (2×50 mL). The organic phases were combined, washed with brine (50 mL) and dried over sodium sulfate. After filtration and concentration, compound 10.3a was obtained as a white solid (110 mg, 87% yield). ¹H NMR (CDCl₃, 400 MHz) δ 1.46 (s, 9H), 1.50-1.58 (m, 2H), 1.73-1.81 (m, 1H), 1.83-1.93 (m, 1H), 1.95-2.02 (m, 1H), 2.12-2.17 (m, 1H), 2.43-2.84 (m, 6H), 3.43-3.51 (m, 1H), 4.00 (s, 2H), 4.10-4.35 (m, 2H).

### (S)-tert-Butyl 3-(N-(2-oxo-2-(((R)-2′-oxo-1,1′,2′,3-tetrahydrospiro[indene-2,3′-pyrrolo [2,3-b]pyridin]-5-yl)amino)ethyl)-1-(trifluoromethyl)cyclobutanecarboxamido)piperidine-1-carboxylate 10.4a

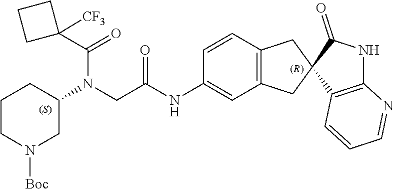

To a solution of compound 10.3a (100 mg, 0.24 mmol.) in DMF (2 mL) was added DIEA (79 mg, 0.61 mmol), HOAt (43 mg, 0.32 mmol), EDCI (61 mg, 0.32 mmol) and Intermediate B (61 mg, 0.24 mmol). The mixture was stirred at 25° C. for 2 h, poured into water (50 mL) and extracted with ethyl acetate (3×100 mL). The organic phases were combined, washed with brine (100 mL) and dried over sodium sulfate. After filtration and concentration, compound 10.4a was obtained as a light-yellow solid (130 mg, crude). LC-MS Method 10: rt 0.981 min, (664.4 [M+Na]⁺).

### Example 12

### N-(2-Oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)-1-(trifluoromethyl)cyclobutanecarboxamide 10.5a

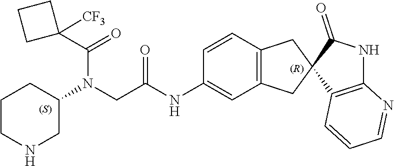

To a solution of compound 10.4a (100 mg, 0.16 mmol) in dichloromethane (10 mL) was added TFA (1 mL). The mixture was stirred at 25° C. for 30 min and concentrated under vacuum. The residue was purified by prep-HPLC (column: Phenomenex Synergi C18 150×25 mm, 10 μm; mobile phase: [solvent A: water (0.1% TFA), solvent B: MeCN]; B %: 15-45%, 5 min). After lyophilisation, compound 10.5a was obtained as a white solid (89.10 mg, 84.9% yield, TFA salt, 97.4% purity). ¹H NMR (CD₃OD, 400 MHz) δ 1.72-2.24 (m, 6H), 2.51-2.68 (m, 2H), 2.75-2.96 (m, 3H), 3.08-3.16 (m, 2H), 3.19-3.22 (m, 0.5H), 3.34-3.40 (m, 1H), 3.51-3.57 (m, 3H), 3.63-3.67 (m, 0.5H), 3.85-3.96 (m, 1H), 4.09-4.28 (m, 2H), 6.92 (dd, 1H), 7.17-7.19 (m, 1H), 7.25-7.30 (m, 1H), 7.40-7.44 (m, 1H), 7.55-7.64 (m, 1H), 8.07-8.08 (m, 1H). LC-MS Method 4: rt 2.001 min, (542.1[M+H]⁺).

### (S)-tert-Butyl 3-(N-(2-ethoxy-2-oxoethyl)-4,4,4-trifluoro-2,2-dimethylbutanamido) piperidine-1-carboxylate 10.2b

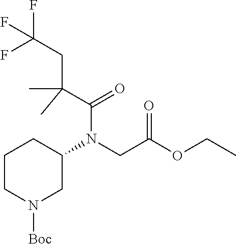 [SMILES: CCOC(=O)CN(C(=O)C(C)(C)CC(F)(F)F)[C@H]1CCCN(C(=O)OC(C)(C)C)C1]

To a solution of 4,4,4-trifluoro-2,2-dimethyl-butanoic acid (10.1b) (370 mg, 2.17 mmol) in DCM (4 mL) was added 1-chloro-N,N,2-trimethyl-prop-1-en-1-amine (406 mg, 3.04 mmol) at 20° C. The mixture was stirred at 20° C. for 4 h and added to a solution of compound 7.2 (623 mg, 2.17 mmol) and triethylamine (660 mg, 6.52 mmol) in DCM (6 mL) at 20° C. The resulting mixture was stirred at 20° C. for 2 h, poured into water (10 mL) and extracted with ethyl acetate (3×20 mL). The organic phases were combined, washed by brine (2×20 mL) and dried over anhydrous sodium sulfate. After filtration and concentration, the residue was purified by prep-HPLC (column: Luna C18 150×25 mm, 5 μm; mobile phase: [solvent A: water (0.075% TFA), solvent B: MeCN]; B %: 52-82%, 9 min). After lyophilisation, compound 10.2b was obtained as colourless oil (35 mg, 3% yield, 92% purity). LC-MS Method 1: rt 1.006 min, [M+Na]⁺461.3.

### Sodium (S)-2-(N-(1-(tert-butoxycarbonyl)piperidin-3-yl)-4,4,4-trifluoro-2,2-dimethyl butanamido) acetate 10.3b

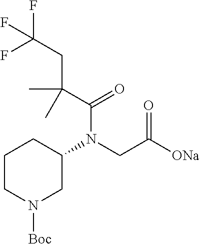

To a solution of compound 10.2b (35 mg, 0.080 mmol) in methanol (1.5 mL) and water (0.5 mL) was added sodium hydroxide (6.39 mg, 0.16 mmol) at 20° C. The mixture was stirred at 20° C. for 12 h, adjusted to pH8 with 1M of hydrochloric acid and concentrated under vacuum to afford compound 10.3b (Na salt) (35 mg, crude) as a yellow solid, which used directly for next step. LC-MS Method 1: rt 0.881 min, [M+H]⁺ 433.1

### (S)-tert-Butyl 3-(4,4,4-trifluoro-2,2-dimethyl-N-(2-oxo-2-(((R)-2′-oxo-1,1′,2′,3-tetrahydrospiro[indene-2,3′-pyrrolo[2,3-b]pyridin]-5-yl)amino)ethyl)butanamido)piperidine-1-carboxylate 10.4b

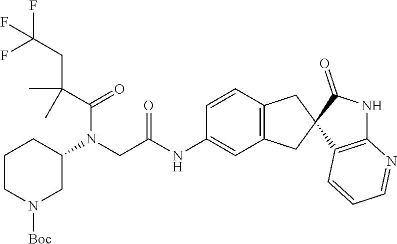 [SMILES: CC(C)(C)OC(=O)N1CCC[C@H](N(CC(=O)Nc2ccc3c(c2)C[C@@]2(C3)C(=O)Nc3ncccc32)C(=O)C(C)(C)CC(F)(F)F)C1]

To a solution of compound 10.3b (Na salt) (35 mg, 0.081 mmol), EDCI (23 mg, 0.12 mmol) and HOAt (16 mg, 0.12 mmol) in DMF (1 mL) was added DIEA (21 mg, 0.16 mmol) followed by Intermediate B (20 mg, 0.081 mmol) at 20° C. The mixture was stirred at 20° C. for 12 h, poured into water (20 mL) and extracted with ethyl acetate (3×20 mL). The organic phases were combined, washed by 0.1M hydrochloric acid (20 mL) and brine (4×20 mL) and dried over anhydrous sodium sulfate. After filtration and concentration, the residue was purified by prep-TLC (ethyl acetate) to provide compound 10.4b as a white solid (20 mg, 37% yield, 96.7% purity). LC-MS Method 1: rt 0.947 min, [M+H]⁺ 644.

### Example 13

### 4,4,4-Trifluoro-2,2-dimethyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)butanamide 10.5b

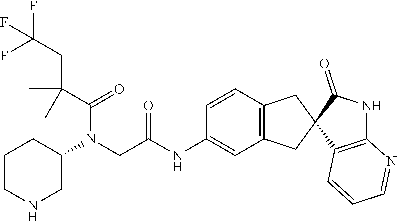 [SMILES: CC(C)(CC(F)(F)F)C(=O)N(CC(=O)Nc1ccc2c(c1)C[C@@]1(C2)C(=O)Nc2ncccc21)[C@H]1CCCNC1]

To a solution of compound 10.4b (20 mg, 0.031 mmol) in DCM (2 mL) was added trifluoroacetic acid (320 μL) at 20° C. The mixture was stirred for 30 min and concentrated in vacuo to give a residue, which was purified by prep-HPLC (column: Luna C18 150×25 mm, 5 μm; mobile phase: [solvent A: water (0.075% TFA), solvent B: MeCN]; B %: 12-42%, 9 min). After lyophilisation, compound 10.5b was obtained as an off-white solid (5 mg, 23% yield, TFA salt, 96.7% purity). ¹H NMR (CD₃OD, 400 MHz) δ 1.43 (s, 6H), 1.73-1.89 (m, 1H), 1.92-2.13 (m, 3H), 2.58-2.76 (m, 2H), 2.91 (t, 1H), 3.09 (dd, 2H), 3.33-3.40 (m, 2H), 3.45-3.59 (m, 3H), 3.90-4.49 (m, 3H), 6.89 (dd, 1H), 7.15 (d, 1H), 7.24 (d, 1H), 7.40 (d, 1H), 7.56 (s, 1H), 8.06 (d, 1H). LC-MS (Long acidic method 3): rt Method 9: 2.445 min, [M+H]⁺ 544.3.

Carboxylic acid 10.1c was prepared according to Scheme 10A and used in the synthesis of Example 14 by General Route B

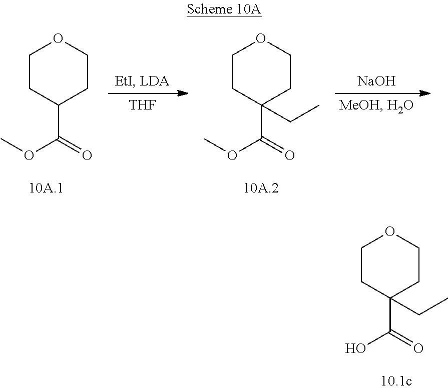

### Methyl 4-ethyltetrahydro-2H-pyran-4-carboxylate 10A.2

To a solution of compound 10A.1 (3.00 g, 20.8 mmol) in THF (30 mL) was added 2M LDA (13.53 mL) dropwise at −70° C. under nitrogen. The mixture was stirred at −70° C. for 1 h under nitrogen. Iodoethane (4.87 g, 31.2 mmol) was added into the mixture dropwise at −70° C. The resulting mixture was stirred at −70° C. for 30 min, allowed to warm to 20° C. and stirred for another 2 h. The reaction mixture was poured into water (20 mL) and extracted with ethyl acetate (2×50 mL). The combined organic layers were washed with brine (50 mL) and dried over sodium sulfate. After filtration and concentration, the residue was purified by silica gel column, eluting with petroleum ether:ethyl acetate=1:0˜5:1 to provide compound 10A.2 as colourless oil (3.50 g, 98% yield). ¹H NMR (CDCl₃, 400 MHz) δ 0.79 (t, 3H), 1.41-1.50 (m, 2H), 1.55 (q, 2H), 2.04 (dd, 2H), 3.40 (td, 2H), 3.69 (s, 3H), 3.80 (dt, 2H).

### 4-Ethyltetrahydropyran-4-carboxylic acid 10.1c

To a solution of compound 10A.2 (3.50 g, 20.3 mmol) in methanol (30 mL) was added a solution of sodium hydroxide (813 mg, 20.3 mmol) in water (10 mL) at 20° C. The mixture was heated to 80° C. and stirred for 16 h. It was poured into water (20 mL), adjusted to pH4 with 1M hydrochloric acid, and extracted with ethyl acetate (2×50 mL). The organic layers were combined, washed with brine (50 mL) and dried over sodium sulfate. After filtration and concentration, compound 10.1c was obtained as a white solid (2.10 g, 65% yield). ¹H NMR (CDCl₃, 400 MHz) δ 0.90 (t, 3H), 1.47-1.57 (m, 2H), 1.63 (q, 2H), 2.06 (dd, 2H), 3.52 (td, 2H), 3.87 (dt, 2H).

### tert-Butyl (3S)-3-[(2-ethoxy-2-oxo-ethyl)-(4-ethyltetrahydropyran-4-carbonyl)amino]piperidine-1-carboxylate 10.2c

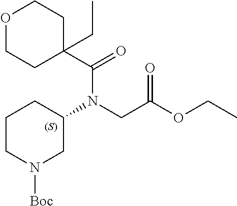

To a solution of compound 10.1c (414.3 mg, 2.62 mmol) and DMF (7.66 mg, 0.10 mmol) in dichloromethane (5 mL) was added thionyl chloride (1.87 g, 15.7 mmol) at 20° C. and stirred for 1 h. The reaction mixture was concentrated under vacuum. The residue was dissolved with dichloromethane (3 mL) and added into a solution of compound 7.2 (300 mg, 1.05 mmol) and triethylamine (636 mg, 6.29 mmol) in dichloromethane (3 mL) at 0° C. The mixture was stirred at 20° C. for 2 h, poured into water (30 mL) and extracted with ethyl acetate (3×30 mL). The combined organic phases were washed by 0.1M hydrochloric acid (30 mL), brine (2×20 mL) and dried over anhydrous sodium sulfate. After filtration and concentration, the residue was purified by silica gel column, eluting with petroleum ether:ethyl acetate=20:1˜1:3 to provide compound 10.2c as a colourless oil (90 mg, 17% yield, 83.2% purity). ¹H NMR (CD₃OD, 400 MHz) δ 0.95 (t, 3H), 1.29 (t, 3H), 1.46 (s, 9H), 1.59-1.80 (m, 9H), 1.89-1.98 (m, 1H), 2.11-2.30 (m, 2H), 2.42-2.80 (m, 2H), 3.59-3.71 (m, 2H), 3.76-3.96 (m, 4H), 3.98-4.08 (m, 1H), 4.19 (q, 2H).

### 2-[[(3S)-1-tert-Butoxycarbonyl-3-piperidyl]-(4-ethyltetrahydropyran-4-carbonyl)amino]acetic acid 10.3c

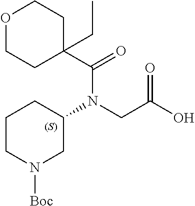

To a solution of compound 10.2c (90 mg, 0.18 mmol) in methanol (3 mL) was added a solution of sodium hydroxide (35 mg, 0.88 mmol) in water (1 mL) at 20° C. The mixture stirred for 2 h, poured into water (20 mL), acidified to pH4 with 1M hydrochloric acid and extracted with ethyl acetate (3×20 mL). The organic phases were combined, washed by brine (2×20 mL) and dried over anhydrous sodium sulfate. After filtration and concentration, compound 10.3c was obtained as yellow gum (70 mg, 95% yield, 95.1% purity). LC-MS Method 1: rt 0.864 min, (421.2 [M+Na]⁺).

### (S)-tert-Butyl 3-(4-ethyl-N-(2-oxo-2-(((R)-2′-oxo-1,1′,2′,3-tetrahydrospiro[indene-2,3′-pyrrolo[2,3-b]pyridin]-5-yl)amino)ethyl)tetrahydro-2H-pyran-4-carboxamido)piperidine-1-carboxylate 10.4c

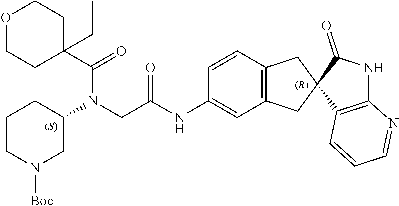

To a solution of compound 10.3c (70 mg, 0.18 mmol), EDCI (50 mg, 0.26 mmol) and HOAt (36 mg, 0.26 mmol) in DMF (2 mL) was added DIEA (68 mg, 0.53 mmol) followed by Intermediate B (44 mg, 0.18 mmol) at 20° C. The mixture was stirred at 20° C. for 12 h, poured into water (10 mL) and extracted with ethyl acetate (3×20 mL). The organic phases were combined, washed by 0.1M hydrochloric acid (20 mL), brine (2×20 mL) and dried over anhydrous sodium sulfate. After filtration and concentration, compound 10.4c was obtained as a yellow solid (90 mg, 73% yield, 90.3% purity). ¹H NMR (CD₃OD, 400 MHz) δ 0.96 (t, 3H), 1.47 (s, 9H), 1.51-1.62 (m, 3H), 1.70-1.84 (m, 4H), 1.89-1.97 (m, 1H), 2.15-2.22 (m, 1H), 2.25-2.35 (m, 1H), 2.56-2.75 (m, 1H), 2.87-2.98 (m, 1H), 3.07 (dd, 2H), 3.52 (dd, 2H), 3.63-3.84 (m, 4H), 3.92-4.11 (m, 4H), 4.20 (d, 1H), 6.88 (dd, 1H), 7.14 (dd, 1H), 7.23 (d, 1H), 7.37 (dd, 1H), 7.58 (s, 1H), 8.05 (dd, 1H). LC-MS Method 1: rt 0.934 min, (632.4 [M+H]⁺).

### Example 14

### 4-Ethyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)tetrahydro-2H-pyran-4-carboxamide 10.5c

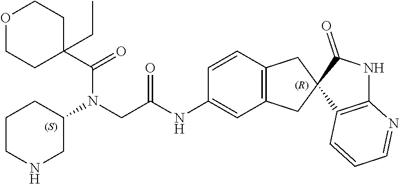

To a solution of compound 10.4c (70 mg, 0.11 mmol) in dichloromethane (2 mL) was added TFA (0.4 mL) at 20° C., the mixture stirred for 30 min and concentrated under vacuum. The residue was purified by prep-HPLC (column: Phenomenex Synergi C18 150×25 mm, 10 μm; mobile phase: [solvent A: water (0.1% TFA), solvent B: MeCN]; B %: 10-40%, 10 min). After lyophilisation, compound 10.5c was obtained as a white solid (41 mg, 57% yield, TFA salt, 99.8% purity). ¹H NMR (CD₃OD, 400 MHz) δ 0.93 (t, 3H), 1.51-1.66 (m, 2H), 1.70-2.11 (m, 6H), 2.12-2.27 (m, 2H), 2.91 (td, 1H), 3.09 (dd, 2H), 3.15-3.30 (m, 1H), 3.34-3.40 (m, 1H), 3.46-3.57 (m, 3H), 3.63 (q, 2H), 3.72-3.84 (m, 2H), 3.91-4.60 (m, 3H), 6.91 (dd, 1H), 7.17 (d, 1H), 7.24 (d, 1H), 7.39 (d, 1H), 7.56 (s, 1H), 8.06 (dd, 1H). LC-MS Method 4: rt 1.820 min, (532.2 [M+H]⁺).

Synthesis of Intermediates D and E

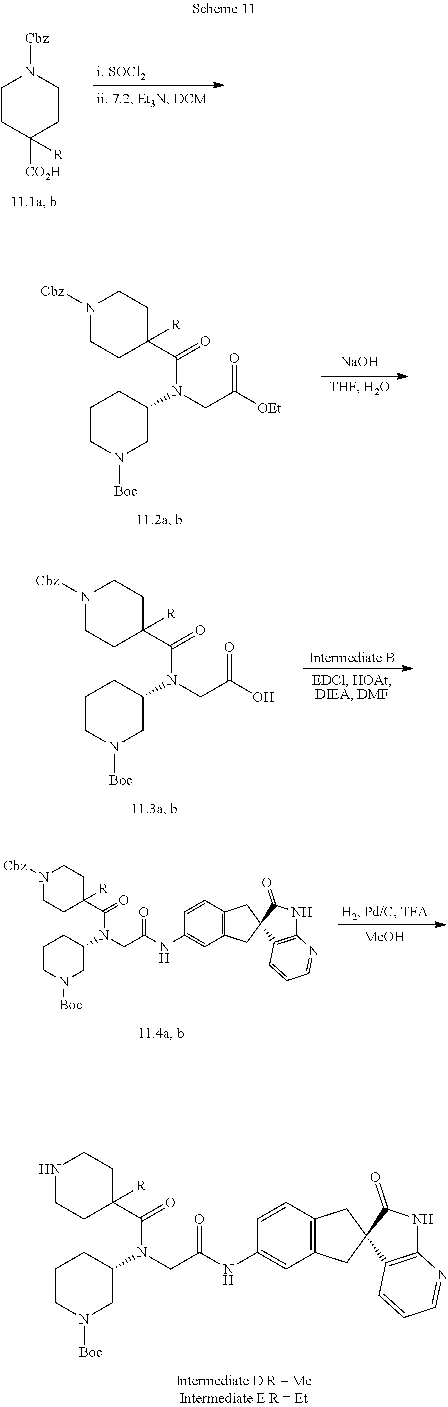

### 4-((1-(tert-Butoxycarbonyl)piperidin-3-yl)(2-ethoxy-2-oxoethyl)carbamoyl)-4-methylpiperidine-1-carboxylate 11.2a

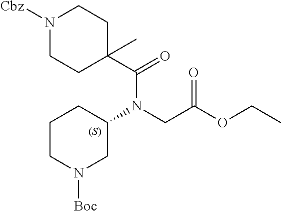

To a solution of compound 11.1a (730 mg, 2.63 mmol) in dichloromethane (8 mL) was added DMF (19 mg, 0.26 mmol) and thionyl chloride (1.91 mL). The mixture was stirred at 15° C. for 1 h and concentrated. The residue was dissolved in dichloromethane (5 mL) and added to a solution of compound 7.2 (660 mg, 2.30 mmol) and triethylamine (727 mg, 7.18 mmol) in dichloromethane (5 mL). The mixture was stirred at 10° C. for 32 h, poured into water (50 mL) and extracted with ethyl acetate (2×50 mL). The combined organic layers were washed with brine (50 mL) and dried over anhydrous sodium sulfate. After filtration and concentration, the residue was purified by reverse flash chromatography [solvent A: water (0.1% TFA), solvent B: MeCN] B %: 0-95% to afford compound 11.2a as colourless oil (400 mg, 30% yield, 93.9% purity). ¹H NMR (CDCl₃, 400 MHz) δ 1.27 (t, 3H), 1.34 (s, 3H), 1.45 (s, 9H), 1.51-1.60 (m, 6H), 1.78-1.95 (m, 2H), 2.13-2.25 (m, 2H), 2.53-2.68 (m, 2H), 3.29 (br. s, 2H), 3.74-3.95 (m, 4H), 3.95-4.05 (m, 1H), 4.18 (q, 2H), 5.12 (s, 2H), 7.31-7.36 (m, 5H).

### (S)-2-(1-((Benzyloxy)carbonyl)-N-(1-(tert-butoxycarbonyl)piperidin-3-yl)-4-methylpiperidine-4-carboxamido)acetic acid 11.3a

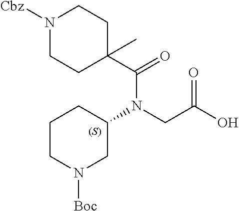

To a solution of compound 11.2a (760 mg, 1.39 mmol) in tetrahydrofuran (3 mL), methanol (0.5 mL) and water (0.5 mL) was added sodium hydroxide (111 mg, 2.79 mmol). The mixture was stirred at 70° C. for 30 min, adjusted to pH4 with 1M hydrochloric acid and extracted with ethyl acetate (2×50 mL). The organic phases were combined, washed with brine (50 mL) and dried over sodium sulfate. After filtration and concentration, compound 11.3a was obtained as light-yellow oil (720 mg, 96% yield, 95.7% purity). LC-MS Method 1: rt 0.848 min, (518.4 [M+H]⁺).

### Benzyl 4-(((S)-1-(tert-butoxycarbonyl)piperidin-3-yl)(2-oxo-2-(((R)-2′-oxo-1,1′,2′,3-tetrahydrospiro[indene-2,3′-pyrrolo[2,3-b]pyridin]-5-yl)amino)ethyl)carbamoyl)-4-methylpiperidine-1-carboxylate 11.4a

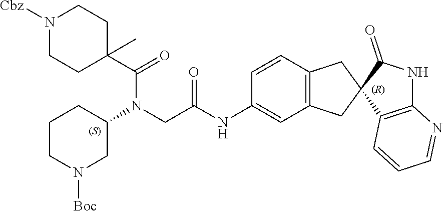

To a solution of compound 11.3a (720 mg, 1.39 mmol.) in DMF (10 mL) was added DIEA (539 mg, 4.17 mmol, 3 eq.), HOAt (246 mg, 1.81 mmol, 1.3 eq.), EDCI (347 mg, 1.81 mmol, 1.3 eq.) and Intermediate B (400 mg, 1.59 mmol). The mixture was stirred at 25° C. for 16 h, poured into water (50 mL) and extracted with ethyl acetate (2×50 mL). The organic layers were combined, washed with brine (3×40 mL) and dried over anhydrous sodium sulfate. After filtration and concentration, the residue was purified by prep-HPLC (column: Phenomenex Synergi C18 150×25 mm, 10 μm; mobile phase: [solvent A: water (0.225% TFA), solvent B: MeCN]; B %: 45-75%, 10 min). After extraction with ethyl acetate (3×50 mL), compound 11.4a was obtained as white solid (460 mg, 44% yield). ¹H NMR (CDCl₃, 400 MHz) δ 1.39 (s, 3H), 1.44 (s, 9H), 1.50-1.61 (m, 3H), 1.81-1.90 (m, 3H), 2.14-2.27 (m, 2H), 2.60-2.95 (m, 2H), 3.03 (dd, 2H), 3.30-3.36 (m, 2H), 3.62 (dd, 2H), 3.75-3.88 (m, 2H), 3.93-4.04 (m, 2H), 4.06-4.20 (m, 3H), 5.12 (s, 2H), 6.84 (dd, 1H), 7.10 (dd, 1H), 7.17-7.19 (m, 1H), 7.21-7.26 (m, 1H), 7.33-7.36 (m, 5H), 7.50 (br. s, 1H), 8.07-8.09 (m, 1H), 8.62-9.00 (m, 2H).

Intermediate D

### (S)-tert-Butyl 3-(4-methyl-N-(2-oxo-2-(((R)-2′-oxo-1,1′,2′,3-tetrahydrospiro[indene-2,3′-pyrrolo[2,3-b]pyridin]-5-yl)amino)ethyl)piperidine-4-carboxamido)piperidine-1-carboxylate

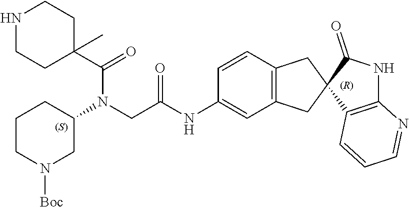

To a solution of compound 11.4a (500 mg, 0.67 mmol) in methanol (5 mL) was added TFA (76 mg, 0.67 mmol) and 10% Pd/C (50 mg). The mixture was degassed and purged with hydrogen three times and stirred at 25° C. for 16 h under a hydrogen balloon. The catalyst was removed by filtration and the filtrate was concentrated to afford Intermediate D as a white solid (410 mg, 99% yield, TFA salt, 99.3% purity). 1H NMR (CD₃OD, 400 MHz) δ 1.45 (s, 3H), 1.49 (s, 9H), 1.55-1.84 (m, 6H), 1.97 (m, 1H), 2.41 (d, 1H), 2.44-3.06 (m, 4H), 3.11 (d, 2H), 3.25-3.31 (m, 1H), 3.38-3.41 (m, 1H), 3.54 (dd, 2H), 4.03-4.21 (m, 5H), 6.91 (dd, 1H), 7.16 (dd, 1H), 7.26 (d, 1H), 7.40 (d, 1H), 7.65 (s, 1H), 8.08 (dd, 1H). LC-MS Method 1: rt 0.744 min, (617.5 [M+H]⁺).

### Example 15

### 4-Methyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)piperidine-4-carboxamide

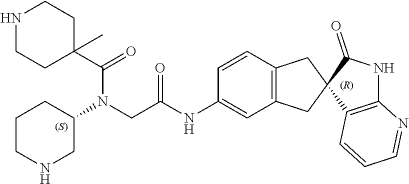

To a solution of compound Intermediate D (40 mg, 0.065 mmol) in dichloromethane (5 mL) was added TFA (0.5 mL). The mixture was stirred at 25° C. for 30 min, concentrated and the residue was purified by prep-HPLC (column: Luna C18 150×25 mm, 5 μm; mobile phase: [solvent A: water (0.1% TFA), solvent B: MeCN]; B %: 10-27%, 7 min). After lyophilisation, Example 15 was obtained as a white solid (21 mg, 43% yield, bis-TFA salt, 99.0% purity). ¹H NMR (CD₃OD, 400 MHz) δ 1.46 (s, 3H), 1.70-2.05 (m, 6H), 2.36-2.41 (m, 2H), 2.91-2.96 (m, 1H), 3.10 (dd, 2H), 3.17-3.31 (m, 5H), 3.38-3.41 (m, 1H), 3.53 (dd, 3H), 4.11-4.47 (m, 3H), 6.91 (dd, 1H), 7.16 (dd, 1H), 7.25 (d, 1H), 7.39 (d, 1H), 7.60 (s, 1H), 8.08 (dd, 1H). LC-MS Method 6: rt 1.013 min, (517.3 [M+H]⁺).

Intermediate E

The procedures described in detail above were applied to the analogous synthesis of Intermediate E, for which data and key procedural details are given below.

### Benzyl 4-[[(3S)-1-tert-butoxycarbonyl-3-piperidyl]-(2-ethoxy-2-oxo-ethyl)carbamoyl]-4-ethyl-piperidine-1-carboxylate 11.2b

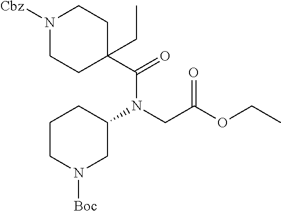 [SMILES: CCOC(=O)CN(C(=O)C1(CC)CCN(C(=O)OCc2ccccc2)CC1)[C@H]1CCCN(C(=O)OC(C)(C)C)C1]

Final purification by silica gel column chromatography, eluting with petroleum ether:ethyl acetate=12:1˜3:1 gave compound 11.2b as a yellow oil. ¹H NMR (CDCl₃, 400 MHz) δ 0.96 (t, 3H), 1.27 (t, 3H), 1.44-1.47 (m, 9H), 1.55-1.80 (m, 9H), 1.89-1.94 (m, 1H), 2.20-2.32 (m, 2H), 2.51-2.69 (m, 2H), 3.10-3.30 (m, 2H), 3.86-4.03 (m, 5H), 4.20 (q, 2H), 5.12 (s, 2H), 7.28-7.38 (m, 5H).

### 2-[(1-Benzyloxycarbonyl-4-ethyl-piperidine-4-carbonyl)-[(3S)-1-tert-butoxycarbonyl-3-piperidyl]amino]acetic acid 11.3b

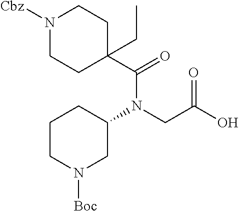 [SMILES: CCC1(C(=O)N(CC(=O)O)[C@H]2CCCN(C(=O)OC(C)(C)C)C2)CCN(C(=O)OCc2ccccc2)CC1]

Crude 11.3b was obtained as a yellow solid. ¹H NMR (CDCl₃, 400 MHz) δ 0.94 (t, 3H), 1.38-1.47 (m, 12H), 1.56-1.75 (m, 5H), 1.93 (dd, 2H), 2.23 (dd, 2H), 2.50-2.75 (m, 2H), 3.05-3.25 (m, 2H), 3.83-4.05 (m, 5H), 5.12 (s, 2H), 7.28-7.38 (m, 5H).

### Benzyl 4-[[(3S)-1-tert-butoxycarbonyl-3-piperidyl]-[2-oxo-2-[[(3R)-2-oxospiro[1H-pyrrolo[2,3-b]pyridine-3,2′-indane]-5′-yl]amino]ethyl]carbamoyl]-4-ethyl-piperidine-1-carboxylate 11.4b

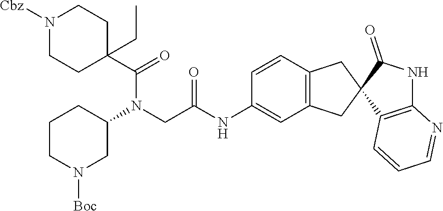 [SMILES: CCC1(C(=O)N(CC(=O)Nc2ccc3c(c2)C[C@@]2(C3)C(=O)Nc3ncccc32)[C@H]2CCCN(C(=O)OC(C)(C)C)C2)CCN(C(=O)OCc2ccccc2)CC1]

Purification by silica gel column chromatography, eluting with petroleum ether:ethyl acetate=3:1˜0:1 gave compound 11.4b as a yellow oil. ¹H NMR (CDCl₃, 400 MHz) δ 0.94 (t, 3H), 1.40-1.47 (m, 12H), 1.66-1.94 (m, 7H), 2.23-2.36 (m, 2H), 2.50-2.70 (m, 1H), 2.99-3.08 (m, 3H), 3.15-3.25 (m, 2H), 3.58-3.64 (m, 2H), 3.95-4.11 (m, 5H), 5.11 (s, 2H), 6.82 (dd, 1H), 7.09 (dd, 1H), 7.15-7.17 (m, 1H), 7.29-7.37 (m, 5H), 7.44-7.52 (m, 1H), 8.02 (s, 1H), 8.06 (d, 1H), 8.81 (br. s, 1H).

Intermediate E

### tert-Butyl (3S)-3-[(4-ethylpiperidine-4-carbonyl)-[2-oxo-2-[[(3R)-2-oxospiro[1H-pyrrolo[2,3-b]pyridine-3,2′-indane]-5′-yl]amino]ethyl]amino]piperidine-1-carboxylate

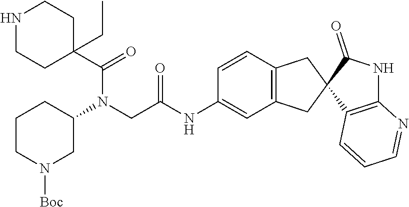 [SMILES: CCC1(C(=O)N(CC(=O)Nc2ccc3c(c2)C[C@@]2(C3)C(=O)Nc3ncccc32)[C@H]2CCCN(C(=O)OC(C)(C)C)C2)CCNCC1]

Intermediate E was isolated, without purification, as a yellow oil.

General Route C

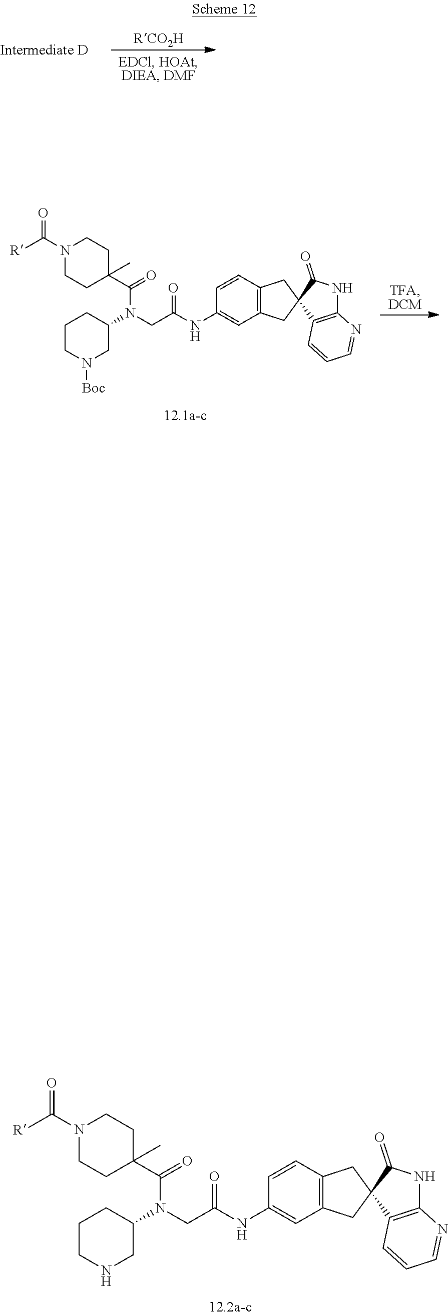

### Example 16

### 1-Acetyl-4-methyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)piperidine-4-carboxamide 12.2a

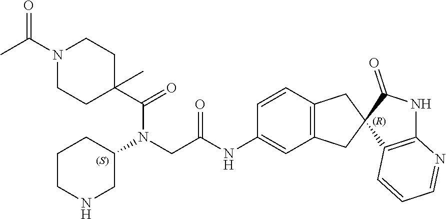

To a solution of acetic acid (10 mg, 0.16 mmol) in DMF (1 mL) was added diisopropylethylamine (52 mg, 0.40 mmol), EDCI (32 mg, 0.16 mmol) and HOAt (22 mg, 0.16 mmol). Intermediate D (50 mg, 0.81 mmol) was added, the mixture stirred at 20° C. for 12 h, quenched with water (10 mL), and extracted with ethyl acetate (3×20 mL). The combined organic layers were washed with brine (3×20 mL) and dried over anhydrous sodium sulfate. After filtration and concentration, compound 12.1a was obtained as yellow oil, which was dissolved in dichloromethane (1 mL) and trifluoroacetic acid (0.2 mL) added. The mixture was stirred at 20° C. for 5 min, concentrated in vacuum and the residue was purified by prep-HPLC (column: Luna C18 150×25 mm, 5 μm; mobile phase: [solvent A: water (0.1% TFA), solvent B: MeCN]; B %: 15-32%, 7 min). After lyophilisation, compound 12.2a (24 mg, 45% yield, TFA salt, 100% purity) was obtained as a white solid. ¹H NMR (CD₃OD, 400 MHz) δ 1.41 (s, 3H), 1.53-1.58 (m, 2H), 1.78-1.90 (m, 1H), 1.97-2.15 (m, 6H), 2.17-2.30 (m, 2H), 2.91-2.97 (m, 1H), 3.10 (dd, 2H), 3.16-3.30 (m, 2H), 3.38 (d, 1H), 3.47-3.57 (m, 4H), 3.68-3.71 (m, 1H), 3.97-4.52 (m, 4H), 6.91 (dd, 1H), 7.16-7.18 (m, 1H), 7.26 (d, 1H), 7.40-7.43 (m, 1H), 7.57 (s, 1H), 8.07 (dd, 1H). LC-MS Method 4: rt 1.777 min, (559.2 [M+H]⁺).

### Example 17

### 4-Methyl-N-(2-oxo-2-(((R)-2′-oxo-1,1′,2′,3-tetrahydrospiro[indene-2,3′-pyrrolo[2,3-b]pyridin]-5-yl)amino)ethyl)-1-picolinoyl-N—((S)-piperidin-3-yl)piperidine-4-carboxamide 12.2b

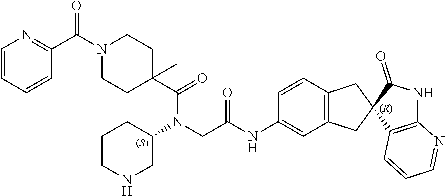

Compound 12.2b was prepared according to General Route C, using the method described for compound 12.2a and starting from Intermediate D (50 mg). Compound 12.1b was used directly, without purification. The final purification by prep-HPLC (column: Luna C18 150×25 mm, 5 μm; mobile phase: [solvent A: water (0.1% TFA), solvent B: MeCN]; B %: 17-34%, 7 min) and lyophilisation afforded 12.2b as a white solid (23 mg, 34% yield, TFA salt, 100.0% purity). ¹H NMR (CD₃OD, 400 MHz) δ 1.44 (s, 3H), 1.56-1.71 (m, 2H), 1.80-1.91 (m, 1H), 1.96-2.21 (m, 4H), 2.29-2.37 (m, 1H), 2.93 (t, 1H), 3.11 (d, 2H), 3.35-3.60 (m, 8H), 4.00-4.55 (m, 4H), 6.92 (dd, 1H), 7.17-7.19 (m, 1H), 7.25 (d, 1H), 7.41 (d, 1H), 7.53-7.61 (m, 3H), 7.98 (td, 1H), 8.08 (dd, 1H), 8.60 (d, 1H). LC-MS Method 4: rt 1.860 min, (622.2 [M+H]⁺).

### Example 18

### 4-Methyl-N-(2-oxo-2-(((R)-2′-oxo-1,1′,2′,3-tetrahydrospiro[indene-2,3′-pyrrolo[2,3-b]pyridin]-5-yl)amino)ethyl)-1-(2-(piperazin-1-yl)acetyl)-N—((S)-piperidin-3-yl)piperidine-4-carboxamide 12.2c

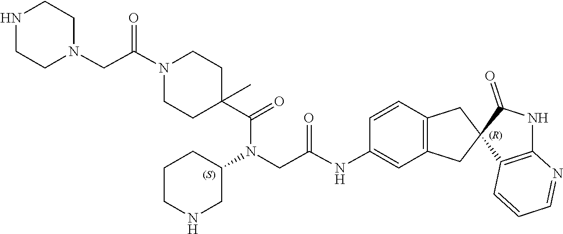

Compound 12.2c was prepared according to General Route C, using the method described for compound 12.2a and starting from Intermediate D (50 mg). Compound 12.1c was used directly, without purification. The final purification by prep-HPLC (column: Phenomenex Synergi C18 150×25 mm, 10 μm; mobile phase: [solvent A: water (0.1% TFA), solvent B: MeCN]; B %: 1-27%, 10 min) and lyophilisation afforded 12.2c as a white solid (29 mg, 36% yield, tris-TFA salt, 97.6% purity). ¹H NMR (CD₃OD, 400 MHz) δ1.40 (s, 3H), 1.48-1.64 (m, 2H), 1.82-2.10 (m, 4H), 2.14-2.31 (m, 2H), 2.86-2.98 (m, 1H), 3.07-3.26 (m, 7H), 3.35-3.64 (m, 11H), 3.70-4.21 (m, 5H), 4.33-4.56 (m, 1H), 6.89-6.92 (m, 1H), 7.16-7.25 (m, 2H), 7.35-7.40 (m, 1H), 7.55-7.59 (m, 1H), 8.05 (d, 1H). LC-MS Method 4: rt 1.472 min, (643.3 [M+H]⁺).

### Example 19

### 1-Acetyl-4-ethyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)piperidine-4-carboxamide

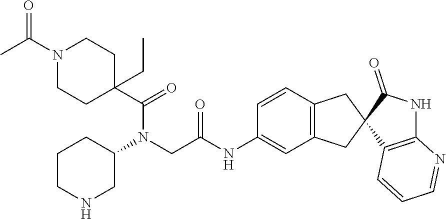 [SMILES: CCC1(C(=O)N(CC(=O)Nc2ccc3c(c2)C[C@@]2(C3)C(=O)Nc3ncccc32)[C@H]2CCCNC2)CCN(C(C)=O)CC1]

The title compound was prepared according to General Route C, using the method described for compound 12.2a, starting from Intermediate E. The final purification by prep-HPLC (column: Phenomenex Luna C18 250×50 mm, 10 μm; mobile phase: [solvent A: water (0.1% TFA), solvent B: MeCN]; B %: 7-32%, 10 min) and lyophilisation afforded the product as a white solid (TFA salt). ¹H NMR (CD₃OD, 400 MHz) δ 0.90-0.99 (m, 3H), 1.39-1.57 (m, 2H), 1.69-1.88 (m, 3H), 1.96-2.13 (m, 6H), 2.20-2.40 (m, 2H), 2.89-2.94 (m, 1H), 3.14-3.11 (m, 3H), 3.32-3.35 (m, 5H), 3.51-3.55 (m, 2H), 3.65-3.77 (m, 1H), 3.95-4.60 (m, 3H), 6.89 (dd, 1H), 7.14 (dd, 1H), 7.20-7.27 (m, 1H), 7.35-7.43 (m, 1H), 7.49-7.60 (m, 1H), 8.05 (dd, 1H). LC-MS Method 8: rt 1.955 min, (573.3 [M+H]⁺).

General Route D

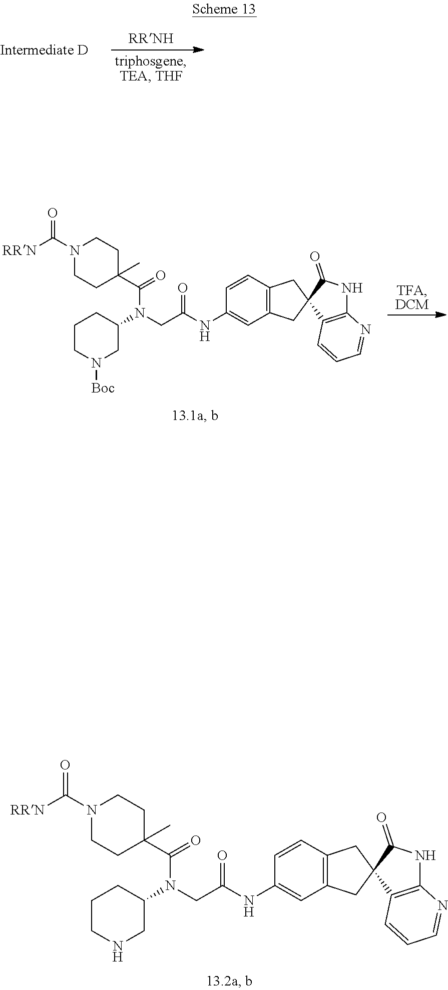

### (S)-tert-Butyl 3-(4-methyl-1-(methylcarbamoyl)-N-(2-oxo-2-(((R)-2′-oxo-1,1′,2′,3-tetrahydrospiro[indene-2,3′-pyrrolo[2,3-b]pyridin]-5-yl)amino)ethyl)piperidine-4-carboxamido)piperidine-1-carboxylate 13.1a

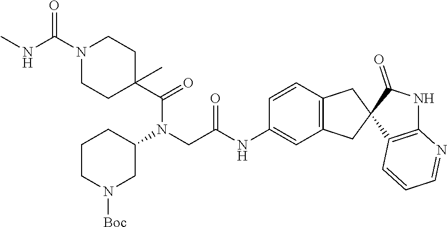 [SMILES: CNC(=O)N1CCC(C)(C(=O)N(CC(=O)Nc2ccc3c(c2)C[C@@]2(C3)C(=O)Nc3ncccc32)[C@H]2CCCN(C(=O)OC(C)(C)C)C2)CC1]

To a solution Intermediate D (70 mg, 0.11 mmol) and triethylamine (46 mg, 0.45 mmol) in THF (1.5 mL) was added triphosgene (30 mg, 0.10 mmol) at 0° C. The mixture was stirred at 20° C. for 30 min. At 0° C., methylamine (31 mg, 0.45 mmol, HCl salt) and triethylamine (57 mg, 0.57 mmol) were added. The mixture was stirred at 20° C. for 10 min, poured into water (20 mL) and extracted with ethyl acetate (3×20 mL). The organic phases were combined, washed with brine (2×20 mL) and dried over anhydrous sodium sulfate. After filtration and concentration, the residue was purified by prep-HPLC (column: Luna C18 150×25 mm, 5 μm; mobile phase: [solvent A: water (0.1% TFA), solvent B: MeCN]; B %: 33-50%, 7 min) to give compound 13.1a as a white solid (35 mg, 41% yield, 89.9% purity). LC-MS Method 1: rt 0.801 min, (674.2[M+H]⁺).

### Example 20

### N1,4-Dimethyl-N4-[2-oxo-2-[[(3R)-2-oxospiro[1H-pyrrolo[2,3-b]pyridine-3,2′-indane]-5′-yl]amino]ethyl]-N4-[(3S)-3-piperidyl]piperidine-1,4-dicarboxamide 13.2a

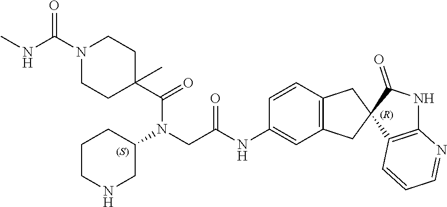

To a solution of compound 13.1a (35 mg, 0.052 mmol) in dichloromethane (1 mL) was added TFA (0.1 mL). The mixture was stirred at 25° C. for 30 min, concentrated and the residue was purified by prep-HPLC (column: Phenomenex Synergi C18 150×25 mm, 10 μm; mobile phase: [solvent A: water (0.1% TFA), solvent B: MeCN]; B %: 6-33%, 10 min). Lyophilisation afforded compound 13.2a as a white solid (13 mg, 43 yield, TFA salt, 98.6% purity). ¹H NMR (CD₃OD, 400 MHz) δ 1.38 (s, 3H), 1.45-1.61 (m, 2H), 1.73-1.91 (m, 1H), 1.95-2.51 (m, 5H), 2.70 (s, 3H), 2.86-2.97 (m, 1H), 3.08 (d, 2H), 3.17-3.29 (m, 3H), 3.36-3.40 (m, 1H), 3.47-3.67 (m, 5H), 4.07-4.43 (m, 3H), 6.90 (t, 1H), 7.16 (d, 1H), 7.24 (d, 1H), 7.39 (d, 1H), 7.55 (s, 1H), 8.05 (d, 1H). LC-MS Method 6: rt 1.457 min, (574.4[M+H]⁺).

### Example 21

### 4-Methyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)-1-(pyrrolidine-1-carbonyl)piperidine-4-carboxamide 13.2b

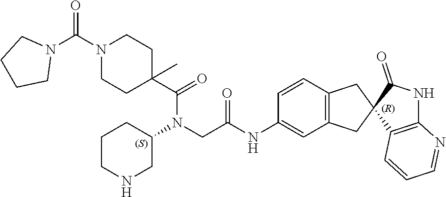

Compound 13.2b was prepared according to General Route D, using the method described for compound 13.2a and using pyrrolidine as the amine component (RR′NH). Final purification by prep-HPLC (column: Phenomenex Synergi C18 150×25 mm, 10 μm; mobile phase: [solvent A: water (0.1% TFA), solvent B: MeCN]; B %: 3-39%, 10 min) and lyophilization afforded compound 13.2b as a white solid (23 mg, 56% yield, TFA salt, 100% purity). ¹H NMR (CD₃OD, 400 MHz) δ 1.38 (s, 3H), 1.50-1.66 (m, 2H), 1.76-1.96 (m, 6H), 2.02-2.16 (m, 4H), 2.92 (t, 1H), 3.08 (d, 2H), 3.16-3.25 (m, 3H), 3.35-3.48 (m, 6H) 3.49-3.55 (m, 4H), 4.12-4.41 (m, 3H), 6.87-6.91 (m, 1H), 7.14 (d, 1H), 7.23 (d, 1H), 7.39 (d, 1H), 7.55 (s, 1H), 8.02-8.07 (m, 1H). LC-MS Method 4: rt 2.028 min, (614.2[M+H]⁺).

General Route E

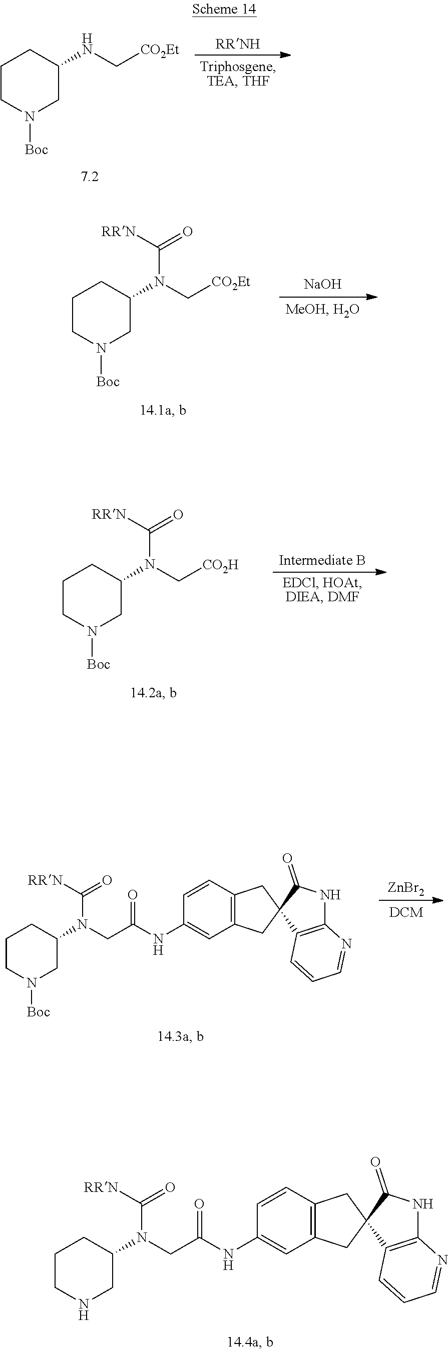

### (S)-tert-Butyl 3-(N-(2-ethoxy-2-oxoethyl)pyrrolidine-1-carboxamido)piperidine-1-carboxylate 14.1a

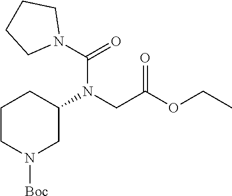 [SMILES: CCOC(=O)CN(C(=O)N1CCCC1)[C@H]1CCCN(C(=O)OC(C)(C)C)C1]

To a solution of compound 7.2 (150 mg, 0.52 mmol) and triethylamine (185 mg, 1.83 mmol) in tetrahydrofuran (6 mL) was added a solution of triphosgene (155 mg, 0.52 mmol) in tetrahydrofuran (1 mL) at 0° C. The mixture was stirred at 0° C. for 30 min. Pyrrolidine (149 mg, 2.10 mmol) then triethylamine (159 mg, 1.57 mmol) were added at 0° C. The mixture was stirred at 20° C. for 2 h, poured into water (20 mL) and extracted with ethyl acetate (3×20 mL). The organic phases were combined, washed with 1M hydrochloric acid (20 mL) and brine (2×20 mL), and dried over anhydrous sodium sulfate. After filtration and concentration, the residue was purified by silica gel column (petroleum ether:ethyl acetate=20:1˜1:2) to provide compound 14.1a as a colourless oil (175 mg, 69% yield, 79.1% purity). ¹H NMR (CDCl₃, 400 MHz) δ 1.27 (t, 3H), 1.45 (s, 9H), 1.49-1.61 (m, 2H), 1.72-1.75 (m, 1H), 1.81-1.87 (m, 4H), 1.98 (d, 1H), 2.54 (t, 1H), 2.73 (t, 1H), 3.35-3.41 (m, 5H), 3.47-3.51 (m, 1H), 3.80-3.99 (q, 2H), 4.03-4.09 (m, 1H), 4.18 (q, 2H).

### (S)-2-(N-(1-(tert-Butoxycarbonyl)piperidin-3-yl)pyrrolidine-1-carboxamido)acetic acid 14.2a

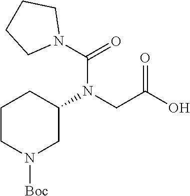 [SMILES: CC(C)(C)OC(=O)N1CCC[C@H](N(CC(=O)O)C(=O)N2CCCC2)C1]

To a solution of compound 14.1a (175 mg, 0.36 mmol) in methanol (4.5 mL) was added a solution of sodium hydroxide (58 mg, 1.44 mmol) in water (1.5 mL) at 20° C. The mixture was stirred for 1 h, poured into water (20 mL), adjusted to pH4 with 1M hydrochloric acid and extracted with ethyl acetate (2×50 mL). The organic layers were combined, washed with brine (50 mL) and dried over sodium sulfate. After filtration and concentration, compound 14.2a was obtained as a white solid (143 mg, 88% yield, 79.1% purity). LC-MS Method 1: rt 0.818 min, (356.2 [M+H]⁺).

### (S)-tert-Butyl 3-(N-(2-oxo-2-(((R)-2′-oxo-1,1′,2′,3-tetrahydrospiro[indene-2,3′-pyrrolo [2,3-b]pyridin]-5-yl)amino)ethyl)pyrrolidine-1-carboxamido)piperidine-1-carboxylate 14.3a

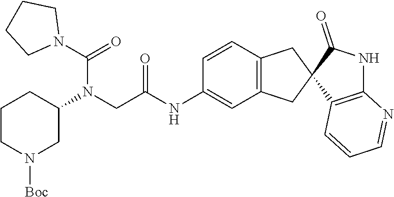 [SMILES: CC(C)(C)OC(=O)N1CCC[C@H](N(CC(=O)Nc2ccc3c(c2)C[C@@]2(C3)C(=O)Nc3ncccc32)C(=O)N2CCCC2)C1]

To a solution of compound 14.2a (80 mg, 0.18 mmol), EDCI (51 mg, 0.27 mmol) and HOAt (36 mg, 0.27 mmol) in DMF (2 mL) was added DIEA (69.03 mg, 0.53 mmol) followed by Intermediate B (45 mg, 0.18 mmol) at 20° C. The mixture was stirred at 20° C. for 12 h, poured into water (20 mL), adjusted to pH4 with 1M hydrochloric acid, and extracted with ethyl acetate (2×50 mL). The combined organic layers were washed with brine (50 mL) and dried over sodium sulfate. After filtration and concentration, compound 14.3a was obtained as a white solid (100 mg, 70% yield, 73.7% purity). LC-MS Method 1 rt 0.829 min, (589.4 [M+H]⁺).

### Example 22

### N-(2-Oxo-2-(((R)-2′-oxo-1,1′,2′,3-tetrahydrospiro[indene-2,3′-pyrrolo [2,3-b]pyridin]-5-yl)amino)ethyl)-N—((S)-piperidin-3-yl)pyrrolidine-1-carboxamide 14.4a

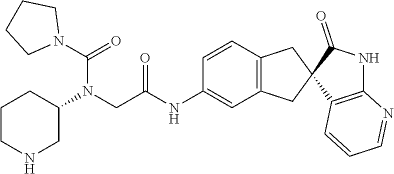 [SMILES: O=C(CN(C(=O)N1CCCC1)[C@H]1CCCNC1)Nc1ccc2c(c1)C[C@@]1(C2)C(=O)Nc2ncccc21]

To a solution of compound 14.3a (50 mg, 0.085 mmol) in DCM (6 mL) was added zinc bromide (478 mg, 2.12 mmol) at 20° C. The mixture was stirred for 15 h, poured into water (20 mL), adjusted to pH4 with 1M hydrochloric acid and extracted with ethyl acetate (2×50 mL). The organic layers were combined, washed with brine (50 mL) and dried over sodium sulfate. After filtration and concentration, the residue was purified by prep-HPLC (column: Luna C18 150×25 mm, 5 μm; mobile phase: [solvent A: water (0.075% TFA), solvent B: MeCN]; B %: 5-35%, 9 min). After lyophilisation, compound 14.4a was obtained as a yellow solid (7 mg, 14% yield, TFA salt, 98.5% purity). ¹H NMR (CD₃OD, 400 MHz) δ 1.78-1.94 (m, 6H), 2.03-2.06 (m, 2H), 2.89 (t, 1H), 3.08 (dd, 2H), 3.09 (t, 1H), 3.31-3.33 (m, 1H), 3.37-3.40 (m, 4H), 3.48-3.55 (m, 3H), 3.91-3.97 (m, 1H), 4.11 (s, 2H), 6.90 (dd, 1H), 7.16 (dd, 1H), 7.24 (d, 1H), 7.39 (d, 1H), 7.55 (s, 1H), 8.06 (dd, 1H). LC-MS Method 4 rt 1.817 min, (489.1 [M+H]⁺).

### tert-Butyl 4-acetylpiperazine-1-carboxylate

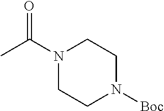 [SMILES: CC(=O)N1CCN(C(=O)OC(C)(C)C)CC1]

To a solution of tert-butyl piperazine-1-carboxylate (1.00 g, 5.37 mmol) and triethylamine (815 mg, 8.05 mmol) in DCM (10 mL) was added acetyl chloride (464 mg, 5.91 mmol) dropwise at 0° C. The mixture was stirred at 0° C. for 1 h, poured into water (20 mL) and extracted with ethyl acetate (2×50 mL). The organic layers were combined, washed with 1M hydrochloric acid (20 mL), saturated sodium bicarbonate (20 mL). The resulting organic phase was washed with brine (50 mL) and dried over sodium sulfate. After filtration and concentration, the title compound was obtained as colourless oil (1.40 g) and used without further purification. ¹H NMR (CDCl₃, 400 MHz) δ 1.47 (s, 9H), 2.22 (s, 3H), 3.39-3.44 (m, 6H), 3.57-3.60 (m, 2H).

### 1-Acetylpiperazine hydrochloride salt

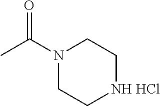

tert-Butyl 4-acetylpiperazine-1-carboxylate (1.40 g, 6.13 mmol) in 4M HCl/dioxane (20 mL) was stirred at 20° C. for 1 h. The reaction mixture was concentrated under reduced pressure to give the title compound as a white solid (1.00 g, 99% yield, HCl salt). ¹H NMR (CD₃OD, 400 MHz) δ 2.16 (s, 3H), 2.22-2.30 (m, 4H), 3.82-3.84 (m, 4H).

### Example 23

### 4-Acetyl-N-(2-oxo-2-(((R)-2′-oxo-1,1′,2′,3-tetrahydrospiro[indene-2,3′-pyrrolo[2,3-b]pyridin]-5-yl)amino)ethyl)-N—((S)-piperidin-3-yl)piperazine-1-carboxamide 14.4b

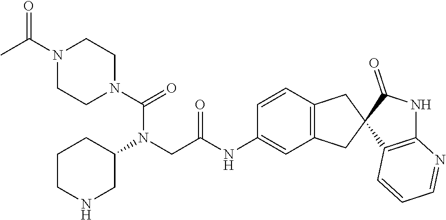 [SMILES: CC(=O)N1CCN(C(=O)N(CC(=O)Nc2ccc3c(c2)C[C@@]2(C3)C(=O)Nc3ncccc32)[C@H]2CCCNC2)CC1]

Compound 14.4b was prepared according to General Route E, using 1-acetylpiperazine as RR′NH and the procedures detailed for compound 14.4a. The final product was purified by prep-HPLC (column: Luna C18 150×25 mm, 5 μm; mobile phase: [solvent A: water (0.075% TFA), solvent B: MeCN]; B %: 2-32%, 9 min), followed by lyophilization to afford compound 14.4b as a yellow gum (TFA salt, 96.8% purity). ¹H NMR (CD₃OD, 400 MHz) δ 1.81-2.04 (m, 4H), 2.10 (s, 3H), 2.89 (t, 1H), 3.11 (dd, 2H), 3.16-3.26 (m, 4H), 3.33-3.34 (m, 2H), 3.48-3.61 (m, 7H), 3.87-3.93 (m, 1H), 4.13 (s, 2H), 6.89 (dd, 1H), 7.15 (dd, 1H), 7.24 (d, 1H), 7.38-7.40 (m, 1H), 7.56 (s, 1H), 8.06 (d, 1H). LC-MS (Long acidic method 1): rt 1.795 min, (546.2 [M+H]⁺).

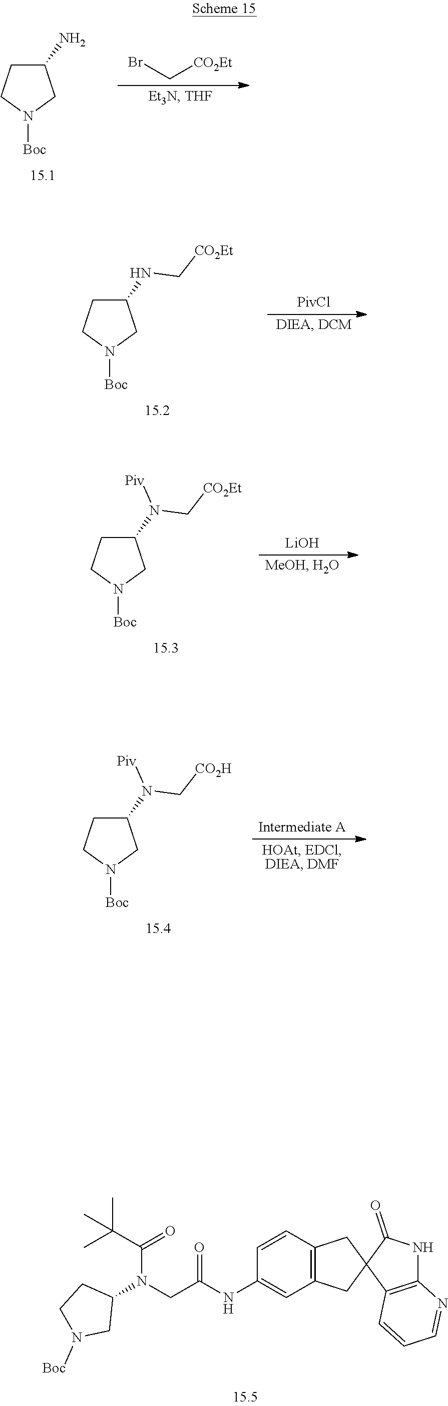

### tert-Butyl (3S)-3-[(2-ethoxy-2-oxo-ethyl)amino]pyrrolidine-1-carboxylate 15.2

To a solution of compound 15.1 (1.00 g, 5.37 mmol) in THF (15 mL) was added ethyl 2-bromoacetate (986 mg, 5.91 mmol) followed by triethylamine (1.36 g, 13.42 mmol) at 20° C. The mixture was stirred at 20° C. for 12 h, poured into water (60 mL) and extracted with ethyl acetate (3×50 mL). The organic phases were combined, washed with brine (2×60 mL) and dried over anhydrous sodium sulfate. After filtration and concentration, the residue was purified by silica gel column chromatography, eluting with petroleum ether:ethyl acetate=20:1˜1:1, to provide compound 15.2 as colourless oil (1.04 g, 71% yield). ¹H NMR (CDCl₃, 400 MHz) δ 1.29 (t, 3H), 1.46 (s, 9H), 1.72-1.79 (m, 1H), 1.96-2.05 (m, 1H), 3.05-3.20 (m, 1H), 3.28-3.39 (m, 2H), 3.39-3.43 (m, 2H), 3.44-3.56 (m, 2H), 4.19 (q, 2H).

### tert-Butyl (3S)-3-[2,2-dimethylpropanoyl-(2-ethoxy-2-oxo-ethyl)amino]pyrrolidine-1-carboxylate 15.3

To a solution of compound 15.2 (500 mg, 1.84 mmol) in dichloromethane (8 mL) was added DIEA (711 mg, 5.51 mmol) followed by 2,2-dimethylpropanoyl chloride (266 mg, 2.20 mmol) at 20° C. The mixture was stirred at 20° C. for 12 h, poured into water (50 mL) and extracted with ethyl acetate (3×40 mL). The organic phases were combined, washed with 0.2M hydrochloric acid (2×40 mL), saturated sodium bicarbonate (40 mL) and brine (2×50 mL), and dried over anhydrous sodium sulfate. After filtration and concentration, compound 15.3 was obtained as a yellow gum (610 mg, 93% yield). ¹H NMR (CDCl₃, 400 MHz) δ 1.29 (t, 3H), 1.33 (s, 9H), 1.47 (s, 9H), 1.87-2.01 (m, 1H), 2.10-2.19 (m, 1H), 3.15 (dd, 1H), 3.22-3.36 (m, 1H), 3.44-3.71 (m, 2H), 3.72-3.86 (m, 1H), 3.87-4.01 (m, 1H), 4.19 (q, 2H), 4.81-4.93 (m, 1H).

### 2-[[(3S)-1-tert-Butoxycarbonylpyrrolidin-3-yl]-(2,2-dimethylpropanoyl)amino]acetic acid 15.4

To a solution of compound 15.3 (300 mg, 0.84 mmol) in methanol (5 mL) was added a solution of lithium hydroxide hydrate (177 mg, 4.21 mmol) in water (2 mL). The mixture was stirred at 20° C. for 12 h, diluted with water (30 mL) and extracted with ethyl acetate (30 mL). The aqueous phase was adjusted to pH4 with 1M hydrochloric acid and extracted with ethyl acetate (3×30 mL). The organic phases were combined and washed with brine (2×40 mL) and dried over anhydrous sodium sulfate. After filtration and concentration, compound 15.4 was obtained as a yellow gum (270 mg, 98% yield). ¹H NMR (CDCl₃, 400 MHz) δ 1.34 (s, 9H), 1.47 (s, 9H), 1.91-2.04 (m, 1H), 2.11-2.18 (m, 1H), 3.14-3.24 (m, 1H), 3.25-3.36 (m, 1H), 3.48-3.75 (m, 2H), 3.91 (q, 2H), 4.81-4.92 (m, 1H).

### tert-Butyl (3S)-3-[2,2-dimethylpropanoyl-[2-oxo-2-[(2-oxospiro[1H-pyrrolo[2,3-b] pyridine-3,2′-indane]-5′-yl)amino]ethyl]amino]pyrrolidine-1-carboxylate 15.5

To a solution of compound 15.4 (82 mg, 0.25 mmol), EDCI (69 mg, 0.36 mmol) and HOAt (42 mg, 0.31 mmol) in DMF (2 mL) was added DIEA (123 mg, 0.96 mmol) followed by Intermediate A (60 mg, 0.24 mmol) at 20° C. Then the mixture was stirred at 25° C. for 6 h, poured into water (20 mL) and filtered. The filter cake was dissolved in ethyl acetate (20 mL) and dried over anhydrous sodium sulfate. After filtration and concentration, compound 15.5 was obtained as a yellow solid (80 mg, 51% yield). ¹H NMR (CD₃OD, 400 MHz) δ 1.34 (s, 9H), 1.45 (s, 9H), 2.06-2.25 (m, 2H), 3.08 (dd, 2H), 3.34-3.41 (m, 2H), 3.47-3.59 (m, 3H), 3.64 (dd, 1H), 3.95-4.08 (m, 2H), 4.92-5.06 (m, 1H), 6.88 (dd, 1H), 7.12 (dd, 1H), 7.23 (d, 1H), 7.38 (d, 1H), 7.57 (s, 1H), 8.05 (dd, 1H).

### Example 24

### N-(2-Oxo-2-((2′-oxo-1,1′,2′,3-tetrahydrospiro[indene-2,3′-pyrrolo[2,3-b]pyridin]-5-yl)amino)ethyl)-N—((S)-pyrrolidin-3-yl)pivalamide

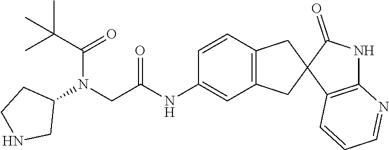 [SMILES: CC(C)(C)C(=O)N(CC(=O)Nc1ccc2c(c1)CC1(C2)C(=O)Nc2ncccc21)[C@H]1CCNC1]

To a solution of compound 15.5 (80 mg, 0.12 mmol) in dichloromethane (2 mL) was added zinc bromide (412 mg, 1.83 mmol) at 20° C. and stirred for 20 h. The mixture was dissolved in methanol (20 mL), poured into saturated sodium bicarbonate (20 mL) and extracted with ethyl acetate (8×40 mL). The organic phases were combined, washed with brine (40 mL) and dried over anhydrous sodium sulfate. After filtration and concentration, the residue was purified by prep-HPLC (column: Boston pH-lex 150×25 mm, 10 μm; mobile phase: [solvent A: water (0.1% TFA), solvent B: MeCN]; B %: 18-38%, 8 min) and prep-HPLC (column: Phenomenex Synergi C18 150×25 mm, 10 μm; mobile phase: [solvent A: water (0.1% TFA), solvent B: MeCN]; B %: 18-48%, 9 min). After lyophilisation, Example 24 (27 mg, 38% yield, TFA salt, 99.3% purity) was obtained as a white solid. ¹H NMR (CD₃OD, 400 MHz) δ 1.29 (s, 9H), 2.15-2.30 (m, 1H), 2.37-2.51 (m, 1H), 3.03-3.06 (m, 3H), 3.38-3.75 (m, 5H), 3.87-4.72 (m, 3H), 6.87-6.94 (m, 1H), 7.14-7.20 (m, 1H), 7.25 (d, 1H), 7.35-7.42 (m, 1H), 7.59 (s, 1H), 8.06 (dd, 1H). LC-MS Method 6: rt 1.385 min, (462.1 [M+H]⁺).

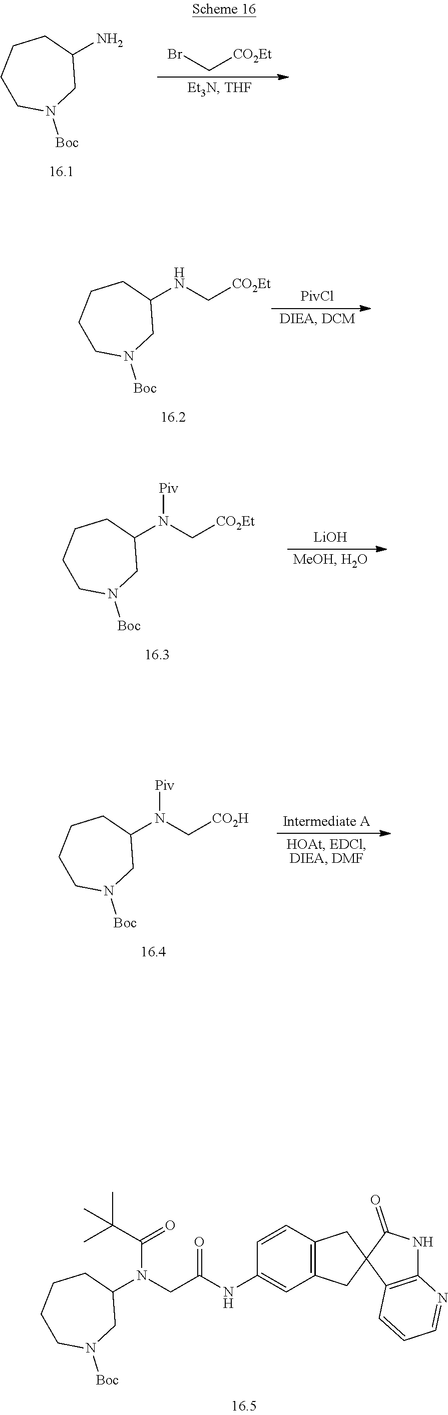

### tert-Butyl 3-[(2-ethoxy-2-oxo-ethyl)amino]azepane-1-carboxylate 16.2

To a solution of compound 16.1 (200 mg, 0.93 mmol) in THF (2 mL) was added triethylamine (284 mg, 2.80 mmol) and ethyl 2-bromoacetate (172 mg, 1.03 mmol). The mixture was stirred at 30° C. for 12 h, poured into water (10 mL) and extracted with ethyl acetate (3×20 mL). The combined organic layers were washed with brine (3×20 mL) and dried with anhydrous sodium sulfate. After filtration and concentration, the residue was purified by silica gel column chromatography, eluting with petroleum ether:ethyl acetate=30:1˜5:1 to give the compound 16.2 as yellow oil (180 mg, 64% yield). ¹H NMR (CDCl₃, 400 MHz) δ 1.28 (t, 3H), 1.45-1.47 (m, 9H), 1.51-1.63 (m, 2H), 1.72-1.89 (m, 4H), 2.61-2.91 (m, 2H), 3.11-3.20 (m, 1H), 3.45-3.49 (m, 2H), 3.54-3.80 (m, 2H), 4.20 (q, 2H).

### tert-Butyl 3-[2,2-dimethylpropanoyl-(2-ethoxy-2-oxo-ethyl)amino]azepane-1-carboxylate 16.3

To a solution of compound 16.2 (180 mg, 0.60 mmol) in dichloromethane (2 mL) was added diisopropylethylamine (387 mg, 3.00 mmol) and pivaloyl chloride (94 mg, 0.78 mmol). The mixture was stirred at 25° C. for 12 h, poured into water (10 mL) and extracted with dichloromethane (3×20 mL). The organic layers were combined, washed with brine (3×20 mL) and dried over anhydrous sodium sulfate. After filtration and concentration, compound 16.3 was obtained as yellow oil (220 mg, crude).

### tert-Butyl 3-[2,2-dimethylpropanoyl-(2-ethoxy-2-oxo-ethyl)amino]azepane-1-carboxylate 16.4

To a solution of compound 16.3 (220 mg, 0.57 mmol) in methanol (2 mL) was added the solution of hydrated lithium hydroxide (120 mg, 2.86 mmol) in water (2 mL). The mixture was stirred at 25° C. for 2 h, diluted with water (20 mL) and extracted with ethyl acetate (3×30 mL). The organic layers were discarded. Then the aqueous phase was acidified by 1M hydrochloric acid (5 mL) and extracted with ethyl acetate (3×30 mL). Organic layers were combined, washed with brine (3×30 mL) and dried over anhydrous sodium sulfate. After filtration and concentration, compound 16.4 was obtained as yellow oil (180 mg, crude). ¹H NMR (CDCl₃, 400 MHz) δ 1.34 (s, 9H), 1.47 (s, 9H), 1.58-1.69 (m, 2H), 1.78-1.91 (m, 4H), 3.15-3.35 (m, 2H), 3.41-3.50 (m, 1H), 3.85-3.93 (m, 3H), 4.22-4.32 (m, 1H).

### tert-Butyl 3-(N-(2-oxo-2-((2′-oxo-1,1′,2′,3-tetrahydrospiro[indene-2,3′-pyrrolo[2,3-b]pyridin]-5-yl)amino)ethyl)pivalamido)azepane-1-carboxylate 16.5

To a solution of compound 16.4 (100 mg, 0.28 mmol) in DMF (2 mL) was added diisopropylethylamine (142 mg, 1.40 mmol), EDCI (81 mg, 0.42 mmol), and HOAt (58 mg, 0.42 mmol). Then Intermediate A (71 mg, 0.28 mmol) was added and the mixture was stirred at 25° C. for 12 h. The reaction was quenched with water (10 mL), and the mixture extracted with ethyl acetate (3×20 mL). The combined organic layers were washed with brine (3×20 mL) and dried over anhydrous sodium sulfate. After filtration and concentration, the residue was purified by prep-TLC (petroleum ether:ethyl acetate=0:1) to give the compound 16.5 as a yellow solid (102 mg, 61% yield, 99.2% purity). ¹H NMR (CDCl₃, 400 MHz) δ 1.39 (s, 9H), 1.46 (s, 9H), 1.59-1.67 (m, 2H), 1.83-1.97 (m, 4H), 2.99-3.09 (m, 2H), 3.15-3.40 (m, 2H), 3.58-3.66 (m, 3H), 3.87-4.01 (m, 2H), 4.15-4.40 (m, 2H), 6.81-6.84 (m, 1H), 7.05-7.10 (m, 1H), 7.17-7.20 (m, 1H), 7.22-7.25 (m, 1H), 7.46-7.67 (m, 1H), 7.87-7.93 (m, 1H), 8.09-8.13 (m, 1H), 8.91-9.01 (m, 1H). LC-MS Method 7: rt 0.953 min, (590.4 [M+H]⁺).

### Example 25

### N-(Azepan-3-yl)-N-(2-oxo-2-((2′-oxo-1,1′,2′,3-tetrahydrospiro[indene-2,3′-pyrrolo[2,3-b]pyridin]-5-yl)amino)ethyl)pivalamide

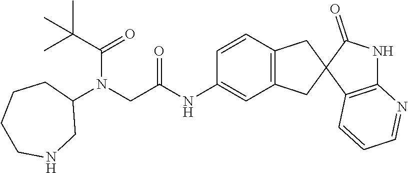 [SMILES: CC(C)(C)C(=O)N(CC(=O)Nc1ccc2c(c1)CC1(C2)C(=O)Nc2ncccc21)C1CCCCNC1]

To a solution of compound 16.5 (100 mg, 0.17 mmol) in dichloromethane (2 mL) was added zinc bromide (573 mg, 2.54 mmol) and the mixture was stirred at 25° C. for 12 h. The mixture was dissolved with methanol (2 mL) and water (10 mL) added. The mixture was basified with saturated sodium bicarbonate (3 mL) and extracted with ethyl acetate (3×20 mL). The organic layers were combined, washed with brine (3×20 mL) and dried over anhydrous sodium sulfate. After filtration and concentration, the residue was purified by prep-HPLC (column: Phenomenex Synergi C18 150×25 mm, 10 μm; mobile phase: [solvent A: water (0.1% TFA), solvent B: MeCN]; B %: 23-53%, 9 min). After lyophilisation, Example 25 was obtained as a white solid (30 mg, 30% yield, TFA salt, 99.7% purity). ¹H NMR (CD₃OD, 400 MHz) δ 1.28 (s, 9H), 1.35-1.55 (m, 1H), 1.77-1.87 (m, 1H), 1.90-2.05 (m, 3H), 2.17-2.20 (m, 1H), 2.83-3.00 (m, 1H), 3.11 (d, 2H), 3.33-3.34 (m, 1H), 3.40-3.46 (m, 1H), 3.48-3.55 (m, 4H), 3.75-3.90 (m, 1H), 4.21 (d, 1H), 6.87-6.91 (m, 1H), 7.13-7.20 (m, 1H), 7.25 (d, 1H), 7.35-7.40 (m, 1H), 7.58 (d, 1H), 8.06 (dd, 1H). LC-MS rt 2.068 min, (490.3 [M+H]⁺).

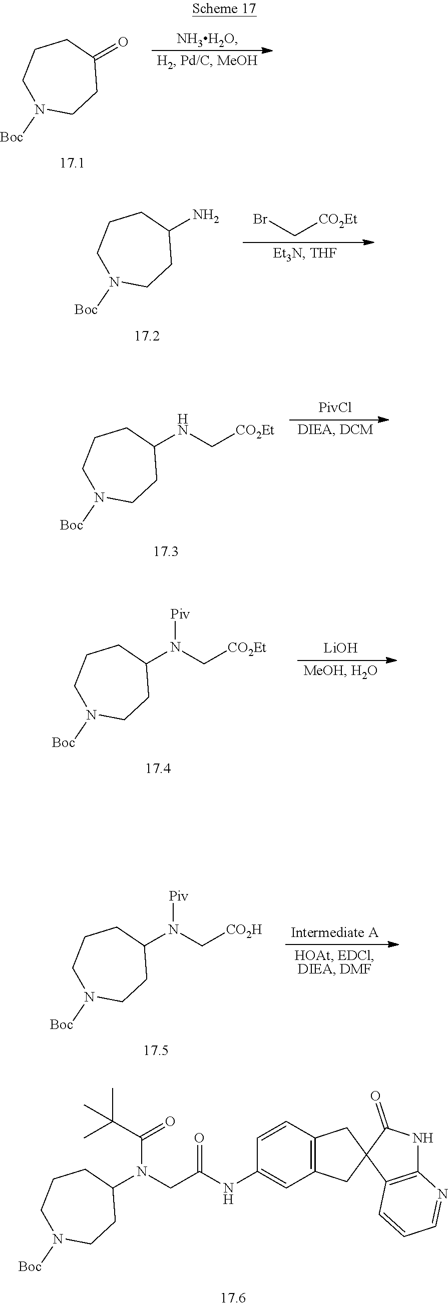

### tert-Butyl 4-aminoazepane-1-carboxylate 17.1

To a solution of compound 17.1 (500 mg, 2.34 mmol) and 25% aqueous ammonium hydroxide (2.46 g, 17.58 mmol) in methanol (10 mL) was added 10% Pd/C (80 mg). The mixture was degassed under vacuum and purged with hydrogen three times. The resulting mixture was stirred at 20° C. for 12 h under a hydrogen atmosphere (15 psi). The mixture was diluted with methanol (30 mL), filtered, and the filtrate concentrated in vacuo to give compound 17.2 as yellow oil (480 mg, 95% yield). ¹H NMR (CDCl₃, 400 MHz) δ1.47 (s, 9H), 1.51-1.66 (m, 3H), 1.73-1.95 (m, 3H), 2.87-2.96 (m, 1H), 3.12-3.35 (m, 2H), 3.39-3.44 (m, 1H), 3.49-3.60 (m, 1H).

### tert-Butyl 4-[(2-ethoxy-2-oxo-ethyl)amino]azepane-1-carboxylate 17.3

To a solution of tert-butyl 4-aminoazepane-1-carboxylate 17.2 (250 mg, 1.17 mmol) and triethylamine (295 mg, 2.92 mmol) in tetrahydrofuran (4 mL) was added ethyl 2-bromoacetate (214 mg, 1.28 mmol) at 20° C. The mixture was stirred at 20° C. for 12 h, poured into water (20 mL) and extracted with ethyl acetate (3×20 mL). The organic phases were combined, washed with brine (2×20 mL), dried over anhydrous sodium sulfate, filtered and concentrated in vacuo. The residue was purified by column chromatography (petroleum ether:ethyl acetate=5:1 to 0:1) to afford compound 17.3 as yellow oil (210 mg, 59.9% yield). ¹H NMR (CDCl₃, 400 MHz) 1.29 (t, 3H), 1.46 (s, 9H), 1.49-1.57 (m, 2H), 1.70-1.95 (m, 4H), 2.57-2.64 (m, 1H), 3.18-3.40 (m, 2H), 3.41 (s, 2H), 3.43-3.57 (m, 2H), δ 4.20 (q, 2H).

### tert-Butyl 4-[2,2-dimethylpropanoyl-(2-ethoxy-2-oxo-ethyl)amino]azepane-1-carboxylate 17.4

To a solution of compound 17.3 (210 mg, 0.70 mmol) and N,N-diisopropylethylamine (226 mg, 1.75 mmol) in dichloromethane (3 mL) was added 2,2-dimethylpropanoyl chloride (101 mg, 0.84 mmol) at 20° C. The mixture was stirred at 20° C. for 12 h, poured into water (20 mL) and extracted with ethyl acetate (3×20 mL). The organic phases were combined, washed with 0.2M hydrochloric acid (30 mL) and brine (2×20 mL), and dried over anhydrous sodium sulfate. Filtration and concentration afforded compound 17.4 as yellow gum (260 mg, 97% yield, 100% purity). ¹H NMR (CDCl₃, 400 MHz) δ1.27-1.28 (m, 3H), 1.30 (d, 9H), 1.48 (d, 9H), 1.57-1.72 (m, 3H), 1.85-1.92 (m, 1H), 1.98-2.03 (m, 1H), 2.09-2.20 (m, 1H), 3.09-3.19 (m, 1H), 3.38-3.50 (m, 2H), 3.59-3.83 (m, 3H), 4.12-4.21 (m, 3H).

### 2-[(1-tert-Butoxycarbonylazepan-4-yl)-(2,2-dimethylpropanoyl)amino]acetic acid 17.5

To a solution of 17.4 (260 mg, 0.68 mmol) in methanol (3 mL) was added a solution of sodium hydroxide (162 mg, 4.06 mmol) in water (1 mL). The mixture was stirred at 20° C. for 4 h, poured into water (30 mL) and washed with ethyl acetate (30 mL). The aqueous phase was acidified to pH4 with 1M hydrochloric acid and extracted with ethyl acetate (3×40 mL). The organic phases were combined, washed with brine (2×40 mL), dried over anhydrous sodium sulfate, filtered and concentrated in vacuo to afford compound 17.5 as a yellow gum (210 mg, 0.59 mmol, 87% yield). ¹H NMR (CDCl₃, 400 MHz) δ1.31 (d, 9H), 1.48 (s, 9H), 1.66-1.97 (m, 5H), 2.09-2.20 (m, 1H), 3.07-3.15 (m, 1H), 3.40-3.50 (m, 2H), 3.63-3.95 (m, 3H), 4.12-4.24 (m, 1H).

### tert-Butyl 4-(N-(2-oxo-2-((2′-oxo-1,1′,2′,3-tetrahydrospiro[indene-2,3′-pyrrolo[2,3-b]pyridin]-5-yl)amino)ethyl)pivalamido)azepane-1-carboxylate 17.6

To a solution of compound 17.5 (104 mg, 0.29 mmol), EDCI (80 mg, 0.42 mmol) and HOAt (49 mg, 0.36 mmol) in N,N-dimethylformamide (2 mL) was added N,N-diisopropylethylamine (126 mg, 0.98 mmol) and Intermediate A (70 mg, 0.28 mmol) at 20° C. The mixture was stirred at 20° C. for 3 h, poured into water (20 mL) and filtered. The precipitate was collected by filtration, dissolved in ethyl acetate (40 mL), and dried over anhydrous sodium sulfate. Filtration and concentration in vacuo to afforded compound 17.6 as a yellow solid (140 mg, 77% yield, 90% purity). ¹H NMR (CD₃OD, 400 MHz) δ1.32 (d, 9H), 1.48 (d, 9H), 1.93-1.69 (m, 4H), 2.12-2.04 (m, 1H), 2.28-2.16 (m, 1H), 3.06 (dd, 2H), 3.20-3.12 (m, 1H), 3.44-3.35 (m, 1H), 3.51 (dd, 2H), 3.75-3.57 (m, 2H), 4.03-3.87 (m, 2H), 4.30-4.17 (m, 1H), 6.88 (dd, 1H), 7.12 (dd, 1H), 7.22 (d, 1H), 7.40-7.38 (m, 1H), 7.56 (d, 1H), 8.05 (dd, 1H). LC-MS Method 1: rt 0.946 min, (612 [M+Na]⁺).

### Example 26

### N-(Azepan-4-yl)-N-(2-oxo-2-((2′-oxo-1,1′,2′,3-tetrahydrospiro[indene-2,3′-pyrrolo[2,3-b]pyridin]-5-yl)amino)ethyl)pivalamide

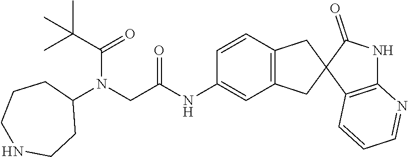 [SMILES: CC(C)(C)C(=O)N(CC(=O)Nc1ccc2c(c1)CC1(C2)C(=O)Nc2ncccc21)C1CCCNCC1]

To a solution of compound 17.6 (140 mg, 0.21 mmol) in dichloromethane (3 mL) was added zinc bromide (723 mg, 3.21 mmol). The mixture was stirred at 20° C. for 12 h, dissolved in methanol (15 mL), poured into saturated sodium bicarbonate (30 mL) and extracted with ethyl acetate (8×40 mL). The organic phases were combined was washed with brine (40 mL), dried over anhydrous sodium sulfate, filtered and concentrated in vacuo. The residue was purified by prep-HPLC (column: Phenomenex Synergi C18 150×25 mm, 10 μm; mobile phase: [solvent A: water (0.1% TFA), solvent B: MeCN]; B %: 10-40%, 9 min) to afford Example 26 as a white solid (32 mg, 25% yield, TFA salt, 98.7% purity). ¹H NMR (CD₃OD, 400 MHz) δ1.32 (s, 9H), 1.99-1.79 (m, 2H), 2.25-2.04 (m, 3H), 2.38-2.26 (m, 1H), 3.08 (dd, 2H), 3.28-3.15 (m, 2H), 3.37-3.34 (m, 1H), 3.46-3.39 (m, 1H), 3.52 (dd, 2H), 4.01 (br s, 2H), 4.44 (br s, 1H), 6.91 (dd, 1H), 7.17 (d, 1H), 7.23 (d, 1H), 7.38 (t, 1H), 7.57 (d, 1H), 8.06 (dd, 1H). LC-MS Method 1: rt 0.717 min.

Note for Examples 27, 28 and 29: Relative stereochemistry is indicated by block bold- and dashed-bonds, and the absolute stereochemistry of is indicated by wedge bold- and dashed-bonds. Thus, the stereochemistry around the piperidine rings is relative and the stereochemistry derived from Intermediate B is absolute. While one isomer is illustrated, Examples 27, 28 and 29 are mixtures of diastereomers. Relative stereochemistry is denoted in compound names by S* and R*.

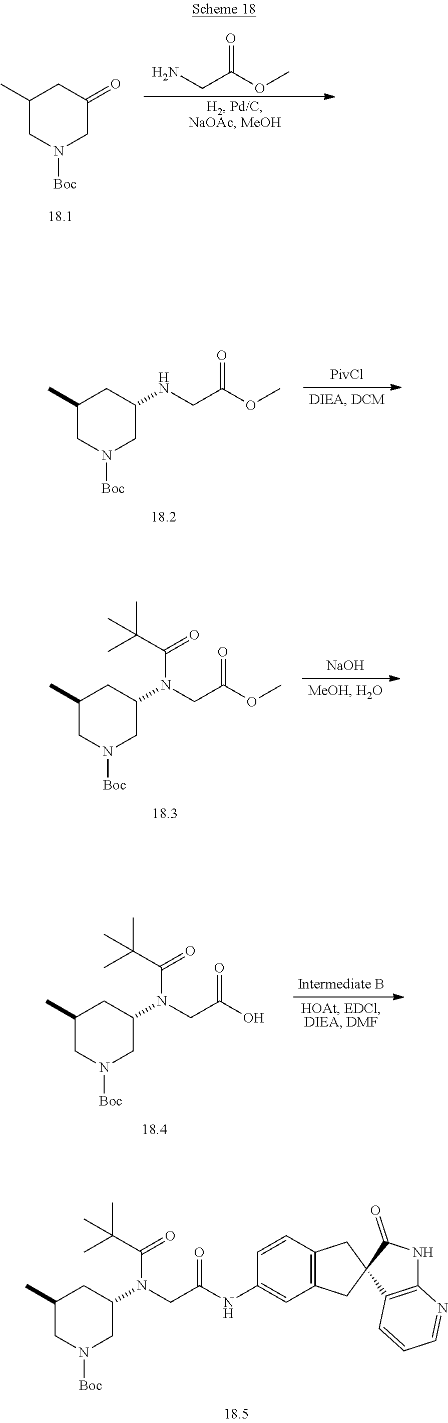

### (3S*,5S*)-tert-Butyl 3-((2-methoxy-2-oxoethyl)amino)-5-methylpiperidine-1-carboxylate 18.2

To a solution of compound 18.1 (200 mg, 0.94 mmol) in methanol (5 mL) was added 10% Pd/C (10 mg), sodium acetate (192 mg, 2.34 mmol) and methyl 2-aminoacetate (235 mg, 1.88 mmol, HCl salt). The mixture was degassed and purged with hydrogen three times and stirred at 25° C. for 16 h under a hydrogen balloon. The catalyst was removed by filtration and the filtrate was poured into water (20 mL). 1M Hydrochloric acid (10 mL) was added and the aqueous mixture washed with ethyl acetate (2×20 mL). The aqueous phase was adjusted to pH8 with sodium carbonate and extracted with ethyl acetate (2×20 mL). The organic phases were combined, washed with brine (20 mL) and dried over sodium sulfate. After filtration and concentration, only rac-trans-isomer compound 18.2 was obtained as a yellow oil (150 mg, 56% yield). ¹H NMR (CDCl₃, 400 MHz) δ 0.89 (d, 3H), 1.30-1.38 (m, 1H), 1.46 (s, 9H), 1.71-1.76 (m, 1H), 1.92-1.98 (m, 1H), 2.25-2.50 (m, 1H), 2.80 (br. s, 1H), 3.00-3.23 (m, 1H), 3.39-3.60 (m, 3H), 3.73 (s, 3H), 3.80-3.93 (m, 1H).

### (3S*,5S*)-tert-Butyl 3-(N-(2-methoxy-2-oxoethyl)pivalamido)-5-methylpiperidine-1-carboxylate 18.3

To a solution of compound 18.2 (170 mg, 0.59 mmol) in dichloromethane (5 mL) was added DIEA (192 mg, 1.48 mmol) and pivaloyl chloride (143 mg, 1.19 mmol) at 0° C. The mixture was stirred at 25° C. for 1 h, quenched by the addition water (50 mL) and extracted with ethyl acetate (2×30 mL). The combined organic layers were washed with brine (20 mL) and dried over anhydrous sodium sulfate. After filtration and concentration, compound 18.3 was obtained as a yellow oil (218 mg, crude).

### 2-(N-((3S*,5S*)-1-(tert-Butoxycarbonyl)-5-methylpiperidin-3-yl)pivalamido)acetic acid 18.4

To a solution of compound 18.3 (210 mg, 0.57 mmol) in methanol (6 mL) and water (2 mL) was added sodium hydroxide (45 mg, 1.13 mmol). The mixture was stirred at 25° C. for 16 h, poured into water (30 mL) and washed with dichloromethane (20 mL). The aqueous phase was acidified with 1M hydrochloric acid (10 mL) and extracted with ethyl acetate (2 15×30 mL). The organic phases were combined, washed with brine (20 mL) and dried over sodium sulfate. After filtration and concentration, compound 18.4 was obtained as yellow oil (200 mg, 99% yield). ¹H NMR (CDCl₃, 400 MHz) δ 1.08 (dd, 3H), 1.34 (s, 9H), 1.45 (m, 10H), 1.78-1.86 (m, 2H), 2.13-2.18 (m, 1H), 2.67-2.87 (m, 2H), 3.81-3.85 (m, 1H), 3.93-3.99 (m, 1H), 4.16-4.32 (m, 2H).

### (3S*,5S*)-tert-Butyl 3-methyl-5-(N-(2-oxo-2-(((R)-2′-oxo-1,1′,2′,3-tetrahydrospiro[indene-2,3′-pyrrolo[2,3-b]pyridin]-5-yl)amino)ethyl)pivalamido)piperidine-1-carboxylate 18.5

To a solution of compound 18.4 (180 mg, 0.50 mmol) in DMF (5 mL) was added DIEA (163 mg, 1.26 mmol), HOAt (89 mg, 0.66 mmol), EDCI (126 mg, 0.66 mmol) and Intermediate B (127 mg, 0.50 mmol). The mixture was stirred at 25° C. for 1 h. The reaction mixture was quenched by the addition of water (50 mL) and extracted with ethyl acetate (2×30 mL). The combined organic layers were washed with brine (20 mL) and dried over anhydrous sodium sulfate. After filtration and concentration, the residue was purified by silica gel column chromatography, eluting with petroleum ether:ethyl acetate=10:1˜1:1 to provide compound 18.5 as a white solid (170 mg, 54% yield, 95.1% purity). ¹H NMR (CD₃OD, 400 MHz) δ 1.09 (d, 3H), 1.35 (s, 9H), 1.46 (s, 9H), 1.75-1.88 (m, 1H), 1.95-2.05 (m, 1H), 2.09-2.18 (m, 1H), 2.86-2.96 (m, 2H), 3.08 (dd, 2H), 3.52 (dd, 2H), 3.79-3.87 (m, 1H), 4.01 (br. s, 2H), 4.28-4.41 (m, 2H), 6.88 (dd, 1H), 7.13 (dd, 1H), 7.22 (d, 1H), 7.36-7.38 (m, 1H), 7.56 (s, 1H), 8.05 (dd, 1H). LCMS Method 1: rt 0.975 min, (590.2 [M+H]⁺).

### Example 27

### N-((3S*,5S*)-5-Methylpiperidin-3-yl)-N-(2-oxo-2-(((R)-2′-oxo-1,1′,2′,3-tetrahydrospiro[indene-2,3′-pyrrolo[2,3-b]pyridin]-5-yl)amino)ethyl)pivalamide

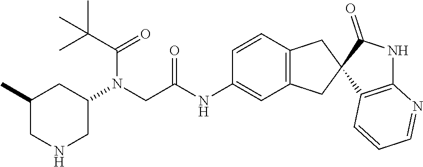 [SMILES: C[C@@H]1CNC[C@@H](N(CC(=O)Nc2ccc3c(c2)C[C@@]2(C3)C(=O)Nc3ncccc32)C(=O)C(C)(C)C)C1]

To a solution of compound 18.5 (80 mg, 0.14 mmol) in dichloromethane (5 mL) was added zinc bromide (458 mg, 2.03 mmol). The mixture was stirred at 25° C. for 16 h. The reaction was quenched by the addition of water (50 mL), and the mixture adjusted to pH10 with sodium carbonate powder. The suspension was extracted with ethyl acetate (2×30 mL). The organic layers were combined, washed with brine (20 mL) and dried over anhydrous sodium sulfate. After filtration and concentration, the residue was purified by prep-HPLC (column: Luna C18 150×25 mm, 5 μm; mobile phase: [solvent A: water (0.075% TFA), solvent B: MeCN]; B %: 10-40%, 9 min). After lyophilisation, Example 27 was obtained as a white solid (24 mg, 29% yield, TFA salt, 97.1% purity). ¹H NMR (CD₃OD, 400 MHz) δ 1.05-1.26 (m, 3H), 1.33 (s, 9H), 1.57-1.94 (m, 1H), 2.05-2.22 (m, 1H), 2.26-2.64 (m, 1H), 3.06-3.11 (m, 2H), 3.12-3.30 (m, 2H), 3.42-3.60 (m, 3H), 3.89-4.84 (m, 4H), 6.89 (dd, 1H), 7.15 (dd, 1H), 7.24 (d, 1H), 7.35-7.43 (m, 1H), 7.57 (d, 1H), 8.06 (dd, 1H). LCMS Method 4: rt 1.964 min, (490.2 [M+H]⁺).

### Example 28

### N-((3S*,6S*)-6-Methyl piperidin-3-yl)-N-(2-oxo-2-(((R)-2′-oxo-1,1′,2′,3-tetrahydrospiro[indene-3′-pyrrolo[2,3-b]pyridin]-5-yl)amino)ethyl)pivalamide

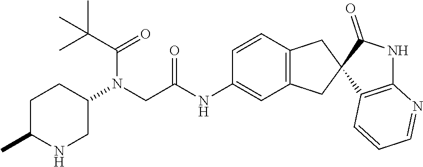 [SMILES: C[C@H]1CC[C@H](N(CC(=O)Nc2ccc3c(c2)C[C@@]2(C3)C(=O)Nc3ncccc32)C(=O)C(C)(C)C)CN1]

The target was prepared using the procedures described for Example 27, starting from 2-methyl-2-propanyl 2-methyl-5-oxo-1-piperidinecarboxylate. Final purification by prep-HPLC (column: Luna C18 150×25 mm, 5 μm; mobile phase: [solvent A: water (0.075% TFA), solvent B: MeCN]; B %: 12-42%, 9 min) gave Example 28 as a white solid (40 mg, 39% yield, TFA salt, 98.8% purity). ¹H NMR (CD₃OD, 400 MHz) δ 1.32-1.34 (m, 12H), 1.59-1.63 (m, 1H), 2.02-2.12 (m, 3H), 3.10 (dd, 2H), 3.18-3.22 (m, 1H), 3.48-3.57 (m, 3H), 3.96-4.21 (m, 2H), 4.33-4.77 (m, 2H), 6.89 (dd, 1H), 7.15 (d, 1H), 7.23 (d, 1H), 7.39 (dd, 1H), 7.59 (d, 1H), 8.06 (dd, 1H). LC-MS Method 4: rt 1.87 min, (490.2 [M+H]⁺).

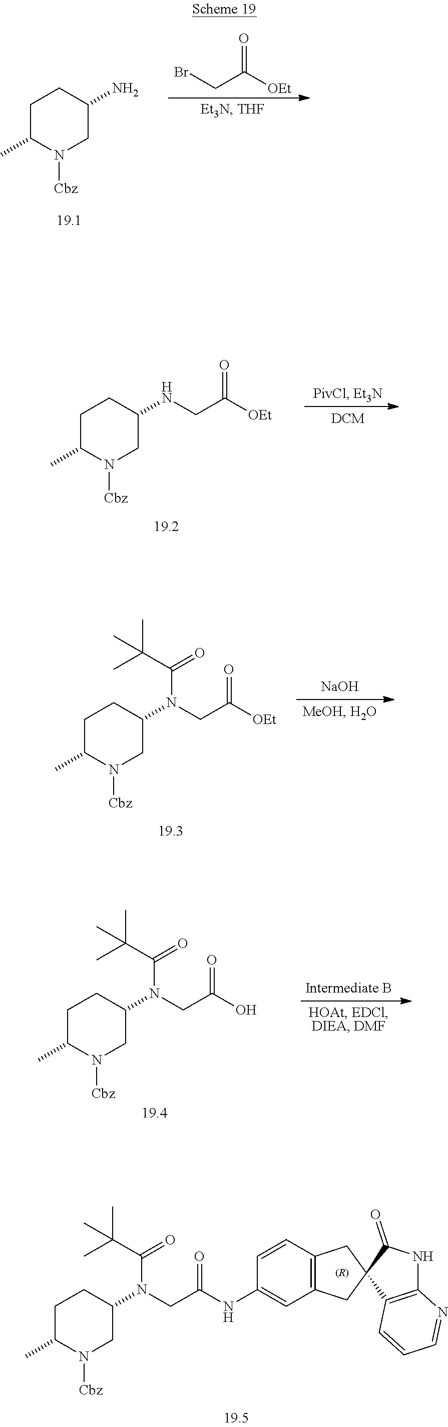

### (2R*,5S*)-Benzyl 5-((2-ethoxy-2-oxoethyl)amino)-2-methylpiperidine-1-carboxylate 19.2

To a solution of compound 19.1 (300 mg, 1.21 mmol) in tetrahydrofuran (6 mL) was added triethylamine (183 mg, 1.81 mmol) and ethyl 2-bromoacetate (222 mg, 1.33 mmol) at 20° C. The mixture was stirred at 20° C. for 15 h, poured into water (20 mL) and extracted with ethyl acetate (3×20 mL). The organic layers were combined, washed with brine (50 mL) and dried over anhydrous sodium sulfate. After filtration and concentration, the residue was purified by silica gel column chromatography, eluting with petroleum ether:ethyl acetate=1:0 to 1:1 to give compound 19.2 as a colourless oil (301 mg, 74% yield). ¹H NMR (CDCl₃, 400 MHz) δ 1.15 (d, 3H), 1.27 (t, 3H), 1.40-1.57 (m, 2H), 1.66-1.85 (m, 2H), 2.44-2.67 (m, 2H), 2.96-3.23 (m, 1H), 3.45 (s, 2H), 4.06-4.24 (m, 3H), 4.38-4.54 (m, 1H), 5.14 (s, 2H), 7.28-7.41 (m, 5H).

### (2R*,5S*)-Benzyl 5-(N-(2-ethoxy-2-oxoethyl)pivalamido)-2-methylpiperidine-1-carboxylate 19.3

To a solution of compound 19.2 (300 mg, 0.90 mmol) and triethylamine (272 mg, 2.69 mmol) in dichloromethane (5 mL) was added pivaloyl chloride (195 mg, 1.61 mmol) at 0° C. The mixture was stirred at 20° C. for 2 h, poured into water (20 mL) and extracted with ethyl acetate (3×50 mL). The organic phases were combined, washed with brine (50 mL) and dried over anhydrous sodium sulfate. After filtration and concentration, the residue was purified by silica gel column chromatography, eluting with petroleum ether:ethyl acetate=1:0 to 1:1 to give compound 19.3 as a colourless oil (240 mg, 64% yield). ¹H NMR (CDCl₃, 400 MHz) δ 1.16-1.21 (m, 3H), 1.24-1.33 (m, 12H), 1.64-1.88 (m, 4H), 2.81-2.99 (m, 1H), 3.74-4.08 (m, 3H), 4.14-4.26 (m, 3H), 4.39-4.60 (m, 1H), 5.00-5.24 (m, 2H), 7.29-7.41 (m, 5H).

### 2-(N-((3S*,6R*)-1-((Benzyloxy)carbonyl)-6-methylpiperidin-3-yl)pivalamido)acetic acid 19.4

To a solution of compound 19.3 (210 mg, 0.50 mmol) in methanol (4.5 mL) was added a solution of sodium hydroxide (110 mg, 2.75 mmol) in water (1.5 mL) at 20° C. The mixture was stirred at 20° C. for 1 h, poured into water (20 mL), the pH adjusted to pH4 with hydrochloric acid and the resulting mixture was extracted with ethyl acetate (3×20 mL). The organic layers were combined, washed with brine (20 mL) and dried over anhydrous sodium sulfate. After filtration and concentration, compound 19.4 was obtained as a white solid (140 mg, 71% yield). ¹H NMR (CD₃OD, 400 MHz) δ 1.20-1.33 (m, 12H), 1.65-2.03 (m, 4H), 2.96-3.16 (m, 1H), 3.90-4.22 (m, 4H), 4.42-4.50 (m, 1H), 5.05-5.20 (m, 2H), 7.21-7.42 (m, 5H).

### (2R*,5S*)-Benzyl 2-methyl-5-(N-(2-oxo-2-(((R)-2′-oxo-1,1′,2′,3-tetrahydrospiro[indene-2,3′-pyrrolo[2,3-b]pyridin]-5-yl)amino)ethyl)pivalamido)piperidine-1-carboxylate 19.5

To a solution of compound 19.4 (70 mg, 0.18 mmol), EDCI (52 mg, 0.27 mmol) and HOAt (37 mg, 0.27 mmol) in DMF (2 mL) was added N,N-diisopropylethylamine (93 mg, 0.72 mmol) followed by Intermediate B (45 mg, 0.17 mmol) at 20° C. The mixture was stirred at 20° C. for 12 h, poured into water (20 mL) and extracted with ethyl acetate (3×50 mL). The organic layers were combined, washed with 0.1M hydrochloric acid (20 mL), brine (50 mL) and dried over anhydrous sodium sulfate. After filtration and concentration, the residue was purified by silica gel column chromatography, eluting with petroleum ether:ethyl acetate=1:0 to 1:5 to give compound 19.5 as a white solid (74 mg, 66% yield, 99.7% purity). ¹H NMR (CD₃OD, 400 MHz) δ 1.22-1.34 (m, 12H), 1.67-1.89 (m, 3H), 2.01-2.08 (m, 1H), 3.01-3.19 (m, 3H), 3.52 (dd, 2H), 3.97-4.11 (m, 3H), 4.18-4.27 (m, 1H), 4.40-4.50 (m, 1H), 5.00-5.23 (m, 2H), 6.88 (dd, 1H), 7.12 (d, 1H), 7.22 (d, 1H), 7.27-7.40 (m, 6H), 7.56 (s, 1H), 8.05 (dd, 1H). LC-MS Method 1: rt 0.929 min, (624.4 [M+H]⁺).

### Example 29

### N-((3S*,6R*)-6-Methylpiperidin-3-yl)-N-(2-oxo-2-(((R)-2′-oxo-1,1′,2′,3-tetrahydrospiro[indene-2,3′-pyrrolo[2,3-b]pyridin]-5-yl)amino)ethyl)pivalamide

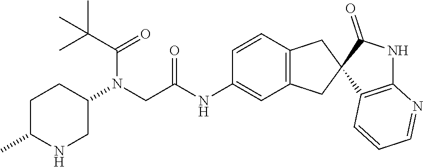 [SMILES: C[C@@H]1CC[C@H](N(CC(=O)Nc2ccc3c(c2)C[C@@]2(C3)C(=O)Nc3ncccc32)C(=O)C(C)(C)C)CN1]

To a solution of compound 19.5 (74 mg, 0.12 mmol) and trifluoroacetic acid (14 mg, 0.12 mmol) in methanol (3 mL) was added 10% Pd/C (20 mg). The mixture was degassed under vacuum and purged with hydrogen three times. The resulting mixture was stirred at 20° C. for 3 h under a hydrogen balloon (15 psi). The catalyst was removed by filtration, and the filtrate was concentrated under reduced pressure. The residue was purified by prep-HPLC (column: Luna C18 150×25 mm, 5 μm; mobile phase: [solvent A: water (0.075% TFA), solvent B: MeCN]; B %: 10-40%, 9 min). After lyophilisation, Example 29 was obtained as a white solid (23 mg, 33% yield, TFA salt, 99.1% purity). ¹H NMR (CD₃OD, 400 MHz) δ 1.32 (s, 9H), 1.42 (d, 3H), 1.82-2.24 (m, 4H), 3.09 (dd, 2H), 3.35-3.42 (m, 1H), 3.46-3.62 (m, 4H), 3.90-4.58 (m, 3H), 6.90 (dd, 1H), 7.16 (dd, 1H), 7.24 (d, 1H), 7.38 (d, 1H), 7.59 (s, 1H), 8.06 (dd, 1H). LC-MS Method 4: rt 1.996 min, (490.2 [M+H]⁺).

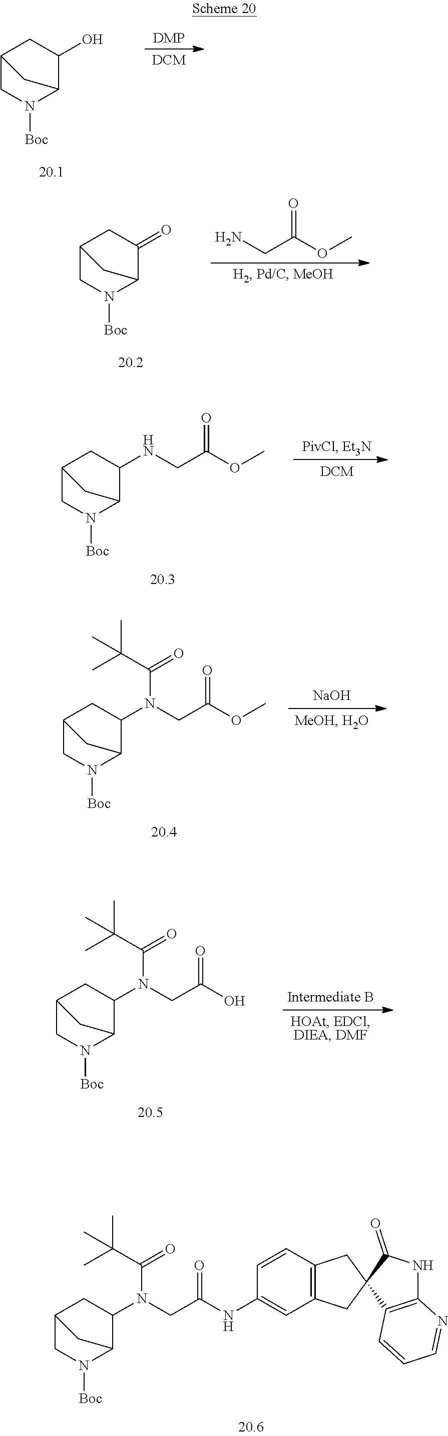

### tert-Butyl 6-oxo-2-azabicyclo[2.2.1]heptane-2-carboxylate 20.2

To a solution of compound 20.1 (200 mg, 0.94 mmol) in dichloromethane (5 mL) was added Dess-Martin periodinane (477 mg, 1.13 mmol) at 20° C. The mixture was stirred at 20° C. for 1 h, poured into saturated aqueous sodium sulfite (20 mL) and extracted with ethyl acetate (3×20 mL). The organic phases were combined, washed with saturated aqueous sodium bicarbonate (20 mL) and brine (2×20 mL) and dried over sodium sulfate. After filtration and concentration, the residue was purified by silica gel column chromatography, eluting with petroleum ether:ethyl acetate=1:0 to 10:1, to give compound 20.2 as a white solid (129 mg, 65% yield). ¹H NMR (CDCl₃, 400 MHz) δ 1.46 (s, 9H), 1.72 (d, 1H), 1.85-1.95 (m, 1H), 2.01 (dd, 1H), 2.18-2.27 (m, 1H), 2.84 (s, 1H), 3.06-3.26 (m, 1H), 3.43-3.49 (m, 1H), 4.04-4.34 (m, 1H).

### tert-Butyl 6-((2-methoxy-2-oxoethyl)amino)-2-azabicyclo[2.2.1]heptane-2-carboxylate 20.3

To a solution of compound 20.2 (129 mg, 0.61 mmol) and sodium acetate (110 mg, 1.34 mmol) in methanol (3 mL) was added 10% Pd/C (20 mg) and glycine methyl ester hydrochloride (109 mg, 1.22 mmol) at 20° C. The mixture was degassed and purged with hydrogen three times and stirred at 25° C. for 16 h under a hydrogen balloon (15 psi). The catalyst was removed by filtration and the filtrate was poured into water (20 mL). The mixture was adjusted to pH4 with 1M hydrochloric acid and extracted with ethyl acetate (2×50 mL). The aqueous phase was adjusted to pH10 with saturated aqueous sodium carbonate. The resulting mixture was extracted with ethyl acetate (2×50 mL). The organic phases were combined, washed with brine (50 mL) and dried over sodium sulfate. After filtration and concentration, compound 20.3 was obtained as a white solid (155 mg, 89% yield). ¹H NMR (CDCl₃, 400 MHz) δ 0.91 (dt, 1H), 1.11-1.53 (m, 10H), 1.62-1.71 (m, 1H), 2.04-2.14 (m, 1H), 2.40-2.51 (m, 1H), 2.99 (dd, 1H), 3.08-3.21 (m, 1H), 3.25-3.45 (m, 2H), 3.53-3.78 (m, 4H), 4.20 & 4.30 (s, 1H).

### tert-Butyl 6-(N-(2-methoxy-2-oxoethyl)pivalamido)-2-azabicyclo[2.2.1]heptane-2-carboxylate 20.4

To a solution of compound 20.3 (155 mg, 0.55 mmol) and triethylamine (165 mg, 1.64 mmol) in dichloromethane (5 mL) was added pivaloyl chloride (13 mg, 1.09 mmol) at 0° C. The mixture was stirred at 20° C. for 12 h, poured into water (20 mL) and extracted with ethyl acetate (3×50 mL). The organic layers were combined, washed with 1M hydrochloric acid (20 mL) and brine (50 mL) and dried over sodium sulfate. After filtration and concentration, the residue was purified by silica gel column chromatography, eluting with petroleum ether:ethyl acetate=1:0 to 5:1 to give compound 20.4 as a yellow oil (179 mg, 89% yield). ¹H NMR (CDCl₃, 400 MHz) δ 1.28-1.33 (m, 9H), 1.45 (s, 9H), 1.52-1.61 (m, 3H), 2.07-2.18 (m, 1H), 2.52-2.66 (m, 1H), 2.93-3.06 (m, 1H), 3.30-3.65 (m, 2H), 3.71 (s, 3H), 4.20 & 4.30 (s, 1H), 4.60 (s, 2H).

### 2-(N-(2-(tert-Butoxycarbonyl)-2-azabicyclo[2.2.1]heptan-6-yl)pivalamido)acetic acid 20.5

To a solution of compound 20.4 (179 mg, 0.49 mmol) in methanol (4.5 mL) was added sodium hydroxide (78 mg, 1.94 mmol) in water (1.5 mL) at 20° C. The mixture was stirred at 20° C. for 15 h, poured into water (20 mL) and adjusted to pH4 with 1M hydrochloric acid. The resulting mixture was extracted with ethyl acetate (3×20 mL). The organic layers were combined, washed with brine (50 mL) and dried over anhydrous sodium sulfate. After filtration and concentration, compound 20.5 was isolated as a colourless oil (145 mg, 84% yield). ¹H NMR (CD₃OD, 400 MHz) δ 1.26-1.34 (m, 9H), 1.40-1.49 (m, 9H), 1.50-1.66 (m, 2H), 1.68-1.79 (m, 1H), 2.05-2.19 (m, 1H), 2.61 (s, 1H), 2.98-3.17 (m, 1H), 3.34-3.43 (m, 1H), 4.01-4.20 (m, 1H), 4.22-4.38 (m, 1H), 4.60-4.78 (m, 2H).

### tert-Butyl 6-(N-(2-oxo-2-(((R)-2′-oxo-1,1′,2′,3-tetrahydrospiro[indene-2,3′-pyrrolo[2,3-b]pyridin]-5-yl)amino)ethyl)pivalamido)-2-azabicyclo[2.2.1]heptane-2-carboxylate 20.6

To a solution of 20.5 (145 mg, 0.41 mmol), EDCI (117.64 mg, 0.61 mmol) and HOAt (84 mg, 0.61 mmol) in DMF (4 mL) was added DIEA (211 mg, 1.64 mmol) and Intermediate B (113 mg, 0.45 mmol) at 20° C. The mixture was stirred at 20° C. for 4 h, poured into water (20 mL) and extracted with ethyl acetate (3×20 mL). The organic layers were combined, washed with 1M hydrochloric acid (20 mL) and brine (50 mL) and dried over sodium sulfate. After filtration and concentration, compound 20.6 was obtained as a white solid (210 mg, 87% yield, 94.4% purity). LC-MS Method 1: rt 0.908 min, (588.3 [M+H]⁺).

### Example 30

### N-(2-Azabicyclo[2.2.1]heptan-6-yl)-N-(2-oxo-2-(((R)-2′-oxo-1,1′,2′,3-tetrahydrospiro[indene-2,3′-pyrrolo[2,3-b]pyridin]-5-yl)amino)ethyl)pivalamide

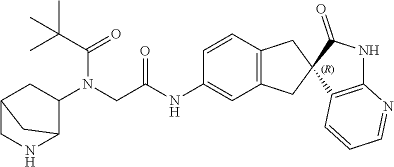

To a solution of compound 20.6 (100 mg, 0.17 mmol) in dichloromethane (5 mL) was added zinc bromide (958 mg, 4.25 mmol) at 20° C. The mixture was stirred at 20° C. for 12 h and the volatiles removed in vacuo. The residue was dissolved in methanol (10 mL) and poured into water (10 mL). The suspension was adjusted to pH10 with saturated aqueous sodium carbonate and extracted with ethyl acetate (5×50 mL). The organic layers were combined, dried over anhydrous sodium sulfate, filtered and concentrated under vacuum. The residue was purified by prep-HPLC (column: Luna C18 150×25 mm, 5 μm; mobile phase: [solvent A: water (0.075% TFA), solvent B: MeCN]; B %: 12-42%, 9 min). After lyophilisation, Example 30 was obtained as an off-white solid (30 mg, 28% yield, TFA salt, 96.3% purity). ¹H NMR (CD₃OD, 400 MHz) δ 1.28-1.34 (m, 9H), 1.66-1.80 (m, 2H), 1.95-2.02 (m, 1H), 2.10-2.23 (m, 1H), 2.73 (s, 1H), 3.07-3.20 (m, 3H), 3.31-3.34 (m, 1H), 3.52 (dd, 2H), 4.03-4.11 (m, 1H), 4.25-4.88 (m, 2H), 4.65 & 4.62 (s, 1H), 6.91 (dd, 1H), 7.17 (d, 1H), 7.28 (d, 1H), 7.42 (t, 1H), 7.62 (d, 1H), 8.07 (dd, 1H). LC-MS Method 4: rt 1.988 min, (488.2 [M+H]⁺).

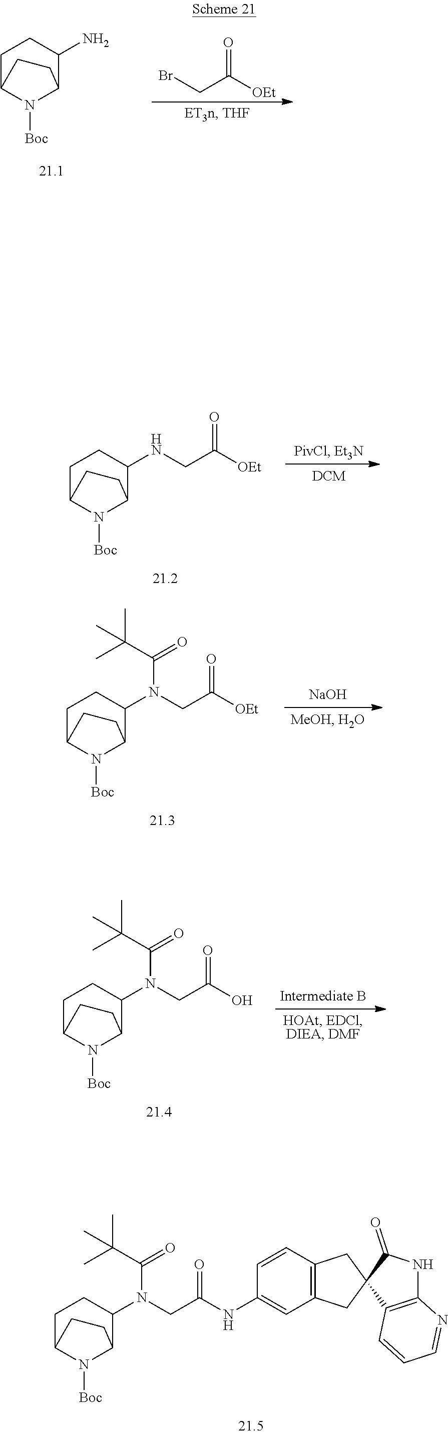

### tert-Butyl 2-((2-ethoxy-2-oxoethyl)amino)-8-azabicyclo[3.2.1]octane-8-carboxylate 21.2

To a solution of compound 21.1 (150 mg, 0.66 mmol) in THF (3 mL) was added triethylamine (80 mg, 0.80 mmol) and ethyl 2-bromoacetate (122 mg, 0.73 mmol). The mixture was stirred at 25° C. for 16 h, poured into water (10 mL) and extracted with ethyl acetate (2×10 mL). The organic layers were combined, washed with brine (20 mL) and dried over sodium sulfate. After filtration and concentration, compound 21.2 was obtained as a yellow oil (188 mg, crude). ¹H NMR (CDCl₃, 400 MHz) δ 1.28 (t, 3H), 1.47 (s, 9H), 1.65-2.05 (m, 8H), 3.43 (s, 2H), 3.98-4.33 (m, 5H).

### tert-Butyl 2-(N-(2-ethoxy-2-oxoethyl)pivalamido)-8-azabicyclo[3.2.1]octane-8-carboxylate 21.3

To a solution of compound 21.2 (188 mg, 0.60 mmol) in dichloromethane (3 mL) was added DIEA (101 mg, 0.78 mmol) and pivaloyl chloride (87 mg, 0.72 mmol). The mixture was stirred at 25° C. for 1 h, poured into water (10 mL) and extracted with ethyl acetate (2×20 mL). The organic layers were combined, washed with brine (50 mL) and dried over sodium sulfate. After filtration and concentration, compound 21.3 was obtained as a yellow oil (207 mg, crude). ¹H NMR (CDCl₃, 400 MHz) δ 1.25 (t, 3H), 1.35 (s, 9H), 1.45 (s, 9H), 1.83-1.98 (m, 8H), 3.87 (d, 1H), 4.16-4.23 (m, 3H), 4.35-4.40 (m, 3H).

### 2-(N-(8-(tert-Butoxycarbonyl)-8-azabicyclo[3.2.1]octan-2-yl)pivalamido)acetic acid 21.4

To a solution of compound 21.3 (200 mg, 0.50 mmol) in methanol (4 mL) and water (2 mL) was added sodium hydroxide (81 mg, 2.02 mmol). The mixture was stirred at 20° C. for 15 h, poured into water (10 mL), adjusted to pH4 with 1M hydrochloric acid and extracted with ethyl acetate (3×10 mL). The organic layers were combined, washed with brine (10 mL) and dried over sodium sulfate. After filtration and concentration, compound 21.4 was obtained as a yellow oil (99 mg, crude). ¹H NMR (CDCl₃, 400 MHz) δ 1.36 (s, 9H), 1.46 (s, 9H), 1.82-2.01 (m, 8H), 3.95-4.53 (m, 5H).

### tert-Butyl 2-(N-(2-oxo-2-(((R)-2′-oxo-1,1′,2′,3-tetrahydrospiro[indene-2,3′-pyrrolo[2,3-b]pyridin]-5-yl)amino)ethyl)pivalamido)-8-azabicyclo[3.2.1]octane-8-carboxylate 21.5

To a solution of compound 21.4 (80 mg, 0.22 mmol) in DMF (2 mL) was added DIEA (70 mg, 0.54 mmol), EDCI (50 mg, 0.26 mmol), HOAt (35 mg, 0.26 mmol) and Intermediate B (56 mg, 0.22 mmol). The mixture was stirred at 25° C. for 1 h, poured into water (10 mL), acidified to pH4 with 1M hydrochloric acid and extracted with ethyl acetate (3×10 mL). The organic layers were combined, washed with brine (3×20 mL) and dried over sodium sulfate. After filtration and concentration, the residue was purified by silica gel column chromatography, eluting with petroleum ether:ethyl acetate=10:1˜5:1, to afford compound 21.5 as a yellow solid (86 mg, 66% yield). ¹H NMR (CDCl₃, 400 MHz) δ 1.40 (s, 9H), 1.46 (s, 9H), 1.68-2.05 (m, 8H), 3.00-3.07 (m, 2H), 3.59-3.69 (m, 2H), 4.13-4.53 (m, 5H), 6.80-6.84 (m, 1H), 7.05-7.10 (m, 1H), 7.16-7.24 (m, 2H), 7.41-7.65 (m, 1H), 8.11 (d, 1H), 8.46 (br. s, 1H).

### Examples 31A and 31B

### N-(8-Azabicyclo[3.2.1]octan-2-yl)-N-(2-oxo-2-(((R)-2′-oxo-1,1′,2′,3-tetrahydrospiro[indene-2,3′-pyrrolo[2,3-b]pyridin]-5-yl)amino)ethyl)pivalamide

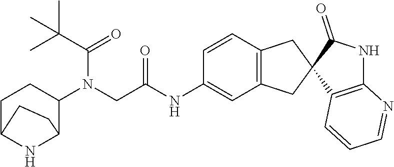 [SMILES: CC(C)(C)C(=O)N(CC(=O)Nc1ccc2c(c1)C[C@@]1(C2)C(=O)Nc2ncccc21)C1CCC2CCC1N2]

To a solution of compound 21.5 (106 mg, 0.18 mmol) in dichloromethane (5 mL) was added zinc bromide (595 mg, 2.64 mmol). The mixture was stirred at 25° C. for 12 h, poured into brine (10 mL), adjusted to pH10 with saturated aqueous sodium carbonate and extracted with 5:1 ethyl acetate/methanol (6×20 mL). The organic layers were combined and dried over sodium sulfate. After filtration and concentration, the residue was purified by prep-HPLC (column: Luna C18 150×25 mm, 5 μm; mobile phase: [solvent A: water (0.075% TFA), solvent B: MeCN]; B %: 10-40%, 9 min). After lyophilisation, Example 31A (26 mg, 24% yield, TFA salt, 97.2% purity)—the first peak (diastereoisomer), shorter LCMS retention time—and Example 31B (12 mg, 14% yield, TFA salt, 98.5% purity)—the second peak (diastereoisomer), longer LCMS retention time—were obtained as white solids. Example 31A ¹H NMR (CD₃OD, 400 MHz) δ 1.32 (s, 9H), 1.83-2.15 (m, 8H), 3.09 (dd, 2H), 3.52 (dd, 2H), 4.03 (d, 1H), 4.07-4.17 (m, 1H), 4.39-4.42 (m, 1H), 4.59 (d, 1H), 4.72-4.81 (m, 1H), 6.89 (dd, 1H), 7.16 (d, 1H), 7.25 (d, 1H), 7.36 (t, 1H), 7.56 (d, 1H), 8.06 (dd, 1H). LCMS (Method 6): rt 1.588 min, (502.3 [M+H]⁺). Example 31B ¹H NMR (CD₃OD, 400 MHz) δ 1.31 (s, 9H), 1.60-1.64 (m, 1H), 1.90-2.26 (m, 7H), 3.14 (dd, 2H), 3.53 (dd, 2H), 3.64-3.67 (m, 1H), 4.02-4.09 (m, 1H), 4.27-4.37 (m, 1H), 4.46 (d, 1H), 4.62 (d, 1H), 6.89 (dd, 1H), 7.17 (dd, 1H), 7.28 (d, 1H), 7.39 (d, 1H), 7.62 (s, 1H), 8.06 (dd, 1H). LCMS (Method 6): rt 1.671 min, (502.3 [M+H]⁺).

Biological Assays

The following assays can be used to measure the effects of the compounds of the present invention.

cAMP/Agonist-Antagonist Competition Assays in Cell Lines

Compounds were assessed for their ability to inhibit ligand-induced elevations of cAMP, using the Perkin Elmer LANCE cAMP assay using commercially-available cells expressing a specific receptor of interest using the following general procedure:

Compound Preparation

Compounds were prepared in dimethyl sulfoxide (DMSO—99.9% pure) (Sigma Aldrich, Cat #: D4540) added to powder stocks to produce a 20 mM solution (100% DMSO) that was sonicated at 37° C. for 10 minutes to fully dissolve the compounds. The 20 mM stocks were diluted further in DMSO to produce a 2 mM solution that was sonicated at 37° C. for 10 minutes. 2 mM stocks were dissolved in assay/stimulation buffer to produce a 400 μM solution that was sonicated at 37° C. for 10 minutes for all cAMP assays. All stocks were stored at −20° C. Then serial dilution (Dilution factor: 10) was performed to achieve the desire experimental concentrations.

Assay Protocol

Competition assays were performed according to the manufactures instructions using LANCE® TR-FRET cAMP assay kit (Perkin Elmer, Cat #: AD0264). Serial dilutions (3 μl/well) of the molecules were plated in a 384-well OptiPlate (Perkin Elmer, Cat #: 6007299) in duplicates. Appropriate controls (100% stimulation: Forskolin and 0% stimulation: Vehicle control) (6 μl/well) were included in each plate for data normalization. Following the compound addition, 6 μl of G-protein coupled receptor overexpressing cells/Alexa Fluor antibody solution (1:100 dilution) was added in each well at a desired density of 2500 cells/well. The overexpressing cell lines were purchased from DiscoveRx, Birmingham, UK. After spinning the plate at 1000 rpm for 1 minute and vortexing briefly, the cells were pre-incubated with the compounds for 30 minutes at room temperature (covered). Then 3 μl of the equivalent peptide ligand (EC₅₀ dose) was added to all the wells except vehicle and forskolin controls. The plate were then spun down at 1000 rpm for 1 minute and once finished they were vortexed briefly and covered. Cells were stimulated in the presence of the ligands for 15 minutes at room temperature. After stimulation 12 μl of detection mix (Europium-Chelate streptavidin/biotinylated cAMP tracer solution) was added to all wells and incubated for 60 minutes at room temperature. The plate was then read on the Enspire multimode Plate reader (Perkin Elmer), at; 320/340 nm excitation and 615/665 nm emission was recorded.

Assay/Stimulation Buffer (30 mL)—pH 7.4
28 mL Hank's Balanced salt solution (+MgCl₂, +CaCl₂))—(Thermo Fisher Cat #:14170112)
150 μl HEPES (1M)—(Thermo Fisher Cat #:15630080)
400 μl Stabilizer (DTPA) Purified BSA (7.5%)—(Perkin Elmer, Cat #: CR84-100)
60 μl IBMX (250 mM)—(Sigma Aldrich, Cat #: 15879)

Specific cAMP/Agonist-Antagonist Competition Assays

The following specific assays were run using the procedure above

AM₂ Receptor Inhibition

The ability of a compound to inhibit the AM induced cAMP activation in AM₂ receptor-expressing cells (1321N1 cells transfected with CALCRL+RAMP3, sourced from DiscoverX catalogue number 95-0169C6) was assessed using the protocol above.

The activity of compounds in this assay are set out in Table 4.

AM₁ Receptor Inhibition

The ability of a compound to inhibit AM induced activation of AM₁ receptor-expressing cells (CHO-K1 cells transfected with CALCRL+RAMP2, sourced from DiscoverX catalogue number 93-0270C₂) was assessed using the general protocol above.

Compounds tested in this assay generally exhibited a pIC₅₀ in the range of 5 to 5.7.

AMY₃ Receptor Inhibition

The ability of a compound to inhibit AMY induced activation of AMY₃R-expressing cells (1321N1 cells transfected with CALCR+RAMP-3 sourced from DiscoverX, catalogue number 95-0166C6) was assessed using the general protocol above.

Compounds tested in this assay generally exhibited a pIC₅₀ in the range of 3.5 to 6.6.

Cell Viability Assays

Cell viability assays were performed according to the manufacturer's instructions using RealTime-Glo™ MT Cell Viability Assay kit (Promega, Cat #: G9712). These assays demonstrated the test compounds' (3 μM) ability to inhibit cell survival and growth by between 40% and 70%.

All cell lines used were purchased from ATCC Virginia, USA (Table 1). Cells were seeded at a desired density in complete growth media into white clear-bottom 96-well plates (Corning, Cat #: 3610). Plates were incubated for 15 mins at room temperature (to ensure even settling of the cells) before incubated overnight at 37° C. in 5% CO₂. The next day the viability assay kit reagents (enzyme and substrate) were equilibrated in a 37° C. water bath alongside suboptimal growth media (assay buffer) for 10-15 mins. A reagent solution was then made containing 1:1000 of each reagent in the suboptimal growth media of each cell line (vortex well prior to use). The complete growth media was then removed from the wells and replaced with 100 μl of the reagent solution. Plates were then incubated at 37° C. in 5% CO₂ for at least 1 hour before reading untreated baseline. Reagents were replaced every 3 days the wells were washed once with PBS and fresh reagents were added as above for longer duration of treatments. After reading the baseline, the wells were treated with the appropriate concentration of test molecules the plates were centrifuged at 110×g for 1 min to ensure wells with even compound distribution, then incubated at 37° C. in 5% CO₂. Plates were treated once-daily (for 9 days) after luminescence measurements were taken using Enspire multimode Plate reader (Perkin Elmer).

TABLE 1
Cell Lines and corresponding complete growth
media, suboptimal media and seeding density

| | | | Seeding |
|---|---|---|---|
| | | | Density |
| Cell Line | Complete Growth Media | Suboptimal Media | (per well) |
| MDA-MB-231 | RPMI + 10% FBS (Sigma) | RPMI + 1% FBS (Sigma) | 2,000 |
| 178-2 BMA | DMEM + 10% FBS | DMEM + 2% FBS | 2,000 |
| | (Gibco) + 0.01M HEPES | (Gibco) + 0.01M HEPES | |
| ASPC-1 | RPMI + 15% FBS (Gibco) | RPMI + 5% FBS (Gibco) | 2,000 |
| BxPC-3 | RPMI + 10% FBS (Gibco) | RPMI + 5% FBS (Gibco) | 2,000 |
| Capan-2 | McCoy's + 10% FBS (Sigma) | McCoy's + 5% FBS (Sigma) | 2,000 |
| CFPAC-1 | DMEM + 10% FBS (Gibco) | DMEM + 5% FBS (Gibco) | 2,000 |
| HPAF-II | RPMI + 10% FBS (Gibco) | RPMI + 5% FBS (Gibco) | 2,000 |
| Panc10.05 | RPMI + 15% FBS (Gibco) | RPMI + 5% FBS (Gibco) | 2,000 |
| SW1990 | DMEM + 10% FBS (Gibco) | DMEM + 1% FBS (Gibco) | 2,000 |

In-Vivo Effects: Xenograft Mouse Model

The in-vivo efficacy of a compound can be assessed using the following xenograft mouse model

Tumour Inoculation

All cell lines used in the in-vivo experiments were purchased from ATCC Virginia, USA (Table 2). Cells were cultured in complete growth media in T500 TripleFlasks (Thermo Fisher, Cat #: 132913). When 80-90% confluency was reached, cells were detached from the flasks using TrypLE Express Enzyme dissociation buffer (Thermo Fisher, Cat #: 12605). Cells were counted using Countess II Automated Cell Counter and then were centrifuged at 110×g for 5 mins. The pellet was re-suspended in the appropriate volume of ice cold PBS (depending on the cell number). To ensure tumour inoculation, cells (500 μL) were mixed with 500 μL of ice cold matrigel (Corning, Cat #: 354234) using chilled pipette tips (pipette slowly to ensure uniform mixing and prevent air bubbles forming in matrigel). Matrigel/cell suspension and syringes were kept on ice before injection into mice. 100 μL of cell suspension (5×10⁶ cells in 50% PBS+50% Matrigel) was injected subcutaneously into 27-week old female Balb/c nude mice for each experiment (10 treatment group and 10 vehicle control group).

TABLE 2
Cell lines and corresponding complete growth media

| Cell Line | Complete Growth Media |
|---|---|
| MDA-MB-231 | RPMI + 10% FBS (Sigma) |
| Capan-2 | McCoy's + 10% FBS (Sigma) |
| CFPAC-1 | DMEM + 10% FBS (Gibco) |
| HPAF-II | RPMI + 10% FBS (Gibco) |
| Panc10.05 | RPMI + 15% FBS (Gibco) |

Compound Preparation

Powder-form compounds were diluted in 100% DMSO (Sigma Aldrich, Cat #: D4540) according to the following formula:

$$Volume of DMSO = 0.06 \times \frac{Mass of compound ( mg )}{8 \text{mg/mL}} .$$

The compounds were then sonicated at 37° C. for 10 mins. Then the appropriate volume of solvent (Table 4) was added to yield 6% DMSO/94% solvent solution according to the following formula:

$$Volume of solvent = 0.94 \times \frac{Mass of compound ( mg )}{8 \text{mg/mL}} .$$

The compounds were then sonicated at 37° C. for 10 mins.

TABLE 3
Recipe for compound solvent

| Reagent | Ratio |
|---|---|
| Kolliphor HS15 | 1 (weight in g) |
| Kollisolv PCGE400 | 3 (volume in mL) |
| PBS | 6 (volume in mL) |

In-Vivo Treatment with Test Compounds

Before treatment each compound vial is diluted with equal part solvent resulting in 4 mg/mL compound in 3% DMSO and then sonicated at 37° C. for 10 mins. The mice are suitably treated daily intraperitoneally with 100 μL of treatment (20 mg/kg) or vehicle control. Doses of e.g. 5 mg/kg or 10 mg/kg of test compound may also be used. Tumour size and mouse weights are measured once a week.

### Biological Data

The compounds shown in Table 4 exhibited the following activity in the AM₂ LANCE cAMP assay described above.

TABLE 4

| | AM2 D2 |
|---|---|
| Example | protocol: |
| Number | pIC50 (M) |
| 1 | 7.84 |
| 2 | 8.52 |
| 3 | 6.13 |
| 4 | 6.6 |
| 5 | 6.77 |
| 6 | 5.08 |
| 7 | 6.02 |
| 8 | 6.84 |
| 9 | 7.36 |
| 10 | 7.3 |
| 11 | 7.07 |
| 12 | 6.58 |
| 13 | 7.2 |
| 14 | 6.54 |
| 15 | 7.3 |
| 16 | 6.95 |
| 17 | 7.73 |
| 18 | 7.17 |
| 19 | 7.85 |
| 20 | 7.25 |
| 21 | 7.56 |
| 22 | 6.61 |
| 23 | 5.54 |
| 24 | 6.88 |
| 25 | 6.89 |
| 26 | 6.32 |
| 27 | 7.29 |
| 28 | 6.73 |
| 29 | 6.75 |
| 30 | 5.52 |
| 31A | 6.5 |
| 31B | 6.7 |

### In-Vivo Xenograft Data

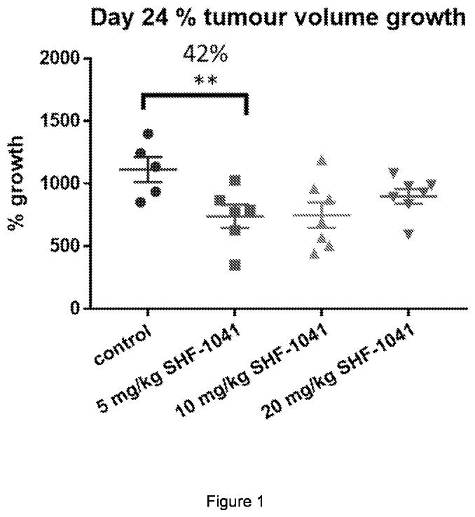

The compound SHF-1041, one of the compounds exemplified herein, was tested in the mouse xenograft model described above, wherein the mice were inoculated with CFPAC-1 cells (cells derived from a ductal adenocarcinoma (ex. ATCC)). The SHF-1041 test compound was administered to treatment mice groups intraperitoneally once-daily at doses of 5 mg/kg, 10 mg/kg and 20 mg/kg. The effect on % tumour volume growth compared to the control group after 24 days dosing of SHF-1041 is illustrated in FIG. 1. At a dose of 5 mg/kg, SHF-1041 inhibited tumour volume growth by 42% compared to the control group.

### Sezary Cell Viability

The effect of the AM2 receptor inhibitor compound SHF-1038 on the viability of Sézary cells was tested. SHF-1038 is a small molecule AM2 receptor inhibitor which is outside the scope of the claims of this invention, but has a pIC50 in the AM2 D2 assay described herein of >8.

A suspension of HUT-78 Sézary cells were seeded in 48-well plates in DMEM with 2% fetal calf serum (2,500 cells/mL, 1 mL/well). Cells were treated daily with an AM2 receptor inhibitor compound (SHF-1038) at a final concentration of 3 μM (or vehicle-control) for 9 days. Fresh media (800 μL per well) was replaced gently every 3 days. Cells were counted at days 5, 7 and 9 using trypan blue exclusion method. 10 μL of cell suspension was added to 10 μL of trypan blue. This mixture was transferred to a disposable counting slide and counted using Countess II Automated Cell-Counter (Thermo Fisher). Cell viability for each treatment condition was normalised to vehicle-treated cells as 100% viable.

The test compound SHF-1038 reduced the cell viability by 68% after the 9 day treatment period.

1. A compound formula (I), or a pharmaceutically acceptable salt thereof: 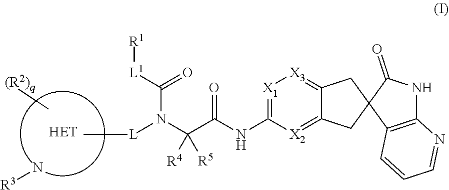
wherein
HET is a 4 to 9 membered saturated or partially saturated heterocyclyl containing 1 ring nitrogen heteroatom and optionally 1 additional ring heteroatom selected from O, S and N;
L is absent or is —C(R^{A})₂—;
each R^{A} is independently selected from H and C₁₋₃ alkyl;
X₁ is N or CR^{B};
X₂ and X₃ are each independently N or CH, provided that no more than one of X1, X₂ and X₃ is N;
L¹ is absent or is selected from: —O— and —N(R⁷)—
R¹ is selected from: H, C₁₋₆ alkyl, C₂₋₆ alkenyl, C₂₋₆ alkynyl, C₁₋₆ haloalkyl and Q¹-L²-, wherein said C₁₋₆ alkyl, C₂₋₆ alkenyl or C₂₋₆ alkynyl is optionally substituted by one or more R⁸;
Q¹ is selected from: C₃₋₁₂ cycloalkyl, C₃₋₁₂ cycloalkenyl, 4 to 12 membered heterocyclyl, C₆₋₁₀ aryl and 5 to 10 membered heteroaryl,
wherein said cycloalkyl, cycloalkenyl and heterocyclyl is optionally substituted by one or more R⁹, and
wherein said aryl and heteroaryl is optionally substituted by one or more R¹⁰;
L² is absent or is selected from: C₁₋₆ alkylene, C₂₋₆ alkenylene and C₂₋₆ alkynylene, wherein L² is optionally substituted by one or more R¹¹
R² is at each occurrence independently selected from: halo, ═O, C₁₋₄ alkyl, C₁₋₄ haloalkyl and —OR^{A12}, or
an R² group forms a C₁₋₆ alkylene bridge between the ring atom to which the R² group is attached and another available ring atom in HET;
R³ is selected from: H and C₁₋₄ alkyl;
R⁴ and R⁵ are independently selected from: H, C₁₋₄ alkyl and C₁₋₄ haloalkyl, or
R⁴ and R⁵ together with the carbon to which they are attached form a C₃₋₆ cycloalkyl;
R⁶ is selected from: H, halo, C₁₋₆ alkyl and C₁₋₆ haloalkyl;
R⁷ is selected from: H, C₁₋₆ alkyl, C₁₋₆ haloalkyl and —OR^{A1};
R⁸, R⁹ and R¹¹ are at each occurrence independently selected from:
halo, ═O, ═NR^{A2}, ═NOR^{A2}, —CN, —NO₂, C₁₋₆ alkyl, C₂₋₆ alkenyl, C₂₋₆ alkynyl, C₁₋₆ haloalkyl, -L³-Q², —OR¹⁶, —S(O)ₓR¹⁶ (wherein x is 0, 1, or 2), —NR¹⁶R^{B2}, —C(O)R¹⁶, —OC(O)R¹⁶, —C(O)OR¹⁶, —NR^{B2}C(O)R¹⁶, —NR^{B2}C(O)OR¹⁶, —C(O)NR¹⁶R^{B2}, —OC(O)NR¹⁶R^{B2}, —NR^{B2}SO₂R¹⁶, —SO₂NR¹⁶R^{B2}, —NR^{A2}C(O)NR¹⁶R^{B2}, —NR^{A2}C(═NR^{A2})R^{B2}, —C(═NR^{A2})R^{B2}, —C(═NR^{A2})NR^{A2}R^{B2}, —NR^{A2}C(═NR^{A2})NR^{A2}R^{B2}, —NR^{A2}C(═NCN)NR^{A2}R^{B2}, —ONR^{A2}R^{B2} and —NR^{A2}OR^{B2},
wherein said C₁₋₆ alkyl, C₂₋₆ alkenyl and C₂₋₆ alkynyl is optionally substituted by 1 or more R¹², and
wherein R¹⁶ is selected from: H, C₁₋₆ alkyl and C₁₋₆ haloalkyl, wherein said C₁₋₆ alkyl is optionally substituted by one or more R¹⁸;
R¹⁰ is at each occurrence independently selected from:
halo, —CN, —NO₂, C₁₋₆ alkyl, C₂₋₆ alkenyl, C₂₋₆ alkynyl, C₁₋₆ haloalkyl, -L⁴-Q³, —OR¹⁷, —S(O)ₓR¹⁷ (wherein x is 0, 1, or 2), —NR¹⁷R^{B3}, —C(O)R¹⁷, —OC(O)R¹⁷, —C(O)OR¹⁷, —NR^{B3}C(O)R¹⁷, —NR^{B3}C(O)OR¹⁷, —C(O)NR¹⁷R^{B3}, —OC(O)NR¹⁷R^{B3}, —NR^{B3}SO₂R¹⁷, —SO₂NR¹⁷R^{B3}, —NR^{A3}C(O)NR¹⁷R^{B3}, —NR^{A3}C(═NR^{A3})R^{A3}, —C(═NR^{A3})R^{B3}, —C(═NR^{A3})NR^{A3}R^{B3}, —NR^{A3}C(═NR^{A3})NR^{A3}R^{B3}, —NR^{A3}C(═NCN)NR^{A3}R^{B3}, —ONR^{A3}R^{B3} and —NR^{A3}OR^{B3},
wherein said C₁₋₆ alkyl, C₂₋₆ alkenyl and C₂₋₆ alkynyl is optionally substituted by 1 or more R¹³, and
wherein R¹⁷ is selected from: H, C₁₋₆ alkyl and C₁₋₆ haloalkyl, wherein said C₁₋₆ alkyl is optionally substituted by one or more R¹⁹;
Q² and Q³ are at each occurrence independently selected from: C₃₋₁₂ cycloalkyl, C₃₋₁₂ cycloalkyl-C₁₋₃ alkyl, C₃₋₁₂ cycloalkenyl, C₃₋₁₂ cycloalkenyl-C₁₋₃ alkyl, 4 to 12 membered heterocyclyl, 4 to 12 membered heterocyclyl-C₁₋₃ alkyl, C₆₋₁₀ aryl, C₆₋₁₀ aryl-C₁₋₃ alkyl, 5 to 10 membered heteroaryl and 5 to 10 membered heteroaryl-C₁₋₃ alkyl,
wherein said C₃₋₁₂ cycloalkyl, C₃₋₁₂ cycloalkyl-C₁₋₃ alkyl, C₃₋₁₂ cycloalkenyl, C₃₋₁₂ cycloalkenyl-C₁₋₃ alkyl, 4 to 12 membered heterocyclyl and 4 to 12 membered heterocyclyl-C₁₋₃ alkyl is optionally substituted by one or more R¹⁴, and
wherein said C₆₋₁₀ aryl, C₆₋₁₀ aryl-C₁₋₃ alkyl, 5 to 10 membered heteroaryl and 5 to 10 membered heteroaryl-C₁₋₃ alkyl is optionally substituted by one or more R¹⁵;
L³ and L⁴ are independently absent or independently selected from: —O—, —CH₂O—, —NR^{A4}—, —CH₂NR^{A4}—, —S(O)ₓ—, —CH₂S(O), (wherein x is 0, 1 or 2), —C(═O)—, —CH₂C(═O)—, —NR^{A4}C(═O)—, —CH₂NR^{A4}C(═O)—, —C(═O)NR^{A4}—, —CH₂C(═O)NR^{A4}—, —S(O)₂NR^{A4}—, —CH₂S(O)₂NR^{A4}—, —NR^{A4}S(O)₂—, CH₂NR^{A4}S(O)₂—, —OC(═O)—, —CH₂OC(═O)—, —C(═O)O— and —CH₂—C(═O)O—;
R¹², R¹³, R¹⁴, R¹⁸ and R¹⁹ are at each occurrence independently selected from:
halo, ═O, —CN, —NO₂, C₁₋₄ alkyl, C₁₋₄ haloalkyl, -L⁵-Q⁴, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, —NR^{B5}C(O)OR^{A5}, —C(O)NR^{A5}R^{B5}, —NR^{B5}SO₂R^{A5} and —SO₂NR^{A5}R^{B5};
wherein said C₁₋₄alkyl is optionally substituted by 1 or 2 substituents selected from:
halo, ═O, —CN, —OR^{A6}, —NR^{A6}R^{B6} and —SO₂R^{A6};
R¹⁵ is at each occurrence independently selected from:
halo, —CN, —NO₂, C₁₋₄ alkyl, C₁₋₄ haloalkyl, -L⁶-Q⁵, —OR^{A7}, —S(O)₂R^{A7}, —NR^{A7}R^{B7}, —C(O)R^{A7}, —OC(O)R^{A7}, —C(O)OR^{A7}, —NR^{B7}C(O)R^{A7}, —NR^{B7}C(O)OR^{A7}, —C(O)NR^{A7}R^{B7}, —NR^{B7}SO₂R^{A7} and —SO₂NR^{A7}R^{B7}—;
wherein said C₁₋₄alkyl is optionally substituted by 1 or 2 substituents selected from:
halo, —CN, —OR^{A8}, —NR^{A8}R^{B8} and —SO₂R^{A8};
Q⁴ and Q⁵ are at each occurrence independently selected from: phenyl, phenyl-C₁₋₃ alkyl, 5- or 6-membered heteroaryl, 5- or 6-membered heteroaryl-C₁₋₃ alkyl-, C₃₋₆ cycloalkyl, C₃₋₆ cycloalkyl-C₁₋₃ alkyl-, 4 to 6-membered heterocyclyl and 4 to 6-membered heterocyclyl-C₁₋₃ alkyl,
wherein said C₃₋₆ cycloalkyl, C₃₋₆ cycloalkyl-C₁₋₃ alkyl-, 4 to 6-membered heterocyclyl and 4 to 6-membered heterocyclyl-C₁₋₃ alkyl of Q⁴ and Q⁵ are each independently optionally substituted by 1 or 2 substituents selected from: C₁₋₄ alkyl, C₁₋₄ haloalkyl, halo, ═O, —CN, —OR^{A9}, —NR^{A9}R^{B9}, —SO₂R^{A9} and C₁₋₄ alkyl substituted by 1 or 2 substituents selected from: halo, —CN, —OR^{A10}, —NR^{A10}R^{B10} and —SO₂R^{A10}, and
wherein said of phenyl, phenyl-C₁₋₃ alkyl, 5- or 6-membered heteroaryl and 5- or 6-membered heteroaryl-C₁₋₃ alkyl- of Q⁴ and Q⁵ are each independently optionally substituted by 1 or 2 substituents selected from: halo, C₁₋₄alkyl, C₁₋₄ haloalkyl, —CN, —OR^{A9}, —NR^{A9}R^{B9}, —SO₂R^{A9} and C₁₋₄alkyl substituted by 1 or 2 substituents selected from: halo, —CN, —OR^{A10}, —NR^{A10}R^{B10} and —SO₂R^{A10};
L⁵ and L⁶ are independently absent or independently selected from: —O—, —NR^{A11}—, —S(O)₂—, —C(═O)—, —NR^{A11}C(═O)—, —C(═O)NR^{A11}—, —S(O)₂NR^{A11}—, —NR^{A11}S(O)₂—, —OC(═O)— and —C(═O)O—;
R^{A1}, R^{A2}, R^{B2}, R^{A3}, R^{B3}, R^{A4}, R^{A5}, R^{B5}, R^{A6}, R^{B6}, R^{A7}, R^{B7}, R^{A8}, R^{B8}, R^{A9}, R^{B9}, R^{A10}, R^{B10}, R^{A11} and R^{A12} are each independently selected from: H, C₁₋₄ alkyl and C₁₋₄ haloalkyl, or any —NR^{A2}R^{B2}, —NR¹⁶R^{B2}, —NR^{A3}R^{B3}, —NR¹⁷R^{B3}, —NR^{A5}R^{B5}, —NR^{A6}R^{B6}, —NR^{A7}R^{B7}, —NR^{A8}R^{B8}, —NR^{A9}R^{B9} and —NR^{A10}R^{B10} within a substituent may form a 4 to 6 membered heterocyclyl, wherein said 4 to 6 membered heterocyclyl is optionally substituted by one or more substituents selected from: halo, ═O, C₁₋₄ alkyl and C₁₋₄ haloalkyl; and
q is an integer selected from 0, 1, 2, 3 and 4.

2. The compound of claim 1, wherein L is absent.

3. The compound of claim 1 or claim 2, wherein L¹ is absent.

4. The compound of any one of claims 1 to 3, wherein R⁴ is selected from: H and C₁₋₄ alkyl, and R⁵ is H; optionally wherein R⁴ and R⁵ are both H.

5. The compound of any one of claims 1 to 4, wherein X₁, X₂ and X₃ are CH.

6. The compound of any one of claims 1 to 5, wherein HET is selected from: 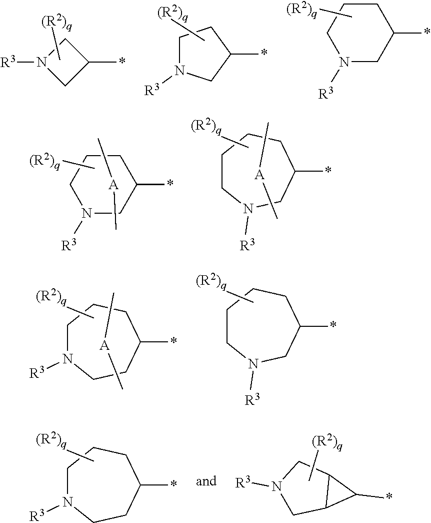
wherein A is C₁₋₄ alkylene, and * shows the point of attachment to the remainder of the compound.

7. The compound of any one of claims 1 to 5, wherein HET is: 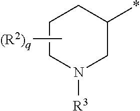
preferably 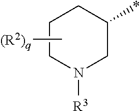
wherein * shows the point of attachment to the remainder of the compound.

8. The compound of any one of claims 1 to 7, wherein R² is at each occurrence independently selected from: ═O and C₁₋₄ alkyl; optionally wherein q is 0, 1 or 2.

9. The compound of any one of claims 1 to 7, wherein q is 0.

10. The compound of any one of claims 1 to 9, wherein R³ is H.

11. The compound of any one of claims 1 to 10, wherein
R¹ is selected from: C₁₋₆ alkyl, C₁₋₆ haloalkyl and Q¹-L²-, wherein said C₁₋₆ alkyl is optionally substituted by one or more R⁸;
Q¹ is selected from: C₃₋₁₂ cycloalkyl, 4 to 7 membered saturated or partially saturated heterocyclyl containing 1 or 2 ring heteroatoms selected from 0, S and N,
wherein said cycloalkyl and heterocyclyl is optionally substituted by one or more R⁹,
L² is absent or is selected from C₁₋₄ alkylene;
R⁸ and R⁹ are at each occurrence independently selected from: halo, ═O, —CN, C₁₋₆ alkyl, C₁₋₆ haloalkyl, -L³-Q², —OR^{16A}, —SO₂R¹⁶, —NR^{16A}R^{B2}, —C(O)R¹⁶, —C(O)NR^{16A}R^{B2}, —SO₂NR^{16A}R^{B2} and —C(O)OR^{16A},
wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},
wherein R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, —C(O)OR^{A5}, —NR^{B5}C(O)R^{A5}, —C(O)NR^{A5}R^{B5}, —NR^{B5}SO₂R^{A5} and —SO₂NR^{A5}R^{B5},
R^{16A} is selected from: H, C₁₋₆ alkyl, C₁₋₆ haloalkyl,
C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
C₂₋₆ alkyl substituted by 1 substituent selected from: —OR^{A5} and —NR^{A5}R^{B5};
Q² is at each occurrence independently selected from: C₃₋₆ cycloalkyl, C₃₋₆ cycloalkyl-C₁₋₃ alkyl, Q⁷, Q⁷-C₁₋₃ alkyl, phenyl, phenyl-C₁₋₃ alkyl, 5 or 6 membered heteroaryl and 5 or 6 membered heteroaryl-C₁₋₃ alkyl,
wherein Q⁷ is selected from azetidinyl, oxetanyl, pyrrolidinyl, tetrahydrofuranyl, piperidinyl, piperazinyl, tetrahydropyranyl and morpholinyl,
wherein said C₃₋₆ cycloalkyl, C₃₋₆ cycloalkyl-C₁₋₃ alkyl, Q⁷ and Q⁷-C₁₋₃ alkyl, is optionally substituted by one or more R¹⁴, and
wherein said phenyl, phenyl-C₁₋₃ alkyl, 5 or 6 membered heteroaryl and 5 or 6 membered heteroaryl-C₁₋₃ alkyl is optionally substituted by one or more R¹⁵;
L³ is absent or is selected from: —O—, —NR^{A4}—, —SO₂—, —C(═O)—, —NR^{A4}C(═O)—, —C(═O)NR^{A4}—, —S(O)₂NR^{A4}—, —NR^{A4}S(O)₂— and —C(═O)O—;
R¹⁴ and are at each occurrence independently selected from:
halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and
R¹⁵ is at each occurrence independently selected from:
halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7}.

12. The compound of any of claims 1 to 10, wherein R¹ is a 4 to 7 membered heterocyclyl, for example a saturated 4 to 7 membered heterocyclyl selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl, each of which is optionally substituted by one or more substituents (for example 1 or 2) selected from: halo, C₁₋₄ alkyl, C₁₋₄ haloalkyl, ═O, —C(O)R^{16A}, —C(O)OR^{16A}, —C(O)NR^{A2}R^{B2}, —SO₂R^{16A}, —SO₂Q²², —SO₂CH₂Q²², —C(O)Q²², —C(O)CH₂Q²², —C(O)NR^{B2}Q²², —C(O)NR^{B2}CH₂Q²², —SO₂NR^{A2}R^{B2}, —SO₂NR^{B2}Q²² and —SO₂NR^{B2}CH₂Q²²
R^{16A} is selected from: C₁₋₄ alkyl and C₁₋₄ alkyl substituted by —OR^{A5}, —S(O)₂R^{A5}, —NR^{A5}R^{B5}, —C(O)R^{A5}, —C(O)OR^{A5}, —C(O)NR^{A5}R^{B5},
Q²² is selected from: C₃₋₆ cycloalkyl, azetidinyl, pyrrolidinyl, piperidinyl, piperazinyl, morpholinyl, phenyl and 5 or 6-membered heteroaryl,
wherein Q²² is optionally substituted by one or more (e.g. 1 or 2) substituents selected from: halo, C₁₋₄ alkyl, C₁₋₄ haloalkyl, —OR^{A5}, —NR^{A5}R^{B5}, —C(O)R^{A5}, —C(O)NR^{A5}R^{B5} and —C(O)OR^{A5}.

13. The compound of any of claims 1 to 10, wherein R¹ is: 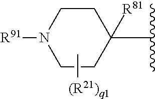
R⁹¹ is selected from: H, C₁₋₆ alkyl, C₁₋₆ haloalkyl, -L³-Q², —SO₂R¹⁶, —C(O)R¹⁶, —C(O)NR^{16A}R^{B2}, —SO₂NR^{16A}R^{B2} and —C(O)OR^{16A},
wherein said C₁₋₆ alkyl is optionally substituted by 1 or 2 substituents selected from: halo, —CN, —OR^{A5}, —S(O)₂R^{A5}, —NR^{A5}R^{B5}, —C(O)NR^{A5}R^{B5} and —C(O)OR^{A5},
R¹⁶ is selected from: H, C₁₋₆ alkyl and C₁₋₆ haloalkyl, wherein said C₁₋₆ alkyl is optionally substituted by one or more substituents selected from: halo, —CN, —OR^{A5}, —S(O)₂R^{A5}, —NR^{A5}R^{B5}, —C(O)R^{A5}, —OC(O)R^{A5}, C(O)OR A⁵, —NR^{B5}C(O)R^{A5}, C(O)NR^{A5}R^{B5}, —NR^{B5}SO₂R^{A5} and —SO₂NR^{A5}R^{B5},
R^{16A} is selected from:
H, C₁₋₆ alkyl, C₁₋₆ haloalkyl,
C₁₋₆ alkyl substituted by 1 or 2 substituents selected from: halo, —CN, —S(O)₂R^{A5}, —C(O)R^{A5}, —C(O)OR^{A5}, —C(O)NR^{A5}R^{B5} and —SO₂NR^{A5}R^{B5}, and
C₂₋₆ alkyl substituted by 1 substituent selected from: —OR^{A5} and —NR^{A5}R^{B5};
Q² is selected from:
Q⁶, Q⁶-C₁₋₃ alkylene-, Q⁷, Q⁷-C₁₋₃ alkylene-, Q³ and Q⁸-C₁₋₃ alkylene-,
wherein
Q⁶ is C₃₋₆ cycloalkyl;
Q⁷ is selected from: azetidinyl, oxetanyl, tetrahydrofuranyl, tetrahydropyranyl, pyrrolidinyl, piperidinyl, piperazinyl, morpholinyl, thiomorpholinyl, homopiperidinyl and homopiperazinyl;
Q³ is selected from: phenyl, pyrrolyl, furanyl, thienyl, imidazolyl, oxazolyl, oxadiazolyl, isoxazolyl, thiazolyl, isothiazolyl, pyrazolyl, pyridyl, pyrazinyl, pyridazinyl and pyrimidinyl;
wherein said Q⁶, Q⁶-C₁₋₃ alkylene-, Q⁷ and Q⁷-C₁₋₃ alkylene- are each optionally substituted by 1 to 4 R¹⁴, and Q⁸ and Q⁸-C₁₋₃ alkylene- are each optionally substituted by 1 to 4 R¹⁵;
L³ is absent or is selected from: —SO₂—, —C(═O)—, *—C(═O)NR^{A4}—, *—S(O)₂NR^{A4} and *—C(═O)O—, wherein * indicates the point of attachment to the ring nitrogen in R¹;
R¹⁴ at each occurrence is independently selected from: halo, ═O, —CN, C₁₋₄ alkyl, C₁₋₄ haloalkyl, —OR^{A5}, —S(O)₂R^{A5}, —NR^{A5}R^{B5}, —C(O)R^{A5}, —C(O)OR^{A5}, —C(O)NR^{A5}R^{B5} and —SO₂NR^{A5}R^{B5}; and
R¹⁵ at each occurrence is independently selected from: halo, —CN, C₁₋₄ alkyl, C₁₋₄ haloalkyl, —OR^{A7}, —S(O)₂R^{A7}, —NR^{A7}R^{B7}, —C(O)R^{A7}, —C(O)OR^{A7}, —C(O)NR^{A7}R^{B7} and —SO₂NR^{A7}R^{B7};
provided that when L³ is absent Q² is bonded to the ring nitrogen atom in R¹ via a ring carbon atom in Q²;
R²¹ at each occurrence is independently selected from: halo, ═O and C₁₋₄ alkyl;
R⁸¹ is selected from: H, C₁₋₄ alkyl, C₁₋₄ haloalkyl and C₃₋₆ cycloalkyl-C₁₋₃alkyl; and
q1 is an integer selected from 0, 1 and 2.

14. The compound of claim 13, wherein R⁸¹ is selected from: C₁₋₄ alkyl, C₁₋₄ haloalkyl and C₃₋₆ cycloalkyl-C₁₋₃alkyl.

15. The compound of claim 13, wherein R⁸¹ is methyl or ethyl.

16. The compound of any one of claims 13 to 15, wherein L³ is absent or —C(═O)—.

17. The compound of any one of claims 13 to 16, wherein q1 is 0.

18. The compound of any one of claims 13 to 17, wherein R⁹¹ is not H.

19. The compound of any one of claims 13 to 17 wherein R⁹¹ is H.

20. The compound of any one of claims 13 to 17, wherein R⁹¹ is selected from: —C(O)R¹⁶, —C(O)NR^{16A}R^{B2}; R¹⁶ is C₁₋₄alkyl; R^{16A} is selected from: H and C₁₋₄alkyl; and R^{B2} is selected from: H and C₁₋₄alkyl.

21. The compound of any one of claims 1 to 10, wherein R¹ is C₁₋₆ alkyl.

22. The compound of any one of claims 1 to 10, wherein R¹ is tert-butyl.

23. The compound of any one of claims 1 to 22, wherein the group of the formula:

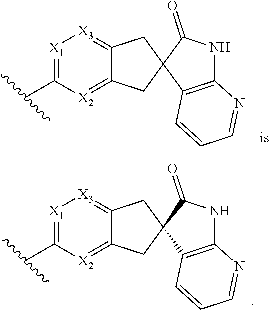

24. The compound of any one of claims 1 to 23, wherein X₁, X₂ and X₃ are CH.

25. The compound according to claim 1 selected from any one of the compounds shown in List 1 in the description, or a pharmaceutically acceptable salt thereof.

26. A pharmaceutical composition comprising a compound of any of claims 1 to 25, or a pharmaceutically acceptable salt thereof, and a pharmaceutically acceptable excipient.

27. A compound of any one of claims 1 to 25, or a pharmaceutically acceptable salt thereof, for use as a medicament.

28. A compound of any one of claims 1 to 25, or a pharmaceutically acceptable salt thereof, for use in the treatment of a disease or medical condition mediated by adrenomedullin receptor subtype 2 receptors (AM₂).

29. A compound of any one of claims 1 to 25, or a pharmaceutically acceptable salt thereof, for use in the treatment of a proliferative disease, particularly a cancer; optionally wherein the cancer is selected from: pancreatic cancer, colorectal cancer, breast cancer, lung cancer and a bone cancer.

30. A compound of any one of claims 1 to 25, or a pharmaceutically acceptable salt thereof, for use in the treatment of Sézary syndrome.

31. A method of treating a disease or medical condition mediated by AM₂ in a subject in need thereof, the method comprising administering to the subject an effective amount of a compound of any one of claims 1 to 25, or a pharmaceutically acceptable salt thereof.

32. The method of claim 31, wherein the disease is a proliferative disease, particularly a cancer; optionally wherein the cancer is selected from: pancreatic cancer, colorectal cancer, breast cancer, lung cancer and a bone cancer.

33. The compound for the use of claim 28 or claim 29, or the method of claim 31 or claim 32, wherein the compound is administered to a subject with elevated expression of AM, AM₂, CLR, and/or RAMP3 compared to controls, for example wherein the subject has elevated expression levels of AM or AM₂ in a serum sample.

34. A compound for the use or the method of any one of claims 28 to 33, wherein the compound is administered in combination with one or more additional anti-cancer agent and/or radiotherapy.

35. A compound selected from a compound of the formula (IX), (XI) or (XII): 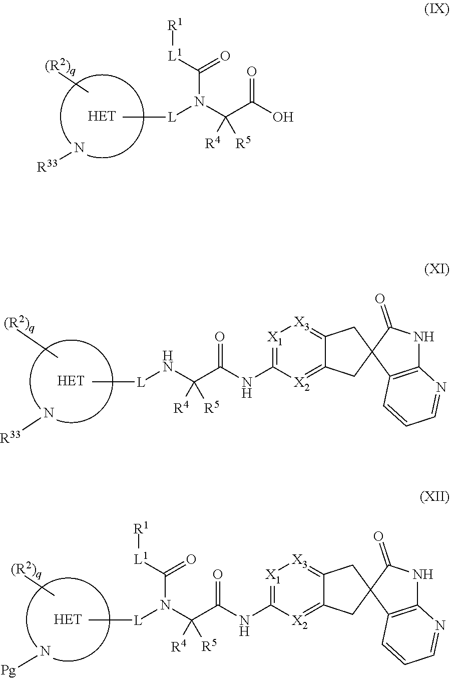
wherein HET, R¹, R², R⁴, R⁵, L, L¹, X₁, X₂, X₃ and q are as defined in claim 1;
R³³ is R³ as defined in claim 1, or R³³ is an amino protecting group (e.g. BOC); and
Pg is an amino protecting group (e.g. BOC).
